(19) United States
(12) Patent Application Publication
(10) Pub. No.: US 2023/0324404 A1
(43) Pub. Date: Oct. 12, 2023

(54) METHOD FOR DETECTION OF ZIKA VIRUS SPECIFIC ANTIBODIES

(71) Applicant: Takeda Vaccines, Inc., Cambridge, MA (US)

(72) Inventors: Erick PEREZ-GUZMAN, Cambridge, MA (US); Eduardo NASCIMENTO, Cambridge, MA (US); Hansi DEAN, Cambridge, MA (US); Ralph BRAUN, Cambridge, MA (US)

(73) Assignee: Takeda Vaccines, Inc., Cambridge, MA (US)

(21) Appl. No.: 17/999,192

(22) PCT Filed: May 19, 2021
(86) PCT No.: PCT/US2021/033264

§ 371 Date: Nov. 17, 2022

Related U.S. Application Data
(60) Provisional application No. 63/027,508, filed on May 20, 2020.

(30) Foreign Application Priority Data
Mar. 19, 2021 (WO) PCT/US2021/023275

(51) Int. Cl. G01N 33/68; G01N 33/533; C07K 16/10

(52) U.S. Cl. CPC G01N 33/6854; G01N 33/533; C07K 16/10; C07K 2317/565

(57) ABSTRACT

The present invention is directed to a method, i.e. an immunoassay, for determining the presence and/or the amount of anti-zika Anti-ZIKV #1 virus antibodies, i.e. zika virus-specific antibodies in a sample. Therefore, the present invention is directed to a microsphere complex comprising a microsphere coupled to a zika virus like particle, as well as to a kit comprising said microsphere complex and an amount of reporter antibody that binds to the zika virus like particle. The present invention further relates to a method for determining an antibody correlate of protection against zika virus infection for a zika virus vaccine. Moreover, the present invention is directed to a method for diagnosing the protection of a human or non-human subject against a zika virus infection.

### CROSS REFERENCE TO RELATED APPLICATIONS

This International PCT Application claims priority to and the benefit of U.S. Provisional Application No. 63/027,508 filed on 20 May 2020, as well as priority to and the benefit of International PCT Application No. PCT/US2021/023275 filed on 19 Mar. 2021, the contents of which is herein incorporated by reference in its entirety.

### STATEMENT REGARDING FEDERALLY SPONSORED RESEARCH

This invention was made with Government support under Contract No. HHSO100201600015C awarded by the Department of Health and Human Services, Office of the Assistant Secretary for Preparedness and Response, Biomedical Advanced Research and Development Authority. The Government has certain rights in the invention.

### SEQUENCE LISTING

This application incorporates by reference in its entirety the Sequence Listing entitled “T08499WO2_PCTSequenceListing.txt” created on May 19, 2021 at 1:23 pm that is 416 KB and filed electronically herewith.

### FIELD OF THE INVENTION

The present invention relates to a method for determining Zika virus (ZIKV) specific antibodies (Abs)—developed by natural infection or vaccination—wherein the method does not cross-react to dengue virus (DENV) and/or other flavivirus antibody (Ab) responses.

### BACKGROUND OF THE INVENTION

A Zika virus (ZIKV) is an arthropod-borne virus (arbovirus) in the genus Flavivirus (family Flaviviridae) which also includes the West Nile virus (WNV), dengue virus (DENV), tick-borne encephalitis virus (TBEV), and yellow fever virus (YFV). It is thought to be principally transmitted to humans by the Aedes genus, i.e. by the mosquito Aedes aegypti. The potential effect of ZIKV as a public health threat increased due to isolated outbreaks in Southeast Asia during 2007 and 2013 (Duffy et al., N Engl J Med. 2009, 360, 2536-2543; Hancock et al., Emerg. Infect. Dis. 2014, 20(11):1960). The largest ZIKV outbreak occurred in recent years when the spread reached to Brazil and throughout The Americas (Metsky et al., Nature 2017, 546(7658):411-415). ZIKV is classified into African and Asian genotypes by phylogenetic analysis.

ZIKV is associated with neurological sequelae and a broad spectrum of clinical manifestations and neonatal abnormalities known as the Congenital ZIKV Syndrome (CZS; Costello et al., Bull World Health Organ. 2016, 94(69):406-406A; Cao-Lormeau et al., Lancet 2016, Apr. 9; 387(10027): 1531-9). No treatment is approved yet for ZIKV, although multiple vaccine candidates are currently evaluated in clinical trials (Poland et al., Mayo Clinic Proceedings 2019, 94, 2572-2586).

Flaviviruses are enveloped, with icosahedral and spherical geometries. The diameter is around 50 nm. Genomes (10-11 kb bases) consists of linear positive-sense RNA and are non-segmented. The RNA is complexed with multiple copies of the capsid protein (C), surrounded by an icosahedral shell consisting of 180 copies each of the envelope glycoprotein (E protein; ˜500 amino acids), and the membrane protein (M protein; ˜75 amino acids) or precursor membrane protein (prM protein; ˜165 amino acids), all anchored in a lipid membrane. The genome also codes for seven non-structural proteins (NS1, NS2A, NS2B, NS3, NS4A, NS4B and NS5; WO2018010789).

As E protein is the main surface protein that participates in host cell receptor attachment and virus lipid bilayer fusion, it is the major target of host neutralizing antibodies (Abs) against viral infection. The E protein is composed of an amino terminal ectodomain, two amphipathic α-helices and two carboxy terminal membrane-spanning α-helices. The surface-exposed ectodomain consists of three structurally distinct domains rich in β-sheets: a β-barrel domain I (EDI), a finger-like domain II (EDII), and a C-terminal domain III (EDIII).

As several epitopes are conserved among flaviviruses, antibody (Ab) responses to flavivirus infections are cross-reactive, hampering the diagnosis of a specific flavivirus infection for example by the determination of a flavivirus specific immune response. Cross-reactivity between ZIKV and other flavivirus Abs has been reported, particularly with DENV serocomplex due to high homology (54-59%) of the E protein. This comes even more into play as in a majority of ZIKV endemic regions, there are also DENV, including DENV serotypes 1 (DENV1), 2 (DENV2), 3 (DENV3), and 4 (DENV4), and other flaviviruses present, increasing the risk of multiple infections and therefore production of cross-reactive Abs. Further, the development of similar clinical manifestations by different flavivirus infections is of particular concern.

Diagnostic methods for detection of a flavivirus infection rely on the determination of the presence of viral components or host immune response. Detection of the viral genome in blood by quantitative reverse transcription-polymerase chain reaction (qRT-PCR) is an efficient technique to discriminate flaviviruses based on genomic divergence (Cantero et al., The American Journal of Tropical Medicine and Hygiene 2020, 102: 625-628). However, short viremia period in these acute infections limits the use of qRT-PCR methods for diagnosis and sample panel characterization, even more when approximately 80% of the ZIKV infection are asymptomatic and the viremic window can be easily missed. Long-lasting immunity produced during the convalescent phase of infection enables extending the infection identification window by serological methods as alternative diagnostic tools (Gouel-Cheron et al., Antiviral Research 2019, 172:104638). However, cross-reactivity between ZIKV and other flaviviruses, particularly with DENV serocomplex, have compromised accurate diagnosis based on serology (Heinz and Stiasny, Microbiology and molecular biology reviews (MMBR) 2017, 81; Wen and Shresta, Current Opinion in Immunology 2019, 59:1-8).

Efficacy (protection against infection) of a specific vaccine usually needs to be tested against a control or placebo in a phase III clinical study in a sufficient number of subjects in endemic areas, i.e. areas with high virus transmission. Although zika transmission persists, it is currently at very low levels prohibiting a direct efficacy measurement. If available, efficacy may be measured in the form of a surrogate parameter, which correlates with protection. Although neutralizing Abs are proposed as a correlate of protection against ZIKV infection, neutralization assays such as the reporter virus particle (RVP) test as well as the plaque reduction neutralization test (PRNT) usually cannot distinguish between ZIKV and other flavivirus infections solely based on neutralizing antibody titers (Calvert et al., Journal of Clinical Microbiology 2018, 56; Lindsey et al., Journal of Clinical Microbiology 2018, 56; Shan et al., J. Clin. Microbio. 2017, 55, 3028-3036).

Multiple assays based on the enzyme linked immunosorbent assay (ELISA) format have been developed to measure total IgM/IgG binding, but in most cases sensitivity and specificity are compromised depending on the sample panel used (Tyson et al., J. Clin. Microbio. 2019, Zaidi et al., Acta tropica 2020, 201, 105201; Rodriguez-Barraquer et al., Science 2019, 363, 607-610). Recently, a competitive ELISA has been developed using ZIKV EDIII as plate-immobilized antigen to determine anti-ZIKV Abs in sera from mice immunized with EDIII (WO 2020/087038). Interestingly, these results showed that the data from the competition set-up correlated well with protection of the mice against ZIKV infection. In addition, competitive ELISAs using ZIKV NS1 as plate-immobilized antigen have been used for the detection of anti-ZIKV Abs in presence of previous flavivirus infections (Balmaseda et al., PNAS 2017, 114(31):8384-8389; Nascimento et al., Am. J. Trop. Med. Hyg. 2019, 101(3):708-715). However, it is known that specificity and sensitivity of ELISA is reduced in ZIKV-immune samples with complex immunological background resulting for instance from multiple prior exposures to DENV.

Microsphere immunoassays (MIAs) using antigen-coupled microspheres provide numerous advantages over conventional assays such as ELISA. The MIA approach increases sensitivity and specificity, among other advantages such as providing flexibility to single- or multiplex antigens from different viruses in one single experiment, the possibility for high-throughput, cost-effectiveness, and short turnaround times. However, the feasibility of different MIA set-ups largely depends on the immobilized antigen applied. In the past, MIAs have been used to determine total anti-ZIKV Ab levels (Taylor et al., Viruses 2018, 10, 253, Young et al., Scientific Reports 2020, 10:3488). However, both total Ab binding ELISA and MIA still require well-characterized sample panel and development of complex algorithms that integrate multiple serologic tests in order to improve results interpretation due to cross-reactivity of flavivirus induced Abs (Tyson et al., J. Clin. Microbio. 2019, 57; Taylor et al., Viruses 2018, 10, 253; Mendoza et al., Diagnostic Microbiology and Infectious Disease 2019, 94, 140-146). Moreover, the benefits of a microsphere assay set-up have not been utilized for detection of ZIKV specific Abs, for instance, in order to differentiate ZIKV and flavivirus infections, as MIAs measuring total anti-ZIKV Ab levels cannot distinguish between ZIKV specific and cross-reactive Abs.

### OBJECTS AND SUMMARY

It is an object of the present invention to provide an immobilizable binding partner for anti-ZIKV Abs.

It is a further object of the present invention to provide a selective couple including an immobilizable binding partner for anti-ZIKV Abs and a corresponding selective ligand capable of selecting anti-ZIKV Abs, in particular neutralizing anti-ZIKV Abs, which are not cross-reactive with DENV.

It is a further object of the present invention to provide a method for measuring the presence and/or amount of anti-ZIKV Abs, the method providing good specificity.

It is a further object of the present invention to provide a method of measuring the presence and/or amount of anti-ZIKV Abs, the method being capable of selectively distinguishing samples comprising anti-ZIKV Abs from samples comprising other anti-flaviviruses Abs, such as anti-DENV Abs, anti-WNV Abs, anti-JEV Abs, anti-SLEV Abs, and/or anti-TBEV Abs.

It is a further object of the present invention to provide a method of measuring the presence and/or amount of anti-ZIKV Abs, which are not cross-reactive with DENV.

It is a further object of the present invention to provide a method of measuring the presence and/or amount of neutralizing anti-ZIKV Abs, which are not cross-reactive with DENV.

It is a further object of the present invention to provide a method of measuring the presence and/or amount of anti-ZIKV Abs, the method providing good sensitivity.

It is a further object of the present invention to provide a method of measuring the presence and/or amount of anti-ZIKV Abs, the method providing flexibility to single- or multiplex antigens from different viruses in one single experiment.

It is a further object of the present invention to provide a method of measuring the presence and/or amount of anti-ZIKV Abs, the method providing the possibility for high-throughput, cost-effectiveness, and short turnaround times.

It is a further object of the present invention to provide a method for establishing a correlate of protection.

It is a further object of the present invention to measure protection against zika virus infection.

It is a further object of the present invention to measure protection against zika virus infection effected by a zika vaccine.

It is a further object of the present invention to provide a method of diagnosing a zika infection.

It is a further object of the present invention to provide a method of diagnosing a zika infection after the period of viremia.

It is a further object of the present invention to provide a method for preventing zika disease in a human subject.

The present invention is therefore directed to a microsphere complex comprising a microsphere coupled to a zika virus like particle.

The present invention is further directed to a kit comprising an amount of such a microsphere complex and an amount of a reporter antibody that binds to the zika virus like particle of the microsphere complex.

The present invention is further directed to a method for detecting a signal from a reporter antibody indicative for the presence and/or amount of anti-zika virus antibodies in a sample from a subject comprising the steps of:
Step 1: providing a kit comprising an amount of a microsphere complex comprising a microsphere coupled to a zika virus like particle and an amount of a reporter antibody that binds to the zika virus like particle of the microsphere complex,
Step 2: contacting the amount of said microsphere complex and the amount of said reporter antibody of step 1 with the sample to allow binding of anti-zika virus antibodies in the sample to the zika virus like particles coupled to the microspheres in the microsphere complex while competing with the reporter antibody, and
Step 3: detecting a signal from the reporter antibody bound to the zika virus like particles coupled to the microspheres in the microsphere complex in step 2.

The present invention is further directed to such a method for detecting the presence and/or amount of anti-zika virus antibodies in a sample from a subject comprising the further steps of
Step 4: determining the presence and/or the amount of the reporter antibody bound to the zika virus like particles coupled to the microspheres in the microsphere complex from the signal of step 3, and
Step 5: determining the presence and/or the amount of anti-zika virus antibodies in the sample based on the presence and/or amount of the reporter antibody determined in step 4.

The present invention is therefore directed to a method for determining an antibody correlate of protection against zika virus infection for a zika virus vaccine in a type of non-human subjects comprising the steps of:
Step 1: selecting a group of said subjects which are zika virus naive,
Step 2: dividing the group of subjects into at least two subgroups, wherein one subgroup functions as control group and at least one subgroup functions as inoculation group,
Step 3: inoculating said at least one inoculation group with a dose of the zika virus vaccine,
Step 4: challenging all subjects with an infectious amount of the zika virus,
Step 5: determining the amount of anti-zika virus antibodies for each subject as described above at least after inoculation with the zika virus vaccine and before challenging with the infectious amount of the zika virus,
Step 6: determining presence or absence of viremia in all subjects after challenging with the infectious amount of the zika virus,
Step 7: repeating steps 3 to 6 with further inoculation groups with increasing vaccine doses until absence of viremia is determined in all subjects of one inoculation group in step 6, and
Step 8: determining the amount of anti-zika virus antibodies after inoculation with the zika virus vaccine and before challenging with the infectious amount of the zika virus associated with absence of viremia after challenging with the infectious amount of zika virus as antibody correlate of protection.

The present invention is further directed to a method for determining an antibody correlate of protection against zika infection in human subjects by mathematically modeling the correlate of protection of the non-human subjects as determined by the method as described above to fit human subjects.

The present invention is further directed to a method for diagnosing the protection of a human subject against a zika virus infection comprising the steps of:
Step 1: providing a sample from a human subject outside the human body,
Step 2: determining the amount of anti-zika virus antibodies in the sample from the human subject as described above, and
Step 3: determining protection by comparing the amount of anti-zika virus antibodies to the antibody correlate of protection against zika virus infection in human subjects, optionally determined as described above.

The present invention is further directed to a method for diagnosing the protection of a non-human subject against a zika virus infection comprising the steps of:
Step 1: providing a sample from a non-human subject outside the non-human body,
Step 2: determining the amount of anti-zika virus antibodies in the sample from the non-human subject as described above, and
Step 3: determining protection by comparing the amount of anti-zika virus antibodies determined in step 2 to the antibody correlate of protection determined in this type of non-human subjects.

The present invention is further directed to a method for diagnosing a zika virus infection in a subject comprising the steps of:
Step 1: providing a sample from a subject outside the subject body,
Step 2: determining the amount of anti-zika virus antibodies in the sample as described above,
Step 3: determining infection by comparing said amount of anti-zika virus antibodies to established amounts of anti-zika virus antibodies in zika virus infected subjects.

The present invention is alternatively directed to a kit comprising an amount of a microsphere complex coupled to a zika antigen and an amount of a reporter antibody that binds to the zika antigen of the microsphere complex and corresponding methods.

The present invention is further directed to a method for detecting a signal from a detection antibody indicative for the presence and/or amount of anti-zika virus antibodies in a sample from a subject comprising the steps of:
Step 1: contacting an amount of a microsphere complex comprising a microsphere coupled to a zika virus like particle with the sample to allow binding of anti-zika virus antibodies in the sample to the zika virus like particles coupled to the microspheres in the microsphere complex,
Step 2: contacting an amount of a detection antibody with the microsphere complex and the sample of step 1 to allow binding of the detection antibody to the heavy chain constant region of the anti-zika virus antibodies bound to the zika virus like particles coupled to the microspheres in the microsphere complex, wherein the detection antibody binds to the anti-zika virus antibodies with the variable region of the detection antibody and wherein the detection antibody is attached to at least one detectable label, and
Step 3: detecting a signal from the detection antibody bound to the anti-zika virus antibodies in step 2.

The present invention is further directed to such a method for determining the presence and/or amount of anti-zika virus antibodies in a sample from a subject, wherein the method comprises the further steps of:
Step 4: determining the presence and/or amount of the detection antibody bound to the anti-zika virus antibodies from the signal of step 3, and
Step 5: determining the presence and/or amount of anti-zika virus antibodies in the sample from the presence and/or amount of the detection antibody determined in step 4.

The present invention is further directed to a method for preventing zika disease in a human subject comprising the steps of:
Step 1: obtaining a sample from the human subject,
Step 2: determining the amount of anti-zika virus antibodies in the sample from the human subject according to the methods described above,
Step 3: determining whether the human subject has an amount of anti-zika virus antibodies to confer protection by comparing the amount of anti-zika virus antibodies determined in step 2 to the antibody correlate of protection against zika virus infection in human subjects, which optionally has been determined as described above, and
Step 4: administering to the human subject a zika virus vaccine if the human subject has an amount of anti-zika antibodies that is lower than the antibody correlate of protection against zika virus infection in human subjects, which optionally has been determined as described above.

### Abbreviations and Definitions

### Abbreviations

“PE” stands for phycoerythrin. “ZIKV” refers to zika or zika virus. “DENV” refers to dengue or dengue virus. “DENV1” refers to dengue virus serotype 1. “DENV2” refers to dengue virus serotype 2. “DENV3” refers to dengue virus serotype 3. “DENV4” refers to dengue virus serotype 4. “WNV” refers to West Nile virus. “YFV” refers to Yellow Fever Virus. “SLEV” refers to St. Louis Encephalitis virus. “TBEV” refers to Tick-borne encephalitis virus. “PIZV” refers to purified inactivated ZIKV vaccine. “FV” refers to flavivirus. “VLP” refers to virus like particle. “E protein” refers to envelope glycoprotein. “EDI”, “EDII”, “EDIII” refer to domain I, II, and III of the E protein. “M protein” refers to membrane protein. “prM” refers to precursor membrane protein. “MFI” refers to median fluorescent intensity. “Ab” stands for antibody. “Abs” stands for antibodies. “Ig” stands for immunoglobulin. “mAb” stands for monoclonal antibody. “Anti-ZIKV Ab” refers to an Ab that binds to a ZIKV antigen. “Anti-DENV Ab” refers to an Ab that binds to a DENV antigen. “Anti-JEV Ab” refers to an Ab that binds to a JEV antigen. “Anti-WNV Ab” refers to an Ab that binds to a WNV antigen. “Anti-TBEV Ab” refers to an Ab that binds to a TEBV antigen. “Anti-YFV Ab” refers to an Ab that binds to a YFV antigen. “Anti-SLEV Ab” refers to an Ab that binds to a SLEV antigen. “MIA” stands for microsphere immunoassay. “cMIA” stands for competitive microsphere immunoassay. “CDR” stands for complementary determining region. “RVP” refers to reporter virus particle. “PRNT” refers to plaque reduction neutralization test, “MNT” refers to microneutralization test, “FFA” refers to focus forming assay, “ELISA” refers to enzyme linked immunosorbent assay.

### Definitions

As used in the present disclosure and claims, the singular forms “a,” “an,” and “the” are to be construed to cover both the singular and the plural forms unless the context clearly dictates otherwise.

The term “and/or” as used in a phrase such as “A and/or B” herein is intended to include “A and B”, “A”, and “B”.

Open terms such as “include”, “including”, “contain”, “containing” and the like mean “comprising.” These open-ended transitional phrases are used to introduce an open ended list of elements, method steps, or the like that does not exclude additional, unrecited elements or method steps.

As used herein, the terms “antibody (Ab)” or “antibodies (Abs)” refer to an immunoglobulin (Ig) molecule, generally comprised of four polypeptide chains, two heavy (H) chains and two light (L) chains interconnected by disulfide bonds (full length Ab) and includes any naturally occurring, enzymatically obtainable, synthetic, or genetically engineered polypeptide or glycoprotein that specifically binds an antigen to form a complex. Abs can be obtained using standard recombinant DNA techniques. In a full length Ab, each heavy chain is comprised of a heavy chain variable region (VH) and a heavy chain constant region (CH). The heavy chain constant region is comprised of three domains, CH1, CH2 and CH3. Each light chain is comprised of a light chain variable region (VL) and a light chain constant region (CL). The light chain constant region is comprised of one domain. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, and FR4. In certain embodiments of the present invention, the FRs of the Ab may be identical to the human germline sequences, or may be naturally or artificially modified. The terms Ab or Abs may also refer to any functional fragment, mutant, variant, or derivative thereof. Such functional fragment, mutant, variant, or derivative antibody formats are known in the art. Ab fragments such as Fab or F(ab′)2 fragments, can be prepared from full length Abs using conventional techniques such as papain or pepsin digestion, respectively, of full length Abs. Functional fragments are in particular (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546, Winter et al., PCT publication WO 90/05144 A1), which comprises a single variable domain; and (vi) an isolated CDR. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). In certain embodiments, scFv molecules may be incorporated into a fusion protein. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger, P., et al. (1993) Proc. Natl. Acad. Sci. USA 90:6444-6448; Poljak, R. J., et al. (1994) Structure 2:1121-1123). Such functional fragments are known in the art (Kontermann and Dubel eds., Antibody Engineering (2001) Springer-Verlag. New York. 790 pp. (ISBN 3-540-41354-5)). The Ab may be described by the term “anti-antigen Ab” to express to which antigen the Ab is able to bind. For instance, an “anti-ZIKV Ab” refers to an Ab that binds to a ZIKV antigen. Ab or Abs may be mono-specific, bi-specific, or multi-specific. Multi-specific Abs may specifically bind different epitopes of one antigen or may specifically bind two or more unrelated antigens. See, e.g., Tutt et al., 1991, J. Immunol. 147:60-69; Kufer et al., 2004, Trends Biotechnol. 22:238-244. Abs including any of the multi-specific antigen-binding molecules of the present invention, or variants thereof, may be constructed using standard molecular biological techniques (e.g., recombinant DNA and protein expression technology), as will be known to a person of ordinary skill in the art, for instance intracellular expression systems. Abs may be multivalent Abs comprising two or more antigen binding sites. Substitution of one or more CDR residues or omission of one or more CDRs is also possible. Abs have been described in the scientific literature where one or two CDRs can be dispensed with barely an effect for binding. Analysis of the contact regions between Abs and their antigens, based on published crystal structures, revealed that only about one fifth to one third of CDR residues actually contact the antigen. Moreover, many Abs have one or two CDRs were no amino acids are in contact with an antigen (Padlan et al. FASEB J. 1995, 9: 133-139, Vajdos et al., J Mol Biol 2002, 320:415-428). CDR residues not contacting antigen can be identified based on previous studies (for example residues H60-H65 in CDR2 of the heavy chain are often not required), from regions of Kabat CDRs lying outside Chothia CDRs, by molecular modeling and/or empirically. If a CDR or residue(s) thereof is omitted, it is usually substituted with an amino acid occupying the corresponding position in another human Ab sequence or a consensus of such sequences. Positions for substitution within CDRs and amino acids to substitute can also be selected empirically. Empirical substitutions can be conservative or non-conservative substitutions. The terms Ab or Abs may refer to Ab or Abs that originate from certain origin species that for example include rabbit, mouse, human, monkey, or rat (rabbit Ab, mouse Ab, human Ab, monkey Ab, or rat Ab). For instance, rabbit origin may be intended to include Abs having variable and constant regions derived from rabbit germline immunoglobulin sequences. Abs may comprise one or more amino acid substitution, insertion, and/or deletion as compared to corresponding germline sequences. The Abs may also include amino acid residues not encoded by the origin species germline immunoglobulin sequences (e.g. mutations introduced by random or site-specific mutagenesis in vitro or in vivo), for example in the CDRs. As used herein, an Ab or Abs originating from a certain origin species (e.g. rabbit) may also refer to an Ab or Abs in which CDR or other sequences derived from the germline of another mammalian species (e.g. mouse) have been grafted onto the origin species (e.g. rabbit) framework region (FR) sequences. Abs may be chimeric Abs. Chimeric Abs may encompass sequences derived from the germline of different species and may also include further amino acid substitutions or insertions. Abs may be humanized Abs that are human immunoglobulins that contain minimal non-human (e.g., murine) sequences. Typically, in humanized antibodies residues from the human CDR are replaced by residues from the CDR of a non-human species (e.g., mouse, rat, rabbit, and hamster, etc.; Jones et al., Nature 1986; 321:522-525; Riechmann et al., Nature 1988, 332:323-327; Verhoeyen et al., Science 1988, 239:1534-153). Non-limiting examples of methods used to generate humanized antibodies are described in U.S. Pat. No. 5,225,539; Roguska et al., Proc. Natl. Acad. Sci. 1994, USA 91:969-973; and Roguska et al., Protein Eng. 1996; 9:895-904. Abs can be of any class (e.g., IgG, IgE, IgM, IgD, IgA and IgY) and subclass (isotype) (e.g., IgG1, IgG2, IgG3, IgG4, IgA1 and IgA2). In some embodiments, the immunoglobulin is an IgG1 isotype. In some embodiments, the immunoglobulin is an IgG2 isotype. The different classes of immunoglobulins have different and well-known subunit structures and three-dimensional configurations. Abs may comprise sequences from more than one class or subclass. Abs may be free of other Abs having different antigenic specificities (e.g. an Ab that binds ZIKV is substantially free of Abs that bind antigens other than ZIKV). The Ab may be free of other cellular material and/or chemicals. The terms Ab or Abs may refer to a neutralizing or non-neutralizing Ab. The terms Ab or Abs may refer to a monoclonal Ab. The terms Ab or Abs may refer to a recombinant Ab. The term Ab or Abs may refer to a reporter Ab. The term Ab or Abs may refer to a control Ab.

As used herein, the term “constant region” of an Ab refers to the heavy chain constant region (CH) and/or the light chain constant region (CL).

As used herein, the term “variable region” of an Ab refers to the heavy chain variable region (VH) and/or the light chain variable region (VL).

As used herein, the term “binds to”, “is binding to”, or “capable of binding to” refers within the context of an Ab that binds to or is binding to or is capable of binding to, to an Ab that is able to bind a certain antigen. Ability of binding to a certain antigen can be investigated by methods well known in the art including ELISA, or bio-layer interferometry (BLI). Thereby, the Ab provides a signal above the background or noise of the method when tested for binding to the antigen. In preferred embodiments, the Ab provides a signal when tested for binding to the antigen, which is at least 10%, at least 25%, at least 35%, at least 50%, at least 60%, at least 75%, at least 85%, at least 90%, at least 95%, or 100% higher than the signal the Ab provides when tested for binding to comparable antigens. In a specific embodiment, the antigen is a ZIKV antigen (i.e. a ZIKV VLP) and the comparable antigens are DENV antigens. In a preferred embodiment, the Ab is able to bind to the antigen with the Ab variable region.

As used herein, the term “allow binding” refers within the context of an Ab to a situation, wherein an Ab is incubated with a certain molecule e.g. an antigen like a ZIKV VLP coupled to a microsphere for a certain time to enable the Ab to bind to the molecule. If an Ab does not bind to a certain molecule, no binding will occur.

As used herein, the term “is bound to” refers within the context of an Ab that is bound to, to an Ab that is bound to a molecule e.g. an antigen. The Ab can be bound to said molecule with the antibody constant or variable region. In the case that the molecule is an antigen the Ab is bound to the antigen with the antibody variable region.

As used herein, the term “complementary determining region (CDR)” refers to the CDR within the Ab variable sequences. There are three CDRs in each of the variable regions of the heavy chain (VH) and the light chain (VL), which are designated CDR1, CDR2 and CDR3 (or specifically VH-CDR1, VH-CDR2, VH-CDR3, VL-CDR1, VL-CDR2, and VL-CDR3), for each of the variable regions. The term CDR may refer to a group of three CDRs that occur in a single variable region capable of binding the antigen. The exact boundaries of these CDRs can be defined differently according to different systems. The system described by Kabat (Kabat et al., Sequences of Proteins of Immunological Interest (National Institutes of Health, Bethesda, Md. (1987) and (1991)) refers to an unambiguous residue system applicable to any variable region of an antibody, but also provides precise residue boundaries defining the three CDRs. These CDRs may be referred to as Kabat CDRs. For the VH region, the hypervariable region ranges from amino acid positions 31 to 35 for VH-CDR1, amino acid positions 50 to 65 for VH-CDR2, and amino acid positions 95 to 102 for VH-CDR3. For the VL region, the hypervariable region ranges from amino acid positions 24 to 34 for VL-CDR1, amino acid positions 50 to 56 for VL-CDR2, and amino acid positions 89 to 97 for VL-CDR3. Chothia and coworkers (Chothia &Lesk, J. Mol. Biol. 196:901-917 (1987) and Chothia et al., Nature 342:877-883 (1989)) found that certain sub-portions within Kabat CDRs adopt nearly identical peptide backbone conformations, despite having great diversity at the level of amino acid sequence. These sub-portions were designated as L1, L2 and L3 or H1, H2 and H3 where the “L” and the “H” designates the light chain and the heavy chains regions, respectively. These regions may be referred to as Chothia CDRs, which have boundaries that overlap with Kabat CDRs. Other boundaries defining CDRs overlapping with the Kabat CDRs have been described by Padlan (FASEB J. 9:133-139 (1995)) and MacCallum (J Mol Biol 262(5):732-45 (1996)). Still other CDR boundary definitions may not strictly follow one of the above systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, although preferred embodiments use Kabat or Chothia defined CDRs.

As used herein, the term “framework”, “framework region (FR)” or “framework sequence” refers to the remaining sequences of a variable region minus the CDRs. Because the exact definition of a CDR sequence can be determined by different systems, the meaning of a framework sequence is subject to correspondingly different interpretations. The six CDRs (VL-CDR1, VL-CDR2, and VL-CDR3 and VH-CDR1, VH-CDR2, and VH-CDR3) also divide the framework regions on the light chain (L) and the heavy chain (H) into four sub-regions (FR1, FR2, FR3 and FR4) on each chain, in which CDR1 is positioned between FR1 and FR2, CDR2 between FR2 and FR3, and CDR3 between FR3 and FR4. Without specifying the particular sub-regions as FR1, FR2, FR3, or FR4, a framework region, as referred by others, represents the combined FR's within the variable region of a single, naturally occurring immunoglobulin chain. As used herein, a FR represents one of the four sub-regions, and FRs represents two or more of the four sub-regions constituting a framework region.

A “recombinant Ab”, as used herein, refers to an Ab, which is created, expressed, isolated or obtained by technologies or methods known in the art as recombinant DNA technology which include, e.g. DNA splicing and transgenic expression. The term may refer to Abs expressed in a non-human mammal (including transgenic non-human mammals e.g. transgenic mice), or a cell (e.g. CHO cells) expression system or isolated from a recombinant combinatorial human antibody library.

A “neutralizing Ab”, as used herein, is intended to refer to an Ab which provides a titer above the lower limit of detection and/or the background in a microneutralization test (MNT), plaque reduction neutralization test (PRNT), focus forming assay (FFA) and/or reporter virus particle (RVP) test. A neutralizing Ab may be used alone or in combination as prophylactic or therapeutic agent with other anti-viral agents upon appropriate formulation, or in association with active vaccination, or as a diagnostic tool. The term neutralizing Ab may refer to an Ab which prevents, inhibits, reduces, impedes, or interferes with the ability of a pathogen e.g. a ZIKV to initiate and/or perpetuate an infection in a host. The epitope to which a neutralizing Ab binds to may be referred to as a “neutralizing epitope”.

As used herein, the term “antibody titer” refers to a certain amount of Ab within a sample. The sample may be a blood plasma, urine, blood, or serum sample. An antibody titer can be expressed as the inverse of the highest dilution (in a serial dilution row) that still gives a positive test result. Consequently the term “neutralizing antibody titer” refers to a certain amount of neutralizing Abs within a sample. An Ab titer or neutralizing Ab titer can be determined by various method well known in the art including enzyme linked immunosorbent assay (ELISA), microsphere immunoassay, RVP assay, MNT, FFA, or PRNT.

As used herein, the term “reporter virus particle (RVP)” refers to particles that retain the antigenic determinants of wild-type virions and include capsid (C), envelope (E), pre-membrane (prM) and membrane (M) proteins. Upon infection of cells with RVPs a reporter gene e.g. Renilla luciferase or firefly luciferase is expressed. RVPs enable tracking of a virus infection over time and quantifying events such as virus cellular entry and replication.

As used herein, the term “reporter virus particle assay (RVP assay)” or “reporter virus particle test (RVP test)” refers to an assay for determining neutralizing Ab titers in a sample. Thereby, cells as for instance Vero cells, are incubated with the sample, followed by the addition of RVPs. The half-maximal effective concentration (EC₅₀ RVP) titer of neutralizing Abs is determined by addition of a suitable substrate that is converted by the reporter gene expressed upon RVP infection to a detectable signal. For instance, upon conversion of the substrate coelenetrazine, luciferase produces a luminescence signal that can be detected. Reduction of the luminescence signal compared to a control lacking the sample, is an indicator for the presence and/or the amount of neutralizing Abs within the sample.

As used herein, the term “cytophatic effects (CPE)” refers to visible changes induced upon virus infection of monolayer culture cells as for instance Vero cells. CPE include for instance rounding and detaching of cells from the culture plate. CPE can be observed with a light microscope or by a spectrometric readout. The spectrometric readout is based on the fact that cell death upon virus infection causes the cell media pH to change. This pH change can be visualized by the application of indicators within the cell media (e.g. phenol red) and detected by measuring the absorbance at about 560 nm and about 420 nm and comparing these two values.

As used herein, the term “microneutralization test (MNT)” refers to a method for determining neutralizing Ab titers in a sample. By mixing the virus with a serial dilution of the sample, the reduction of CPE can be observed and thereby the amount of neutralizing Abs within the sample can be determined.

As used herein, the term “plaque reduction neutralization test (PRNT)” refers to a test for determining neutralizing Ab titers for a virus. Therefore, the sample (e.g. serum) is diluted and mixed with a certain amount of virus. Afterwards, the mixture is applied onto confluent monolayer cells (e.g. Vero cells). The surface of the cell layer is subsequently covered with a layer of semisolid overlay medium as for instance agar to prevent the virus from spreading indiscriminately. The concentration of plaque forming units (PFU) can be estimated by the number of plaques (regions of lysed cells) formed after a few days. Depending on the virus, the plaque forming units are measured by microscopic observation and/or specific dyes that react with infected cells or solely stain living cells (e.g. crystal violet). The concentration of sample to reduce the number of plaques by 50% compared to the control, wherein the cells are infected with virus only (lacking sample) is denoted as the “PRNT50” value.

As used herein, the term “focus forming assay (FFA)” refers to a variation of the PRNT assay, wherein the regions of infected cells (foci) are detected by fluorescent antibodies specific for a viral antigen to detect infected host cells and infectious virus particle. The FFA is particularly useful for quantifying classes of viruses that do not lyse the cell membranes, as these viruses would not be amenable to the plaque assay. The FFA is reported as focus forming units (FFU).

As used herein, the term “plaque forming units (PFU)” refers to the number of virus particles capable of forming plaques (regions of lysed cells) per unit volume.

As used herein, the term “focus forming units (FFU)” refers to the number of virus particles capable of forming foci (regions of infected cells) per unit volume.

As used herein, the term “enzyme linked immunosorbent assay (ELISA)” refers to an immunoassay for the measurement of Abs or antigens depending on the ELISA set-up. A key feature of all ELISA set-ups is the application of a plate on which Abs or antigens are immobilized. For instance, in order to determine Abs within a sample, a corresponding antigen to which the Abs bind to is immobilized on the plate. In another set-up, Abs are immobilized on the plate to detect antigens within a sample. The signal of an ELISA is generated by an enzymatic reaction, producing a signal that can be for instance detected by spectrophotometric methods. A common example of an enzyme applied is horseradish peroxidase. Common ELISA set-ups include direct ELISA, sandwich ELISA, competitive ELISA, and reverse ELISA.

As used herein, the term “monoclonal Ab” (“mAb”) refers to an Ab obtained from a population of substantially homogenous Abs that bind to the same antigenic determinants (epitopes). “Substantially homogeneous” means that the individual Abs are identical except for possibly naturally-occurring mutations that may be present in minor amounts. This is in contrast to polyclonal antibodies that typically include different antibodies directed against various, different antigenic determinants (epitopes). A monoclonal Ab may be generated by hybridoma technology according to methods known in the art (Köhler and Milstein, Nature 1975, 256:495-497), phage selection, recombinant expression, and transgenic animals.

As used herein, the term “does not cross-react” refers to an Ab that does not bind to a certain antigen e.g. a flavivirus or a DENV. “Does not bind” within that context means that the Ab shows a binding signal when tested for binding to flavivirus or DENV that is 20% or less, more preferable 10% or less, even more preferable 5% or less of the binding signal when the Ab is tested for binding to ZIKV. In a preferred embodiment, “does not bind” within that context means that the Ab does not show a binding signal above the background signal when tested for binding to flavivirus or DENV. For instance, suitable methods for detecting a binding signal include ELISA or MIA using the corresponding antigen.

As used herein, the term “reporter Ab” is an Ab, which is part of a kit comprising the microsphere complex comprising a microsphere coupled to a ZIKV VLP and is selected to bind to the ZIKV VLP and refers to an Ab that is directly or indirectly attached to at least one detectable label. In a preferred embodiment the at least one detectable label is directly or indirectly attached to the heavy chain constant region of the reporter Ab.

A “reporter Ab indirectly attached to at least one detectable label” refers to a reporter Ab, which reacts with a secondary reporter Ab directly attached to at least one detectable label. In preferred embodiments, the reporter Ab reacts with its heavy chain constant region with the secondary reporter Ab directly attached to at least one detectable label.

As used herein, the term “secondary reporter Ab” refers to an Ab that is directly attached to at least one detectable label and that is capable of binding the reporter Ab heavy chain constant region with the secondary reporter Ab heavy and light chain variable region. In preferred embodiments, the secondary reporter Ab is directly attached to at least one detectable label by its heavy chain constant region. When the secondary reporter Ab is bound to the reporter Ab constant region, the reporter Ab can be indirectly detected by the at least one detectable label of the secondary reporter Ab. For instance, for detection of a reporter Ab generated in mouse, an anti-mouse secondary reporter Ab generated in rabbit may be used.

As used herein, the term “detection Ab” refers to an Ab that is directly attached to at least one detectable label, preferably by its heavy chain constant region, and that is capable of binding to the heavy chain constant region of certain antibody classes and/or subclasses (isotypes) of an origin (e.g. rabbit, human, mouse), which is different than the origin of the detection Ab. For instance, a detection Ab generated in mouse or rabbit, may be used for detection of human antibodies in a sample. The detection Ab may be capable of binding, for instance, to IgG antibodies.

The term “detectable label”, as used herein, refers to any compound or moiety that comprises one or more appropriate chemical substances or enzymes, which directly or indirectly generate a detectable compound or signal in a chemical, physical or enzymatic reaction. Labeling can be achieved by methods well known in the art (see, for example, Lottspeich, F., and Zorbas H., Springer Spektrum 2012, Bioanalytik).

As used herein, the term “detection system” refers to any system which is suitable for determining values indicative for the presence and/or amount of reporter antibody captured on a support member, e.g. a microsphere. The detection system may also be able to determine values indicative for the presence and/or amount of a microsphere. The microsphere may be by individually identified by the specific feature of the microsphere.

As used herein, a “ZIKV specific reporter Ab” or “reporter Ab that specifically binds ZIKV” refers to a reporter Ab that provides an EC₅₀ and/or EC₂₅ value towards the microsphere complex comprising a microsphere coupled to a zika virus like particle (which is part of the kit comprising the microsphere complex and the reporter antibody) which is lower than each EC₅₀ and/or EC₂₅ value which said reporter Ab provides when tested in binding towards other microsphere complexes comprising DENV VLPs. Other microsphere complexes comprising DENV VLPs within that meaning comprise microsphere complexes comprising microspheres coupled to DENV1 VLP, and/or DENV2 VLP, and/or DENV3 VLP, and/or DENV4 VLP.

As used herein, the term “ZIKV specific Ab” or “Ab that specifically binds ZIKV” refers to an Ab that is able to compete with a reporter Ab for binding to the ZIKV VLP coupled to the microsphere within the microsphere complex, wherein the reporter Ab and the microsphere complex are part of the kit comprising the microsphere complex and the reporter Ab. The ZIKV specific Ab is able to displace a certain amount of the reporter Ab bound to the ZIKV VLPs. Vice versa, a certain amount of the ZIKV specific Ab bound to the ZIKV VLPs cannot be displaced by the reporter Ab.

As used herein, the terms “EC₅₀ value” and “EC₂₅ value” refer to the concentration of an Ab required to achieve 50% or 25%, respectively, maximal binding at saturation to an antigen, such as e.g. a ZIKV or DENV VLP, coupled to microspheres in the microsphere complex. The EC₅₀ and EC₂₅ values are a measure for the affinity of an Ab towards the ZIKV or DENV VLP. Within the meaning of the invention, the EC₅₀ and EC₂₅ values indicated for a ZIKV specific reporter Ab (see, for instance, Table 5) refer to twice the concentration of the ZIKV specific reporter Ab required to achieve 50% or 25%, respectively, maximal binding at saturation to an antigen, such as e.g. a ZIKV or DENV VLP, coupled to microspheres in the microsphere complex (2-fold effective concentration).

As used herein, the term “control Ab” or “isotype control” refers to an Ab that enables the evaluation of unspecific signal or binding of the reporter Ab to the antigen-coupled microspheres. The control Ab preferably corresponds to the origin species and the class and subclass of the reporter Ab of which unspecific signal or binding shall be investigated (e.g. human IgG1 Ab, rabbit IgG Ab, mouse IgG1 Ab). The control Ab is preferably a mAb.

A “recombinant protein”, as used herein, refers to a protein which is created, expressed, isolated or obtained by technologies or methods known in the art such as recombinant DNA technology which include, e.g. polymerase chain reaction (PCR), DNA splicing and transgenic expression. The term may refer to proteins expressed in a non-human mammal (including transgenic non-human mammals e.g. transgenic mice), or a cell (e.g. human embryonic kidney cells (HEK293), Chinese hamster ovary (CHO) cells, or bacterial cells like Escherichia coli) expression system. The recombinant protein may be purified by protein purification methods known in the art such as immobilized metal affinity chromatography (IMAC; e.g. His-purification) and size-exclusion chromatography. The protein may be characterized by methods known in the art such as e. g. Bradford or bicinchoninic acid (BCA) assays for determination of protein concentration, or biolayer interferometry (BLI) for determination of binding properties of the protein.

As used herein, the terms “microsphere” or “microspheres” refer to a small particles to which molecules like antigens (i.e. VLPs or EDIII) can be attached to for use in the methods of the present invention. The terms microsphere, microparticle, bead, or microbead can be used interchangeably and bear equivalent meanings. A microsphere may be identified by a specific feature. A microsphere may be part of a microsphere set.

The term “specific feature” refers to a specific property of the microsphere, which allows it to be identified by a detection instrument. Identification of a microsphere likewise allows identification of the antigen, which is attached to the microsphere. The specific feature may be that the microsphere comprises one or more fluorescent dyes having a specific emission spectrum and/or one or more fluorescent dyes at a specific concentration and/or is of a certain size. The microsphere may be identified by determining the size of a microsphere and/or recording the emission of the microsphere after exciting with a certain wavelength and/or the intensity of the emission signal of the microsphere after exciting with a certain wavelength.

As used herein, the term “microsphere set” refers to a plurality of microspheres that share the same specific feature. Microspheres of other sets are characterized by different specific features as one or more fluorescent dyes having another specific emission spectrum and/or one or more fluorescent dyes at another specific concentration and/or another certain size. By detection of their specific feature, microspheres can be identified as part of a set and distinguished from microspheres of other sets. Distinguishing can be carried out by determining the size of a microsphere and/or recording the emission of the microsphere after exciting with a certain wavelength and/or the intensity of the emission signal of the microsphere after exciting with a certain wavelength.

As used herein, the term “multiplexing” refers to the simultaneous detection of multiple different analytes such as antigens or Abs in one single experiment. For instance, application of microspheres of different microsphere sets coupled to different antigens allows the microspheres to be mixed and incubated with a sample containing different Abs directed to the different antigens and thereby to simultaneously detect the different Abs. Consequently, the term “singleplexing” refers to the detection of one analyte in one single experiment. The term “multiplex antigens” refers to the simultaneous detection of multiple different antigens in one single experiment. The term “singleplex antigens” refers to the detection of one antigen in one single experiment.

As used herein, the term “microsphere complex” refers to a complex of microsphere and antigen. The antigen may be covalently attached to the microsphere. The antigen may be a VLP i.e. a ZIKV or DENV1-4 VLP. The antigen may be attached to the microsphere by carbodiimide coupling using 1-Ethyl-3-(3-dimethylaminopropyl)-carbodiimide (EDC) and N-Hydroxysulfosuccinimide (Sulfo-NHS).

As used herein, the term “immunoassay” refers to an assay that detects, determines, identifies, characterizes, quantifies, or otherwise measures the presence and/or concentration of a molecule through the use of an Ab or antigen. The molecule detected by the immunoassay can be present in biological samples (e.g. serum or blood plasma). The molecule detected by the immunoassay may be itself an Ab or antigen. The immunoassay may include, for example, direct or competitive binding assays using techniques such as ELISA, immunoprecipitation assays, or MIAs.

As used herein, the term “microsphere immunoassay” refers to an assay that detects, determines, identifies, characterizes, quantifies, or otherwise measures the presence and/or concentration of Abs with the use of microspheres coupled to an antigen to which the Abs are able to bind. The Abs detected by the microsphere immunoassay can be present in biological samples (e.g. serum or blood plasma).

The term “competing” or “competes with”, as used herein, refers to a situation in which a first Ab competes with a second Ab for a binding site on an antigen (i.e. a ZIKV VLP). The term includes situations in which the Abs are applied concomitantly to the antigen or one after another. One of the two Abs may be a ZIKV specific reporter Ab and the other of the two Abs may be present within a sample. Specifically, in a first orientation, the first Ab is allowed to bind to a ZIKV VLP followed by assessment of binding of the second Ab to the ZIKV VLP. In a second orientation, the second Ab is allowed to bind to a ZIKV VLP followed by assessment of binding of the first Ab to the ZIKV VLP. In a third orientation, the first and the second Ab are concomitantly allowed to bind to a ZIKV VLP. The Abs may be allowed to bind under saturating conditions. As will be appreciated by a person of ordinary skill in the art, the first Ab that competes for binding with the second Ab may not necessarily bind to the same epitope as the second Ab, but may sterically block binding of the second Ab by binding an overlapping or adjacent epitope. Two Abs bind to the same or overlapping epitope if each competitively inhibits (blocks) binding of the other to the antigen. Alternatively, two Abs have the same epitope if essentially all amino acid mutations in the antigen that reduce or eliminate binding of one Ab reduce or eliminate binding of the other. Two Abs have overlapping epitopes if some amino acid mutations that reduce or eliminate binding of one antibody reduce or eliminate binding of the other. The capability of one Ab to inhibit (block) binding of an antigen by another Ab is a measure of the ratio of the affinities of the two Abs for the antigen. If one Ab strongly inhibits (blocks) binding of another Ab to an antigen, the affinity of the one Ab for the antigen is higher than the affinity of the other Ab for the antigen. For instance, a ZIKV specific reporter Ab that shows an EC₅₀ value towards the ZIKV VLP of 0.05 μg/mL (which is a measure for affinity for the ZIKV VLP) will strongly inhibit (block) binding of another anti-ZIKV Ab that sows an EC₅₀ value towards the ZIKV VLP of 1 μg/mL if the Abs bind to the same or overlapping epitopes.

The term “competitive microsphere immunoassay (cMIA)” refers to an microsphere immunoassay that detects, determines, identifies, characterizes, quantifies, or otherwise measures the presence and/or concentration of Abs with the use of microspheres coupled to an antigen to which the Abs are able to bind and a reporter Ab that competes with the Abs for binding to the antigen. The reporter Ab may be a ZIKV specific reporter Ab.

As used herein, the term “antigen” refers to any substance which can be bound by an Ab. Antigens may induce an immune response within a subject. An antigen may have one or more epitopes. An antigen may be a protein, polypeptide, carbohydrate, polynucleotide, lipid, or combinations thereof. As used herein, antigen may refer to a ZIKV VLP, ZIKV E protein, ZIKV EDIII, DENV1 VLP, DENV2 VLP, DENV3 VLP, DENV4 VLP. The ZIKV EDIII may be recombinant and attached to different tags as 6×His, 6×His-SUMO, or human IgG1 Fc-portion. The Fc portion of an Ab can be generated by e.g. papain digestion of a full-length Ab and comprises the heavy chain constant region domains 2 and 3 (CH2 and CH3).

As used herein, the terms “virus like particle (VLP)” or “virus like particles (VLPs)” refer to molecules that closely resemble viruses, but are non-infectious because they do not contain viral genetic material. VLPs can be prepared recombinant through the expression of viral structural proteins, which can then self-assemble into the VLPs. Suitable expression systems include eukaryotic expression systems like mammalian or insect expression systems.

As used herein, the term “ZIKV VLP” refers to a VLP comprising at least one of the structural proteins (prM, M, and E protein) of ZIKV strains. The structural proteins may be at least 70%, or at least 75%, or at least 80%, or at least 85%, or at least 90%, or at least 95% or 100% identical to sequences of naturally occurring ZIKV strains as described within this section under the definition “ZIKV”. “ZIKV VLP is derived from ZIKV strain” means that the ZIKV VLP was created by expressing the sequence of at least one structural protein of the ZIKV strain or a sequence that may be at least 70%, or at least 75%, or at least 80%, or at least 85%, or at least 90%, or at least 95% identical to the sequence of at least one structural protein of the ZIKV strain in a suitable expression system. “ZIKV VLP” and “zika VLP” bare equivalent meanings.

As used herein, the term “DENV VLP” refers to a VLP comprising at least one of the structural proteins (prM, M, and E protein) of DENV strains. The term DENV VLP may refer to VLP comprising at least one of the structural proteins of DENV1, DENV2, DENV3, and DENV4 strains, consequently being described by the terms DENV1 VLP, DENV2 VLP, DENV3 VLP, and DENV4 VLP. The structural proteins may be at least 70%, or at least 75%, or at least 80%, or at least 85%, or at least 90%, or at least 95% or 100% identical to sequences of naturally occurring DENV strains as described within this section under the definition “dengue serotype”. In preferred embodiments DENV structural proteins are at least 70%, or at least 75%, or at least 80%, or at least 85%, or at least 90%, or at least 95% or 100% identical to sequences of DENV1 strain Puerto Rico/US/BID-V853/1998 (GenBank accession No. EU482592.1; SEQ ID NO: 179 and/or SEQ ID NO: 180), DENV2 strain Thailand/16681/84 (EMBL-EBI accession No: U87411.1; SEQ ID NO: 181 and/or SEQ ID NO: 182), DENV3 strain Sri Lanka D3/H/IMTSSA-SRI/2000/1266 (GenBank accession No. AY099336.1; SEQ ID NO: 183 and/or SEQ ID NO: 184), and DENV4 strain Dominica/814669/1981 (EMBL-EBI accession No: AF326825.1; SEQ ID NO: 185 and/or SEQ ID NO: 186) and DENV VLPs are produced in HEK293 cells. In certain embodiments, for production of DENV VLPs, the C-terminal 20% of the DENV E protein of DENV1 strain Puerto Rico/US/BID-V853/1998 (GenBank accession No. EU482592.1; SEQ ID NO: 179 and/or SEQ ID NO: 180), DENV2 strain Thailand/16681/84 (EMBL-EBI accession No: U87411.1; SEQ ID NO: 181 and/or SEQ ID NO: 182), DENV3 strain Sri Lanka D3/H/IMTSSA-SRI/2000/1266 (GenBank accession No. AY099336.1; SEQ ID NO: 183 and/or SEQ ID NO: 184), and DENV4 strain Dominica/814669/1981 (EMBL-EBI accession No: AF326825.1; SEQ ID NO: 185 and/or SEQ ID NO: 186) were replaced by the corresponding Japanese encephalitis virus (JEV) SA-14 sequence (EMBL-EBI accession No: M55506.1, SEQ ID NO: 177 and/or SEQ ID NO: 178; E protein amino acids 399-497 (DENV1 VLP), 397-495 (DENV2 VLP), 399-492 (DENV3 VLP), 400-495 (DENV4 VLP)). The replaced sequence corresponds to the transmembrane and intraparticle portion of the protein. “DENV VLP is derived from DENV strain” means that the DENV VLP was created by expressing the sequence of at least one structural protein of the DENV strain or a sequence that may be at least 70%, or at least 75%, or at least 80%, or at least 85%, or at least 90%, or at least 95% identical to the sequence of at least one structural protein of the DENV strain in a suitable expression system. “DENV VLP” and “dengue VLP” bare equivalent meanings.

As used herein, the term “E protein” refers to the envelope glycoprotein (E). Consequently “ZIKV E protein” refers to ZIKV envelope glycoprotein (E). The E protein may be a recombinant protein. The amino acid sequence of ZIKV E protein is part of the viral polyprotein encoded by a ZIKV strain. In particular, the amino acid sequence of ZIKV E protein (SEQ ID NO: 3) is part of the viral polyprotein (E protein corresponds to amino acids 291-794; GenBank accession No. AWH65849.1) encoded by the ZIKV strain PRVABC59 (GenBank accession No. MH158237.1).

As used herein, the term “EDIII” refers to ZIKV carboxyl (C)-terminal domain III of the E protein ectodomain. The EDIII may be a recombinant protein. The EDIII recombinant protein may carry a tag at the C- or amino (N)-terminus, for instance 6×His, 6×His-SUMO or human IgG1 Fc. “Carry” within that context means that the polypeptide chain of EDIII is in-frame connected to the polypeptide chain of the tag. The amino acid sequence of EDIII protein is part of the E protein, which is part of the viral polyprotein encoded by a ZIKV strain. For instance, the amino acid sequence of EDIII is encoded by V593 to L933 (GenBank accession No. ALU33341.1) of ZIKV strain SPH2015 (GenBank accession No. KU321639.1), is encoded within SEQ ID NO: 3 of ZIKV strain PRVABC59 (GenBank Accession No. MH158237.1), or is encoded by SEQ ID NO: 4 of ZIKV strain H/PF/2013 (GenBank Accession No. KJ776791).

As used herein, the term “epitope” or “antigenic determinant” refers to the part of an antigen that interacts with a specific antigen-binding site in the variable region of an Ab molecule known as a paratope. Conversely, the “epitope” can also interact with a specific cellular receptor or binding site on a host. A single antigen may have more than one epitope. Thus, different Abs may bind to different areas on an antigen and may have different biological effects. For example, the term “epitope” also refers to a site on an antigen to which B and/or T cells respond. Epitopes may be defined as structural or functional. Functional epitopes are generally a subset of the structural epitopes and have those residues that directly contribute to the affinity of the interaction. The epitope to which the antibodies bind may consist of a single contiguous sequence of 2 or more (e.g., 2, 3, 4, 5, 6, 7, 8, 9, 10, 11, 12, 13, 14, 15, 16, 17, 18, 19, 20 or more) amino acids located within an antigen i.e. a linear epitope for instance in a domain of a ZIKV E protein. Epitopes may also be conformational, that is, composed of a plurality of non-contiguous amino acids, i.e., non-linear amino acid sequence. A conformational epitope typically includes at least 3 amino acids, and more commonly, at least 5 amino acids, e.g., 7-10 amino acids in a unique spatial conformation. In certain embodiments, epitopes may include determinants that are chemically active surface groupings of molecules such as amino acids, sugar side chains, phosphoryl groups, or sulfonyl groups, and, in certain embodiments, may have specific charge characteristics. Epitopes formed from contiguous amino acids are typically retained on exposure to denaturing solvents, whereas epitopes formed by tertiary folding are typically lost on treatment with denaturing solvents. Various techniques known to persons of ordinary skill in the art can be used to determine whether an antibody interacts with one or more amino acids within a polypeptide or protein. Exemplary techniques include, for example, site-directed mutagenesis (e.g., alanine scanning mutational analysis). Other methods include routine cross-blocking assays (such as that described in Antibodies, Harlow and Lane, Cold Spring Harbor Press, Cold Spring Harbor, NY), peptide blot analysis (Reineke (2004) Methods Mol. Biol. 248: 443-63), peptide cleavage analysis crystallographic studies and NMR analysis. In addition, methods such as epitope excision, epitope extraction and chemical modification of antigens can be employed (Tomer (2000) Prot. Sci. 9: 487-496). Another method that can be used to identify the amino acids within a polypeptide with which an antibody interacts is hydrogen/deuterium exchange detected by mass spectrometry. In general terms, the hydrogen/deuterium exchange method involves deuterium-labeling the protein of interest, followed by binding the antibody to the deuterium-labeled protein. Next, the protein/antibody complex is transferred to water and exchangeable protons within amino acids that are protected by the antibody complex undergo deuterium-to-hydrogen back-exchange at a slower rate than exchangeable protons within amino acids that are not part of the interface. As a result, amino acids that form part of the protein/antibody interface may retain deuterium and therefore exhibit relatively higher mass compared to amino acids not included in the interface. After dissociation of the antibody, the target protein is subjected to protease cleavage and mass spectrometry analysis, thereby revealing the deuterium-labeled residues that correspond to the specific amino acids with which the antibody interacts. See, e.g., Ehring (1999) Analytical Biochemistry 267: 252-259; Engen and Smith (2001) Anal. Chem. 73: 256A-265A. Modification-Assisted Profiling (MAP), also known as Antigen Structure-based Antibody Profiling (ASAP) may be used to sort Abs binding the same antigen into groups of Abs binding different epitopes. MAP is a method that categorizes large numbers of Abs directed against the same antigen according to the similarities of the binding profile of each antibody to chemically or enzymatically modified antigen surfaces (see US 2004/0101920). Each category may reflect a unique epitope either distinctly different from or partially overlapping with epitope represented by another category. This technology allows rapid filtering of genetically identical antibodies, such that characterization can be focused on genetically distinct antibodies. When applied to hybridoma screening, MAP may facilitate identification of rare hybridoma clones that produce mAbs having the desired characteristics.

As used herein, the term “flavivirus” refers to viruses belonging to the genus Flavivirus of the family Flaviviridae. According to virus taxonomy, about 50 viruses including ZIKV, DENV, SLEV, TBEV, YFV, JEV, WNV, and related flaviviruses are members of this genus. The viruses belonging to the genus Flavivirus are referred to herein as flaviviruses. Currently, these viruses are predominantly in East, Southeast and South Asia and Africa, although they may be found in other parts of the world.

As used herein, the term “Zika virus (ZIKV)” refers to a flavivirus, which has been linked to microcephaly and other developmental abnormalities in the fetuses of pregnant women exposed to the virus (Schuler-Faccini et al., MMWR Morb. Mortal. Wkly. Rep. 2016, 65:59-62) as well as Guillian-Barre syndrome in adults (Cao-Lormeau et al., Lancet 2016, 387(10027):1531-9). The ZIKV may be from African or Asian genotype. A ZIKV possesses a positive sense, single-stranded RNA genome encoding both structural and nonstructural polypeptides. The genome also contains non-coding sequences at both the 5′- and 3′-terminal regions that play a role in virus replication. Structural polypeptides encoded by these viruses include, without limitation, capsid (C), precursor membrane (prM), membrane (M), and envelope (E) protein. Non-structural (NS) polypeptides encoded by these viruses include, without limitation, NS1, NS2A, NS2B, NS3, NS4A, NS4B, and NS5. The term “ZIKV” includes strains of ZIKV isolated from different ZIKV isolates, including ZikaSPH (Brazil 2015, GenBank accession No. KU321639.1), Brazil-ZKV (Brazil 2015, GenBank accession No. KU497555.1), PRVABC59 (Puerto Rico 2015, GenBank accession No. KU501215.1), Haiti1225 (Haiti 2014, GenBank accession No. KU509998.1), Natal RGN (Brazil, GenBank accession No. KU527068.1), SV0127-14 (Thailand 2014, GenBank accession No. KU681081.3), SPH2015 (GenBank accession No. KU321639.1), CPC-0740 (Philippine 2012, GenBank accession No. KU681082.3), SSABR1 (Brazil, GenBank accession No. KU707826.1), VE_Ganxian (China, GenBank accession No. KU744693.1), MR766-NIID (Uganda, GenBank accession No. LC002520.1), MR 766 (Uganda 1947, GenBank accession No. AY632535.2), and H/PF (French Polynesia 2013, GenBank accession No KJ776791.1) (WO 2017/109225). Further, ZIKV strains include Cambodia 2010 (GenBank accession No JN860885) or Micronesia 2007 (GenBank accession No EU545988) (Mlakar et al., N Engl J Med. 2016 Mar. 10; 374(10):951-8). Further, ZIKV strains include FLR (Colombia 2015) strain (WO 2018/017497), Z1106031 isolated in Suriname (Asian genotype; GenBank accession No KU312314), Z1106027 isolated in Suriname (Asian genotype; GenBank accession No KU312315); Z1106032 isolated in Suriname (Asian genotype; GenBank accession No KU312313), and Z1106033 isolated in Suriname (Asian genotype; Enfissi et al., Lancet 2016, 387(10015):227-228; GenBank Accession No. KU312312.1, SEQ ID NO: 1 and SEQ ID NO: 2).

As used herein, the term “Dengue virus (DENV)” refers to a flavivirus possessing a positive sense, single-stranded RNA genome encoding both structural and nonstructural polypeptides. The genome also contains non-coding sequences at both the 5′- and 3′-terminal regions that play a role in virus replication. Structural polypeptides encoded by these viruses include, without limitation, capsid (C), precursor membrane (prM), membrane (M), and envelope (E) protein. Non-structural (NS) polypeptides encoded by these viruses include, without limitation, NS1, NS2A, NS2B, NS3, NS4A, NS4B, and NS5. DENV can be divided in different dengue serotypes. The term “DENV” may refer to DENV including all dengue serotypes.

The term “dengue serotype” as used herein, refers to a species of dengue virus which is defined by its cell surface antigens and therefore can be distinguished by serological methods known in the art. Four serotypes of dengue virus are known, i.e. dengue serotype 1 (DENV1), dengue serotype 2 (DENV2), dengue serotype 3 (DENV3), dengue serotype 4 (DENV4). The term “dengue serotype” includes strains of DENV isolated from different DENV isolates, for instance DENV1 strain Puerto Rico/US/BID-V853/1998 (GenBank accession No. EU482592.1; SEQ ID NO: 179 and 180), DENV2 strain Thailand/16681/84 (EMBL-EBI accession No: U87411.1; SEQ ID NO: 181 and 182), DENV3 strain Sri Lanka D3/H/IMTSSA-SRI/2000/1266 (GenBank accession No. AY099336.1; SEQ ID NO: 183 and 184), and DENV4 strain Dominica/814669/1981 (EMBL-EBI accession No: AF326825.1; SEQ ID NO: 185 and 186).

As used herein, the term “structural protein” refers to viral proteins that are structural components of the mature virus. Structural proteins include without limitation C, E, prM, and M protein of a flavivirus. The term “structural proteins” may also refer to all of the proteins including without limitation C, E, prM, and M protein of a flavivirus. The flavivirus may be a DENV or a ZIKV.

As used herein, the terms “subject” or “subjects” can include any individual. A subject may be, but is not limited to, a mouse, a primate, a non-human primate (NHP), a human, a rabbit, a cat, a rat, a horse, a sheep. In certain embodiment the subject can be a pregnant mammal, and in particular embodiments a pregnant human female. In some embodiments, the subject is a patient, for whom prophylaxis or therapy is desired. The subject may be flavivirus naive or flavivirus exposed. The subject may be from a flavivirus endemic region.

As used herein, the term “non-human subject” can include any individual that is not a human. A non-human subject may be, but is not limited to, a mouse, a primate, a non-human primate (NHP), a rabbit, a cat, a rat, a horse, a sheep.

The term “flavivirus naive” or “flavivirus negative” as used herein refers to a subject that does not have Abs directed to a flavivirus as determined in a test measuring for instance neutralizing flavivirus Ab titers or total flavivirus Abs. Tests measuring neutralizing flavivirus Ab titers may be a RVP test, a PRNT, a FFA or a MNT test. A test measuring total flavivirus Ab titers may be a MIA. The term may also refer to a subject that has not been exposed to a flavivirus and therefore does not carry Abs directed to the flavivirus. The flavivirus may be a ZIKV or DENV.

The term “ZIKV naive” or “ZIKV negative” as used herein refers to a subject that does not have Abs directed to ZIKV as determined in a test measuring neutralizing ZIKV Ab titers or total ZIKV Abs. Tests measuring neutralizing ZIKV Ab titers may be a RVP test, a PRNT, a FFA or a MNT test. A test measuring total ZIKV Ab titers may be a MIA. The term may also refer to a subject that has not been exposed to a ZIKV and therefore does not carry Abs directed to the ZIKV.

As used herein, the term “flavivirus exposed” or “flavivirus positive” refers to exposure of any subject to an antigen of a flavivirus. The flavivirus may be ZIKV or DENV. Flavivirus exposure can be the result of a flavivirus infection or vaccination with a flavivirus vaccine. A result of flavivirus exposure is the generation of Abs directed to the flavivirus by an immune response of the subject induced upon flavivirus exposure. The Abs may be determined in a test measuring neutralizing flavivirus Ab titers or total flavivirus Abs. Tests measuring neutralizing flavivirus Ab titers may be a RVP test, a PRNT, a FFA or a MNT test. A test measuring total flavivirus Ab titers may be a MIA.

The term “flavivirus infection” as used herein, refers to the disease or condition which results from contact to a flavivirus (e.g. after being bitten by a mosquito harboring the virus), or to an infected animal, or to an infected human patient, or contact with the body fluids or tissues from an animal or human patient having a flavivirus infection. The flavivirus may be a ZIVK or a DENV. A flavivirus infection may also not be accompanied by flavivirus specific symptoms, in such a case the infection may be asymptomatic or inapparent. A flavivirus infection may be acute or convalescent.

The term “ZIKV infection” as used herein, refers to the disease or condition which results from contact to ZIKV (e.g. after being bitten by a mosquito harboring the virus), or to an infected animal, or to an infected human patient, or contact with the body fluids or tissues from an animal or human patient having a ZIKV infection. ZIKV infection can be accompanied by specific symptoms caused by the infection as headache, fever, arthralgia, myalgia, maculopapular rash, fatigue, or body aches. The specific symptoms accompanying a ZIKV infection may be referred to as “zika disease”. The condition resulting from exposure to ZIKV may include microcephaly or developmental abnormalities of a fetus in a pregnant woman who was infected with the ZIKV after being bitten by a mosquito harboring ZIKV. Another condition resulting from exposure to the ZIKV includes Guillain-Barre Syndrome. A ZIKV infection may also not be accompanied by ZIKV specific symptoms, in such a case the infection may be asymptomatic or inapparent. A ZIKV infection may be acute or convalescent.

The term “preventing zika disease” as used herein, refers to a measure, which protects a subject from developing zika disease after a zika virus infection. The measure may be administering to the subject a zika virus vaccine, such as a purified inactivated zika virus vaccine.

As used herein, the term “acute flavivirus infection” refers to a flavivirus infection that is characterized by rapid onset of disease, a relatively brief period of symptoms, and resolution within days. A rapid flavivirus infection is usually accompanied by early production of infectious virions and elimination of infection by the host immune system. Within an acute flavivirus infection Ab titers in body fluids are high compared to a convalescent virus infection. The flavivirus within that context may be a ZIKV (“acute ZIKV infection”). An “acute flavivirus infection” may refer to the period of viremia.

As used herein, the term “convalescent flavivirus infection” refers to a flavivirus infection that has been eliminated by the host immune system. A characteristic of a convalescent flavivirus infection is the existence of memory B-cells encoding for Abs against the flavivirus that has caused the infection. Within a convalescent flavivirus infection Ab titers in body fluids are low compared to an acute flavivirus infection. The flavivirus within that context may be a ZIKV (“convalescent ZIKV infection”). A “convalescent flavivirus infection” may refer to the period after viremia.

As used herein, an “immune response” refers to a subject's immune response to flavivirus exposure. In particular, the immune response includes the formation of Abs to the flavivirus. The term immune response may also include formation of neutralizing Abs to the flavivirus. It may also include the stimulation of a cell-mediated response or the formation of Abs to structural proteins such as E protein. It may also include the stimulation of a cell-mediated response.

As used herein, “endemic region” refers to a region where a disease or infectious agent is constantly present and/or usually prevalent in a population within this region. As used herein, “non-endemic region” refers to a region from which the disease is absent or in which it is usually not prevalent. Accordingly, a “flavivirus endemic region” refers to geographic areas in which an infection with flavivirus is constantly maintained at a baseline level. A “flavivirus non-endemic region” is a geographic area in which an infection with flavivirus is not constantly maintained at a baseline level. Accordingly, subject populations or subjects “from a flavivirus endemic region” or “from a flavivirus non-endemic region” refer to subject populations or subjects living in geographic areas as defined above.

As used herein, the term “sample” refers to any sample. The sample may be derived from a subject. Within the meaning of this invention, the sample is present outside the human or animal body. The sample may be serum, blood plasma, blood, urine, cerebrospinal fluid, lymph fluid. In some embodiments, the sample contains Abs directed to a flavivirus. The said sample can be pre-treated prior to use, such as preparing plasma from blood, diluting fluids, or the like. Methods for pre-treating can involve purification, filtration, distillation, concentration, inactivation of interfering compounds, and the addition of reagents. In some embodiment, the sample is heat-inactivated. Within this invention, the term “plasma” refers to blood plasma.

As used herein, the term “negative control” refers to a sample, which does not contain Abs directed to flaviviruses. The negative sample may be included as a control in immunoassays due to the absence of Abs directed to flaviviruses. Flaviviruses in this context may be DENV and/or ZIKV and/or WNV and/or JEV and/or SLEV and/or YFV and/or TBEV. The negative control may be a human serum sample.

As used herein, the term “correlate of protection” refers to an amount of an immune marker associated with absence of viremia. A correlate of protection can be determined by challenging a non-human animal with an infectious amount of a corresponding virus for instance after vaccination of said non-human animal with a certain dose of a vaccine. A correlate of protection of a subject (i.e. a human) can be alternatively determined by mathematically modelling the correlate of protection of a non-human subject. In preferred embodiments, the immune marker is an amount of Ab (“Ab correlate of protection”). The term “correlate of protection” may also refer to the minimum amount of an immune marker associated with absence of viremia (“minimum correlate of protection”). A correlate of protection for a vaccine may refer to a correlate of protection induced by a certain dose of a vaccine (e.g. a dose of 1 μg of zika vaccine induces an amount of Ab (correlate of protection) associated with the absence of viremia).

As used herein, the term “antibody correlate of protection” refers to an amount of Ab associated with absence of viremia e.g. after challenging with an infectious amount of a corresponding virus. An “Ab correlate of protection” may be determined as for instance described in Young et al., Scientific Reports 2020, 10:3488.

As used herein, the term “protection” refers to a condition wherein the amount of an immune marker, i.e. amount of Ab within a subject is equal to or higher as the corresponding correlate of protection. The corresponding correlate of protection may also be a minimum correlate of protection.

As used herein, the term “viremia” or “presence of viremia” refers to a medical condition were viruses have entered the bloodstream thereby spreading throughout the body. A viremia can be primary or secondary. A primary viremia refers to a virus entering the bloodstream from the first site of infection. A secondary viremia occurs when primary viremia has resulted in infection of additional tissues in which the virus has replicated and once more entered the bloodstream circulation. Viremia may induce typical symptoms caused by an infection with the virus such as fever or headache. Viremia can be determined by monitoring the typical symptoms caused by an infection and/or determining the amount of virus in samples as blood, blood plasma, or urine e.g. by diagnostic methods like RT-PCRs, PRNT, FFA, MNT, or RVP assay.

As used herein, the term “absence of viremia” refers to a medical condition where viruses have not entered the bloodstream thereby spreading throughout the body. “Absence of viremia” may also refer to the absence of typical symptoms caused by an infection with the virus. Absence of viremia can be determined by monitoring the typical symptoms caused by an infection and/or determining the amount of virus in samples as blood, blood plasma, or urine e.g. by diagnostic methods like RT-PCRs, PRNT, MNT, FFA or RVP assay. Absence of viremia may be the result of an amount of immune marker within a subject, that is equal to or higher as the corresponding correlate of protection. Absence of viremia may refer to conditions, wherein upon analyzing a subject's sample the signal from diagnostic methods like RT-PCRs is not higher than a corresponding background signal.

As used herein, the term “immune marker” refers to a molecule within a subject involved in, or influenced by the relationship between vaccination or natural virus infection and protection of a subject. The immune marker changes, i.e. appears or increases in concentration in the course of protection induced by a vaccine or a natural virus infection in a subject. An immune marker may be an Ab produced by the subject. An immune marker may be determined by immunoassays according to the present invention.

As used herein, “inoculating”, “vaccinating”, “inoculation”, or “vaccination” refers to the administration of a vaccine to a subject, with the aim to prevent the subject from viremia, including developing one or more symptoms of a disease. In principle, the method comprises a primary inoculation/vaccination and optionally one or more booster inoculations/vaccinations. The corresponding subject may be refers to as “inoculated” or “vaccinated”.

As used herein, the term “control group” refers to a group of subjects that is proposed to be given or has been given a control sample instead of a vaccine. The control sample may be a buffer (e.g. phosphate buffered saline) or any other placebo. A placebo as used herein refers to an inert substance or treatment which is designed to have no therapeutic value. A control group is included in addition to the inoculation group in order to monitor immune responses by the subject that are induced for instance by challenging said subject with an infectious amount of a virus as “blank immune response”. The “blank immune response” can be compared to immune responses induced by vaccinating a subject and thereby the immunogenicity of a vaccine can be determined.

As used herein, the term “immunogenicity” is the ability of an antigen to induce an immune response in a subject. The immune response can be humoral and/or cell-mediated. In comparison with antigenicity, immunogenicity is measured by in vivo studies administering the antigen as present in vaccines to a subject and monitoring the induced immune response.

As used herein, the term “antigenicity” refers to the capacity of an antigen to bind to Abs and therefore to the availability of certain epitopes. Antigenicity is measured by in vitro conformational methods as for instance ELISA methods.

As used herein, the term “inoculation group” refers to a group of subjects that is proposed to be inoculated or has been inoculated with a certain dose of vaccine. The vaccine may be a ZIKV vaccine.

As used herein, the term “vaccine” refers to a prophylactic material providing at least one antigen capable of introducing an immune response in a subject. The antigen may be derived from any material that is suitable for vaccination. For example, the antigen may be derived from a pathogen, such as from virus particles. In a preferable embodiment the antigen may derive from a ZIKV and is referred to as “ZIKV vaccine”. In a more preferable embodiment the antigen may derive from an inactivated ZIKV and the vaccine is consequently referred to as inactivated ZIKV vaccine (IZV). The antigen stimulates the body's adaptive immune system to provide an immune response.

As used herein, the term “inactivated Zika vaccine (IZV)” refers to a ZIKV vaccine that is consisting of ZIKV particles that have been amplified in culture and then inactivated to lose disease producing capacity. In addition to the inactivation step the ZIKV vaccine may be purified and referred to as “purified inactivated Zika vaccine (PIZV)”. In contrast, live vaccines use viruses that are still alive but mostly attenuated, that is, weakened. Inactivation may occur by incubation of the ZIKV vaccine with formaldehyde. Purification of the ZIKV vaccine may occur by filtration and chromatography. The ZIKV vaccine may be derived from ZIKV strain PRVABC59.

As used herein, the term “dose of ZIKV vaccine” refers to a certain amount of ZIKV vaccine. The term “dose of ZIKV vaccine” may refer to an amount of ZIKV vaccine given by one administration to a subject or an amount of ZIKV vaccine given by all administrations to a subject i.e. including booster administrations.

As used herein, the term “challenging” refers to infecting a subject with a flavivirus in order to monitor absence or presence and degree of viremia including virus infection related symptoms. The virus may be a ZIKV. Infection with the virus may be accompanied by ZIKV specific symptoms caused by the infection such as headache, fever, arthralgia, myalgia, maculopapular rash, fatigue, or body aches.

As used herein, the term “infectious amount” refers to an amount of a flavivirus (i.e. a ZIKV) that is capable of inducing viremia, including flavivirus specific symptoms when infecting a subject.

As used herein, the term “diagnosis” or “diagnosing” refers to methods that can be used to confirm or determine the likelihood of whether a patient is suffering from or had previously suffered from a given disease or condition i.e. a ZIKV or DENV infection.

As used herein, the term “established amounts of anti-ZIKV antibodies” refers to amounts of anti-ZIKV Abs present within subjects that are or have been suffering from a ZIKV infection. The established amounts of anti-ZIKV Abs are an indicator for an acute or convalescent ZIKV infection and can be used for the diagnosis of the ZIKV infection, when comparing the amount of anti-ZIKV Abs of a subject to the established amounts of anti-ZIKV Abs. Within that context ZIKV infected subjects may be humans.

### BRIEF DESCRIPTION OF THE DRAWINGS

FIG. 1 Binding of Anti-ZIKV #1 (A) and #2 (B) to ZIKV VLP and DENV1-4 VLPs. Incubation of rising mAb concentrations with ZIKV VLP and DENV1-4 VLPs coupled to the microspheres at pH 6 (DENV1-4 VLPs) or pH 7 (ZIKV VLP), respectively, was carried out for 1 hour. Median Fluorescent Intensity (MFI) is presented as a function of logarithmized mAb concentration (μg/mL). The mAb concentration presented is the concentration previous to two-fold dilution upon incubation with the microspheres. For final assay mAb concentrations, the presented concentrations need to be divided by two.

FIG. 2 Binding of Anti-ZIKV #3 (A) and #4 (B) to ZIKV VLP and DENV1-4 VLPs. Incubation of rising mAb concentrations with ZIKV VLP and DENV1-4 VLPs coupled to the microspheres at pH 6 (DENV1-4 VLPs) or pH 7 (ZIKV VLP), respectively, was carried out for 1 hour. Median Fluorescent Intensity (MFI) is presented as a function of logarithmized mAb concentration (μg/mL). The mAb concentration presented is the concentration previous to two-fold dilution upon incubation with the microspheres. For final assay mAb concentrations, the presented concentrations need to be divided by two.

FIG. 3 Binding of Anti-ZIKV #5 (A) and #6 (B) to ZIKV VLP and DENV1-4 VLPs. Incubation of rising mAb concentrations with ZIKV VLP and DENV1-4 VLPs coupled to the microspheres at pH 6 (DENV1-4 VLPs) or pH 7 (ZIKV VLP), respectively, was carried out for 1 hour. Median Fluorescent Intensity (MFI) is presented as a function of logarithmized mAb concentration (μg/mL). The mAb concentration presented is the concentration previous to two-fold dilution upon incubation with the microspheres. For final assay mAb concentrations, the presented concentrations need to be divided by two.

FIG. 4 Binding of Anti-ZIKV #7 to ZIKV VLP and DENV1-4 VLPs. Incubation of rising mAb concentrations with ZIKV VLP and DENV1-4 VLPs coupled to the microspheres at pH 6 (DENV1-4 VLPs) or pH 7 (ZIKV VLP), respectively, was carried out for 1 hour. Median Fluorescent Intensity (MFI) is presented as a function of logarithmized mAb concentration (μg/mL). The mAb concentration presented is the concentration previous to two-fold dilution upon incubation with the microspheres. For final assay mAb concentrations, the presented concentrations need to be divided by two.

FIG. 5 Binding of Rabbit IgG Isotype Control (A) and Purified Human IgG1 (B) to ZIKV VLP and DENV1-4 VLPs. Incubation of rising mAb concentrations with ZIKV VLP and DENV1-4 VLPs coupled to the microspheres at pH 6 (DENV1-4 VLPs) or pH 7 (ZIKV VLP), respectively, was carried out for 1 hour. Median Fluorescent Intensity (MFI) is presented as a function of logarithmized mAb concentration (μg/mL). The mAb concentration presented is the concentration previous to two-fold dilution upon incubation with the microspheres. For final assay mAb concentrations, the presented concentrations need to be divided by two.

FIG. 6 Binding of Mouse IgG1 Isotype Control to ZIKV VLP and DENV1-4 VLPs. Incubation of rising mAb concentrations with ZIKV VLP and DENV1-4 VLPs coupled to the microspheres at pH 6 (DENV1-4 VLPs) or pH 7 (ZIKV VLP), respectively, was carried out for 1 hour. Median Fluorescent Intensity (MFI) is presented as a function of logarithmized mAb concentration (μg/mL). The mAb concentration presented is the concentration previous to two-fold dilution upon incubation with the microspheres. For final assay mAb concentrations, the presented concentrations need to be divided by two.

FIG. 7 Binding of Anti-ZIKV #1 (A) and 2 (B) to ZIKV VLP. Incubation of rising mAb concentrations with ZIKV VLP coupled to the microspheres at pH 7 was carried out for 10 min. Median Fluorescent Intensity (MFI) is presented as a function of logarithmized mAb concentration (μg/mL). The mAb concentration presented is the concentration previous to two-fold dilution upon incubation with the microspheres. For final assay mAb concentrations, the presented concentrations need to be divided by two.

FIG. 8 Binding of Anti-ZIKV #3 (A) and 4 (B) to ZIKV VLP. Incubation of rising mAb concentrations with ZIKV VLP coupled to the microspheres at pH 7 was carried out for 10 min. Median Fluorescent Intensity (MFI) is presented as a function of logarithmized mAb concentration (μg/mL). The mAb concentration presented is the concentration previous to two-fold dilution upon incubation with the microspheres. For final assay mAb concentrations, the presented concentrations need to be divided by two.

FIG. 9 Binding of Anti-ZIKV #5 (A) and 6 (B) to ZIKV VLP. Incubation of rising mAb concentrations with ZIKV VLP coupled to the microspheres at pH 7 was carried out for 10 min. Median Fluorescent Intensity (MFI) is presented as a function of logarithmized mAb concentration (μg/mL). The mAb concentration presented is the concentration previous to two-fold dilution upon incubation with the microspheres. For final assay mAb concentrations, the presented concentrations need to be divided by two.

FIG. 10 Binding of Anti-ZIKV #7 (A) and Anti-PanDENV1-4 EDIII (B) to ZIKV VLP. Incubation of rising mAb concentrations with ZIKV VLP coupled to the microspheres at pH 7 was carried out for 10 min. Median Fluorescent Intensity (MFI) is presented as a function of logarithmized mAb concentration (μg/mL). The mAb concentration presented is the concentration previous to two-fold dilution upon incubation with the microspheres. For final assay mAb concentrations, the presented concentrations need to be divided by two.

FIG. 11 Binding of Anti-ZIKV #1 (A) and 2 (B) to rZIKV-EDIII-1. Incubation of rising mAb concentrations with rZIKV-EDIII-1 coupled to the microspheres at pH 6 and 7, respectively, was carried out for 1 hour. Median Fluorescent Intensity (MFI) is presented as a function of logarithmized mAb concentration (μg/mL). The mAb concentration presented is the concentration previous to two-fold dilution upon incubation with the microspheres. For final assay mAb concentrations, the presented concentrations need to be divided by two.

FIG. 12 Binding of Anti-ZIKV #3 (A) and 4 (B) to rZIKV-EDIII-1. Incubation of rising mAb concentrations with rZIKV-EDIII-1 coupled to the microspheres at pH 6 and 7, respectively, was carried out for 1 hour. Median Fluorescent Intensity (MFI) is presented as a function of logarithmized mAb concentration (μg/mL). The mAb concentration presented is the concentration previous to two-fold dilution upon incubation with the microspheres. For final assay mAb concentrations, the presented concentrations need to be divided by two.

FIG. 13 Binding of Anti-ZIKV #5 (A) and 6 (B) to rZIKV-EDIII-1. Incubation of rising mAb concentrations with rZIKV-EDIII-1 coupled to the microspheres at pH 6 and 7, respectively, was carried out for 1 hour. Median Fluorescent Intensity (MFI) is presented as a function of logarithmized mAb concentration (μg/mL). The mAb concentration presented is the concentration previous to two-fold dilution upon incubation with the microspheres. For final assay mAb concentrations, the presented concentrations need to be divided by two.

FIG. 14 Binding of Anti-ZIKV #7 (A) and Anti-PanDENV1-4 EDIII (B) to rZIKV-EDIII-1. Incubation of rising mAb concentrations with rZIKV-EDIII-1 coupled to the microspheres at pH 6 and 7, respectively, was carried out for 1 hour. Median Fluorescent Intensity (MFI) is presented as a function of logarithmized mAb concentration (μg/mL). The mAb concentration presented is the concentration previous to two-fold dilution upon incubation with the microspheres. For final assay mAb concentrations, the presented concentrations need to be divided by two.

FIG. 15 Binding of Anti-ZIKV #1, Anti-PanDENV1-4 EDIII, and rabbit IgG and mouse IgG1 isotype control to rZIKV-EDIII-3. Incubation of rising mAb concentrations with rZIKV-EDIII-3 coupled to the microspheres at pH 5, 6, and 7, respectively, was carried out for 1 hour. Median Fluorescent Intensity (MFI) is presented as a function of logarithmized mAb concentration (μg/mL). The mAb concentration presented is the concentration previous to two-fold dilution upon incubation with the microspheres. For final assay mAb concentrations, the presented concentrations need to be divided by two.

FIG. 16 Binding of Anti-ZIKV #1 (A) and 2 (B) to rZIKV-EDIII-2. Incubation of rising mAb concentrations with rZIKV-EDIII-2 coupled to the microspheres at pH 8 was carried out for 1 hour. Median Fluorescent Intensity (MFI) is presented as a function of logarithmized mAb concentration (μg/mL). The mAb concentration presented is the concentration previous to two-fold dilution upon incubation with the microspheres. For final assay mAb concentrations, the presented concentrations need to be divided by two.

FIG. 17 Binding of Anti-ZIKV #3 (A) and 4 (B) to rZIKV-EDIII-2. Incubation of rising mAb concentrations with rZIKV-EDIII-2 coupled to the microspheres at pH 8 was carried out for 1 hour. Median Fluorescent Intensity (MFI) is presented as a function of logarithmized mAb concentration (μg/mL). The mAb concentration presented is the concentration previous to two-fold dilution upon incubation with the microspheres. For final assay mAb concentrations, the presented concentrations need to be divided by two.

FIG. 18 Binding of Anti-ZIKV #5 (A) and 6 (B) to rZIKV-EDIII-2. Incubation of rising mAb concentrations with rZIKV-EDIII-2 coupled to the microspheres at pH 8 was carried out for 1 hour. Median Fluorescent Intensity (MFI) is presented as a function of logarithmized mAb concentration (μg/mL). The mAb concentration presented is the concentration previous to two-fold dilution upon incubation with the microspheres. For final assay mAb concentrations, the presented concentrations need to be divided by two.

FIG. 19 Binding of Anti-ZIKV #7 (A) and Anti-PanDENV1-4 EDIII (B) to rZIKV-EDIII-2. Incubation of rising mAb concentrations with rZIKV-EDIII-2 coupled to the microspheres at pH 8 was carried out for 1 hour. Median Fluorescent Intensity (MFI) is presented as a function of logarithmized mAb concentration (μg/mL). The mAb concentration presented is the concentration previous to two-fold dilution upon incubation with the microspheres. For final assay mAb concentrations, the presented concentrations need to be divided by two.

FIG. 20 Binding of Rabbit IgG (A) and Mouse IgG1 Isotype Control (B) to rZIKV-EDIII-2. Incubation of rising mAb concentrations with rZIKV-EDIII-2 coupled to the microspheres at pH 8 was carried out for 1 hour. Median Fluorescent Intensity (MFI) is presented as a function of logarithmized mAb concentration (μg/mL). The mAb concentration presented is the concentration previous to two-fold dilution upon incubation with the microspheres. For final assay mAb concentrations, the presented concentrations need to be divided by two.

FIG. 21 Determination of coupling efficiency of rZIKV-EDIII-2 to the microspheres. Coupling efficiency of rZIKV-EDIII-2 to microspheres after 2 hours at room temperature was evaluated at pH 8 and 9 using an anti-His tag phycoerythrin (PE)-conjugated detection Ab. Median Fluorescent Intensity (MFI) is presented for both tested pH values.

FIG. 22 Binding of Anti-Flavivirus #1 (A) and 2 (B) to ZIKV VLP and DENV1-4 VLPs. Incubation of rising mAb concentrations with ZIKV VLP and DENV1-4 VLPs coupled to the microspheres at pH 6 (DENV1-4 VLPs) or pH 7 (ZIKV VLP), respectively, was carried out for 1 hour. Median Fluorescent Intensity (MFI) is presented as a function of logarithmized mAb concentration (μg/mL). The mAb concentration presented is the concentration previous to two-fold dilution upon incubation with the microspheres. For final assay mAb concentrations, the presented concentrations need to be divided by two.

FIG. 23 Binding of Anti-ZIKV E Protein (A) and Anti-PanDENV1-4 EDIII (B) to ZIKV VLP and DENV1-4 VLPs. Incubation of rising mAb concentrations with ZIKV VLP and DENV1-4 VLPs coupled to the microspheres at pH 6 (DENV1-4 VLPs) or pH 7 (ZIKV VLP), respectively, was carried out for 1 hour. Median Fluorescent Intensity (MFI) is presented as a function of logarithmized mAb concentration (μg/mL). The mAb concentration presented is the concentration previous to two-fold dilution upon incubation with the microspheres. For final assay mAb concentrations, the presented concentrations need to be divided by two.

FIG. 24 Analysis of human serum negative control by a reporter virus particle (RVP) assay. Different sample dilutions were examined in the RVP and plotted against the observed raw relative luciferase units (RFU). In addition to the human serum negative control, a RVP positive and RVP negative control was included. “1:X Dilution” refers to the fold-dilution of the sample. For instance, a 10-fold dilution means 1:10 dilution and the corresponding x-axis value is then calculated by log (10).

FIG. 25 Total anti-ZIKV IgG levels examined by the microsphere immunoassay (MIA). Total anti-ZIKV IgG levels in human plasma samples #1 to 4 were examined by the MIA using ZIKV VLP, as well as rZIKV-EDIII-1 as antigens coupled to the microspheres. Median Fluorescence Intensity (MFI) is presented for each analyzed sample. Plasma samples #1 and 2 were expected to be ZIKV low-reactive, plasma samples #3 and 4 to be ZIKV high-reactive from the RVP data. In addition, a negative control was analyzed, lacking anti-flavivirus Abs. Samples were diluted to result in final assay dilutions of 1:10, 1:100, 1:1,000, and 1:10,000. (A) Human plasma samples #1 to 4 showed dose-dependent binding to the ZIKV VLPs. In contrast, negative control did not bind over the entire dilution range. ZIKV VLPs were coupled to the microspheres at pH 7. (B) All plasma samples, as well as the negative control showed dose-dependent binding towards the rZIKV-EDIII-1 coupled to the microspheres at pH 6. “1:X Dilution” refers to the fold-dilution of the sample. For instance, a 10-fold dilution means 1:10 dilution and the corresponding x-axis value is then calculated by log (10).

FIG. 26 Total anti-ZIKV IgG levels examined by the microsphere immunoassay (MIA). Total anti-ZIKV IgG levels in human plasma samples #1 to 4 were examined by the MIA using rZIKV-EDIII-2 and 3 as antigens coupled to the microspheres. Median Fluorescence Intensity (MFI) is presented for each analyzed sample. Plasma samples #1 and 2 were expected to be ZIKV low-reactive, plasma samples #3 and 4 to be ZIKV high-reactive from the RVP data. In addition, a negative control was analyzed, lacking anti-flavivirus Abs. (A) Sample binding was evaluated for rZIKV-EDIII-2 coupled to the microspheres at pH 8 and 9, respectively. The two blank wells containing buffer only instead of plasma sample are presented as well. Except for plasma sample #3, sample binding tendencies were similar independent of the pH value applied for coupling. (B) Sample binding was evaluated for rZIKV-EDIII-3 coupled to the microspheres at pH 5, 6, and 7, respectively. The MFI value resulting from the two blank wells containing buffer only instead of plasma sample is presented as well. Except for negative control, samples binding tendencies were similar independent of the pH value applied for coupling.

FIG. 27 Effect of heat-inactivation of human samples in the microsphere immunoassay (MIA). Total anti-ZIKV IgG levels in human plasma samples #1 to 4 were examined by the MIA using ZIKV VLP as antigen coupled to the microspheres. Median Fluorescence Intensity (MFI) is presented for each analyzed sample in dependency of the sample dilution. In addition, a negative control was analyzed, lacking anti-flavivirus Abs. All samples were analyzed with and without heat-inactivation (HI). “Sample Dilution 1:X” refers to the fold-dilution of the sample. For instance, a 10-fold dilution means 1:10 dilution and the corresponding x-axis value is then calculated by log (10).

FIGS. 28-35 Detection of ZIKV specific Abs by the competitive microsphere immunoassay (cMIA). ZIKV specific Abs were evaluated in human plasma samples #1 to 4, as well as negative control by a cMIA using ZIKV VLP coupled to the microspheres at pH 7 and Anti-ZIKV #1 (FIG. 28), Anti-ZIKV #2 (FIG. 29), Anti-ZIKV #3 (FIG. 30), Anti-ZIKV #4 (FIG. 31), Anti-ZIKV #5 (FIG. 32), Anti-ZIKV #6 (FIG. 33), Anti-ZIKV #7 (FIG. 34), and Anti-PanDENV1-4 EDIII (FIG. 35). Plasma samples #1 and 2 were expected to be ZIKV low-reactive, samples #3 and 4 to be ZIKV high-reactive based on the RVP data. ZIKV high-reactive samples were expected to contain ZIKV specific Abs. Negative control was human serum free of anti-flavivirus Abs. Samples were applied at 5-, 10-, and 20-fold dilutions (1:5, 1:10, and 1:20) previous to diluting with the microspheres. Final assay dilutions are consequently 1:10, 1:20, and 1:40. In addition, MFI values were recorded for a control containing solely assay buffer (no plasma sample, no mAb) which refers to 0% mAb binding, as well as for a control containing mAb, but no plasma sample, which refers to 100% mAb binding. (A) Median Fluorescence Intensity (MFI) is presented for each sample dilution. (B) MFI values were divided by the MFI value corresponding to 100% mAb binding to result in the percentage of blockade of mAb binding (blockade-of-binding) by each plasma sample dilution. Values below 0%, mainly observed for the negative control or ZIKV low-reactive samples, were fixed to 0% for visualization in a 0-100% scale.

FIG. 36 Detection of ZIKV specific Abs by the competitive microsphere immunoassay (cMIA). ZIKV specific Abs were evaluated in human plasma samples #1 to 4, as well as negative control by a cMIA using Anti-ZIKV #1 and 6 and rZIKV-EDIII-3 coupled to the microspheres at pH 6. Plasma samples #1 and 2 were expected to be ZIKV low-reactive, samples #3 and 4 to be ZIKV high-reactive based on the RVP data. ZIKV high-reactive samples were expected to contain ZIKV specific Abs. Negative control was human serum free of anti-flavivirus Abs. Plasma samples were applied at a 10-fold final dilution in the asssay. In addition, MFI values were recorded for a control containing solely assay buffer (no plasma sample, no mAb) which refers to 0% mAb binding, as well as for a control containing mAb, but no plasma sample, which refers to 100% mAb binding. Net Median Fluorescence Intensity (MFI) is presented for each plasma sample dilution.

FIG. 37 Quantitative competitive MIA (cMIA) using anti-ZIKV #7. ZIKV specific Abs were evaluated in human plasma samples #1 to 4, as well as negative control by a cMIA using ZIKV VLP coupled to the microspheres at pH 7. Plasma samples #1 and 2 were expected to be ZIKV low-reactive, samples #3 and 4 to be ZIKV high-reactive based on the RVP data. ZIKV high-reactive samples were expected to contain ZIKV specific Abs. Negative control was human serum free of anti-flavivirus Abs. Median Fluorescence Intensity (MFI) is presented for each plasma sample dilution. In addition, the cut-off line is presented, referring to blocking of mAb binding to 40%. “Sample Dilution 1:X” refers to the fold-dilution of the sample. For instance, a 10-fold dilution means 1:10 dilution and the corresponding x-axis value is then calculated by log (10). FIG. 37A shows the MFI values in dependency of the final sample dilution after the 2-fold dilution with the microspheres and FIG. 37B shows the MFI values in dependency of the sample dilution prior to 2-fold dilution with the microspheres.

FIGS. 38-41A Analysis of human samples in a quantitative cMIA using anti-ZIKV #7. ZIKV specific Abs were evaluated in different human samples comprising anti-ZIKV Abs (+ZIKV #1-5 H), anti-DENV Abs (+DENV #1-3 H), anti-YFV Abs (+YFV H), anti-SLEV Abs (+SLEV #1-2 H), or anti-WNV Abs (+WNV #1-7 H), as well as in human samples comprising both, anti-ZIKV and anti-DENV Abs (+ZIKV/+DENV H) or anti-WNV and anti-DENV Abs (+WNV/+DENV H), respectively. A human sample lacking anti-flavivirus Abs was additionally included for comparison (FV-Naïve control). MFI values were recorded in dependency of the sample dilution. In addition, the cut-off line is presented, referring to blocking of mAb binding to 40%. The numbers (for instance, #1) indicate the sample number analyzed. For instance, five different human plasma samples comprising anti-ZIKV Abs were evaluated. “Sample Dilution 1:X” refers to the fold-dilution of the sample. For instance, a 10-fold dilution means 1:10 dilution and the corresponding x-axis value is then calculated by log (10).

FIG. 41B Analysis of non-human primate samples in a quantitative cMIA using anti-ZIKV #7. ZIKV specific Abs were evaluated in samples from three different rhesus macaques (ZIKV Inf. #1-3 NHP) after primary ZIKV infection. A human sample lacking anti-flavivirus Abs (FV-Naïve control) and a human sample comprising both, anti-ZIKV and anti-DENV Abs (+ZIKV/+DENV), were additionally included for comparison. MFI values were recorded in dependency of the sample dilution. In addition, the cut-off line is presented, referring to blocking of mAb binding to 40%. The sample from animal 1 (ZIKV Inf. #1 NHP) was taken 89 days post infection, the samples from the other two animals 118 days post infection. “Sample Dilution 1:X” refers to the fold-dilution of the sample. For instance, a 10-fold dilution means 1:10 dilution and the corresponding x-axis value is then calculated by log (10).

FIGS. 42-43 Analysis of non-human primate samples in a quantitative cMIA using anti-ZIKV #7. ZIKV specific Abs were evaluated in samples from different rhesus macaques vaccinated with either a PIZV (PIZV #1-2 NHP), an YFV vaccine (YFV Vac. pre PIZV #1-2 NHP), a JEV vaccine (JEV Vac. pre PIZV #1-2 NHP), a WNV vaccine (WNV Vac. pre PIZV #1-2 NHP), or a TBEV vaccine (TBEV Vac. pre PIZV #1-2 NHP). Per vaccine, two animals were vaccinated (designated as #1 and #2, respectively). Samples from the animals vaccinated with the PIZV were taken 90 days post vaccination. A human sample lacking anti-flavivirus Abs (FV-Naïve control) and a human sample comprising both, anti-ZIKV and anti-DENV Abs (+ZIKV/+DENV), were additionally included for comparison. MFI values were recorded in dependency of the sample dilution. In addition, the cut-off line is presented, referring to blocking of mAb binding to 40%. “Sample Dilution 1:X” refers to the fold-dilution of the sample. For instance, a 10-fold dilution means 1:10 dilution and the corresponding x-axis value is then calculated by log (10).

FIGS. 44-45 Analysis of non-human primate samples in a quantitative cMIA using anti-ZIKV #7. ZIKV specific Abs were evaluated in samples from different rhesus macaques first vaccinated with either an YFV vaccine (YFV Vac. post PIZV #1-2 NHP), a JEV vaccine (JEV Vac. post PIZV #1-2 NHP), a WNV vaccine (WNV Vac. post PIZV #1-2 NHP), or a TBEV vaccine (TBEV Vac. post PIZV #1-2 NHP) (see FIGS. 42-43) and subsequently vaccinated with two doses of PIZV. In addition, samples from animals vaccinated with a PIZV (PIZV #1-2 NHP) were included taken 252 days post vaccination. Per vaccine, two animals were vaccinated (designated as #1 and #2, respectively). A human sample lacking anti-flavivirus Abs (FV-Naïve control) and a human sample comprising both, anti-ZIKV and anti-DENV Abs (+ZIKV/+DENV), were additionally included for comparison. MFI values were recorded in dependency of the sample dilution. In addition, the cut-off line is presented, referring to blocking of mAb binding to 40%. “Sample Dilution 1:X” refers to the fold-dilution of the sample. For instance, a 10-fold dilution means 1:10 dilution and the corresponding x-axis value is then calculated by log (10).

FIG. 46 ZIKV-specific blockade titers for human samples (marked with “H”) and non-human primate samples (marked with “NHP”). Titers were determined in human samples comprising anti-ZIKV Abs (+ZIKV H), anti-DENV Abs (+DENV H), anti-YFV Abs (+YFV H), anti-SLEV Abs (+SLEV H), or anti-WNV Abs (+WNV H), as well as in human samples comprising both, anti-ZIKV and anti-DENV Abs (+ZIKV/+DENV H) or anti-WNV and anti-DENV Abs (+WNV/+DENV H). In addition, titers were determined in non-human primate samples from animals after natural ZIKV infection (ZIKV Inf. NHP) and after natural DENV infection (DENV nat NHP), as well as animals after vaccination with a DENV, YFV, JEV, WNV, or TBEV vaccine, respectively (DENV/YFV/JEV/WNV/TBEV vac NHP). In addition, titers were determined in samples from the non-human primates vaccinated with the DENV, YFV, JEV, WNV, or TBEV vaccine, respectively, and subsequently vaccinated with a PIZV (YFV/JEV/WNV/TBEV vac/PIZV NHP). Moreover, a human sample and a non-human primate sample that not contain anti-flavivirus Abs were included (FV-Naïve H, FV-Naïve NHP).

FIGS. 47-49 Neutralizing Ab titers and ZIKV-specific blockade titers in samples from four non-human primates after ZIKV primary infection (designated as ZIKV Inf. #1-4 NHP) in dependency of the days after ZIKV infection. Neutralizing titers are presented as EC₅₀ RVP titer determined in a ZIKV RVP assay. ZIKV-specific blockade titers were determined in a quantitative cMIA using anti-ZIKV #7.

FIGS. 50-52 Analysis of non-human primate (NHP) and human samples in a quantitative cMIA using anti-ZIKV #1-5. ZIKV specific Abs were evaluated in pooled samples from rhesus macaques vaccinated with a purified inactivated zika vaccine (PIZV NHP pool), in pooled samples from rhesus macaques after primary DENV infection (DENV nat NHP pool), and in pooled human samples comprising anti-WNV Abs (+WNV (human pool)). A human sample lacking anti-flavivirus Abs (FV-Naïve control) and a human sample comprising both, anti-ZIKV and anti-DENV Abs (+ZIKV/+DENV), were additionally included for comparison. MFI values were recorded in dependency of the sample dilution. In addition, the cut-off line is presented, referring to blocking of mAb binding to 40%. “Sample Dilution 1:X” refers to the fold-dilution of the sample. For instance, a 10-fold dilution means 1:10 dilution and the corresponding x-axis value is then calculated by log (10).

FIGS. 53-55 Binding of Anti-ZIKV #8 (FIG. 53A), #9 (FIG. 53B), #10 (FIG. 54A), #11 (FIG. 54B), Clone 278-11 (FIG. 55A), and Clone 78-2 (FIG. 55B) to ZIKV VLP and DENV1-4 VLPs. Incubation of rising mAb concentrations with ZIKV VLP and DENV1-4 VLPs coupled to the microspheres at pH 6 (DENV1-4 VLPs) or pH 7 (ZIKV VLP), respectively, was carried out for 2 hours. Median Fluorescent Intensity (MFI) is presented as a function of logarithmized mAb concentration (μg/mL). The mAb concentration presented is the concentration previous to two-fold dilution upon incubation with the microspheres. For final assay mAb concentrations, the presented concentrations need to be divided by two.

### DETAILED DESCRIPTION

### Microsphere Complex

It is an object of the present invention to provide an immobilizable binding partner for anti-ZIKV Abs. Such binding partners are required to bind and thereby to detect anti-ZIKV Abs. These binding partners are immobilized to allow detection of the binding in immunoassays. The usual immobilization is carried out on a plate as is e.g. known from the enzyme linked immunosorbent assay (ELISA) setting. Such a set-up including a plate and an enzyme-based detection, however, has certain disadvantages. These disadvantages include the risk of false results due to insufficient blocking, the risk that the activity of the enzyme used for detection (e.g. horseradish peroxidase) may be hampered by sample constituents, time-consuming operation (multiple steps required including washing procedures), as well as a separate assay requirement for each antigen to be analyzed, thereby requirement of larger amounts of reagents. Moreover, the colorimetric readout of the ELISA often lacks sensitivity as enzyme amplification is required and therefore is prone to variability and errors in the amount of amplification. Therefore, assays based on antigen immobilization on microspheres as in Microsphere Immune Assays (MIAs), such as the Luminex set-up, have been developed, which have the advantages of high specificity and reactivity, flexibility to single- or multiplex antigens from different viruses in one single experiment, the possibility for high-throughput, cost-effectiveness, low sample volumes and short turnaround times. Moreover, the data are more accurate and reliable compared to conventional methods such as ELISA, as the data are calculated from the mean of hundreds of microspheres, each of which functions as an individual replicate. The immobilization of an appropriate antigen on such a microsphere is, however, challenging and the precondition to use the advantages of the MIA setting.

The present invention therefore provides a microsphere complex comprising a microsphere coupled to a zika antigen, in particular in the form of a microsphere coupled to a zika virus like particle (ZIKV VLP).

### Microsphere

The microsphere useful for the invention ranges in the size from about 0.01 to about 100 μm in diameter, more preferably from about 1 to about 20 μm, and most preferably a microsphere has a diameter from about 5 to about 7 μm. In a preferred embodiment the microsphere has a diameter of about 6.5 μm. The size of a microsphere can be determined in practically any flow cytometry apparatus by so-called forward or small-angle scatter light.

The microsphere may be constructed of any material to which molecules like VLPs or EDIII may be attached to. For example, acceptable materials for the construction of microspheres include but are not limited to: polystyrene, polyacrylic acid, polyacrylonitrile, polyacrylamide, polyacrolein, polybutadiene, polydimethylsiloxane, polyisoprene, polyurethane, polyvinylacetate, polyvinylchloride, polyvinylpyridine, polyvinylbenzylchloride, polyvinyltoluene, polyvinylidene chloride, polydivinylbenzene, polymethylmethacrylate, or combinations thereof. In a preferred embodiment of the present invention, microspheres are constructed of polystyrene.

The microsphere may comprise affinity groups for attachment of molecules, such as antigens of the present invention. Said affinity groups may be, but are not limited to, Ni²⁺ (for immobilization of His-tagged molecules like EDIII), Protein A, Protein G, Protein L, anti-human IgG Ab, anti-rabbit IgG Ab, anti-mouse IgG Ab, anti-goat IgG Ab, anti-FLAG Ab, streptavidin, avidin, and glutathione.

The microsphere may comprise functional groups on the surface useful for attachment of molecules, such as the antigens of the present invention. Said functional groups may be, but are not limited to, carboxylates, esters, alcohols, carbamides, aldehydes, amines, sulfur oxides, nitrogen oxides, maleimides, or halides. In a preferred embodiment the microsphere comprises carboxylates on the surface. Molecules like antigens can be covalently coupled to the microspheres using chemical techniques described herein or in the prior art (e.g. Bruckner, Springer Verlag 2010, Organic Mechanisms; Angeloni et al., xMAP Cookbook, Luminex, 4^{th} edition). In a preferred embodiment molecules like antigens (i.e. VLPs or EDIII) can be coupled to the microsphere by carbodiimide coupling using 1-Ethyl-3-(3-dimethylaminopropyl)carbodiimide (EDC) and N-hydroxysulfosuccinimide (Sulfo-NHS). Thereby, the EDC is reacting to an unstable o-acylisourea ester with a carboxylate on the surface of the microspheres. The unstable o-acylisourea ester readily reacts with Sulfo-NHS to form a semi-stable amine reactive NHS-ester. The NHS-ester finally reacts with an amine group provided by an antigen, thereby forming a stable amide bond.

As amine-containing compounds other than those provided by the antigen, glycerol, urea, imidazole, azide, and some detergents may interfere with the carbodiimide coupling, they should be removed from the antigen preparation with a suitable buffer exchange method. For instance, a suitable buffer for carbodiimide coupling is 50 mM 2-(N-morpholino)ethanesulfonic acid (MES) buffer. The pH value for coupling may be between about 5 and about 9. Coupling of the antigen to the microsphere may be carried out by incubation for about 2 hours.

The microsphere may be magnetic. In a preferred embodiment, the microsphere may be superparamagnetic. Magnetic microspheres can be easily captured by a magnetic plate separator for instance to wash the microspheres. A magnetic plate separator can be used for separating the microspheres within the 96-well plate from the solution within the wells of the 96-well plate by magnetic capture and refers to a construction for holding a 96-well plate. A magnetic plate separator enables the user to quickly decant the supernatant within the wells and washing of the wells, while fixing the microspheres at the bottom of the 96-well plate by magnetic capture. Application of a magnetic plate separator as is possible in the MIA set-up reduces the risk that microspheres are getting lost during washing procedures.

The microsphere may be identified by a specific feature. This specific feature refers to a specific property of the microsphere, which allows it to be identified by a detection instrument. Identification of a microsphere likewise allows identification of the antigen, which is coupled to the microsphere. The specific feature may be that the microsphere comprises one or more fluorescent dyes having a specific emission spectrum and/or that the microsphere may comprise one or more fluorescent dyes at a specific concentration and/or that the microsphere is of a certain size.

The microsphere may be part of a microsphere set. A microsphere set refers to a plurality of microspheres that share the same specific feature. Microspheres of other sets are characterized by different specific features as one or more fluorescent dyes having another specific emission spectrum and/or one or more fluorescent dyes at another specific concentration and/or another certain size. By detection of the specific feature, a microsphere can be identified as part of a set and distinguished from microspheres of other sets. If the specific feature is that the microsphere may be of a certain size, all microspheres within the same set should be relatively the same size and all microspheres not part of this specific microsphere set should be of a different size. Microspheres of different microsphere sets can be coupled to different antigens. These antigens may comprise VLPs or EDIII of different flaviviruses. The unique specific features of the microspheres being part of different microsphere sets allow the different microspheres to be distinguished from each other. Moreover, when coupling different antigens to microspheres of different microsphere sets, the microspheres of the sets can be mixed, retaining the ability to be distinguished by their specific feature and thereby to identify the coupled antigen. This allows for multiplexing e.g. the simultaneous determination of the coupled antigens and/or the presence and/or amount of different Abs directed to the different antigens in one single experiment.

In one embodiment, the specific feature is that the microsphere is of a certain size ranging from about 0.01 to about 100 μm in diameter, more preferably from about 1 to about 10 μm in diameter. For instance, microspheres of one microsphere set may be about 6 μm in diameter, microspheres of another microsphere set may be about 6.5 μm in diameter.

In other embodiments, the specific feature is that the microsphere comprises one or more fluorescent dyes having a specific emission spectrum and/or comprises one or more fluorescent dyes at a specific concentration.

For instance, microspheres of one microsphere set may comprise two fluorescent dyes having an emission spectrum with an emission maximum at 675 nm, microspheres of another microsphere set may comprise two fluorescent dyes having an emission spectrum with an emission maximum at 700 nm. In another example, microspheres of one microsphere set may comprise a fluorescent dye at one specific concentration, microspheres of another microsphere set may comprise a fluorescent dye at another specific concentration, therefore providing two emission spectra with different emission intensities.

In other embodiments the specific feature is that the microsphere comprises one or more fluorescent dyes having a specific emission spectrum.

In one embodiment, the fluorescent dyes of the microspheres being part of different microsphere sets allow for excitation of the fluorescent dyes by the same light source (e.g. the fluorescent dyes of microspheres being part of different microsphere sets are excitable by the same wavelength). In specific embodiments, the microspheres being part of different microsphere sets are excitable with a wavelength within the range from about 600 nm to about 650 nm, more preferably with a wavelength of about 615 nm to about 640 nm, and even more preferably with a wavelength of about 620 nm to about 635 nm. In one embodiment, the different microspheres are excitable with a wavelength of about 621 nm. An advantage of such a set-up is, that only one light source is needed for distinguishing all microspheres present within a microsphere mixture and thereby further simplifying a set-up in which multiple virus like particles can be applied in one single experiment (multiplexing set-up).

Microspheres may be one out of the list consisting of MagPlex® microspheres, MicroPlex® microspheres, LumAvidin® microspheres, MagPlex®-Avidin microspheres, and SeroMAP® microspheres produced by the Luminex Corporation (Austin, Texas). In a preferred embodiment, the microspheres are the MagPlex® microspheres, which are superparamagnetic polystyrene microspheres with surface carboxyl groups produced by Luminex Corporation (Austin, Texas). MagPlex® microspheres comprise one or more fluorescent dyes having a specific emission spectrum as the specific feature allowing each microsphere to be identified by a detection instrument as for instance a MAGPIX® instrument as produced by the Luminex Corporation (Austin, Texas). Different MagPlex® microsphere catalog numbers (Luminex Corporation, Austin, Texas) refer to different microsphere sets, wherein the microspheres of the different sets comprise one or more fluorescent dyes having different specific emission spectra. The emission spectra of MagPlex® microspheres allow for excitation of the different fluorescent dyes by the same light source and therefore for simultaneous detection of the microspheres by one excitation wavelength. In specific embodiments, the excitation wavelength is from about 600 nm to about 650 nm, more preferably from about 620 nm to about 640 nm, such as about 621 nm.

### ZIKV VLP

The antigen coupled to the microsphere according to the invention is a ZIKV VLP. The ZIKV VLP is derived from a ZIKV strain. ZIKV strains include but are not limited to ZikaSPH (Brazil 2015, GenBank accession No. KU321639.1), Brazil-ZKV (Brazil 2015, GenBank accession No. KU497555.1), PRVABC59 (Puerto Rico 2015, GenBank accession No. KU501215.1), Haiti1225 (Haiti 2014, GenBank accession No. KU509998.1), Natal RGN (Brazil, GenBank accession No. KU527068.1), SV0127-14 (Thailand 2014, GenBank accession No. KU681081.3), SPH2015 (GenBank accession No. KU321639.1), CPC-0740 (Philippine 2012, GenBank accession No. KU681082.3), SSABR1 (Brazil, GenBank accession No. KU707826.1), VE_Ganxian (China, GenBank accession No. KU744693.1), MR766-NIID (Uganda, GenBank accession No. LC002520.1), MR 766 (Uganda 1947, GenBank accession No. AY632535.2), and H/PF (French Polynesia 2013, GenBank accession No KJ776791.1) (WO 2017/109225). Further, ZIKV strains include Cambodia 2010 (GenBank accession No JN860885) or Micronesia 2007 (GenBank accession No EU545988) (Mlakar et al., N Engl J Med. 2016 Mar. 10; 374(10):951-8). Further, ZIKV strains include FLR (Colombia 2015) strain (WO 2018/017497), Z1106031 isolated in Suriname (Asian genotype; GenBank accession No KU312314), Z1106027 isolated in Suriname (Asian genotype; GenBank accession No KU312315); Z1106032 isolated in Suriname (Asian genotype; GenBank accession No KU312313), and Z1106033 isolated in Suriname (Asian genotype; Enfissi et al., Lancet 2016, 387(10015):227-228; GenBank Accession No. KU312312.1, SEQ ID NO: 1 and SEQ ID NO: 2).

According to one embodiment of the invention the ZIKV VLP is derived from ZIKV strain Z1106033 characterized by SEQ ID NO: 1 and/or SEQ ID NO: 2.

According to one embodiment of the invention the ZIKV VLP comprises structural proteins of ZIKV strain Z1106033 characterized by SEQ ID NO: 1 and/or SEQ ID NO: 2.

According to one embodiment of the invention the ZIKV VLP comprises the envelope glycoprotein, membrane protein, and/or pre-membrane protein of ZIKV strain Z1106033 characterized by SEQ ID NO: 1 and/or SEQ ID NO: 2.

According to one embodiment of the invention the ZIKV VLP comprises the envelope glycoprotein, membrane protein, and pre-membrane protein of ZIKV strain Z1106033 characterized by SEQ ID NO: 1 and/or SEQ ID NO: 2.

According to one embodiment, the ZIKV VLP is produced in human embryonic kidney (HEK293) cells.

In one embodiment, the ZIKV strain is Z1106033 isolated in Suriname (Asian genotype; Enfissi et al., Lancet 2016, 387(10015):227-228; GenBank Accession No. KU312312.1, SEQ ID NO: 1 and SEQ ID NO: 2) and the ZIKV VLP is produced in HEK293 cells.

Within the meaning of this invention, a ZIKV VLP comprising the envelope glycoprotein (E protein), membrane (M) protein, and/or pre-membrane (prM) protein of a ZIKV strain such as the Z1106033 strain refers to a ZIKV VLP comprising an envelope glycoprotein, membrane protein, and/or pre-membrane protein which are at least 70%, or at least 75%, or at least 80%, or at least 85%, or at least 90%, or at least 95% or 100% identical to corresponding parts of the protein sequence of the ZIKV strain such as the Z1106033 strain (SEQ ID NO: 2). Corresponding parts within that context mean parts of the protein sequence (such as SEQ ID NO: 2 for Z1106033 strain) that encode for E protein, M protein, or prM protein, respectively.

### Kit of Microsphere Complex and Reporter Antibody

The present invention is directed to a kit comprising an amount of a microsphere complex as described in the previous chapter with the heading “Microsphere complex” and an amount of a reporter antibody that binds to the zika virus like particle (ZIKV VLP) of the microsphere complex.

Regarding the microsphere complex reference is made to the previous chapter with the heading “Microsphere complex”.

According to one embodiment of the invention, the reporter antibody is an immunoglobulin (Ig) molecule, comprised of four polypeptide chains, two heavy (H) chains and two light (L) chains interconnected by disulfide bonds

According to one embodiment of the invention, the reporter Ab is a zika virus neutralizing antibody.

According to one embodiment of the invention, the reporter Ab is a recombinant Ab.

According to one embodiment the reporter Ab does not cross-react with dengue virus antigens.

According to one embodiment the reporter Ab does not cross-react with flavivirus antigens, such as DENV antigens, WNV antigens, JEV antigens, YFV antigens, SLEV antigens, and/or TBEV antigens.

According to one embodiment of the invention, the reporter antibody is a monoclonal antibody.

According to one embodiment of the invention, the reporter antibody is derived from a non-human origin.

According to one embodiment of the invention, the reporter Ab is attached to at least one detectable label. In preferred embodiments, the reporter Ab is attached to at least one detectable label by the heavy chain constant region of the reporter Ab.

According to one specific embodiment of the invention, the reporter Ab is directly attached to at least one detectable label. In preferred embodiments, the reporter Ab is directly attached to at least one detectable label by the heavy chain constant region of the reporter Ab. In embodiments, wherein the reporter Ab is directly attached to at least one detectable label, no secondary report Ab is necessary for detection of the reporter Ab.

According to another specific embodiment of the invention, the reporter Ab is indirectly attached to at least one detectable label, wherein the reporter Ab reacts with a secondary reporter Ab directly attached to at least one detectable label. In even more specific embodiments, the reporter Ab is indirectly attached to at least one detectable label by the heavy chain constant region of the reporter Ab, wherein the reporter Ab reacts with a secondary reporter Ab directly attached to at least one detectable label.

According to one embodiment of the invention, the detectable label is a compound or moiety that comprises one or more appropriate chemical substances or enzymes, which directly or indirectly generate a detectable compound or signal in a chemical, physical or enzymatic reaction. Labeling can be achieved by methods well known in the art (see, for example, Lottspeich, F., and Zorbas H., Springer Spektrum 2012, Bioanalytik).

According to one embodiment of the invention, the detectable label is selected from the group of fluorescent labels, magnetic labels, enzyme labels, colored labels, chromogenic labels, luminescent labels, radioactive labels, haptens, biotin, metal complexes, metals, and colloidal gold. All these types of labels are well established in the art.

According to one embodiment of the invention, the label is selected from such which provide the emission of fluorescence or phosphorescence upon irradiation or excitation or the emission of X-rays when using a radioactive label.

According to one embodiment of the invention, the label is an enzyme label, which include but are not limited to alkaline phosphatase, horseradish peroxidase (HRP), β-galactosidase, and p-lactamase. Enzyme labels catalyze the formation of chromogenic reaction products.

In specific embodiments, the detectable labels are fluorescent labels. Numerous fluorescent labels are well established in the art and commercially available from different suppliers (see, for example, The Handbook—A Guide to Fluorescent Probes and Labeling Technologies, 10th ed. (2006), Molecular Probes, Invitrogen Corporation, Carlsbad, CA, USA). Examples of fluorescent labels include but are not limited to fluorescein isothiocyanate (FITC), rhodamine, phycoerythrin (PE), cyanine, or coumarin. For detecting such labels any suitable detection system may be used.

According to one embodiment of the invention, the label is a fluorescent label such as PE.

Concerning the detection of such labels with suitable detection systems, reference is made to the following chapter with the heading “Method of detecting anti-zika virus antibodies”.

In specific embodiments, the reporter Ab provides an EC₅₀ value towards the ZIKV VLP coupled to the microsphere within the microsphere complex of less than 0.5 μg/mL, or less than 0.4 μg/mL or less than 0.3 μg/mL or less than 0.2 μg/mL or less than 0.15 μg/mL or less than 0.1 μg/mL or less than 0.09 μg/mL or less than 0.08 μg/mL or less than 0.07 μg/mL or less than 0.05 μg/mL or less than 0.04 μg/mL or less than 0.03 μg/mL or less than 0.01 μg/mL. In other specific embodiments, the reporter Ab provides an EC₂₅ value towards the ZIKV VLP coupled to the microsphere within the microsphere complex of less than 0.05 μg/mL, or less than 0.04 μg/mL or less than 0.03 μg/mL or less than 0.02 μg/mL or less than 0.01 μg/mL or less than 0.008 μg/mL or less than 0.006 μg/mL or less than 0.005 μg/mL or less than 0.004 μg/mL or less than 0.003 μg/mL. Examples of EC₅₀ (and EC₂₅ values) determined for the anti-ZIKV Abs as used herein can be found in Table 5. EC₅₀ (and EC₂₅ values) may be determined after incubation of the reporter Ab with the ZIKV VLP for about 10 min, or for about 60 min, or for about 120 min. In preferred embodiments, EC₂₅ values are determined after incubation of the reporter Ab with the ZIKV VLP for about 120 min.

In other embodiments, the reporter antibody is a ZIKV specific reporter Ab, providing an EC₅₀ value towards the zika VLP coupled to the microsphere within the microsphere complex, which is lower than each EC₅₀ value, which said reporter antibody provides when tested in binding towards other microsphere complexes comprising a microsphere coupled to a DENV VLP, such as a DENV1 VLP, and/or DENV2 VLP, and/or DENV3 VLP, and/or DENV4 VLP. According to such embodiments, the difference between the EC₅₀ values may be at least 2-fold, or 3-fold, or 4-fold, or 5-fold, or 8-fold, or 10-fold, or 12-fold, or 15-fold, or 20-fold.

In specific embodiments, the reporter antibody is a ZIKV specific reporter Ab, providing an EC₅₀ value towards the ZIKV VLP coupled to the microsphere within the microsphere complex which is lower than each EC₅₀ value which said reporter antibody provides when tested in binding towards other microsphere complexes comprising a microsphere coupled to DENV1 VLP, and/or DENV2 VLP, and/or DENV3 VLP, and/or DENV4 VLP. According to such embodiments, the difference between the EC₅₀ values may be at least 2-fold, or 3-fold, or 4-fold, or 5-fold, or 8-fold, or 10-fold, or 12-fold, or 15-fold, or 20-fold.

In more specific embodiments, the reporter antibody is a ZIKV specific reporter Ab, providing an EC₅₀ value towards the ZIKV VLP coupled to the microsphere within the microsphere complex of less than 0.5 μg/mL or less than 0.4 μg/mL or less than 0.3 μg/mL or less than 0.2 μg/mL or less than 0.15 μg/mL or less than 0.1 μg/mL or less than 0.09 μg/mL or less than 0.08 μg/mL or less than 0.07 μg/mL or less than 0.05 μg/mL or less than 0.04 μg/mL or less than 0.03 μg/mL or less than 0.01 μg/mL and an EC₅₀ value towards other microsphere complexes comprising a microsphere coupled to DENV VLP, such as a DENV1 VLP, and/or DENV2 VLP, and/or DENV3 VLP, and/or DENV4 VLP of at least 1 μg/mL or of at least 1.1 μg/mL or of at least 1.2 μg/mL or of at least 1.3 μg/mL or of at least 1.4 μg/mL.

Regarding the ZIKV VLP reference is made to the previous chapter entitled “Microsphere complex”.

In specific embodiments of the invention, the DENV1 VLP is derived from DENV1 strain Puerto Rico/US/BID-V853/1998 (GenBank accession No. EU482592.1; SEQ ID NO: 179 and 180). DENV1 VLP may be produced in HEK293 cells. In more specific embodiments the DENV1 VLP comprises structural proteins from DENV1 strain Puerto Rico/US/BID-V853/1998 (GenBank accession No. EU482592.1; SEQ ID NO: 179 and 180). In even more specific embodiments, the DENV1 VLP comprises the E protein, M protein, and/or prM protein which are at least 70%, or at least 75%, or at least 80%, or at least 85%, or at least 90%, or at least 95% or 100% identical to those encoded by DENV1 strain Puerto Rico/US/BID-V853/1998 (GenBank accession No. EU482592.1; SEQ ID NO: 179 and 180).

In specific embodiments of the invention, the DENV2 VLP is derived from DENV2 strain Thailand/16681/84 (EMBL-EBI accession No: U87411.1; SEQ ID NO: 181 and 182). DENV2 VLP may be produced in HEK293 cells. In more specific embodiments the DENV2 VLP comprises structural proteins from DENV2 strain Thailand/16681/84 (EMBL-EBI accession No: U87411.1; SEQ ID NO: 181 and 182). In even more specific embodiments, the DENV2 VLP comprises the E protein, M protein, and/or prM protein which are at least 70%, or at least 75%, or at least 80%, or at least 85%, or at least 90%, or at least 95% or 100% identical to those encoded by DENV2 strain Thailand/16681/84 (EMBL-EBI accession No: U87411.1; SEQ ID NO: 181 and 182).

In specific embodiments of the invention, the DENV3 VLP is derived from DENV3 strain Sri Lanka D3/H/IMTSSA-SRI/2000/1266 (GenBank accession No. AY099336.1; SEQ ID NO: 183 and 184). DENV3 VLP may be produced in HEK293 cells. In more specific embodiments the DENV3 VLP comprises structural proteins from DENV3 strain Sri Lanka D3/H/IMTSSA-SRI/2000/1266 (GenBank accession No. AY099336.1; SEQ ID NO: 183 and 184). In even more specific embodiments, the DENV3 VLP comprises the E protein, M protein, and/or prM protein which are at least 70%, or at least 75%, or at least 80%, or at least 85%, or at least 90%, or at least 95% or 100% identical to those encoded by DENV3 strain Sri Lanka D3/H/IMTSSA-SRI/2000/1266 (GenBank accession No. AY099336.1; SEQ ID NO: 183 and 184).

In specific embodiments of the invention, the DENV4 VLP is derived from DENV4 strain Dominica/814669/1981 (EMBL-EBI accession No: AF326825.1; SEQ ID NO: 185 and 186). DENV4 VLP may be produced in HEK293 cells. In more specific embodiments the DENV4 VLP comprises structural proteins from DENV4 strain Dominica/814669/1981 (EMBL-EBI accession No: AF326825.1; SEQ ID NO: 185 and 186). In even more specific embodiments, the DENV4 VLP comprises the E protein, M protein, and/or prM protein which are at least 70%, or at least 75%, or at least 80%, or at least 85%, or at least 90%, or at least 95% or 100% identical to those encoded by DENV4 strain Dominica/814669/1981 (EMBL-EBI accession No: AF326825.1; SEQ ID NO: 185 and 186).

In specific embodiments of the present invention for production of DENV1-4 VLPs, the C-terminal 20% of DENV E protein were replaced by the corresponding Japanese encephalitis virus (JEV) SA-14 sequence (EMBL-EBI accession No: M55506.1, SEQ ID NO: 177 and 178; E protein amino acids 399-497 (DENV1 VLP), 397-495 (DENV2 VLP), 399-492 (DENV3 VLP), 400-495 (DENV4 VLP)). The replaced sequence corresponds to the transmembrane and intraparticle portion of the protein.

In a preferred embodiment, the EC₅₀ and EC₂₅ values are determined by incubation of the ZIKV VLP and/or DENV VLPs coupled to the microspheres in the microsphere complex with a serial dilution of the reporter Ab and determining the binding of the reporter Ab towards the ZIKV VLP and/or DENV VLP coupled to the microspheres by detecting the signal from the reporter Ab bound to the ZIKV VLP and/or DENV VLP coupled to the microspheres in the microsphere complex.

Concerning the detection of the signal from the reporter Ab bound to the ZIKV VLP and/or DENV VLP coupled to the microspheres in the microsphere complex reference is made to the following chapter with the heading “Method of detecting anti-zika virus antibodies”.

In certain embodiments, for determining the EC₅₀ and/or EC₂₅ values, the serial dilution of the reporter Ab is incubated with the ZIKV VLP and/or DENV VLPs coupled to the microspheres in the microsphere complex for about 10 min previous to determining the binding of the reporter Ab towards the ZIKV VLP and/or DENV VLP coupled to the microspheres by detecting the signal from the reporter Ab bound to the ZIKV VLP and/or DENV VLP coupled to the microspheres in the microsphere complex.

In certain embodiments, for determining the EC₅₀ and/or EC₂₅ values, the serial dilution of the reporter Ab is incubated with the ZIKV VLP and/or DENV VLPs coupled to the microspheres in the microsphere complex for about 120 min previous to determining the binding of the reporter Ab towards the ZIKV VLP and/or DENV VLP coupled to the microspheres by detecting the signal from the reporter Ab bound to the ZIKV VLP and/or DENV VLP coupled to the microspheres in the microsphere complex.

In certain embodiment, for determining the EC₅₀ and/or EC₂₅ values, the serial dilution of the reporter Ab is incubated with the ZIKV VLP and/or DENV VLPs coupled to the microspheres in the microsphere complex for about 60 min previous to determining the binding of the reporter Ab towards the ZIKV VLP and/or DENV VLP coupled to the microspheres by detecting the signal from the reporter Ab bound to the ZIKV VLP and/or DENV VLP coupled to the microspheres in the microsphere complex.

In one embodiment, the serial dilution of the reporter Ab ranges from about 0.0001 to about 100 μg/mL, more preferable from about 0.001 to about 35 μg/mL, and even more preferable from about 0.001 to about 20 μg/mL, wherein the concentrations of the reporter Ab refer to the concentrations previous to 2-fold dilution with the microspheres (see also Example 2).

In one embodiment, the reporter antibody is incubated with a secondary reporter Ab directly attached to at least one detectable label for detecting the signal from the reporter Ab bound to the ZIKV VLP and/or DENV VLP coupled to the microspheres in the microsphere complex.

In another embodiment, the reporter antibody is incubated with a secondary reporter Ab directly attached to at least one detectable label for about 30 min. In a preferred embodiment the at least one detectable label is a fluorescence label. In an even more preferred embodiment, the fluorescence label is PE.

In other embodiments, the reporter Ab is directly attached to at least one detectable label, preferably to at least one fluorescence label, more preferably to PE. In these embodiments, a signal from the reporter Ab bound to the ZIKV VLP and/or DENV VLP coupled to the microspheres in the microsphere complex can be directly detected without the need of further incubation with a secondary reporter Ab.

Concerning the detection of detectable labels, reference is made to the following chapter with the heading “Method of detecting anti-zika virus antibodies”.

In specific embodiments the detected signal for each serial dilution of the reporter Ab is analyzed using a suitable data analysis software e.g. Prism (GraphPad). The detected signals for each reporter antibody are sigmoidal fitted according to a dose-response curve and the EC₅₀ value is calculated for the reporter Ab. The EC₅₀ value is calculated using mAb concentrations previous to 2-fold dilution by incubation with the microspheres.

In certain embodiments, the EC₅₀ value can be reliably determined when a sufficient number of dilutions of reporter Ab is examined per reporter Ab. A sufficient number of dilutions within that context may mean that the number of dilutions is sufficient to reach a plateau for the minimum signal and the maximum signal in the dose-response curve. A sufficient number of dilutions may be about 10 or more.

It may be possible that the reporter Ab binds to the other microsphere complexes such as microsphere complexes comprising microspheres coupled to DENV1 VLP, DENV2 VLP, DENV3 VLP, or DENV4 VLP to such a less extent that the EC₅₀ value cannot be calculated in a statistically reliable way. In such cases, the EC₅₀ value can be assumed to be at least 1 μg/mL.

According to one embodiment, the reporter antibody is one of the Abs selected from the group consisting of anti-ZIKV #1, anti-ZIKV #2, anti-ZIKV #3, anti-ZIKV #4, anti-ZIKV #5, anti-ZIKV #6, anti-ZIKV #7, anti-ZIKV #8, anti-ZIKV #9, anti-ZIKV #10, or anti-ZIKV #11. For further details and characterization of Abs reference is made to Example 2. The reporter antibody may be characterized by the sequence of the VH-CDR1 and/or VH-CDR2 and/or VH-CDR3 and/or VL-CDR1 and/or VL-CDR2 and/or VL-CDR3. The reporter antibody may alternatively or additionally be characterized by the sequence of the VH and/or VL and/or H and/or L. The sequence referred to may be an amino acid sequence or a nucleic acid sequence encoding the amino acid sequence. The sequences and critical amino acid residues for binding are provided in Table 1 and 2, respectively. Critical residues are those amino acids whose side chains make the highest energetic contribution to the Ab-epitope interaction and whose mutation gave the lowest binding reactivities (<10% of wild-type) by alanine scanning mutagenesis (Bogan and Thorn, J. Mol. Biol. 1998, 280, 1-9; Lo Conte et al., J. Mol. Biol. 1999, 285, 2177-2198).

TABLE 1
Sequence information for reporter Abs. Reporter Abs are presented
together with their amino acid sequences, as well as corresponding
nucleotide sequences (where available) of heavy chain (H), heavy
chain variable region (VH), heavy chain complementary determining
regions 1 to 3 (VH-CDR1-3), light chain (L), light chain variable
region (VL), as well as light chain complementary determining
regions 1 to 3 (VL-CDR1-3). H and L sequences were not available
from the vendor, publications, or databases for commercial reporter
Abs (Anti-ZIKV #6 and 7).

| | | | |
|---|---|---|---|
| | | Amino acid | Nucleic acid |
| | mAb | sequence | sequence |
| mAb | part | (SEQ ID No) | (SEQ ID No) |

Examples

| | | | |
|---|---|---|---|
| Anti-ZIKV #1 | H | 5 | 15 |
| | VH | 6 | 16 |
| | VH-CDR1 | 7 | N/A |
| | VH-CDR2 | 8 | N/A |
| | VH-CDR3 | 9 | N/A |
| | L | 10 | 17 |
| | VL | 11 | 18 |
| | VL-CDR1 | 12 | N/A |
| | VL-CDR2 | 13 | N/A |
| | VL-CDR3 | 14 | N/A |
| Anti-ZIKV #2 | H | 19 | 29 |
| | VH | 20 | 30 |
| | VH-CDR1 | 21 | N/A |
| | VH-CDR2 | 22 | N/A |
| | VH-CDR3 | 23 | N/A |
| | L | 24 | 31 |
| | VL | 25 | 32 |
| | VL-CDR1 | 26 | N/A |
| | VL-CDR2 | 27 | N/A |
| | VL-CDR3 | 28 | N/A |
| Anti-ZIKV #3 | H | 33 | 43 |
| | VH | 34 | 44 |
| | VH-CDR1 | 35 | N/A |
| | VH-CDR2 | 36 | N/A |
| | VH-CDR3 | 37 | N/A |
| | L | 38 | 45 |
| | VL | 39 | 46 |
| | VL-CDR1 | 40 | N/A |
| | VL-CDR2 | 41 | N/A |
| | VL-CDR3 | 42 | N/A |
| Anti-ZIKV #4 | H | 47 | 57 |
| | VH | 48 | 58 |
| | VH-CDR1 | 49 | N/A |
| | VH-CDR2 | 50 | N/A |
| | VH-CDR3 | 51 | N/A |
| | L | 52 | 59 |
| | VI | 53 | 60 |
| | VL-CDR1 | 54 | N/A |
| | VL-CDR2 | 55 | N/A |
| | VL-CDR3 | 56 | N/A |
| Anti-ZIKV #5 | H | 61 | 71 |
| | VH | 62 | 72 |
| | VH-CDR1 | 63 | N/A |
| | VH-CDR2 | 64 | N/A |
| | VH-CDR3 | 65 | N/A |
| | L | 66 | 73 |
| | VL | 67 | 74 |
| | VL-CDR1 | 68 | N/A |
| | VL-CDR2 | 69 | N/A |
| | VL-CDR3 | 70 | N/A |
| Anti-ZIKV #6 | VH | 75 | N/A |
| | VH-CDR1 | 76 | N/A |
| | VH-CDR2 | 77 | N/A |
| | VH-CDR3 | 78 | N/A |
| | VL | 79 | N/A |
| | VL-CDR1 | 80 | N/A |
| | VL-CDR2 | 81 | N/A |
| | VL-CDR3 | 82 | N/A |
| Anti-ZIKV #7 | VH | 33 | 91 |
| | VH-CDR1 | 84 | N/A |
| | VH-CDR2 | 85 | N/A |
| | VH-CDR3 | 86 | N/A |
| | VL | 87 | 92 |
| | VL-CDR1 | 88 | N/A |
| | VL-CDR2 | 89 | N/A |
| | VL-CDR3 | 90 | N/A |
| Anti-ZIKV #8 | H | 93 | 103 |
| | VH | 94 | 104 |
| | VH-CDR1 | 95 | N/A |
| | VH-CDR2 | 96 | N/A |
| | VH-CDR3 | 97 | N/A |
| | L | 98 | 105 |
| | VL | 99 | 106 |
| | VL-CDR1 | 100 | N/A |
| | VL-CDR2 | 101 | N/A |
| | VL-CDR3 | 102 | N/A |
| Anti-ZIKV #9 | H | 107 | 117 |
| and #10 | VH | 108 | 118 |
| | VH-CDR1 | 109 | N/A |
| | VH-CDR2 | 110 | N/A |
| | VH-CDR3 | 111 | N/A |
| | L | 112 | 119 |
| | VL | 113 | 120 |
| | VL-CDR1 | 114 | N/A |
| | VL-CDR2 | 115 | N/A |
| | VL-CDR3 | 116 | N/A |
| Anti-ZIKV #11 | H | 121 | 131 |
| | VH | 122 | 132 |
| | VH-CDR1 | 123 | N/A |
| | VH-CDR2 | 124 | N/A |
| | VH-CDR3 | 125 | N/A |
| | L | 126 | 133 |
| | VL | 127 | 134 |
| | VL-CDR1 | 128 | N/A |
| | VL-CDR2 | 129 | N/A |
| | VL-CDR3 | 130 | N/A |

TABLE 2
Critical amino acid residues in one-letter code from the
ZIKV E protein (SEQ ID NO: 3) important for binding of
Anti-ZIKV #1 to 5 mAbs as evaluated by alanine scanning
mutagenesis. T = Thr, G = Gly, E = Glu, K = Lys, H = His.

| | Critical | E Protein |
|---|---|---|
| mAb | Residues | Domain |
| Anti-ZIKV #1 (Clone 102-1) | T309, G337 | III |
| Anti-ZIKV #2 (Clone 242-3) | E370 | III |
| Anti-ZIKV #3 (Clone 270-12) | E370 | III |
| Anti-ZIKV #4 (Clone 289-3) | E162, G181, G182, K301 | I, III |
| Anti-ZIKV #5 (Clone 306-2) | T397, H398 | III |

### Method of Detecting Anti-Zika Virus Antibodies

Regarding the microsphere complex and the kit reference is made to the respective chapters above.

The present invention is further directed to a method for detecting a signal from a reporter antibody indicative for the presence and/or amount of anti-zika virus antibodies in a sample from a subject comprising the steps of:
Step 1: providing a kit comprising an amount of a microsphere complex comprising a microsphere coupled to a zika virus like particle and an amount of a reporter antibody that binds to the zika virus like particle of the microsphere complex,
Step 2: contacting the amount of said microsphere complex and the amount of said reporter antibody of step 1 with the sample to allow binding of anti-zika virus antibodies in the sample to the zika virus like particles coupled to the microspheres in the microsphere complex while competing with the reporter antibody, and
Step 3: detecting a signal from the reporter antibody bound to the zika virus like particles coupled to the microspheres in the microsphere complex in step 2.

According to one specific embodiment, contacting in step 2 is carried out for about 10 to 100 min.

According to one specific embodiment of the invention, the amount of microsphere complex and the amount of reporter antibody are concomitantly contacted with the sample in step 2.

According to one specific embodiment of the invention the method comprises the steps of:
Step 1: providing a kit comprising an amount of a microsphere complex comprising a microsphere coupled to a zika virus like particle and an amount of a reporter antibody that binds to the zika virus like particle of the microsphere complex,
Step 2.1: contacting the amount of said microsphere complex of step 1 with the sample to allow binding of anti-zika virus antibodies in the sample to the zika virus like particles coupled to the microspheres in the microsphere complex,
Step 2.2: contacting said amount of reporter antibody with said microsphere complex and the sample of step 2.1 to allow binding of the reporter antibody to the zika virus like particles coupled to the microspheres in the microsphere complex, and
Step 3: detecting a signal from the reporter antibody bound to the zika virus like particles coupled to the microspheres in the microsphere complex in step 2.2.

According to another specific embodiment of the invention the method comprises the steps of:
Step 1: providing a kit comprising an amount of a microsphere complex comprising a microsphere coupled to a zika virus like particle and an amount of a reporter antibody that binds to the zika virus like particle of the microsphere complex,
Step 2.1: contacting the amount of said microsphere complex of step 1 with the amount of said reporter antibody to allow binding of the reporter antibody to the zika virus like particles coupled to the microspheres in the microsphere complex,
Step 2.2: contacting the sample with said microsphere complex and said reporter antibody of step 2.1 to allow binding of anti-zika virus antibodies in the sample to the zika virus like particles coupled to the microspheres in the microsphere complex, and
Step 3: detecting a signal from the reporter antibody bound to the zika virus like particles coupled to the microspheres in the microsphere complex in step 2.1.

According to one specific embodiment of the invention the method comprises the steps of:
Step 1: providing a kit comprising an amount of a microsphere complex comprising a microsphere coupled to a zika virus like particle and an amount of a reporter antibody that binds to the zika virus like particle of the microsphere complex,
Step 2.1: contacting the amount of said microsphere complex with the sample to allow binding of anti-zika virus antibodies in the sample to the zika virus like particles coupled to the microspheres in the microsphere complex,
Step 2.2: contacting said amount of reporter antibody with said microsphere complex and the sample of step 2.1 to allow binding of the reporter antibody to the zika virus like particles coupled to the microspheres,
Step 2.3: contacting said amount of reporter antibody, said amount of microsphere complex, and the sample of step 2.2 with a secondary reporter antibody to allow binding of the secondary reporter antibody to the constant region of the reporter antibody, and
Step 3: detecting a signal from the secondary reporter antibody bound to the reporter antibody in step 2.3, wherein the reporter antibody is bound to the zika virus like particles coupled to the microspheres in the microsphere complex in step 2.2.

According to another specific embodiment of the invention the method comprises the steps of:
Step 1: providing a kit comprising an amount of a microsphere complex comprising a microsphere coupled to a zika virus like particle and an amount of a reporter antibody that binds to the zika virus like particle of the microsphere complex,
Step 2.1: contacting the amount of said microsphere complex with the amount of said reporter antibody to allow binding of the reporter antibody to the zika virus like particles coupled to the microspheres in the microsphere complex,
Step 2.2: contacting the sample with said microsphere complex and said reporter antibody of step 2.1 to allow binding of anti-zika virus antibodies in the sample to the zika virus like particles coupled to the microspheres in the microsphere complex,
Step 2.3: contacting said amount of reporter antibody, said amount of microsphere complex, and the sample of step 2.2 with a secondary reporter antibody to allow binding of the secondary reporter antibody to the constant region of the reporter antibody, and
Step 3: detecting a signal from the secondary reporter antibody bound to the reporter antibody in step 2.3, wherein the reporter antibody is bound to the zika virus like particles coupled to the microspheres in the microsphere complex in step 2.1.

According to one embodiment, contacting in step 2.1 is carried out for about 40 min to about 80 min and contacting in step 2.2 is carried out for about 5 min to about 60 min.

According to one embodiment, contacting in step 2.1 is carried out for about 60 min and contacting in step 2.2 is carried out for about 10 min.

According to one embodiment contacting in step 2.1 is carried out for about 40 min to about 80 min and contacting in step 2.2 is carried out for about 5 min to about 60 min and contacting in step 2.3 is carried out for about 10 min to about 50 min.

According to one embodiment contacting in step 2.1 is carried out for about 60 min and contacting in step 2.2 is carried out for about 10 min and contacting in step 2.3 is carried out for about 30 min.

According to one embodiment of the invention, the sample is a sample from the group consisting of blood, urine, serum, plasma, cerebrospinal fluid, and lymph fluid. According to one embodiment, the sample is a blood plasma sample. According to another embodiment, the sample is a serum sample. According to the invention, the sample is provided outside the human or animal body. Within this invention, the term “plasma” refers to blood plasma.

According to a specific embodiment of the invention, the plasma sample is heat-inactivated. Heat-inactivation may occur by incubating the sample for about 30 min at about 56° C.

According to the invention, the sample is from a subject from the group consisting of mouse, primate, non-human primate, human, rabbit, cat, rat, horse, and sheep. According to one embodiment, the subject is human. According to another embodiment, the subject is a non-human primate.

According to one embodiment of the present invention, the signal in step 3 is resulting from the at least one detectable label. According to another embodiment, the signal in step 3 is a fluorescence signal. In a specific embodiment, the fluorescence signal results from phycoerythrin. The signal can be detected by any suitable detection instrument.

According to the invention, the detection system refers to any system, which is suitable for determining values indicative for the presence and/or amount of reporter antibody captured on the microsphere.

According to the invention, the detection system may also be able to determine values indicative for the presence and/or amount of a microsphere by identifying the specific feature of the microsphere.

The selection of a suitable detection system depends on several parameters such as the type of detectable labels used for detection or the kind of analysis performed. Various optical and non-optical detection systems are well established in the art. A general description of detection systems that can be used with the method can be found, e.g., in Lottspeich, F., and Zorbas H., Springer Spektrum 2012, Bioanalytik.

According to one embodiment of the invention, the detection system is an optical detection system. In some embodiments, performing the method involves simple detection systems, which may be based on the measurement of parameters such as fluorescence, optical absorption, resonance transfer, and the like.

According to one embodiment of the invention, the detection system measures fluorescence. Such systems measure the capacity of particular molecules to emit their own light when excited by light of a particular wavelength resulting in a characteristic absorption and emission behavior. In particular, quantitative detection of fluorescence signals is performed by means of modified methods of fluorescence microscopy (for review see, e.g., Lichtman, J. W., and Conchello, J. A. (2005) Nature Methods 2, 910-919; Zimmermann, T. (2005) Adv. Biochem. Eng. Biotechnol. 95, 245-265). Thereby, the signals resulting from light absorption and light emission, respectively, are separated by one or more filters and/or dichroites and imaged on suitable detectors. Data analysis is performed by means of digital image processing. Image processing may be achieved with several software packages well known in the art (such as Mathematica Digital Image Processing, EIKONA, or Image-PRO). Another suitable software for such purposes is the Iconoclust software (Clondiag Chip Technologies GmbH, Jena, Germany). Suitable detection systems may be based on “classical” methods for measuring a fluorescent signal such as epifluorescence or darkfield fluorescence microscopy (reviewed, e.g., in: Lakowicz, J. R. (1999) Principles of Fluorescence Spectroscopy, 2nd ed., Plenum Publishing Corp., NY). Another optical detection system that may be used is confocal fluorescence microscopy, wherein the object is illuminated in the focal plane of the lens by a point light source. Importantly, the point light source, object and point light detector are located on optically conjugated planes. Examples of such confocal systems are described in detail, for example, in Diaspro, A. (2002) Confocal and 2-photon-microscopy: Foundations, Applications and Advances, Wiley-Liss, Hobroken, NJ. The fluorescence-optical system is usually a fluorescence microscope without an autofocus, for example a fluorescence microscope having a fixed focus. Further fluorescence detection methods that may also be used include inter alia total internal fluorescence microscopy (see, e.g., Axelrod, D. (1999) Surface fluorescence microscopy with evanescent illumination, in: Lacey, A. (ed.) Light Microscopy in Biology, Oxford University Press, New York, 399-423), fluorescence lifetime imaging microscopy (see, for example, Dowling, K. et al. (1999) J. Mod. Optics 46, 199-209), fluorescence resonance energy transfer (FRET; see, for example, Periasamy, A. (2001) J. Biomed. Optics 6, 287-291), bioluminescence resonance energy transfer (BRET; see, e.g., Wilson, T., and Hastings, J. W. (1998) Annu. Rev. Cell Dev. Biol. 14, 197-230), and fluorescence correlation spectroscopy (see, e.g., Hess, S. T. et al. (2002) Biochemistry 41, 697-705). In specific embodiments, detection is performed using FRET or BRET, which are based on the respective formation of fluorescence or bioluminescence quencher pairs. The use of FRET is also described, e.g., in Liu, B. et al. (2005) Proc. Natl. Acad. Sci. USA 102, 589-593; and Szollosi, J. et al. (2002) J. Biotechnol. 82, 251-266. The use of BRET is detailed, for example, in Prinz, A. et al. (2006) Chembiochem. 7, 1007-1012; and Xu, Y. et al. (1999) Proc. Natl. Acad. Sci. USA 96, 151-156.

In one embodiment the detection system comprises a first light source, e.g. an argon laser or a light emitting diode (LED), which has an excitation wavelength in the range of 300 to 700 nm and a second light source, e.g. an argon laser or a LED, which has an excitation wavelength in the range of 400 to 700 nm and a suitable detection component as for instance a photodiode such as an avalanche photodiode (APD) in combination with a photomultiplier or a charge-coupled device (CCD) sensor. The first light source may be used for excitation of a detectable label. The second light source may be used for the identification of the specific feature of a microsphere, wherein the specific feature may be that the microsphere comprises one or more fluorescent dyes having a specific emission spectrum, and/or one or more fluorescent dyes at a specific concentration.

In a preferred embodiment, the detection system comprises a first light source, e.g. an argon laser or a LED, which has an excitation wavelength in the range of 500 to 600 nm and a second light source, e.g. an argon laser or a LED, which has an excitation wavelength in the range of 600 to 700 nm. In a more preferred embodiment, the detection system comprises a first light source, e.g. an argon laser or a LED, which has an excitation wavelength in the range of 510 to 540 nm and a second light source, e.g. an argon laser or a LED, which has an excitation wavelength in the range of 615 to 645 nm. In an even more preferred embodiment the first light source, e.g. the argon laser or LED has an excitation wavelength in the range of about 510 to about 535 nm and the second light source, e.g. the argon laser or LED has an excitation wavelength in the range of about 620 to about 635 nm. For instance, the detection system comprises a first light source, e.g. an argon laser or a LED, which has an excitation wavelength of about 525 nm and a second light source, e.g. an argon laser or a LED, which has an excitation wavelength of about 635 nm.

The detection system may be also capable of distinguishing the individual size of a microsphere from one microsphere set from the individual size of a microsphere of another microsphere set, thereby allowing individual identification of the microsphere.

The detection system may be one of the group consisting of MAGPIX®, Luminex 200©, and FLEXMAP 3D® (Luminex Corp. Austin, Tex.). In a preferred embodiment, the detection system is the MAGPIX® (Luminex Corp. Austin, Tex.). These detection systems may be operated by a specific software, including the xPONENT® software (Luminex Corp. Austin, Tex.). These detection systems are capable of detecting both, the signal from the at least one detectable label of the reporter or the detection Ab, as well as the specific feature of the microsphere present in the microsphere complex, wherein the specific feature is that the microsphere comprises one or more fluorescent dyes having a specific emission spectrum, and/or one or more fluorescent dyes at a specific concentration.

The detection system may be capable of analyzing one microsphere after the other thereby identifying the microsphere by detecting the specific feature of the microsphere and detecting the signal from the at least one detectable label of the reporter or detection antibody such as flow cytometry based detection systems (e.g. Luminex 200® and FLEXMAP 3D®). The flow cytometry based detection systems Luminex 200® and FLEXMAP 3D® include two lasers each one for irradiation of the one or more fluorescent dyes of the microsphere (the specific feature of microspheres that can be identified by these specific detection systems) and the at least one detectable label of the reporter or detection Ab. As flow cytometry based detection systems are not capturing the microspheres with a magnet, the Luminex 200® and FLEXMAP 3D® systems are compatible with both, magnetic microspheres such as the MagPlex® microspheres and non-magnetic microspheres such as the Microplex® microspheres. The Luminex 200® and FLEXMAP 3D® systems detect signals from the microspheres and reporter or detection Ab by avalanche photodiodes (APD) in combination with photomultipliers (PMT).

Alternatively, the detection system may be capable of analyzing multiple microspheres at once. Therefore, a monolayer of magnetic microspheres is captured by a magnet and the microspheres are excited with two LEDs, one LED for excitement of the one or more fluorescent dyes of the microspheres (the specific feature of microspheres that can be identified by these specific detection systems) and the other LED for excitement of the at least one detectable label of the reporter or detection Ab. The signals from the microspheres and reporter or detection Ab are recorded by a CCD imager, which allows identification of each microsphere and the corresponding antigen to which the microsphere is coupled to. An example for a LED-based detection system is the MAGPIX® instrument. As analyses with the MAGPIX® instrument involves capture of the microspheres with a magnet, the MAGPIX® instrument is solely compatible with magnetic microspheres such as MagPlex® microspheres.

The present invention is further directed to such a method for detecting the presence and/or amount of anti-zika virus antibodies in a sample from a subject comprising the further steps of:
Step 4: determining the presence and/or the amount of the reporter antibody bound to the zika virus like particles coupled to the microspheres in the microsphere complex from the signal of step 3, and
Step 5: determining the presence and/or the amount of anti-zika virus antibodies in the sample based on the presence and/or the amount of the reporter antibody determined in step 4.

In specific embodiments of the invention, the amount of reporter Ab bound to the ZIKV VLPs coupled to the microspheres in the microsphere complex is determined by comparing the signal of step 3 to a standard curve, wherein the standard curve comprises signals resulting from known amounts of reporter Ab bound to ZIKV VLPs coupled to microspheres in a microsphere complex.

In specific embodiments of the invention, the amount of anti-ZIKV Abs in the sample is determined based on the amount of the reporter Ab determined in step 4 by comparing the amount of reporter Ab to a standard curve, wherein the standard curve comprises amounts of reporter Ab resulting from known amounts of anti-ZIKV Abs present within a sample.

According to another embodiment of the present invention, the anti-ZIKV Abs from the sample of the subject are ZIKV specific Abs.

In further embodiments, the anti-ZIKV Abs from the sample of the subject are ZIKV neutralizing Abs.

Further, the invention refers to a multiplexing method for detecting signals from multiple reporter Abs indicative for the presence and/or amount of Abs directed to different viruses within one sample. Therefore, different microsphere complexes are mixed, wherein the different microsphere complexes comprise microspheres of different microsphere sets coupled to a specific virus antigen (e.g. flavivirus antigens including DENV, ZIKV, WNV, JEV, YFV). The microspheres of a specific microsphere set can be identified by a specific feature, which may be that the microspheres comprise one or more fluorescent dyes having a specific emission spectrum and/or one or more fluorescent dyes at a specific concentration and/or are of a certain size. By identification of the specific feature, the specific virus antigen coupled to the microsphere can be identified simultaneously. For instance, a first microsphere complex comprising a microsphere comprising one or more fluorescent dyes having a specific emission spectrum is coupled to a zika virus like particle and a second microsphere complex comprising a microsphere comprising one or more fluorescent dyes having another specific emission spectrum is coupled to a dengue virus like particle and both microsphere complexes are mixed. The fluorescent dyes of the microspheres may be excited by the same light source i.e. the same wavelength. By detecting the specific feature of the microspheres, the immobilized antigen is concomitantly detected. Further, when the microsphere complex mixture is incubated with a sample comprising anti-ZIKV and anti-DENV Abs, both anti-ZIKV and anti-DENV Abs can be detected within one single experiment by the application of two reporter Abs, wherein one reporter Ab binds to the ZIKV VLP and the other reporter Ab binds to the DENV VLP. Both reporter Abs may be attached to phycoerythrin as the detectable label. By exciting, for instance, one microsphere after the other with two different wavelengths, wherein one wavelength is suitable for exciting the one or more fluorescent dyes of the microsphere and the other wavelength is suitable for exciting the detectable label, a signal indicative for the specific feature of the microsphere (and therefore for the antigen immobilized) can be detected and a signal indicative for the amount of bound reporter Ab can be detected. Both, anti-ZIKV and anti-DENV Abs can be determined in one single experiment using the multiplexing approach.

Method for Determining an Antibody Correlate of Protection Against Zika Virus Infection

The present invention is further directed to a method for determining an antibody correlate of protection against zika virus infection for a zika virus vaccine in a type of non-human subjects, the method comprising the steps of:
Step 1: selecting a group of said subjects which are zika virus naive,
Step 2: dividing the group of subjects into at least two subgroups, wherein one subgroup functions as control group and at least one subgroup functions as inoculation group,
Step 3: inoculating said at least one inoculation group with a dose of the zika virus vaccine,
Step 4: challenging all subjects with an infectious amount of the zika virus,
Step 5: determining the amount of anti-zika virus antibodies for each subject according to the methods as described above at least after inoculation with the zika virus vaccine and before challenging with the infectious amount of the zika virus,
Step 6: determining presence or absence of viremia in all subjects after challenging with the infectious amount of the zika virus,
Step 7: repeating steps 3 to 6 with further inoculation groups with increasing vaccine doses until absence of viremia is determined in all subjects of one inoculation group in step 6, and
Step 8: determining the amount of anti-zika virus antibodies after inoculation with the zika virus vaccine and before challenging with the infectious amount of the zika virus associated with absence of viremia after challenging with the infectious amount of zika virus as antibody correlate of protection.

In specific embodiments of the invention the type of non-human subjects is selected from the group consisting of mice, primates, non-human primates, rabbits, cats, rats, horses, or sheep.

In a more specific embodiment of the invention, the type of non-human subjects is non-human primates.

In further embodiments of the invention, the zika virus vaccine in step 3 is a purified inactivated zika virus vaccine (PIZV).

In more specific embodiments, the zika virus vaccine in step 3 is a PIZV and the dose of the PIZV in step 3 is between about 0.3 μg and about 20 μg. In embodiments, where more than one subgroup is selected in step 2, each subgroup receives a different dose of the zika virus vaccine in step 3. For instance, a first subgroup receives a dose of the PIZV of about 0.4 μg, a second subgroup receives a dose of the PIZV of about 2 μg, and a third subgroup receives a dose of the PIZV of about 10 μg.

In specific embodiments, the zika virus in step 4 is ZIKV strain PRVABC59.

In more specific embodiments, the zika virus in step 4 is ZIKV strain PRVABC59 and the infectious amount in step 4 is a nominal dose of about 104 focus forming units (FFU) present in about 0.5 mL.

In a specific embodiment of the invention, challenging in step 4 is performed between about 60 and about 80 days, more preferable about 71 days, after inoculating the at least one inoculation group with a dose of a ZIKV vaccine.

In another embodiment, the presence of viremia is determined in step 6 by monitoring the typical symptoms caused by an infection with the ZIKV of step 4 and/or determining the amount of ZIKV of step 4 and/or determining the amount of neutralizing Abs directed against ZIKV of step 4. In more specific embodiments, the amount of ZIKV is determined by RT-PCR and/or the amount of neutralizing Abs directed against ZIKV is determined by a RVP assay, a FFA, a MNT, or a PRNT.

In another embodiment, the presence of viremia is determined in step 6 after 0, and/or 1, and/or 2, and/or 3, and/or 4, and/or 5, and/or 6, and/or 7, and/or 8, and/or 9, and/or 10, and/or 11, and/or 12, and/or 13, and/or 14, and/or 15, and/or 20, and/or 30 days after challenging with the ZIKV in step 4.

This invention is further directed to a method for determining an antibody correlate of protection against ZIKV infection in human subjects by mathematically modelling the correlate of protection of a non-human subject like a non-human primate to fit human subjects.

### Method for Diagnosing Protection Against Zika Virus Infection

The present invention is further directed to a method for diagnosing the protection of a human subject against a zika virus infection comprising the steps of:
Step 1: providing a sample from the human subject outside the human body,
Step 2: determining the amount of anti-ZIKV antibodies in the sample from the human subject as described above, and
Step 3: determining protection by comparing the amount of anti-ZIKV antibodies determined in step 2 to the antibody correlate of protection against zika virus infection in human subjects, optionally determined according to the method described above.

In preferred embodiments, the method for diagnosing the protection of a human subject against a zika virus infection comprising the steps of:
Step 1: providing a sample from the human subject outside the human body,
Step 2: determining the amount of anti-ZIKV antibodies in the sample from the human subject as described above, and
Step 3: determining protection by comparing the amount of anti-ZIKV antibodies determined in step 2 to the antibody correlate of protection against zika virus infection in human subjects determined according to the method described above.

The present invention is further directed to a method for diagnosing the protection of a non-human subject against a zika virus infection comprising the steps of:
Step 1: providing a sample from the non-human subject outside the non-human body,
Step 2: determining the amount of anti-ZIKV antibodies in the sample from the non-human subject as described above, and
Step 3: determining protection by comparing the amount of anti-ZIKV antibodies determined in step 2 to the antibody correlate of protection determined in this type of non-human subjects according to the method described above.

In a particular embodiment the sample of step 1 is a blood sample, in particular a blood plasma sample. Method for diagnosing zika virus infection

The present invention is further directed to a method for diagnosing a ZIKV infection in a human subject comprising the steps of:
Step 1: providing a sample from the human subject outside the human body
Step 2: determining the amount of anti-ZIKV antibodies in the sample as described above,
Step 3: determining infection by comparing said amount of anti-ZIKV antibodies to established amounts of anti-ZIKV antibodies in ZIKV infected human subjects.

The present invention is further directed to a method for diagnosing a ZIKV infection in a non-human subject comprising the steps of:
Step 1: providing a sample from the non-human subject outside the non-human body
Step 2: determining the amount of anti-ZIKV antibodies in the sample as described above,
Step 3: determining infection by comparing said amount of anti-ZIKV antibodies to established amounts of anti-ZIKV antibodies in ZIKV infected non-human subjects.

In certain embodiments, the sample is a blood sample, in particular a blood plasma sample.

In certain embodiments, the ZIKV infection is convalescent.

In certain embodiments, the ZIKV infection is acute.

### Method for Assaying the Presence of a Zika Virus Infection

The present invention is further directed to a method for assaying the presence of a zika virus infection in a subject comprising the steps of:
Step 1: obtaining a sample from the subject,
Step 2: determining the amount of anti-zika virus antibodies in the sample as described above under the section “Method of detecting anti-zika virus antibodies”, and
Step 3: determining the presence of a zika virus infection by comparing said amount of anti-zika virus antibodies to established amounts of anti-zika virus antibodies in zika virus infected subjects.

In certain embodiments, the subject is a human.

In certain embodiments, the zika virus infection is acute.

In certain embodiments, the zika virus infection is convalescent.

In certain embodiments, the sample is a blood sample, in particular a blood plasma sample.

### Method for Preventing Zika Disease

The present invention is further directed to a method for preventing zika disease in a human subject, the method comprising the steps of:
Step 1: obtaining a sample from the human subject,
Step 2: determining the amount of anti-zika virus antibodies in the sample from the human subject as described above under the section “Method of detecting anti-zika virus antibodies”,
Step 3: determining whether the human subject has an amount of anti-zika virus antibodies to confer protection by comparing the amount of anti-zika virus antibodies determined in step 2 to the antibody correlate of protection against zika virus infection in human subjects, optionally determined as described above under the section “Method for determining an antibody correlate of protection”, and
Step 4: administering to the human subject a zika virus vaccine if the human subject has an amount of anti-zika antibodies that is lower than the antibody correlate of protection against zika virus infection in human subjects, optionally determined as described above under the section “Method for determining an antibody correlate of protection”.

“Confer protection” within that context means that the amount of anti-zika virus antibodies present in the human subject is sufficient to prevent the human subject from developing zika disease after infection with a zika virus.

In preferred embodiments, the method for preventing zika disease in a human subject comprises the steps of:
Step 1: obtaining a sample from the human subject,
Step 2: determining the amount of anti-zika virus antibodies in the sample from the human subject as described above under the section “Method of detecting anti-zika virus antibodies”,
Step 3: determining whether the human subject has an amount of anti-zika virus antibodies to confer protection by comparing the amount of anti-zika virus antibodies determined in step 2 to the antibody correlate of protection against zika virus infection in human subjects determined as described above under the section “Method for determining an antibody correlate of protection”, and
Step 4: administering to the human subject a zika virus vaccine if the human subject has an amount of anti-zika antibodies that is lower than the antibody correlate of protection against zika virus infection in human subjects determined as described above under the section “Method for determining an antibody correlate of protection”.

In certain embodiments, the human subject is a woman. In certain embodiments, the woman is a woman of childbearing potential.

In certain embodiments, the human subject is living in a zika endemic region. In certain embodiments, the human subject is living in a zika non-endemic region traveling to a zika endemic region.

In certain embodiments, the zika virus vaccine is a purified inactivated zika virus vaccine. A suitable zika virus vaccine is described, for instance, in WO 2019/090228.

### Method for Detecting Total Anti-Zika Virus Antibodies in a Sample

The present invention is further directed to a method for detecting essentially the complete panel of anti-zika virus antibodies (which may be of a certain antibody class, such as IgG) in a sample. When compared to the method for detecting anti-zika virus antibodies as described above, the method for detecting total anti-zika virus antibodies does not include the application of a reporter Ab, which is competing with the anti-zika virus antibodies in a sample. Whereas the method for detecting anti-zika virus antibodies as described above provides a read-out for antibodies in a sample, which are capable of competing with the reporter Ab (ZIKV-specific Abs), the method for detecting total anti-zika virus antibodies as described under this section provides a read-out of essentially the complete panel of anti-zika virus antibodies (which may be of a certain antibody class, such as IgG) in a sample.

Thus, the present invention in one embodiment is further directed to a method for detecting a signal from a detection antibody indicative for the presence and/or amount of anti-zika virus antibodies in a sample from a subject comprising the steps of:
Step 1: contacting an amount of a microsphere complex as described above with the sample to allow binding of anti-zika virus antibodies in the sample to the zika virus like particles coupled to the microspheres in the microsphere complex,
Step 2: contacting an amount of a detection antibody with the microsphere complex and the sample of step 1 to allow binding of the detection antibody to the heavy chain constant region of the anti-zika virus antibodies bound to the zika virus like particles coupled to the microspheres in the microsphere complex, wherein the detection antibody binds to the anti-zika virus antibodies with the variable region of the detection antibody and wherein the detection antibody is attached to at least one detectable label, and
Step 3: detecting a signal from the detection antibody bound to the anti-zika virus antibodies in step 2.

The present invention in one embodiment is further directed to a method for determining the presence and/or amount of anti-zika virus antibodies in a sample from a subject, wherein the method comprises the further steps of:
Step 4: determining the presence and/or amount of the detection antibody bound to the anti-zika virus antibodies from the signal of step 3, and
Step 5: determining the presence and/or amount of anti-zika virus antibodies in the sample from the presence and/or amount of the detection antibody determined in step 4.

The amount of anti-zika virus antibodies in the sample may be determined from the amount of the detection antibody by comparison to a standard curve. For recording the standard curve, samples with known anti-zika antibody amounts may be analyzed in the method. Corresponding signals derived from the detection antibody upon analysis of the samples can then be matched to the corresponding anti-zika antibody amounts in the samples.

In one embodiment, contacting in step 1 is carried out for about 60 min and contacting in step 2 is carried out for about 30 min.

In one embodiment, the detection antibody is attached to the at least one detectable label by the heavy chain constant region. In certain embodiments, the at least one detectable label the detection antibody is attached to is a fluorescence label, such as phycoerythrin.

In certain embodiments, the signal in step 3 is resulting from the at least one detectable label, preferably the signal is a fluorescence signal.

Concerning the detection of the signal from the detection antibody, reference is made to the chapter “Method for detecting anti-zika virus antibodies”.

In certain embodiments, the sample is a sample from the group consisting of blood, urine, serum, blood plasma, cerebrospinal fluid, and lymph fluid, in particular the sample is a serum or blood plasma sample.

In certain embodiments, the subject is from the group consisting of mouse, primate, non-human primate, human, rabbit, cat, rat, horse, and sheep, in particular the subject is a human.

The present invention is further directed to a method for determining an antibody correlate of protection against zika virus infection as described under the chapter “Method for determining an antibody correlate of protection against zika virus infection”, wherein the amount of anti-zika virus antibodies for each subject in step 5 is determined according to the method for detecting total anti-zika virus antibodies in a sample as described under this chapter.

The present invention is further directed to a method for diagnosing the protection of a human subject against a zika virus infection as described under the chapter “Method for diagnosing zika virus protection”,
wherein the amount of anti-zika virus antibodies in the sample in step 2 is determined according to the method for detecting total anti-zika virus antibodies in a sample as described under this chapter, and
wherein protection in step 3 is determined by comparing the amount of anti-zika virus antibodies to the antibody correlate of protection determined in human subjects as described above using the method for detecting total anti-zika virus antibodies in a sample as described under this chapter.

The present invention is further directed to a method for diagnosing the protection of a non-human subject against a zika virus infection as described under the chapter “Method for diagnosing zika virus protection”,
wherein the amount of anti-zika virus antibodies in the sample in step 2 is determined according to the method for detecting total anti-zika virus antibodies in a sample as described under this chapter, and
wherein protection in step 3 is determined by comparing the amount of anti-zika virus antibodies to the antibody correlate of protection determined in this type of non-human subjects as described above using the method for detecting total anti-zika virus antibodies in a sample as described under this chapter.

In certain embodiments, the detection antibody is capable of binding to a certain antibody class or subclass (isotype). In preferred embodiments, the detection antibody is capable of binding to antibodies of class IgG.

### Alternative Zika Antigens Useful for Coupling to the Microspheres

The present invention is alternatively directed to a kit comprising an amount of a microsphere complex coupled to a zika antigen and an amount of a reporter antibody that binds to the zika antigen of the microsphere complex and corresponding methods.

The ZIKV antigen may be a ZIKV structural protein or a ZIKV non-structural protein.

### Microsphere Complex Comprising a Microsphere Coupled to a Dengue Virus Like Particle

The present invention further provides a microsphere complex comprising a microsphere coupled to a dengue virus like particle (DENV VLP). Concerning the properties of the microsphere, reference is made to the chapter “microsphere complex” above.

In one embodiment, the microsphere is coupled to a dengue-1 virus like particle.

In one embodiment, the dengue-1 virus like particle is derived from dengue-1 virus strain Puerto Rico/US/BID-V853/1998 characterized by SEQ ID NO: 179 and/or SEQ ID NO: 180.

In one embodiment, the dengue-1 virus like particle comprises structural proteins of dengue-1 virus strain Puerto Rico/US/BID-V853/1998 characterized by SEQ ID NO: 179 and/or SEQ ID NO: 180.

In one embodiment, the dengue-1 virus like particle comprises the envelope protein, the membrane protein, and/or the pre-membrane protein, which are at least 70%, or at least 75%, or at least 80%, or at least 85%, or at least 90%, or at least 95% or 100% identical to corresponding parts of SEQ ID NO: 180. In another embodiment, the dengue-1 virus like particle comprises the envelope protein, the membrane protein, and the pre-membrane protein, which are at least 70%, or at least 75%, or at least 80%, or at least 85%, or at least 90%, or at least 95% or 100% identical to corresponding parts of SEQ ID NO: 180.

In one embodiment, the microsphere is coupled to a dengue-2 virus like particle.

In one embodiment, the dengue-2 virus like particle is derived from dengue-2 virus strain Thailand/16681/84 characterized by SEQ ID NO: 181 and/or SEQ ID NO: 182.

In one embodiment, the dengue-2 virus like particle comprises structural proteins of dengue-2 virus strain Thailand/16681/84 characterized by SEQ ID NO: 181 and/or SEQ ID NO: 182.

In one embodiment, the dengue-2 virus like particle comprises the envelope protein, the membrane protein, and/or the pre-membrane protein, which are at least 70%, or at least 75%, or at least 80%, or at least 85%, or at least 90%, or at least 95% or 100% identical to corresponding parts of SEQ ID NO: 182. In another embodiment, the dengue-2 virus like particle comprises the envelope protein, the membrane protein, and the pre-membrane protein, which are at least 70%, or at least 75%, or at least 80%, or at least 85%, or at least 90%, or at least 95% or 100% identical to corresponding parts of SEQ ID NO: 182.

In one embodiment, wherein the microsphere is coupled to a dengue-3 virus like particle.

In one embodiment, the dengue-3 virus like particle is derived from dengue-3 virus strain Sri Lanka D3/H/IMTSSA-SRI/2000/1266 characterized by SEQ ID NO: 183 and/or SEQ ID NO: 184.

In one embodiment, the dengue-3 virus like particle comprises structural proteins of dengue-3 virus strain Sri Lanka D3/H/IMTSSA-SRI/2000/1266 characterized by SEQ ID NO: 183 and/or SEQ ID NO: 184.

In one embodiment, the dengue-3 virus like particle comprises the envelope protein, the membrane protein, and/or the pre-membrane protein, which are at least 70%, or at least 75%, or at least 80%, or at least 85%, or at least 90%, or at least 95% or 100% identical to corresponding parts of SEQ ID NO: 184. In another embodiment, the dengue-3 virus like particle comprises the envelope protein, the membrane protein, and the pre-membrane protein, which are at least 70%, or at least 75%, or at least 80%, or at least 85%, or at least 90%, or at least 95% or 100% identical to corresponding parts of SEQ ID NO: 184.

In one embodiment, the microsphere is coupled to a dengue-4 virus like particle.

In one embodiment, the dengue-4 virus like particle is derived from dengue-4 virus strain Dominica/814669/1981 characterized by SEQ ID NO: 185 and/or SEQ ID NO: 186.

In one embodiment, the dengue-4 virus like particle comprises structural proteins of dengue-4 virus strain Dominica/814669/1981 characterized by SEQ ID NO: 185 and/or SEQ ID NO: 186.

In one embodiment, the dengue-4 virus like particle comprises the envelope protein, the membrane protein, and/or the pre-membrane protein, which are at least 70%, or at least 75%, or at least 80%, or at least 85%, or at least 90%, or at least 95% or 100% identical to corresponding parts of SEQ ID NO: 186. In another embodiment, the dengue-4 virus like particle comprises the envelope protein, the membrane protein, and the pre-membrane protein, which are at least 70%, or at least 75%, or at least 80%, or at least 85%, or at least 90%, or at least 95% or 100% identical to corresponding parts of SEQ ID NO: 186.

In one embodiment, the dengue virus like particle is produced in human embryonic kidney (HEK293) cells.

Within the meaning of this invention, a DENV VLP comprising the envelope glycoprotein (E protein), membrane (M) protein, and/or pre-membrane (prM) protein of a DENV strain refers to a DENV VLP comprising an envelope glycoprotein, membrane protein, and/or pre-membrane protein which are at least 70%, or at least 75%, or at least 80%, or at least 85%, or at least 90%, or at least 95% or 100% identical to corresponding parts of the protein sequence of the DENV virus strain. Corresponding parts within that context mean parts of the protein sequence that encode for E protein, M protein, or prM protein, respectively.

### EXAMPLES

The following Examples are included to demonstrate certain aspects and embodiments of the invention as described in the claims. It should be appreciated by those of skill in the art, however, that the following description is illustrative only and should not be taken in any way as a restriction of the invention.

### Example 1: Coupling of ZIKV and DENV Antigens to Microspheres

Microspheres used for coupling were MagPlex® microspheres (Luminex Corporation, Austin, Texas). MagPlex® microspheres are superparamagnetic polystyrene microspheres with surface carboxyl groups. The microspheres were delivered in a volume of 4 to 4.1 mL with an average concentration of 1.2 to 1.3×10⁷ microspheres per mL (microspheres/mL). Microspheres used in this example were catalog numbers MC10014-04 (Product Lot. B75253), MC10025-04 (Product Lot. B76003), MC10033-04 (Product Lot. B71929), MC10045-04 (Product Lot. B76946), MC10047-04 (Product Lot. B75165), and MC10076-04 (Product Lot. B76373). The different microsphere catalog numbers refer to different microsphere sets wherein the microspheres of the sets comprise one or more fluorescent dyes having a specific emission spectrum (the specific feature) for distinguishing the microspheres upon detection. As the coupling mechanism involving the surface carboxyl groups is independent of the specific feature of the microspheres, catalog numbers of the MagPlex® microspheres may be exchanged according to variations in experimental set-ups.

DENV antigens for coupling to microspheres were DENV1 VLP (0.46 mg/mL liquid stock in 10 mM sodium phosphate, 20 mM sodium citrate, 154 mM sodium chloride pH 7.4; The Native Antigen Company, Product Code: DENV1-VLP-500, Batch No. 19040109), DENV2 VLP (0.52 mg/mL liquid stock in 10 mM sodium phosphate, 20 mM sodium citrate, 154 mM sodium chloride pH 7.4; The Native Antigen Company, Product Code: DENV2-VLP-500, Batch No. 19040816), DENV3 VLP (0.72 mg/mL liquid stock in 10 mM sodium phosphate, 20 mM sodium citrate, 154 mM sodium chloride pH 7.4; The Native Antigen Company, Product Code: DENV3-VLP-500, Batch No. 18111415), and DENV4 VLP (0.14 mg/mL liquid stock in Dulbecco's phosphate-buffered saline (DPBS) pH 7.4, 30% sucrose; The Native Antigen Company, Product Code: DENV4-VLP-500, Batch No. 18110614). DENV1-4 VLPs are consisting of DENV prM, M, and E protein produced in human embryonic kidney (HEK 293) cells. For production of DENV1-4 VLPs, the C-terminal 20% of DENV E protein were replaced by the corresponding Japanese encephalitis virus (JEV) SA-14 sequence (EMBL-EBI accession No: M55506.1, SEQ ID NO: 177 and 178; residues replaced: E protein amino acids 399-497 (DENV1 VLP), 397-495 (DENV2 VLP), 399-492 (DENV3 VLP), 400-495 (DENV4 VLP)). The replaced sequence corresponds to the transmembrane and intraparticle portion of the protein. DENV1 VLP was produced using the sequence from strain Puerto Rico/US/BID-V853/1998 (GenBank accession No. EU482592.1; SEQ ID NO: 179 and 180). DENV2 VLP was produced using the sequence from strain Thailand/16681/84 (EMBL-EBI accession No: U87411.1; SEQ ID NO: 181 and 182). DENV3 VLP was produced using the sequence from strain Sri Lanka D3/H/IMTSSA-SRI/2000/1266 (GenBank accession No. AY099336.1; SEQ ID NO: 183 and 184). DENV4 VLP was produced using the sequence from strain Dominica/814669/1981 (EMBL-EBI accession No: AF326825.1; SEQ ID NO: 185 and 186).

ZIKV antigens for coupling to microspheres were ZIKV VLP, and comparative examples, including recombinant 6×His-SUMO-tagged ZIKV EDIII (rZIKV-EDIII-1), recombinant 6×His-tagged ZIKV EDIII (rZIKV-EDIII-2), and recombinant human IgG1 Fc-tagged ZIKV EDIII (rZIKV-EDIII-3).

ZIKV VLPs (0.15 mg/mL liquid stock in DPBS pH 7.4, 20% sucrose; The Native Antigen Company, Product Code: ZIKV-VLP-250, Batch No. 19051017) comprise prM, M, and E protein of ZIKV isolate Z1106033 isolated in Suriname (Asian genotype; Enfissi et al., Lancet 2016, 387(10015):227-228; GenBank Accession No. KU312312.1, SEQ ID NO: 1 and SEQ ID NO: 2) and are produced in HEK 293 cells. [00341] r-ZIKV-EDIII-1 derived from the EDIII (SEQ ID NO: 4) of ZIKV strain H/PF/2013 (GenBank Accession No. KJ776791) and was cloned into a pETite vector (Lucigen, Cat. No. 49003-1). The pETite vector is designed for expression of the target protein as a fusion protein with an amino (N)-terminal 6×His-SUMO tag. r-ZIKV-EDIII-1 was expressed in Escherichia coli HI-Control BL21 (DE3) cells (Lucigen, Cat. No. 60435-1) and purified with immobilized metal affinity chromatography (IMAC) using a His-column. To further evaluate purity and integrity of rZIKV-EDIII-1 after the His-column run, elution fractions were analyzed by sodium dodecyl sulfate-polyacrylamide gel electrophoresis (SDS-PAGE) and analytical size exclusion chromatography (SEC) revealing a monomeric state of the recombinant protein. Finally, antigenicity of rZIKV-EDIII-1 was validated in a Bio-layer interferometry (BLI) assay using anti-ZIKV #1 to 6 and anti-PanDENV1-4 EDIII. The Octet results revealed that rZIKV-EDIII-1 showed good affinity for Anti-ZIKV #1-3 and 5, but solely weak association with anti-ZIKV #4 and it did not associate with Anti-ZIKV #6 and Anti-PanDENV1-4 EDIII mAb. rZIKV-EDIII-2 (Asian strain; The Native Antigen Company, Product Code: REC31775-100, Lot. No.: 20012409) was manufactured in E. coli and provided with an amount of 100 μg in 20 mM carbonate buffer pH 10.0. rZIKV-EDIII-3 (Creative Biolabs, Product ID: VAng-Wyb7346) is derived from ZIKV strain SPH2015 (GenBank accession No. ALU33341.1) and comprises ZIKV EDIII (amino acids V593-L699) with the Fc-region of human IgG1 at the carboxyl (C)-terminus expressed in HEK293 cells. The lyophilized protein (50 μg) was reconstituted to achieve a 0.25 mg/mL stock concentration in phosphate buffered saline (PBS) pH 7.4, 5% trehalose, 5% mannitol, 0.01% Tween-80 according to the Manufacture's protocol.

Antigens were coupled to the microspheres as described in Angeloni S., Das S. Dunbar S., Stone V, Swift S., xMAP Cookbook 4^{th} edition, “A collection of methods and protocols for developing multiplexing assays with xMAP Technology” (Luminex Corporation, Austin, Texas). Different microspheres comprising one or more fluorescent dyes having a specific emission spectrum were applied for coupling of multiple antigens (Luminex Corporation, Austin, Texas; Cat. No. MC10014-04, MC10025-04, MC10033-04, MC10045-04, MC10047-04, MC10076-04) to provide the possibility to distinguish the microspheres according to their coupled antigens when analyzed within one sample. For example, ZIKV VLP was coupled to MagPlex® Cat. No. MC10047-04 and DENV3 VLP to MagPlex® Cat. No. MC10025-04.

The uncoupled stocks of MagPlex® microsphere suspensions (1.2 to 1.3×10⁷ microspheres/mL, Luminex Corporation, Austin, Texas) were resuspended by vortexing (30 sec) and 1 up to 12.5×10⁶ microspheres of each stock were transferred to 1.5 mL microcentrifuge tubes and placed into a 1.5 mL tubes magnetic separator (Life Technologies, Cat. No. 44578578). Separation of the microspheres from the suspension occurred for 30-60 sec. Supernatant was carefully removed without disrupting the microsphere pellet while the tubes were still positioned in the magnetic separator. Afterwards, the tubes were removed from the magnetic separator and the microspheres were resuspended in 100 μL distilled H₂O (dH₂O) by vortexing and sonication for approximately 20 sec. The tubes were again placed into the magnetic separator and separation of the microspheres from the suspension occurred for 30-60 sec. Supernatant was removed without disrupting the microsphere pellet while the tubes were still positioned in the magnetic separator. The microspheres were resuspended in 80 μl of activation buffer (0.1 M sodium phosphate (monobasic) pH 6.2) and mixed by vortexing and sonication for 20 sec. Then, 10 μL of 50 mg/mL M hydroxysulfosuccinimide (Sulfo-NHS; 2 mg of Sulfo-NHS in 40 μL of dH₂O; Thermo Fisher Scientific, Cat. No. A39269, Lot. No. UI284573) were added to each microsphere tube and gentle mixing was carried out by vortexing (5 sec). Further, 10 μL of 50 mg/mL 1-Ethyl-3-(3-dimethylaminopropyl)carbodiimide (EDC; 1 mg EDC in 20 μL of dH₂O; Thermo Fisher Scientific, Cat. No. A35391, Lot. No. UD277513) were added to each microsphere tube and gentle mixing was carried out by vortexing (5 sec). Samples were incubated for 20 min at room temperature with gentle mixing by vortexing after 10 min. The tubes were placed into the magnetic separator and separation of the microspheres from the suspension occurred for 30-60 sec. Supernatant was removed without disrupting the microsphere pellet while the tubes were still positioned in the magnetic separator. 50 mM 2-(A morpholino)ethanesulfonic acid (MES) buffer was prepared by dilution of a stock solution (1 M MES buffer) in dH₂O. The buffer pH varied depending on the optimum pH value required for coupling of each viral antigen. Optimum pH values for each antigen were evaluated by the microsphere immunoassays (MIAs) performed under Example 2. MES buffer at pH 6 (Boston Bioproducts, Cat. No. BBMS-60, Lot. No. F03K118) was used for coupling of DENV1-4 VLP. MES buffer pH of 6 was the optimum for coupling of rZIKV-EDIII-1 (coupling also evaluated in MES buffer at pH 7; Boston Bioproducts, Cat. No. BBMS-70, Lot. No. E02K118) and rZIKV-EDIII-3 (coupling also evaluated in MES buffer at pH 5; Boston Bioproducts, Cat. No. BBMS-50, Lot. No. F10K112 and pH 7). MES buffer at pH 7 was used for coupling of ZIKV VLP. rZIKV-EDIII-2 was coupled using MES buffer at pH 8 (Boston Bioproducts, Cat. No. BBMS-80, Lot. No. A24M126; coupling also evaluated in MES buffer at pH 9; Alfa Aesar, Cat. No. AAJ61646AK, Lot. No. X11E562). The tubes were removed from the magnetic separator and the microspheres were resuspended in 250 μL of corresponding 50 mM MES buffer by vortexing and sonication for 20 sec. The tubes were placed into the magnetic separator and separation of the microspheres from the suspension occurred for 30-60 sec. Supernatant was removed without disrupting the microsphere pellet while the tubes were still positioned in the magnetic separator. The tubes were removed from the magnetic separator and the microspheres were resuspended in 250 μL of corresponding 50 mM MES buffer by vortexing and sonication for 20 sec. The tubes were placed into the magnetic separator and separation of the microspheres from the suspension occurred for 30-60 sec. Supernatant was removed without disrupting the microsphere pellet while the tubes were still positioned in the magnetic separator. The tubes were removed from the magnetic separator and 400 μl of corresponding 50 mM MES buffer were added. Afterwards, 100 μL of a corresponding solution of each antigen (diluted in corresponding 50 mM MES buffer) were transferred to the respective 1.5 mL tube containing the activated microspheres to result in a ratio of 5 μg antigen per 10⁶ microspheres in a total volume of 500 μL. The mixture was vortex for 20 sec. For coupling, samples were incubated for 2 hours under rotation at room temperature. The tubes were placed into the magnetic separator and separation of the microspheres from the suspension occurred for 30-60 sec. Supernatant was removed without disrupting the microsphere pellet while the tubes were still positioned in the magnetic separator. The tubes were removed from the magnetic separator and the microspheres were resuspended in 1 mL of 1% BSA in 1-fold PBS pH 7.4 by vortexing for approximately 20 sec. The tubes were placed into the magnetic separator and separation of the microspheres from the suspension occurred for 30-60 sec. Supernatant was removed without disrupting the microsphere pellet while the tubes were still positioned in the magnetic separator. The tubes were removed from the magnetic separator and the microspheres were resuspended in 1 mL of 1% BSA in 1-fold PBS pH 7.4 by vortexing for approximately 20 sec. The tubes were placed into the magnetic separator and separation of the microspheres from the suspension occurred for 30-60 sec. Supernatant was removed without disrupting the microsphere pellet while the tubes were still positioned in the magnetic separator. The tubes were removed from the magnetic separator and the microspheres were resuspended in 250 μL of 1% BSA in 1-fold PBS pH 7.4 by vortexing for approximately 20 sec. The microspheres were kept in the 1.5 mL tubes or alternatively transferred to larger tubes (i.e. 2 mL tubes) when higher amounts of microspheres were coupled such as 12.5×10⁶ microspheres. In order to count the microspheres recovered after the coupling reaction, the microsphere suspension was diluted 2-fold in 1% BSA in 1-fold PBS pH 7.4 (e.g. 15 μL microsphere suspension diluted with 15 μL of 1% BSA in 1-fold PBS pH 7.4). The number of microspheres recovered after the coupling reaction was determined using an automated cell counter (Countes II, Thermo Fisher Scientific, Cat. No. AMQAX1000) by correlating the determined “dead cells” concentration provided by the cell counter to the microspheres. The coupled microspheres were stored at 2-8° C. in the dark (blocking step). Previous to use, the coupled microspheres were allowed to pre-warm for at least 30 min at room temperature.

### Example 2: Selection of mAbs (Reporter Abs) for Microsphere Immunoassay (MIA)

mAbs tested for binding to antigen-coupled microspheres prepared as described in Example 1 are listed in Table 3 with available sequence information provided in Table 1 and 4.

TABLE 3
mAbs tested for binding to antigen-coupled microspheres.
mAbs are presented together with their species origin.
Anti-ZIKV #9 (Clone 181-4) and anti-ZIKV #10 (Clone
329-2) have the same protein and nucleic acid sequences.

| | |
|---|---|
| Species Origin | mAb |

Examples

| | |
|---|---|
| Rabbit | Anti-ZIKV #1 (Clone 102-1) |
| Rabbit | Anti-ZIKV #2 (Clone 242-3) |
| Rabbit | Anti-ZIKV #3 (Clone 270-12) |
| Rabbit | Anti-ZIKV #4 (Clone 289-3) |
| Rabbit | Anti-ZIKV #5 (Clone 306-2) |
| Mouse | Anti-ZIKV #6 (IgG2c kappa, Clone ZV-67) |
| Mouse | Anti-ZIKV #7 (IgG2a kappa, Clone ZKA-64) |
| Rabbit | Anti-ZIKV #8 (Clone 260-2) |
| Rabbit | Anti-ZIKV #9 (Clone 181-4) |
| Rabbit | Anti-ZIKV #10 (Clone 329-2) |
| Rabbit | Anti-ZIKV #11 (Clone 11-3) |

Comparative Examples

| | |
|---|---|
| Rabbit | Anti-Flavivirus #1 (IgG kappa, Clone D1-4G2-4-15) |
| Mouse | Anti-Flavivirus #2 (IgG2a kappa, Clone D1-4G2-4-15) |
| Human | Anti-ZIKV E Protein (IgG1 kappa, Clone ZKA-78) |
| Mouse | Anti-PanDENV1-4 EDIII (IgG1 kappa, Clone 2D73) |
| Rabbit | Antibody Clone 78-2 |
| Rabbit | Antibody Clone 278-11 |

Controls

| | |
|---|---|
| Rabbit | IgG Isotype Control |
| Human | Purified Human IgG1 |
| Mouse | IgG1 Isotype Control |

TABLE 4
Sequence information for comparative example mAbs anti-ZIKV E
protein, anti-Flavivirus #1 and 2, Antibody Clone 278-11,
and Antibody Clone 78-2 tested for binding to antigen-coupled
microspheres. For comparative example mAb Anti-PanDENV1-4 EDIII,
which is a commercial mAb, sequence information was neither
available from the vendor, nor publications, nor databases.
(H = heavy chain; L = light chain; VH = heavy
chain variable region; VL = light chain variable region;
VH-CDR 1-3 = complementary determining regions 1-3 of heavy
chain variable region; VL-CDR 1-3 = complementary determining
regions 1-3 of light chain variable region).

| | | Amino acid | Nucleic acid |
|---|---|---|---|
| | mAb | sequence | sequence |
| mAb | part | (SEQ ID No) | (SEQ ID No) |
| Anti-ZIKV E Protein | VH | 135 | 143 |
| | VH-CDR 1 | 136 | N/A |
| | VH-CDR 2 | 137 | N/A |
| | VH-CDR 3 | 138 | N/A |
| | VL | 139 | 144 |
| | VL-CDR 1 | 140 | N/A |
| | VL-CDR 2 | 141 | N/A |
| | VL-CDR 3 | 142 | N/A |
| Anti-Flavivirus # 1 | H | 145 | 147 |
| and 2 | L | 146 | 148 |
| Antibody Clone 78-2 | H | 149 | 159 |
| | VH | 150 | 160 |
| | VH-CDR 1 | 151 | N/A |
| | VH-CDR 2 | 152 | N/A |
| | VH-CDR 3 | 153 | N/A |
| | L | 154 | 161 |
| | VL | 155 | 162 |
| | VL-CDR 1 | 156 | N/A |
| | VL-CDR 2 | 157 | N/A |
| | VL-CDR 3 | 158 | N/A |
| Antibody Clone 278-11 | H | 163 | 173 |
| | VH | 164 | 174 |
| | VH-CDR 1 | 165 | N/A |
| | VH-CDR 2 | 166 | N/A |
| | VH-CDR 3 | 167 | N/A |
| | L | 168 | 175 |
| | VL | 169 | 176 |
| | VL-CDR 1 | 170 | N/A |
| | VL-CDR 2 | 171 | N/A |
| | VL-CDR 3 | 172 | N/A |

Anti-ZIKV #1 to 5 mAbs (stock concentrations: 2.2, 1.77, 1.84, 2.4, 2.2 mg/mL, respectively, in phosphate buffer saline (PBS), pH 7.4), anti-ZIKV #8 to 11 mAbs, as well as Antibodies Clone 278-11 and 78-2 were generated and characterized as described in co-pending application PCT/US2019/052189 (WO 2020/106358; Takeda Ig Application). In brief, rabbits were immunized with purified inactivated ZIKV vaccine (PIZV) and ZIKV VLPs. Afterwards, the spleen was isolated for generation of hybridoma cells. Hybridoma supernatants were examined for reactivity towards ZIKV VLPs and ZIKV E protein, as well as cross-reactivity towards inactivated DENV1-4 by enzyme linked immunosorbent assay (ELISA). Moreover, hybridoma supernatants were screened for their neutralizing activity in a microneutralization titer (MNT) as well as a reporter virus particle (RVP) assay. Anti ZIKV #1 to 4, and #8 to 10 showed strong neutralization activity, whereas anti-ZIKV #5 showed weak neutralization activity. Anti-ZIKV #11 and Antibody Clone 78-2 showed no neutralization activity over the tested concentration range. Affinity of hybridoma supernatants towards ZIKV VLPs was determined by a Bio-layer interferometry (BLI) assay. In addition, epitope binning was examined in one experiment for anti-ZIKV #1-5 using a competitive BLI assay, binding a primary mAb to the ZIKV VLP, followed by cross-binding a secondary mAb. Binning experiments showed that anti-ZIKV #1 and 2, anti-ZIKV #3 and 4, and anti-ZIKV #5 built up three different clusters, indicating that mAbs belonging to different clusters bind to different regions of ZIKV VLP. Epitope binning was also examined in another experiment for anti-ZIKV #1-5, #8-11 and Antibody Clone 78-2. Binning experiments showed that anti-ZIKV #1-4 and #8-10 built up one cluster, whereas anti-ZIKV #5, #11, and antibody clone 78-2 each were highly diverse from one another and from the other mAbs (anti-ZIKV #1-4 and #8-10). Further, mAbs were sequenced (comp. Table 1 and Table 4). Finally, amino acid residues within the antigen critical for binding of anti-ZIKV #1-5 mAbs were evaluated using an alanine scanning mutagenesis library. Critical residues are those amino acids whose side chains make the highest energetic contribution to the Ab-epitope interaction and whose mutation gave the lowest binding reactivities (<10% of wild-type; Bogan and Thorn, J. Mol. Biol. 1998, 280, 1-9; Lo Conte et al., J. Mol. Biol. 1999, 285, 2177-2198). All mAbs were shown to bind to ZIKV EDIII. Notably, anti-ZIKV #4 also binds to EDI, an epitope associated with EDIII (comp. Table 2).

Anti-ZIKV #6 (Protein A affinity purified, 1 mg/mL stock concentration in PBS with 0.02% Proclin-300, Absolute Antibody, Cat. No. Ab00812-4.0, Lot. No. T1650A08) and 7 (Protein A purified, 1 mg/mL stock concentration in PBS with 0.02% Proclin-300, Absolute Antibody, Cat. No. Ab00779-2.0, Lot. No. T1839B41), as well as comparative example mAbs anti-Flavivirus #1 (Protein A purified, 1 mg/mL stock concentration in PBS with 0.02% Proclin-300, Absolute Antibody, Cat. No. Ab00230-23.0, Lot. No. T1812A57) and 2 (Protein A purified, 1 mg/mL stock concentration in PBS with 0.02% Proclin-300, Absolute Antibody, Cat. No. Ab00230-2.0, Lot. No. T1650A04), anti-ZIKV E Protein (Absolute Antibody, 1 mg/mL stock concentration, Cat. No. Ab00780-10.0, Lot. No. T1644B07) and anti-PanDENV1-4 EDIII (Absolute Antibody, 1 mg/mL stock concentration, Cat. No. Ab00948-1.1, Lot. No. T1749B16) were commercially available. According to the manufacturer, anti-ZIKV #6 was generated by infecting mice with 1000 focus forming units (FFU) of ZIKV MR-766 (GenBank accession No. AY632535.2) followed by a booster 30 days post infection of ZIKV H/PF/2013 (GenBank Accession No. KJ776791) and a final intravenous boost with live ZIKV EDIII (amino acids 299 to 407 of the ZIKV E protein). According to the manufacturer, anti-ZIKV #7 was selected from Epstein-Barr-Virus-immortalized memory B cells derived from ZIKV-infected, DENV-naïve human donors. According to the manufacturer, anti-Flavivirus #1 and #2 have been produced with DENV2 as the immunogen. According to the manufacturer, anti-ZIKV E Protein was selected from Epstein-Barr-Virus-immortalized memory B cells derived from ZIKV-infected, DENV-naïve human donors. According to the manufacturer, anti-PanDENV1-4 EDIII was generated from BALB/c mice immunized with recombinant DENV3 EDIII. Splenocytes were obtained from immunized mice and fused with the NS-1 myeloma cell line to generate hybridomas. Anti-PanDENV1-4 EDIII reacts with residues 309-319 (DKEMAETQHGT) within DENV EDIII.

Anti-ZIKV #6 (Zhao et al., Cell 2016, 166(4), 1016-1027) and Anti-ZIKV #7 (GenBank accession No. KX496860; Stettler et al., Science 2016, 353(6301), 823-6) were generated and characterized previously. Both anti-ZIKV #6 and 7 were demonstrated to bind to ZIKV EDIII. Anti-Flavivirus #1 and 2 were generated and characterized previously (Gentry et al., Am J Trop Med Hyg 1982, 31(3): 548-555; Nawa et al., J. Virol. Meth. 2001, 92, 65-70). Anti-Flavivirus #1 and 2 recognize the fusion loop within EDII that is conserved among different flaviviruses i.e. DENV, WNV, JEV, ZIKV, and YFV (Aubry et al., Transfusion 2016, 56:33-40). Anti-ZIKV E Protein was generated and characterized previously (Stettler et al., Science 2016, 353(6301), 823-6). Anti-ZIKV E Protein is directed against ZIKV EDI/II and cross-reacts with DENV1-4. Anti-PanDENV1-4 EDIII was generated and characterized previously (Li et al., J. Gen. Virol. 2013, 94, 2191-2201) and shows binding to all four DENV serotypes.

In addition to example and comparative example mAbs, control mAbs were tested for binding to antigen-coupled microspheres. As control mAbs served a rabbit IgG isotype control (Invitrogen, 1 mg/mL stock concentration, Cat. No.: 02-6106, Lot. No.: SJ257848), purified human IgG1 (UniProt ID: P01857; Molecular Innovations, 2.49 mg/mL stock concentration in 20 mM sodium phosphate, 150 mM NaCl, 0.05% sodium azide, pH 7.4, Cat. No.: HU-IGG1-1.0MG, Lot. No.: HU-IGG1M-5284), and mouse IgG1 isotype control (Invitrogen, 1 mg/mL stock concentration in 10 mM PBS, pH 7.4, 0.1& sodium azide, Cat. No.: 02-6100, Lot. No.: UD283794).

Binding Specificity of mAbs Towards Antigen-Coupled Microspheres
Determination of EC₅₀ Values after 10 or 60 Min of Incubation of the mAb with the Antigen-Coupled Microspheres

For evaluation of binding specificity of mAbs towards antigen-coupled microspheres, EC₅₀ values were determined after 10 or 60 min of incubation of the mAb with the antigen-coupled microspheres, respectively.

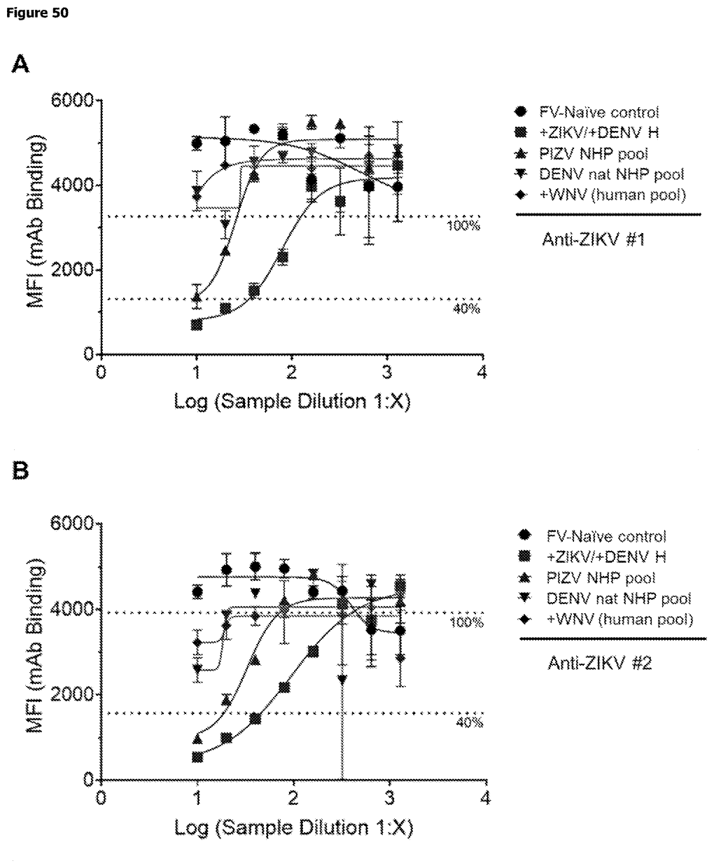

Therefore, microspheres were vortexed for 20 sec. A working microsphere mixture was prepared by diluting the coupled microsphere stock to a final concentration of 25 microspheres/μL in assay buffer (1% (w/v) BSA in 1-fold PBS, pH 7.4) and vortexing for 5 sec. The working mixture was kept at room temperature until 50 μL of the prepared working microsphere mixture were added per well in a black flat bottom 96-well assay plate (Corning Inc., Cat. No. 3915). mAbs were diluted from the corresponding stock concentrations in assay buffer to result in concentrations ranging from 0.001 to 20 μg/mL. mAb concentrations applied in the different experimental set-ups are indicated in the figures. 50 μL of each serially diluted mAb, or 50 μL assay buffer (2 blank wells per plate) were added to the microspheres and the suspension was pipetted up and down three times. Each mAb dilution was examined in duplicates. The plates were covered with a foil sealing sheet and incubated for 10 or 60 min (t 5 min), respectively, at room temperature on a plate shaker at 600 rpm. After incubation, the assay plate was washed two times with PBS-T (0.05% (v/v) Tween-20 in PBS pH 7.4) in a magnetic plate washer (BioTek Instruments, Product Id. 400072). Afterwards, the plate was placed in a 96-well plate magnet (Life Technologies, Product Id. 32513) and incubated for 30 sec while covering the plate. Following the incubation step, the supernatant was removed. For detection, F(ab′)2-goat anti-rabbit IgG (heavy and light chain) cross-adsorbed phycoerythrin (PE)-conjugated secondary Ab (Invitrogen, Cat. No. 31846, Lot. No. TL2684941, 0.5 mg/mL), R-PE AffiniPure F(ab′)₂ fragment goat anti-mouse IgG secondary Ab (heavy and light chain; Jackson ImmunoResearch, Cat. No. 115-116-146, Lot. No. 143867, 0.5 mg/mL), or PE-conjugated goat anti-human IgG secondary antibody (Southern Biotech, Cat. No. 2040-09, Lot. No. B3919-X449B, 0.5 mg/mL) were diluted 1:50 in assay buffer to achieve a final working concentration of 10 μg/mL by vortexing for 5 sec. 50 μL of the corresponding diluted detection Abs were added to each well. The plate was covered with a foil sealing sheet and incubation carried out for 30 min (t 2 min) at room temperature on a plate shaker at 600 rpm. The assay plate was washed two times with PBS-T in the magnetic plate washer. After the washing steps, the plate was placed in the 96-well plate magnet and incubated for 30 sec while covering the plate. Following the incubation step, the supernatant was removed. The microspheres were resuspended in 100 μL assay buffer per well. At this point, storage of the plate sealed with foil sealing sheet overnight at 4° C. is possible. Previous to sample read-out, the plate is allowed to re-equilibrate to room temperature for 20 min (5 min) if stored overnight at 4° C. The plate was put on an orbital shaker at 600 rpm for at least 5 min in order to allow for complete resuspension of immunocomplexed microspheres. Finally, the plate was placed in the multiplexing MAGPIX® plate reader (Luminex Corporation, Austin, Texas). The program used was xPONENT® (Build 4.2.1705.0) and is set-up with sample volume: 50 μL per well; plate protocol: 96-well plate format; and microsphere protocol: Map, BP 50 regions, Type MagPlex.

Determination of EC₅₀ and EC₂₅ Values after 2 Hours of Incubation of the mAb with the Antigen-Coupled Microspheres

In addition, binding specificity of mAbs towards antigen-coupled microspheres was evaluated by determining EC₅₀ and EC₂₅ values after 2 hours of incubation of the mAb with the antigen-coupled microspheres.

Therefore, microspheres were vortexed for 20 sec. A working microsphere mixture was prepared by diluting the coupled microsphere stock to a final concentration of 25 microspheres/μL in assay buffer (1% (w/v) BSA in 1-fold PBS, pH 7.4) and vortexing for 5 sec. The working mixture was kept at room temperature until 50 μL of the prepared working microsphere mixture were added per well in a black flat bottom 96-well assay plate (Corning Inc., Cat. No. 3915). After working microsphere mixture addition, the assay plate was washed two times with PBS-T (0.05% (v/v) Tween-20 in PBS pH 7.4) in a magnetic plate washer (BioTek Instruments, Product Id. 400072). Afterwards, the plate was placed in a 96-well plate magnet (Life Technologies, Product Id. 32513) and incubated for 30 sec while covering the plate. Following the incubation step, the supernatant was removed. mAbs were diluted from the corresponding stock concentrations in assay buffer to result in concentrations ranging from 0.001 to 20 μg/mL. After microsphere wash, 50 μL of each serially diluted mAb, or 50 μL assay buffer (2 blank wells per plate) were added to the microspheres. Each mAb dilution was examined in duplicates. The plates were covered with a foil sealing sheet and incubated for 2 hours at room temperature on a plate shaker at 600 rpm. After incubation, the assay plate was washed two times with PBS-T (0.05% (v/v) Tween-20 in PBS pH 7.4) in the magnetic plate washer. Afterwards, the plate was placed in the 96-well plate magnet and incubated for 30 sec while covering the plate. Following the incubation step, the supernatant was removed. For detection, F(ab′)2-goat anti-rabbit IgG (heavy and light chain) cross-adsorbed phycoerythrin (PE)-conjugated secondary Ab (Invitrogen, Cat. No. 31846, Lot. No. TL2684941, 0.5 mg/mL), R-PE AffiniPure F(ab′)₂ fragment goat anti-mouse IgG secondary Ab (heavy and light chain; Jackson ImmunoResearch, Cat. No. 115-116-146, Lot. No. 143867, 0.5 mg/mL), or PE-conjugated goat anti-human IgG secondary antibody (Southern Biotech, Cat. No. 2040-09, Lot. No. B3919-X449B, 0.5 mg/mL) were diluted 1:50 in assay buffer to achieve a final working concentration of 10 μg/mL by vortexing for 5 sec. 50 μL of the corresponding diluted detection Abs were added to each well. The plate was covered with a foil sealing sheet and incubation carried out for 30 min (t 2 min) at room temperature on a plate shaker at 600 rpm. The assay plate was washed two times with PBS-T in the magnetic plate washer. After the washing steps, the plate was placed in the 96-well plate magnet and incubated for 30 sec while covering the plate. Following the incubation step, the supernatant was removed. The microspheres were resuspended in 100 μL assay buffer per well. At this point, storage of the plate sealed with foil sealing sheet overnight at 4° C. is possible. Previous to sample read-out, the plate is allowed to re-equilibrate to room temperature for 20 min (t 5 min) if stored overnight at 4° C. The plate was put on an orbital shaker at 600 rpm for at least 5 min in order to allow for complete resuspension of immunocomplexed microspheres. Finally, the plate was placed in the multiplexing MAGPIX® plate reader (Luminex Corporation, Austin, Texas). The program used was xPONENT® (Build 4.2.1705.0) and is set-up with sample volume: 50 μL per well; plate protocol: 96-well plate format; and microsphere protocol: Map, BP 50 regions, Type MagPlex.

Determination of EC₅₀ and EC₂₅ Values

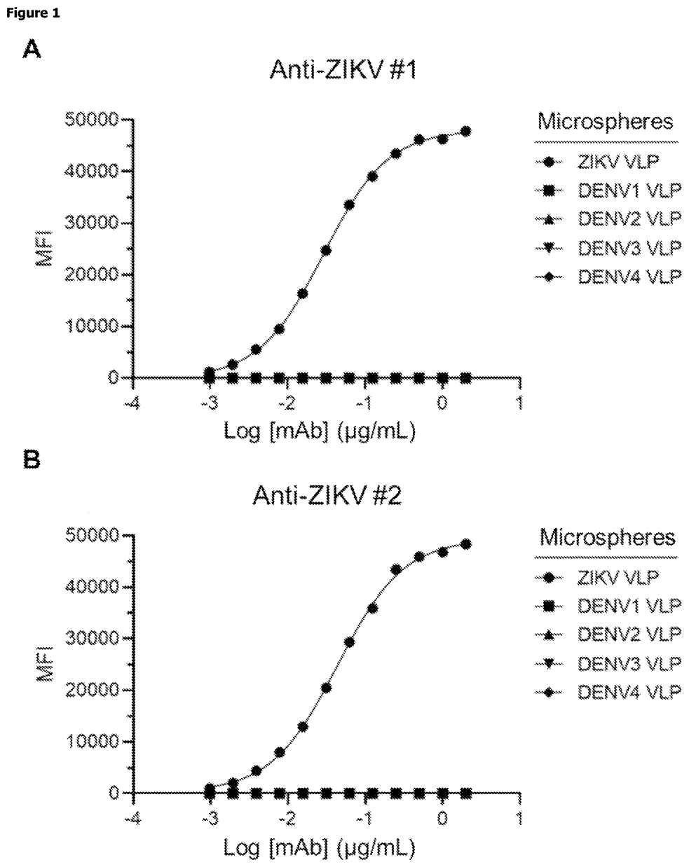

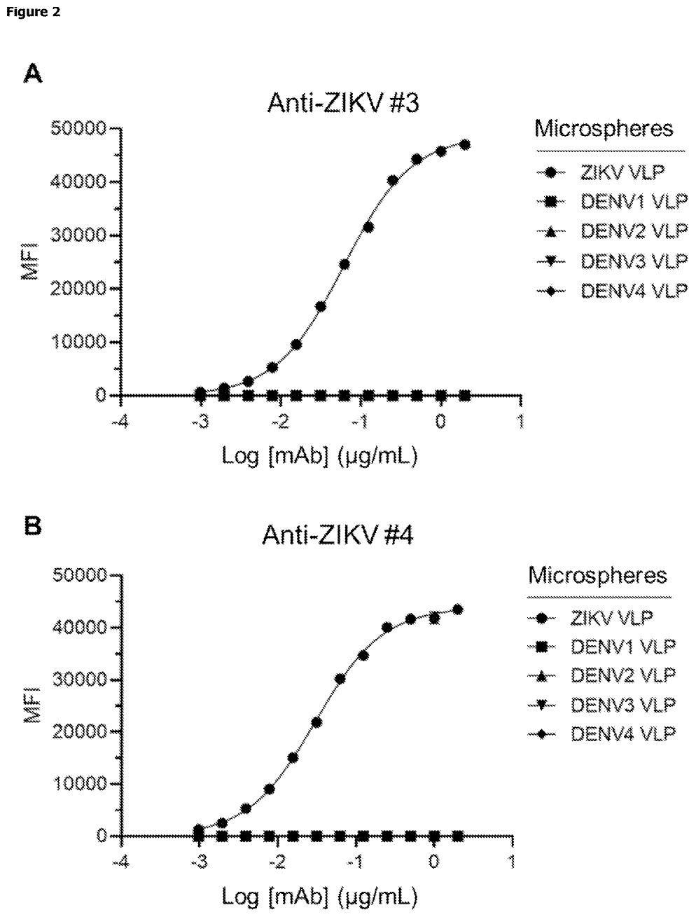

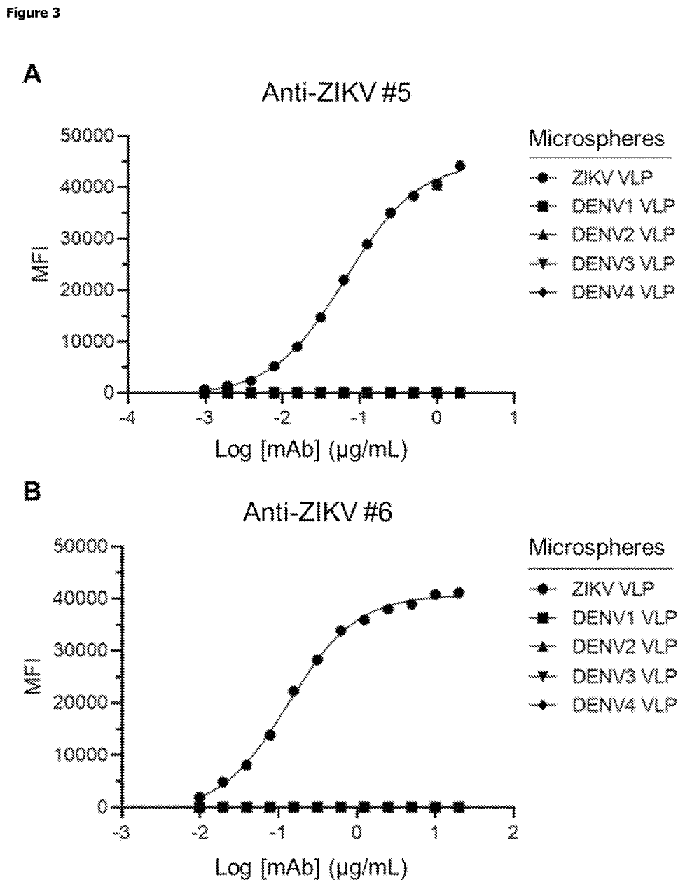

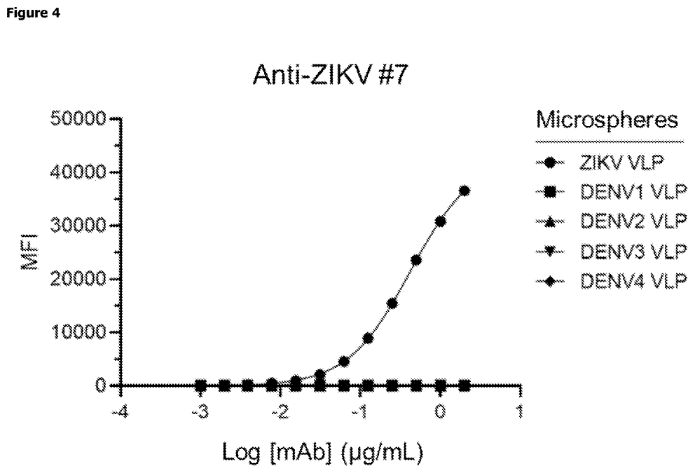

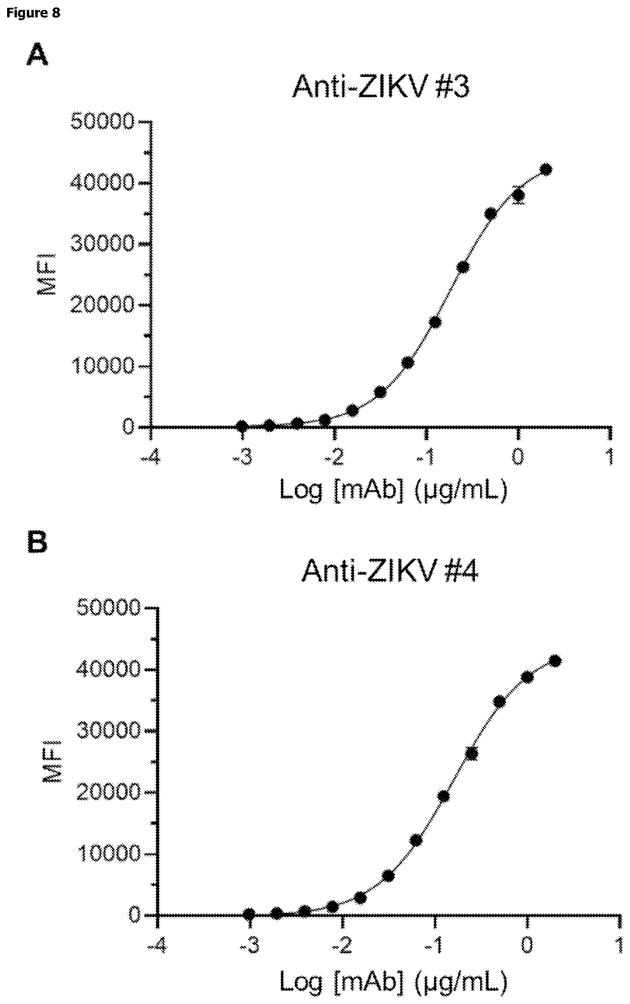

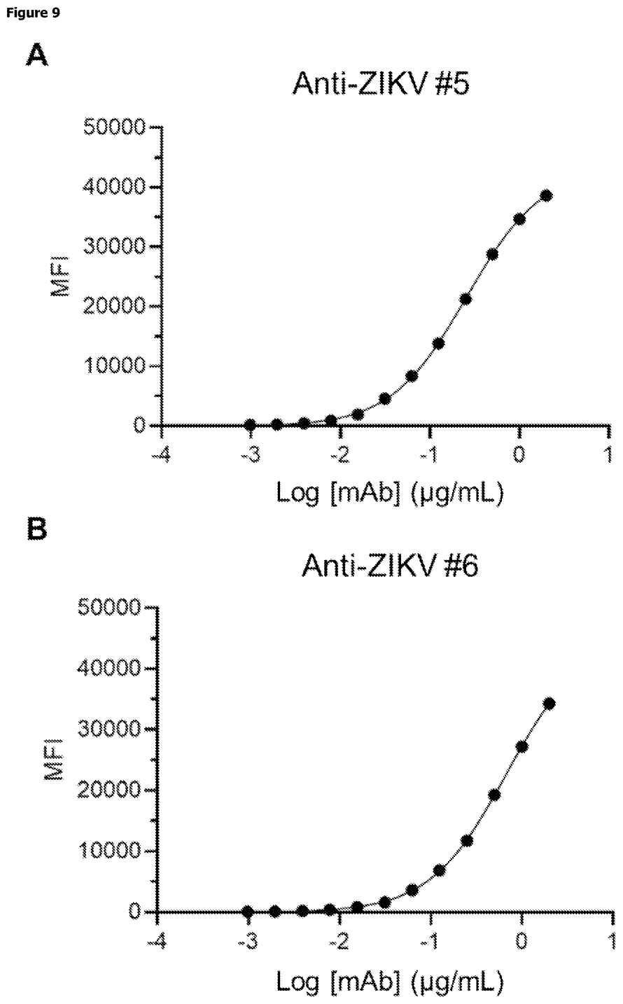

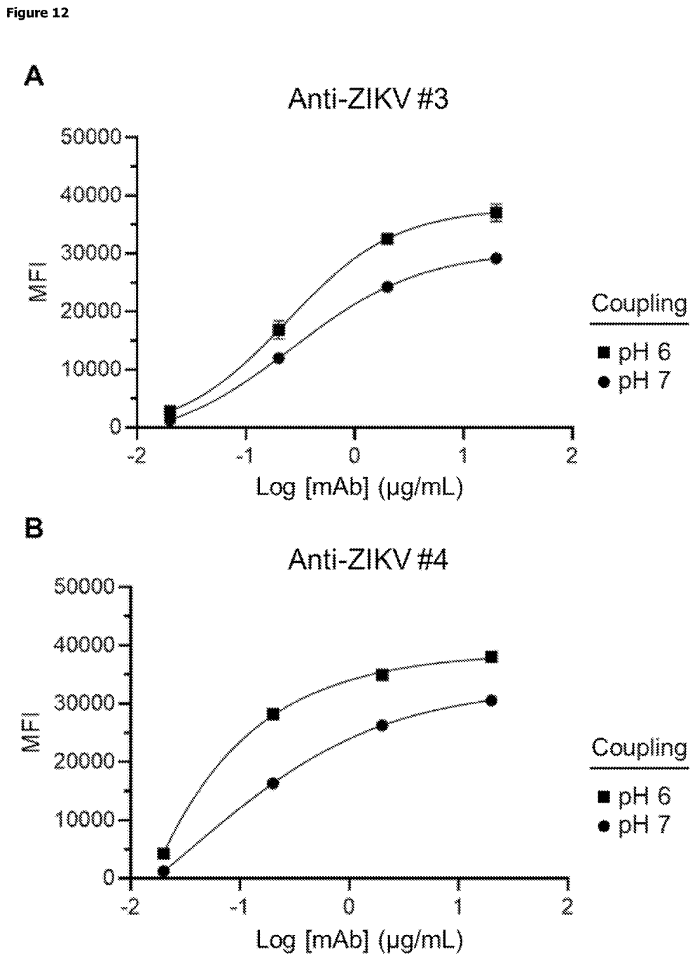

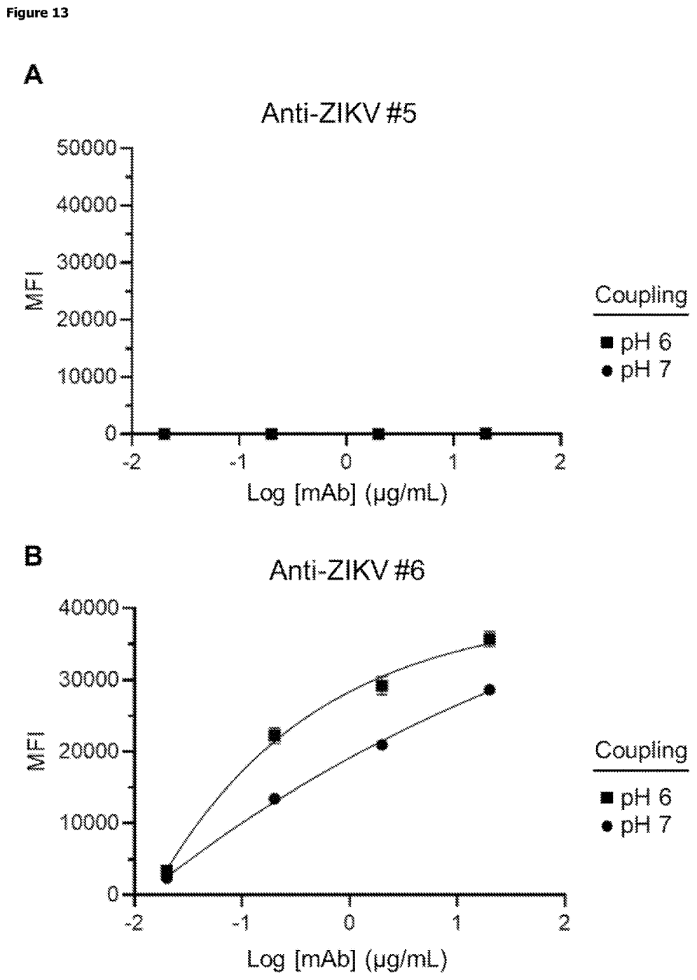

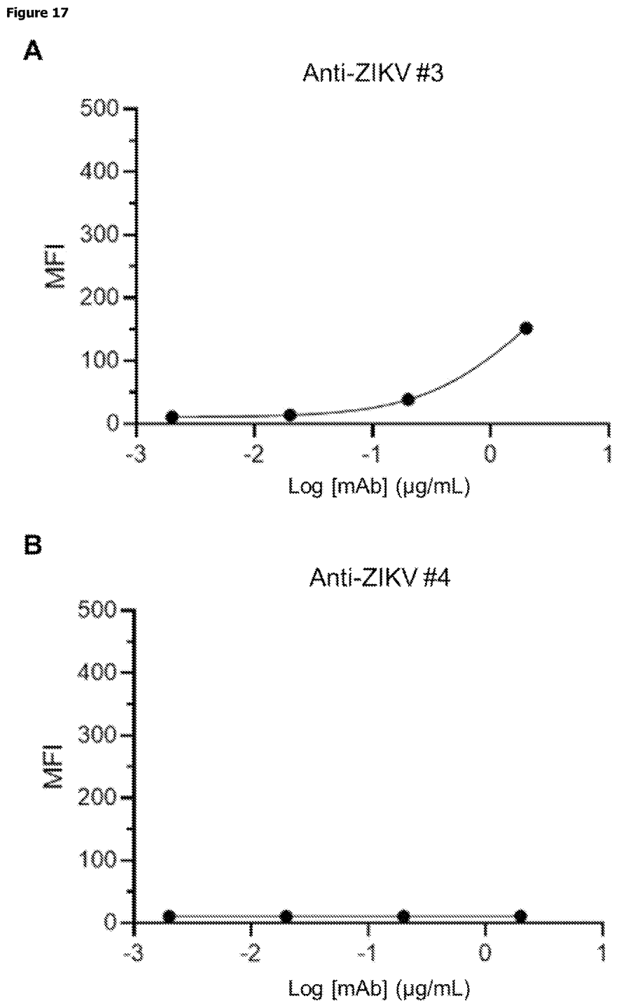

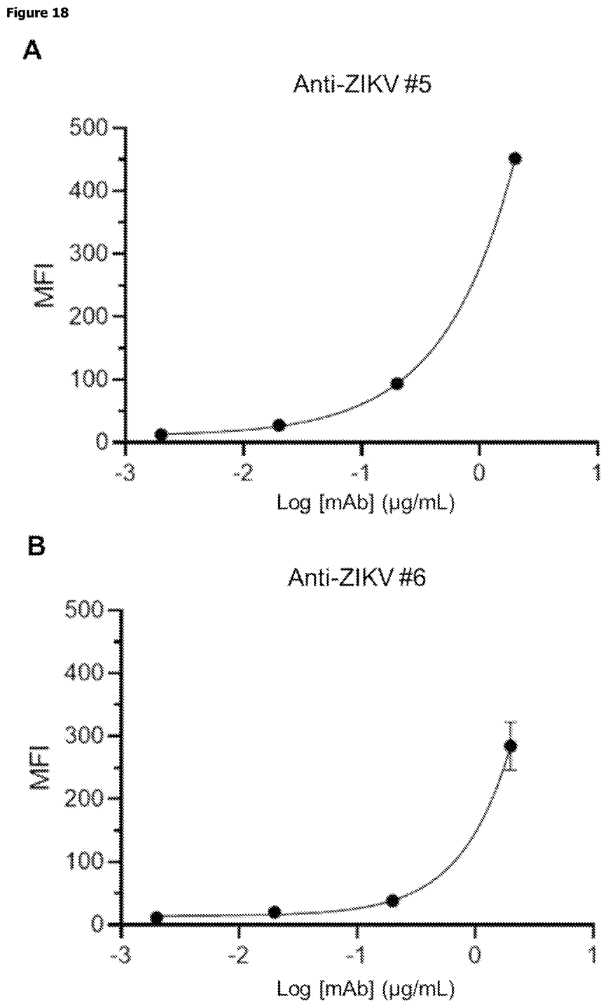

Data were analyzed and plotted using GraphPad Prism 8 version 8.1.0 (GraphPad Software, Inc.; FIG. 1-20, 22-23, 53-55). Sigmoidal fitting according to a dose-response curve (Sigmoidal, 4PL, X=Log(concentration)) carried out by Log-transformation and interpolation analysis of Median Fluorescent Intensity (MFI; =raw data as reported by the MAGPIX® reader, may also be referred to as “Net MFI”). The equation used for the non-linear regression was “log(agonist) vs. response-Variable slope”. EC₅₀ and/or EC₂₅ (2-fold effective concentration at which 25% or 50% of mAb, respectively, bind to the antigen-coupled microspheres) values were calculated for each example mAb in combination with the ZIKV VLP-coupled microspheres after incubation for 10 min, 60 min or 2 hours (Table 5). The mAb concentration used in the microsphere immunoassay (MIA) must be below saturating condition, meaning below the EC₁₀₀ (effective concentration at which 100% of mAb binds to the antigen-coupled microspheres) value. As none of the example mAbs showed any binding towards DENV1-4 VLPs, EC₅₀ and EC₂₅ values towards DENV1-4 VLPs could not be calculated from the data. For the comparative example mAbs, EC₅₀ and EC₂₅ values towards ZIKV and DENV1-4 VLPs were not calculated, except for Anti-PanDENV1-4 EDIII mAb. Anti-PanDENV1-4 EDIII mAb showed EC₅₀ values after 60 min incubation time with DENV1, DENV2, DENV3, and DENV4 VLPs of 3.376, 0.2379, 0.1095, and 0.3197 μg/mL, respectively. In addition, EC₅₀ and EC₂₅ values were not calculated for mAb-binding to rZIKV-EDIII-1-3 coupled to the microspheres. This was due to fewer mAb dilutions were examined and thus reliable EC₅₀ and EC₂₅ value calculation was not possible. Therefore, mAb binding to rZIKV-EDIII-1-3 was solely evaluated in a qualitative manner.

TABLE 5
EC₅₀ and EC₂₅ values for example mAbs incubated with ZIKV VLP for 120, 60 and/or 10 min.

| | EC₂₅ (μg/ml) | EC₅₀ (μg/mL) | | |
|---|---|---|---|---|
| | Incubation time: | Incubation time: | Incubation time: | Incubation time: |
| mAb | 120 min | 120 min | 60 min | 10 min |
| Anti-ZIKV #1 (Clone 102-1) | 0.0021 | 0.0066 | 0.029 | 0.1095 |
| Anti-ZIKV #2 (Clone 242-3) | 0.0024 | 0.0080 | 0.044 | 0.1397 |
| Anti-ZIKV #3 (Clone 270-12) | 0.0045 | 0.0144 | 0.061 | 0.1813 |
| Anti-ZIKV #4 (Clone 289-3) | 0.0025 | 0.0076 | 0.030 | 0.1607 |
| Anti-ZIKV #5 (Clone 306-2) | 0.0047 | 0.0158 | 0.066 | 0.2563 |
| Anti-ZIKV #6 (IgG2c Clone ZV-67) | 0.0081 | 0.0273 | 0.134 | 0.6769 |
| Anti-ZIKV #7 (IgG2a Clone ZKA-64) | 0.0296 | 0.1010 | 0.423 | 1.1760 |
| Anti-ZIKV #8 (Clone 260-2) | 0.0075 | 0.0252 | N/A | N/A |
| Anti-ZIKV #9 (Clone 181-4) | 0.0059 | 0.0156 | N/A | N/A |
| Anti-ZIKV #10 (Clone 329-2) | 0.0043 | 0.0145 | N/A | N/A |
| Anti-ZIKV #11 (Clone 11-3) | 0.0032 | 0.0105 | N/A | N/A |

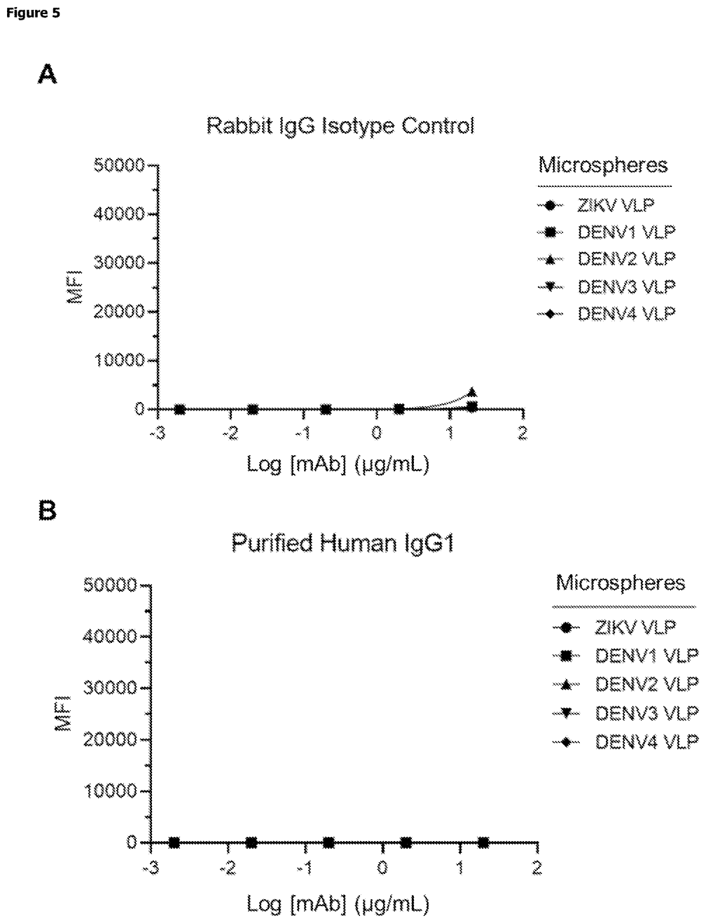

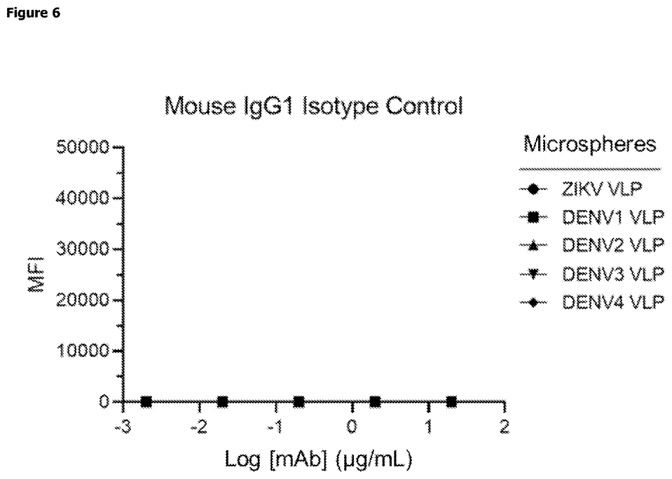

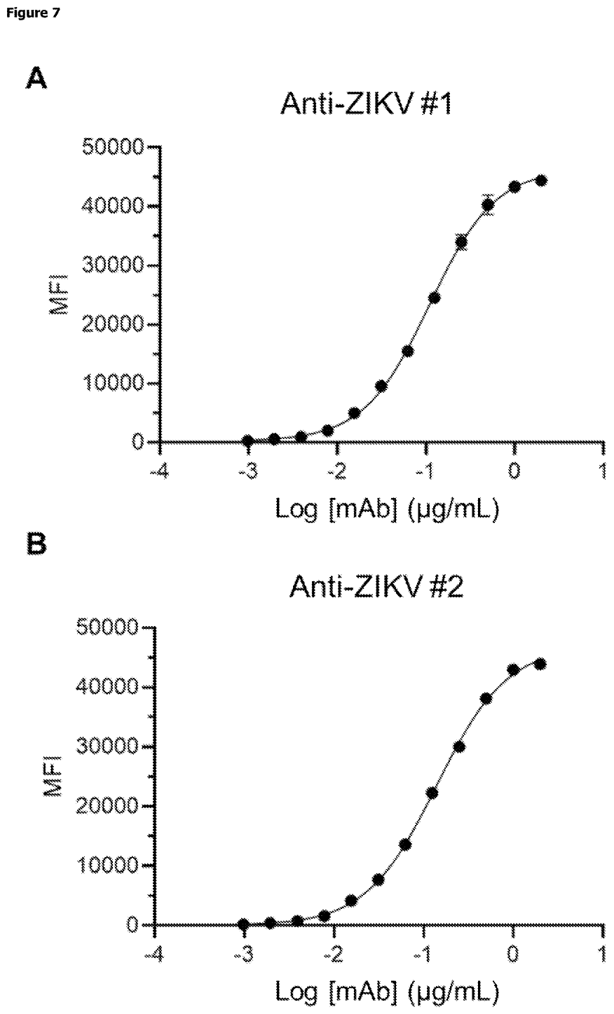

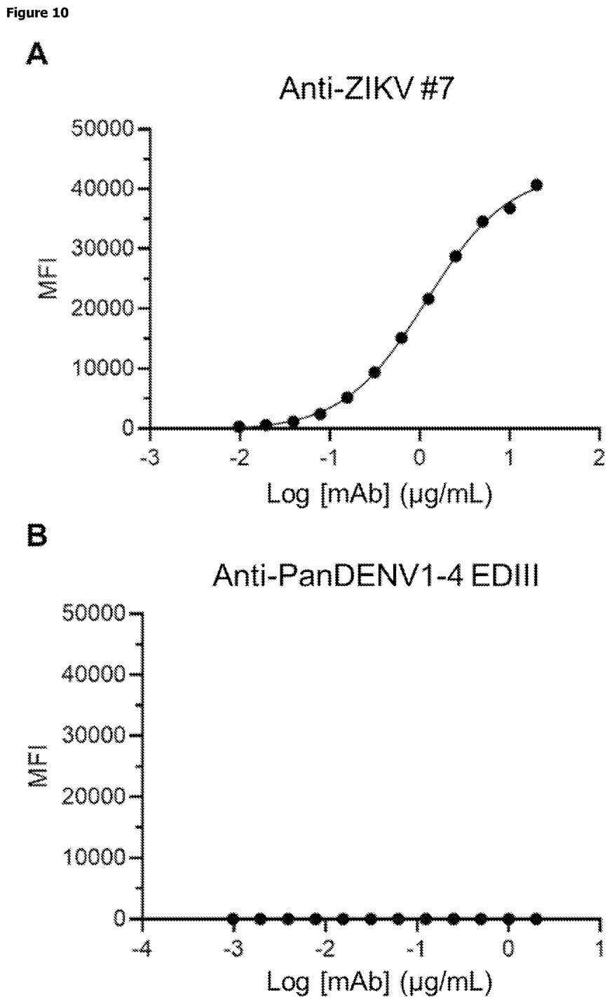

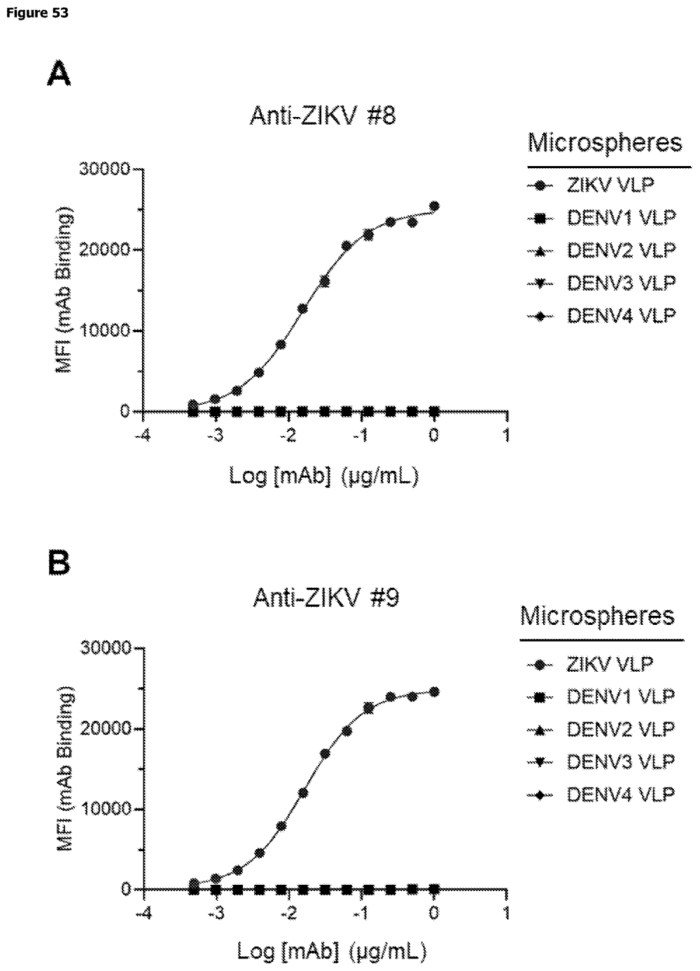

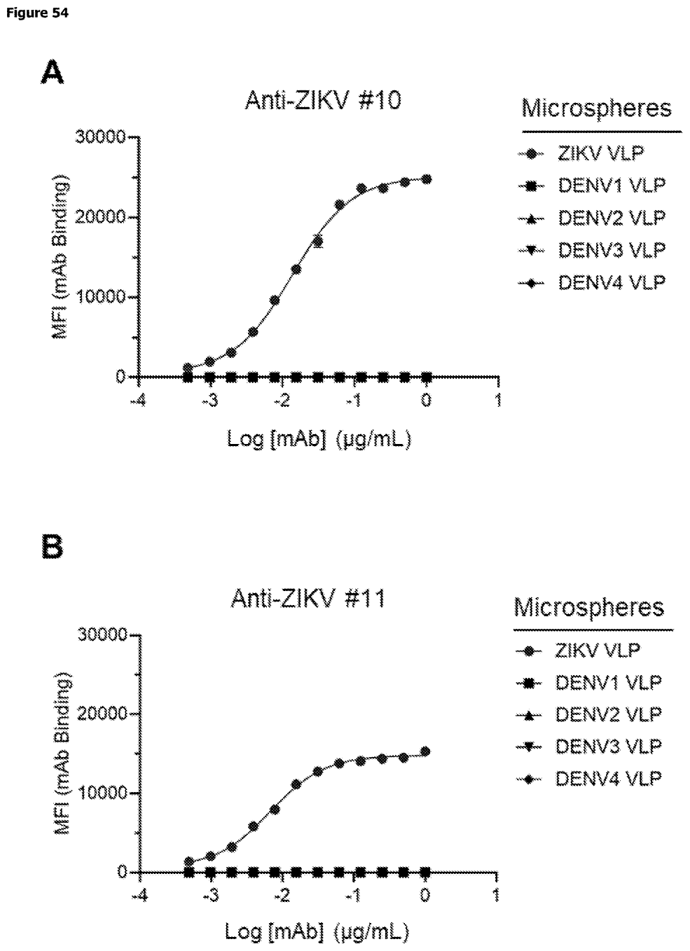

The data show that ZIKV VLP as the antigen coupled to the microspheres resulted in strong binding of all example mAbs anti-ZIKV #1 to 7 after 1 hour incubation of the mAbs with the microspheres (FIG. 1-4). Of note, mAbs anti-ZIKV #1 to 5 prepared in co-pending application (PCT/US2019/052189, WO 2020/106358) showed even lower EC₅₀ values than commercially available anti-ZIKV #6 and 7 demonstrating an improved affinity to the ZIKV VLP-coupled microspheres. In addition, also anti-ZIKV #8-11 showed strong binding to ZIKV VLPs after 120 min incubation of the mAbs with the microspheres, whereas no binding to DENV VLPs was observed (FIG. 53-55). Overall, no background-binding of controls was observed (FIG. 5-6). Moreover, also a shorter incubation time of 10 min of mAbs with the ZIKV VLP-coupled microspheres resulted in strong binding of anti-ZIKV #1 to 7 (FIG. 7-9, FIG. 10A). This indicates that shortening the incubation time is a possibility to improve through-put of the MIA.

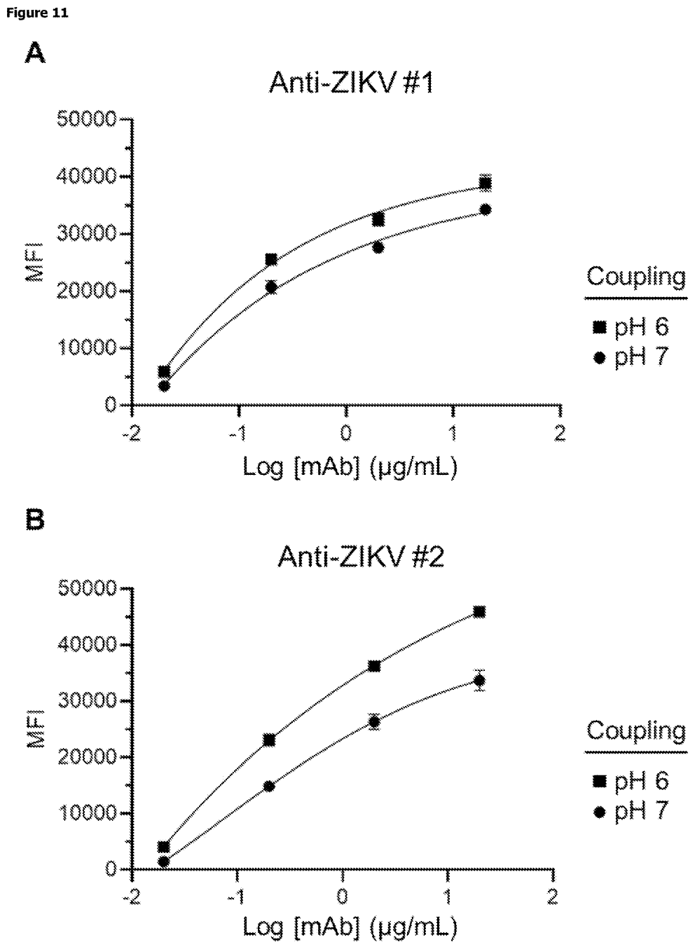

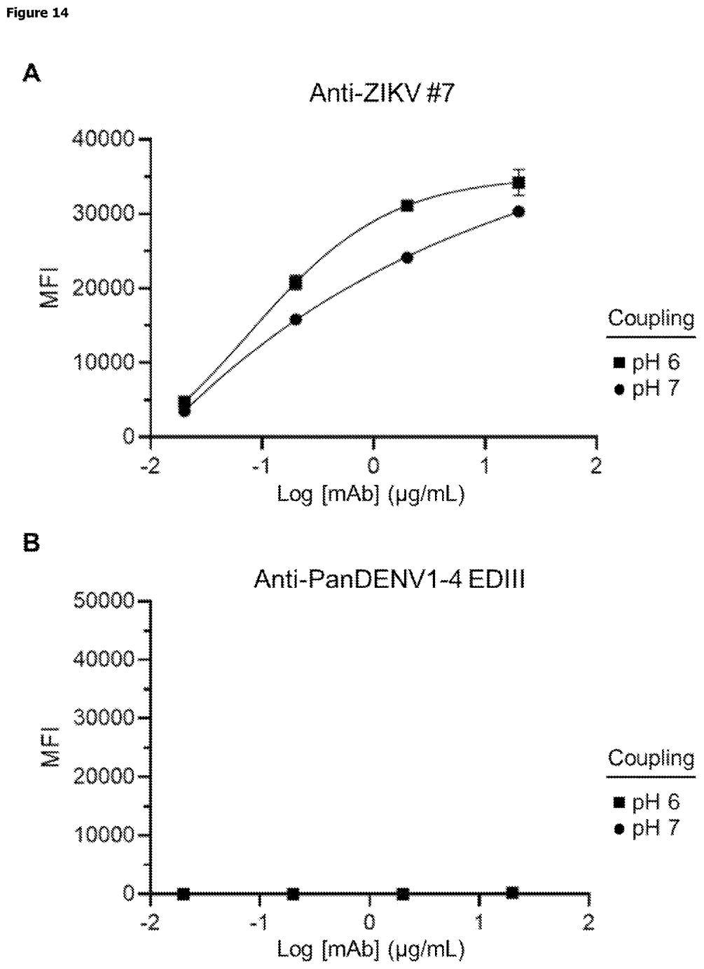

rZIKV-EDIII-1 as the antigen coupled to the microspheres resulted in overall strong binding of anti-ZIKV #1-4 and 6-7 (FIG. 11-13, FIG. 14A). The data indicated that optimum pH for coupling of rZIKV-EDIII-1 to the microspheres was pH 6 (comp. Example 1).

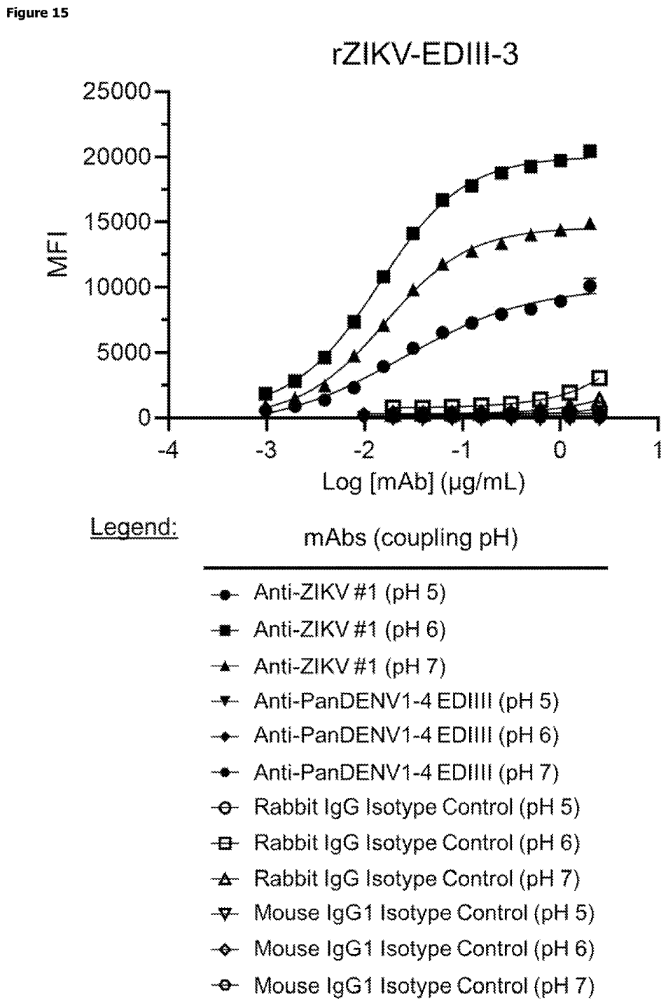

Anti-ZIKV #1 showed overall strong binding towards rZIKV-EDIII-3 as the antigen coupled to the microspheres (FIG. 15). The data indicated that optimum pH for coupling of rZIKV-EDIII-3 to the microspheres was pH 6 (comp. Example 1).

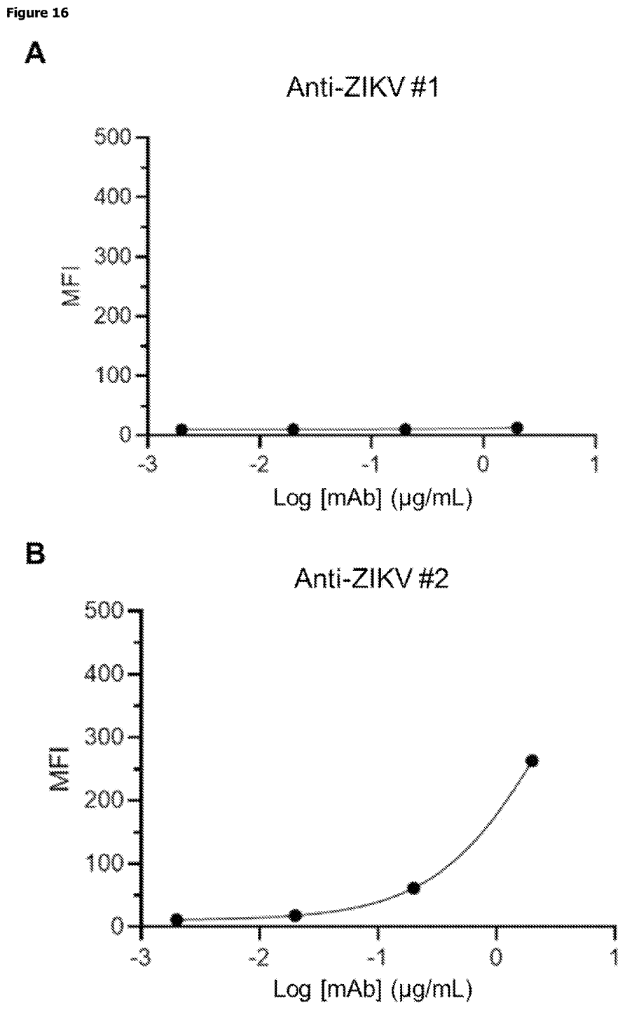

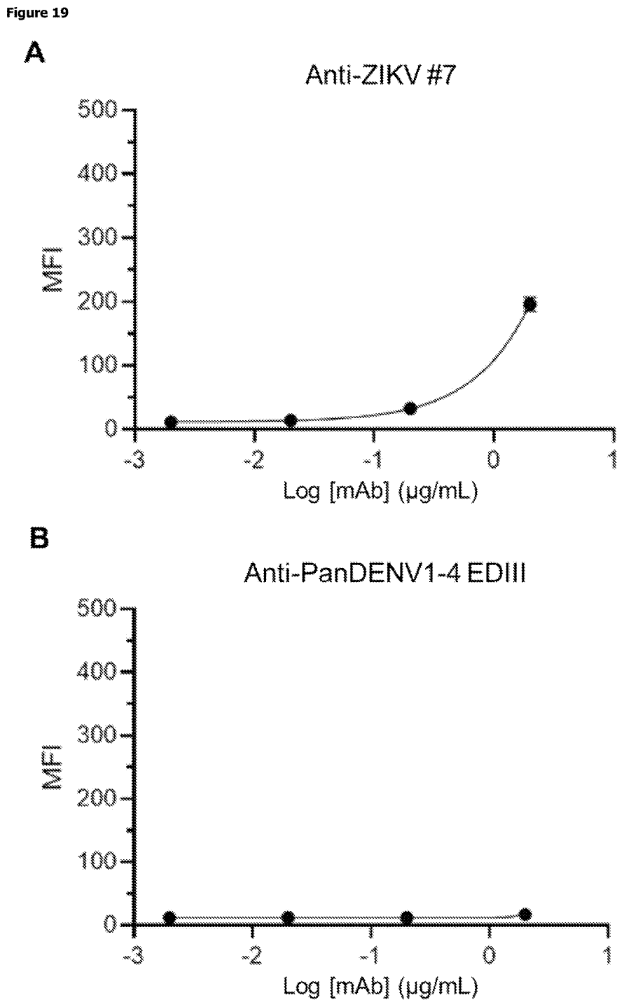

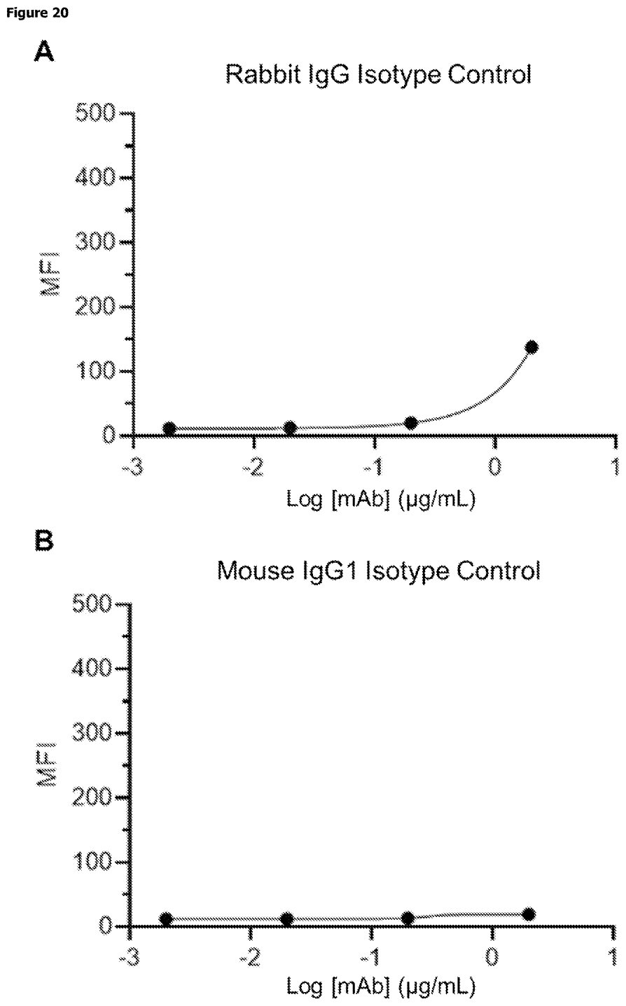

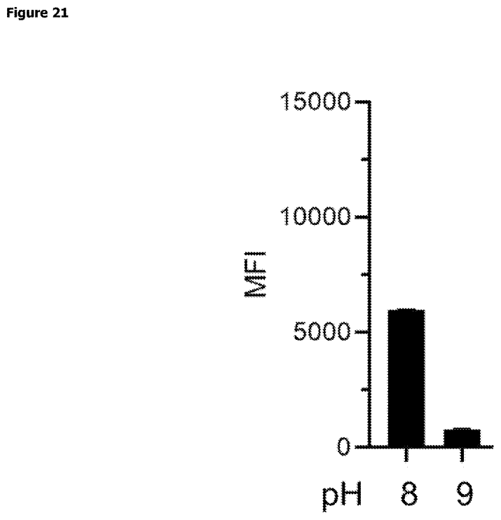

In contrast, binding of mAbs to rZIKV-EDIII-2 was comparatively low (FIG. 16-18, FIG. 19A). No binding of anti-ZIKV #1 and 4 and solely weak binding of anti-ZIKV #3 and 7 were observed for application of the rZIKV-EDIII-2 as antigen coupled to the microspheres. Moreover, although only observed for higher Ab concentrations, rabbit IgG isotype control showed background binding towards rZIKV-EDIII-2 (FIG. 20). In order to evaluate if a low coupling efficiency of rZIKV-EDIII-2 to the microspheres at pH 8.0 (the pH value applied for coupling of rZIKV-EDIII-2 to the microspheres previous to MIA) was the reason for low mAb binding, the presence of antigen His-tag bound to the microspheres after the coupling reaction was evaluated using a mouse anti-His-tag IgG1 Clone AD1.1.10 PE-conjugated detection Ab (R&D Systems, Cat. No. IC050P, Lot. No. LHNO319101). Detection of His-tag was carried out mutatis mutandis as described above for the evaluation of binding specificity of mAbs. The MFI values showed that pH 8 was the optimum for coupling of rZIKV-EDIII-2 to the microspheres, suggesting that the protein was successfully attached to the microspheres in sufficient amounts (FIG. 21). However, low Ab binding might have been the result of a disturbed antigen structure after coupling.

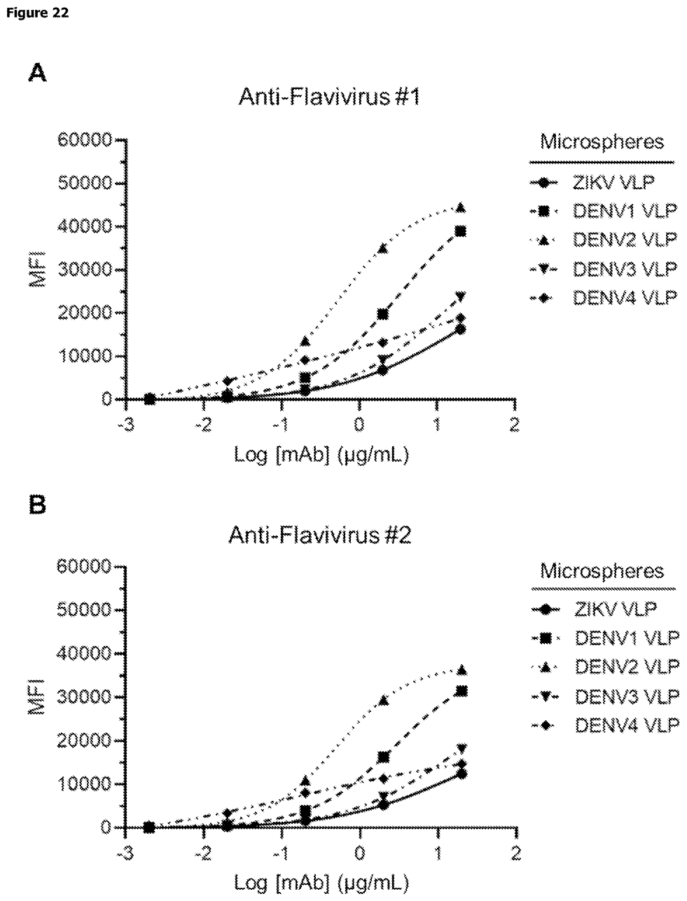

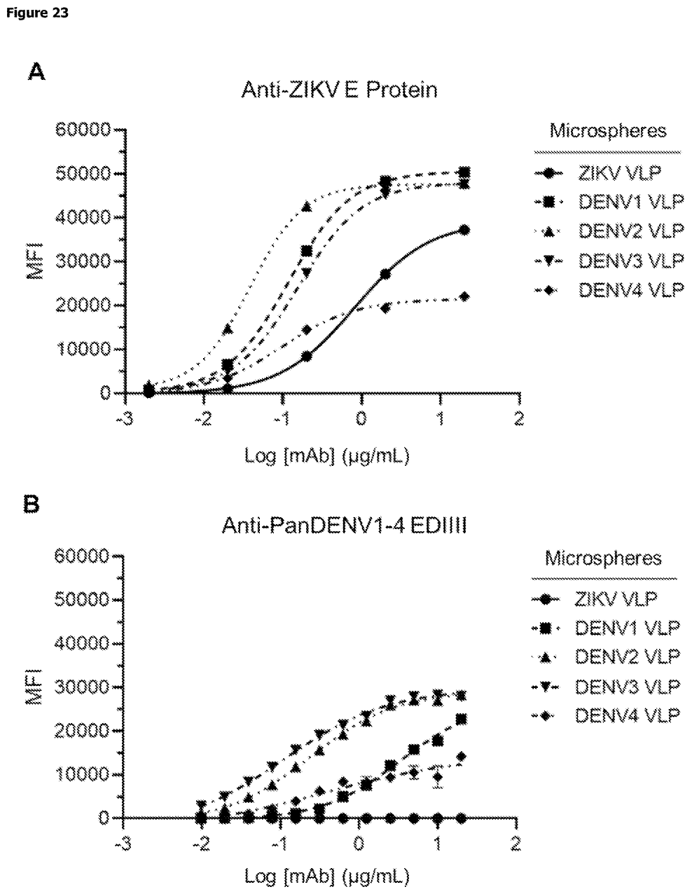

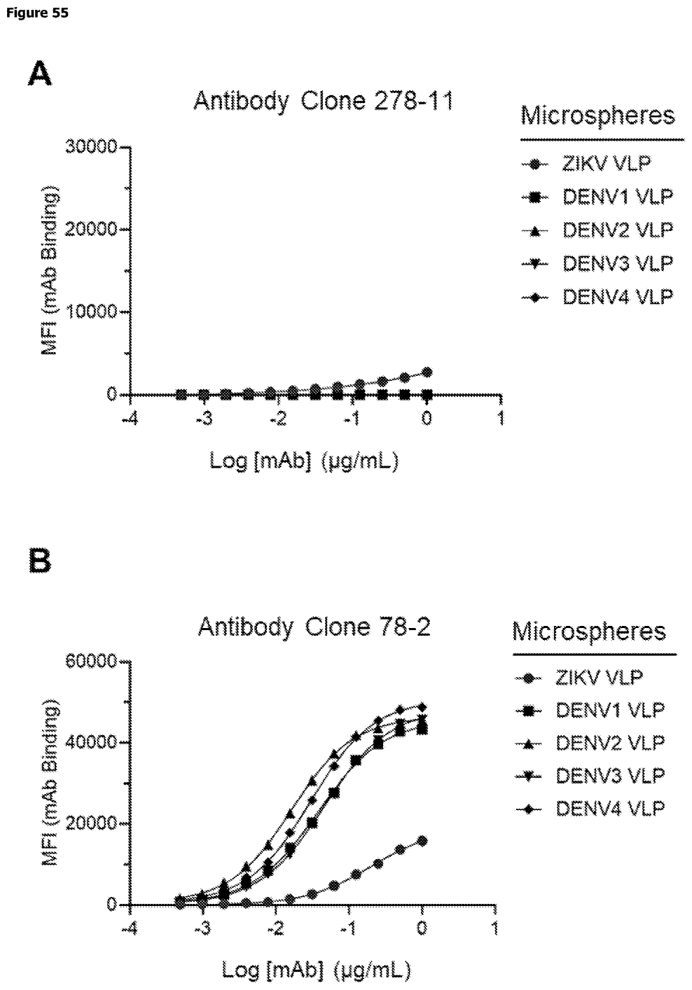

Notably, the comparative example mAb Anti-PanDENV1-4 EDIII did not bind to any of the ZIKV antigens evaluated (FIG. 10B, 14B, 15, 19B, 23B). Moreover, anti-Flavivirus #1 and 2, anti-ZIKV E Protein, and antibody Clone 78-2 showed strong cross-reactivity with DEN1-4 VLPs (FIG. 22, FIG. 23A, and FIG. 55B).

In summary, the ZIKV VLPs coupled to the microspheres showed consistent binding specificity results with all example mAbs. rZIKV-EDIII-2 seemed not suitable for use in the MIA.

### Example 3: Selection and Pre-Characterization of Human Plasma Samples

Human plasma samples #1 to 4 for testing in the MIA were obtained from a commercially available panel (ABO Pharmaceuticals; Table 6). Samples were collected in Colombia from single blood donations.

TABLE 6
Human plasma samples for testing in the microsphere immunoassay
(MIA). Presented are the date of symptoms onset, as well
as the date of specimen collection, ethnicity, gender, and
age of the blood donors at the time point of donation.

| | Symptoms | Specimen | | | |
|---|---|---|---|---|---|
| Sample ID | onset | collection | Ethnicity | Gender | Age |
| Plasma | 11 May 2015 | 6 Jun. 2015 | Hispanic | Male | 27 |
| sample #1 | | | | | |
| Plasma | 29 Apr. 2015 | 22 May 2015 | Hispanic | Female | 41 |
| sample #2 | | | | | |
| Plasma | 11 Apr. 2016 | 14 Apr. 2016 | Hispanic | Male | 30 |
| sample #3 | | | | | |
| Plasma | 2 May 2016 | 3 Jun. 2016 | Hispanic | Female | 31 |
| sample #4 | | | | | |

Plasma sample #1 (Specimen ID: PLA_116, Internal Specimen ID: PLA_116, Source ID: ABOFCOL01, Sample No. 0262), plasma sample #2 (Specimen ID: RBB2560, Internal Specimen ID: 757\hat{ }2, Source ID: ABOFCOL01, Sample No. 0221), plasma sample #3 (Specimen ID: Z_0087_B, Internal Specimen ID: PARS_71, Source ID: ABOFCOL01, Sample No. 1038) and plasma sample #4 (Specimen ID: Z_0096_B, Internal Specimen ID: PARS_81, Source ID: ABOFCOL01, Sample No. 1043) were further characterized with a ZIKV reporter virus particle (RVP) assay as described in Young et al. Sci Rep 2020, 16(3488). In brief, samples were heat-inactivated in a water bath for 30 min at 56° C. (t 0.2° C.) and incubated with ZIKV RVP (Integral Molecular, Cat. No. ZIKV SPH2015 RVP-Renilla, Lot. No. P-229A) for 1 hour at 37° C. The mixture was incubated with Vero cells for 72 hours at 37° C. Renilla-Glo substrate (Promega, Cat. No. E2710) was then added and incubation carried out for 15 min at room temperature. Finally, plates were analyzed in a luminometer. The effective concentration at 50% (EC₅₀ RVP) was determined by a non-linear regression curve fit with the lower asymptote constrained to 0 (GraphPad Prism). The human lower limit of quantification (LLOQ) is log₁₀ EC₅₀ RVP titer <2.0 Relative Luciferase Units (RLU; EC₅₀ RVP titer <100 RLU), below which the matrix interfered with the measurement. Plasma samples #1 and 2 showed lower EC₅₀ RVP titers (1,227 and 519.5 RLU, respectively) when compared to the titers of plasma samples #3 and 4 (15,316 and 3,269 RLU, respectively). Consequently, plasma samples #1-2 and #3-4 were categorized as ZIKV low- or high-reactive samples, respectively. ZIKV low-reactive samples were not expected to contain ZIKV specific Abs, whereas ZIKV high-reactive samples were expected to contain ZIKV specific Abs. ZIKV low-reactive samples were considered to contain anti-DENV Abs due to RVP titers above the human LLOQ.

The results from RVP assay are also in line with the timing of sample collection. ZIKV low-reactive samples were collected between May and June 2015, whereas ZIKV high-reactive samples were collected between April and June 2016. The first cluster of PCR-confirmed locally-acquired ZIKV cases in Colombia were reported to Pan American Health Organization/World Health Organization (PAHO/WHO) in October 2015 (https://www.paho.org/en/documents/zika-epidemiological-report-colombia-2).

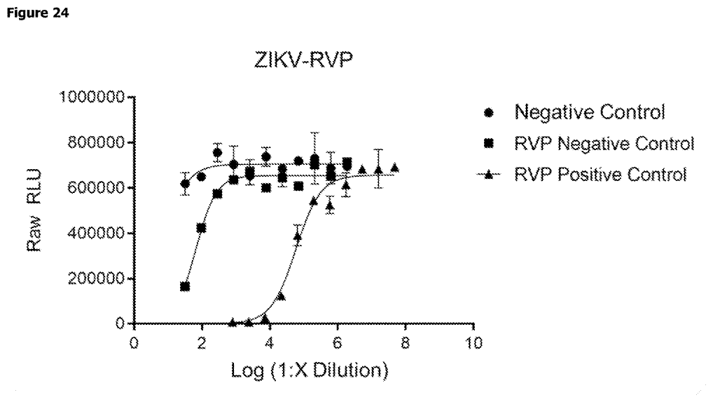

In addition to plasma samples #1 to 4, a human serum sample was included as negative control (Human Serum from a male, 52-aged donor; BioreclamationIVT, Cat. No. HMSRM, Lot. No. BRH1140253). This control did not react or solely reacted to a very low extent with DENV1-4 NS1 proteins in an ELISA. Moreover, in the ZIKV RVP assay (performed as described above) the negative control showed almost no neutralizing capacity compared to a RVP positive control, indicating that the sample does not contain anti-flavivirus Abs (FIG. 24).

For analysis, a certain amount of each sample was heat-inactivated previous to testing in a water bath for 30 min at 56° C. (±0.2° C.).

### Example 4: Selection of Antigen-Coupled Microspheres for MIA

In the next step, suitability of antigen-coupled microspheres for application in the MIA was evaluated using the human plasma samples from Example 3 and determining total anti-ZIKV IgG levels within the samples.

Antigen-coupled microspheres were prepared as described under Example 1 and vortexed for 20 sec. A working microsphere mixture was prepared by diluting the coupled microsphere stock to a final concentration of 25 microspheres/μL in assay buffer (1% (w/v) BSA in 1-fold PBS, pH 7.4) and vortexed for 5 sec. The working mixture was kept at room temperature until 50 μL of the prepared working microsphere mixture were added per well in a black flat bottom 96-well assay plate (Corning Inc., Cat. No. 3915). Heat-inactivated human plasma samples #1 to 4 as well as the negative control human serum sample were prepared as described under Example 3 and diluted 5-fold in assay buffer by pipetting up and down 20 times. From this 5-fold dilution, samples were 10-fold serially diluted in assay buffer in a deep-well 2 mL plate (ThermoFisher Scientific, Cat. No. 278752) by pipetting up and down 5 times between the dilutions to result in final dilutions of 1:5, 1:50, 1:500, and 1:5,000 when referred to the undiluted samples. For ZIKV VLP and rZIKV-EDIII-1 as antigens coupled to the microspheres, 50 μL of each serially diluted sample and assay buffer (2 blank wells per plate) were added per well to the microspheres to result in final sample dilutions of 1:10, 1:100, 1:1,000, and 1:10,000. For rZIKV-EDIII-2 and 3, 50 μL of the 5-fold dilution and assay buffer (2 blank wells per plate) were added per well to the microspheres to result in a final sample dilution of 1:10. Every dilution was examined in duplicates. The samples and the microspheres were mixed by pipetting up and down three times. The plates were covered with a foil sealing sheet and incubated for 60 min (5 min) at room temperature on a plate shaker at 600 rpm. After incubation, the assay plate was washed two times with PBS-T (0.05% (v/v) Tween-20 in PBS pH 7.4) in a magnetic plate washer (BioTek Instruments, Product Id. 400072). Afterwards, the plate was placed in a 96-well plate magnet (Life Technologies, Product Id. 32513) and incubated for 30 sec while covering the plate. Following the incubation step, the supernatant was removed. For detection, a goat anti-human IgG detection Ab conjugated to PE (SouthernBiotech, Cat. No. 2040-09, Lot. No. B3919-X449B) was diluted 1:50 in assay buffer to achieve a final working concentration of 10 μg/mL by vortexing for 5 sec. 50 μL of the diluted detection Ab were added to each well. The plate was covered with a foil sealing sheet and incubation carried out for 30 min (±2 min) at room temperature on a plate shaker at 600 rpm. The assay plate was washed two times with PBS-T in the magnetic plate washer. After the washing steps, the plate was placed in the 96-well plate magnet and incubated for 30 sec while covering the plate. Following the incubation step, the supernatant was removed. The microspheres were resuspended in 100 μL assay buffer per well. At this point, storage of the plate sealed with foil sealing sheet overnight at 4° C. is possible. Before the read-out, the plate is allowed to re-equilibrate to room temperature for 20 min (t 5 min) if stored overnight at 4° C. The plate was put on an orbital shaker at 600 rpm for at least 5 min in order to allow for complete resuspension of immunocomplexed-microspheres. Finally, the plate was placed in the multiplexing MAGPIX® plate reader (Luminex Corporation, Austin, Texas). The program used was xPONENT® (Build 4.2.1705.0) and is set-up with sample volume: 50 μL per well; plate protocol: 96-well plate format; and microsphere protocol: Map, BP 50 regions, Type MagPlex. For an assay to be considered as valid, the MFI values from the blank controls were required to be very low compared to the MFI values resulting from the sample wells.

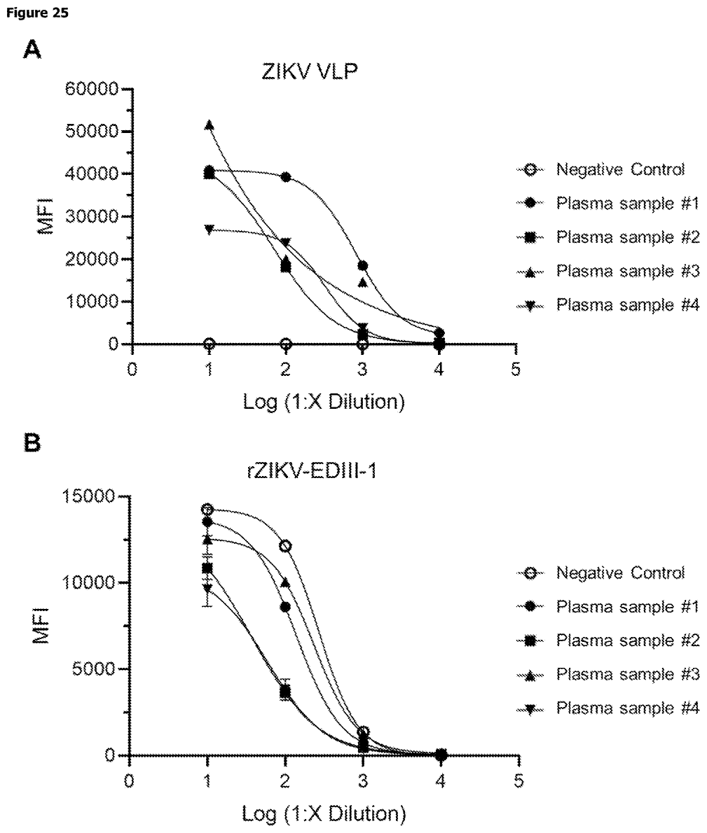

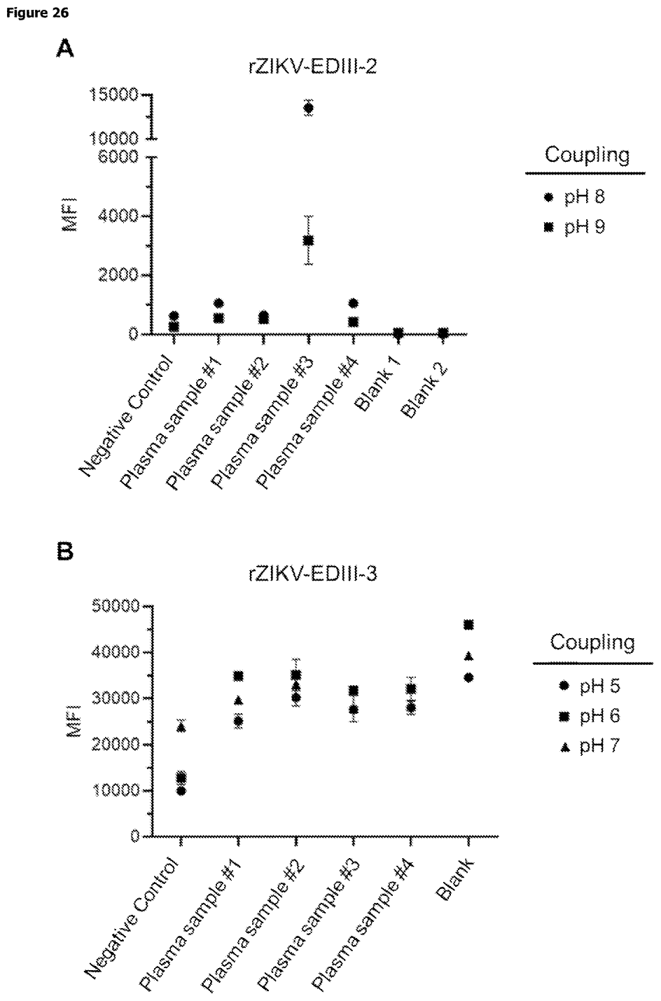

The total anti-ZIKV IgG levels of samples from Example 3 were determined using ZIKV VLPs, as well as rZIKV-EDIII-1-3 as antigens coupled to the microspheres (cf. Example 1 for information on antigens and coupling procedure). FIGS. 25 and 26 show the recorded MFI values for each sample dependent on the antigen applied.

No binding of the negative control to ZIKV VLPs was observed (FIG. 25A). Plasma samples #1 and 2, which are considered to be ZIKV low-reactive bind in a dose-dependent manner to a similar extent to ZIKV VLPs as plasma samples #3 and 4, which are considered to be ZIKV high-reactive. This is in line with the expectations, as plasma samples #1 and 2 are considered DENV positive and therefore to contain cross-reactive Abs which are determined in the total anti-ZIKV IgG MIA as well.

Contrarily, strong background binding of the negative control to rZIKV-EDIII-1 was observed, which might be caused by the 6×His-SUMO-tag and/or a (partially) disturbed antigen structure after coupling (FIG. 25B). Moreover, ZIKV high-reactive plasma sample #4 showed barely a signal when incubated with rZIKV-EDIII-2, indicating hampered binding of plasma anti-ZIKV Abs to rZIKV-EDIII-2 (FIG. 26A). Finally, total anti-ZIKV IgG levels were determined with rZIKV-EDIII-3 coupled to the microspheres. Strong signal was observed from the blank wells, suggesting unspecific binding of the anti-human detection Ab towards the Fc-portion of the immobilized antigen (FIG. 26B). In addition, similar to the observations with rZIKV-EDIII-1, also plasma samples #1 and 2, that are considered ZIKV low-reactive, resulted in a similar signal compared to the ZIKV high-reactive plasma samples #3 and 4 in the MIA using rZIKV-EDIII-3.

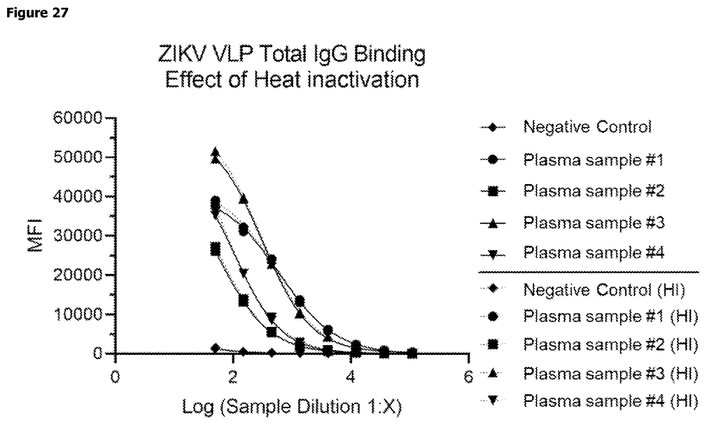

In addition to analysis of heat-inactivated samples, the samples as described under Example 3 were tested in the total anti-ZIKV IgG MIA without previous heat-inactivation. The assay was performed as described above for the heat-inactivated samples using ZIKV VLPs as antigens coupled to the microspheres, with the difference that more sample dilutions were examined. Interestingly, the total IgG MIA resulted in the same results for all examined samples independent of sample heat-inactivation (FIG. 27). This underlines a robust assay set-up, as components present previous to heat-inactivation (such as e.g. enzymes) are not disturbing the MIA and therefore a broad application of the assay independent of the nature of the sample becomes possible. Moreover, sample preparation time can be reduced due to the possibility to skip the inactivation step, which provides another advantage, for instance, in view of a high-throughput application of the method.

In summary, ZIKV VLPs as microsphere-coupled antigens provide a consistent data set. This is in conformity with the mAb-binding data from Example 2.

### Example 5: Evaluation of ZIKV Specific Ab Levels by a Competitive MIA (cMIA)

In the next step, human samples and non-human primate samples were evaluated for the presence of ZIKV specific Abs within the samples by a competitive MIA (cMIA) using antigen-coupled microspheres and mAbs from Examples 1 and 2. In addition to a qualitative proof-of-concept set-up, the cMIA was also performed in a quantitative way.

### Example 5.1: Qualitative Proof-of-Concept Set-Up

Microspheres as prepared under Example 1 were vortexed for 20 sec. A working microsphere mixture was prepared by diluting the coupled microsphere stock to a final concentration of 50 microspheres/μL in assay buffer (1% (w/v) BSA in 1-fold PBS, pH 7.4) and vortexing for 5 sec. The working mixture was kept at room temperature. Each 25 μL of the prepared working microsphere mixture were added per well of a black flat bottom 96-well assay plate (Corning Inc., Cat. No. 3915). Human plasma samples (plasma samples #1-4), as well as the negative control of Example 3 were heat-inactivated as described under Example 3 and were vortexed for 5 sec before transferring them into a deep-well 2 mL plate (ThermoFisher Scientific, Cat. No. 278752) to perform serial dilutions in assay buffer. Serial dilutions were prepared to result in a 5-, 10-, and 20-fold sample dilution for the cMIA using ZIKV VLP as microsphere-coupled antigens. Samples were mixed in between the dilutions by pipetting up and down 20 times (5-fold dilution) or 5 times (10- and 20-fold dilution), respectively. For rZIKV-EDIII-3 as antigen, samples were applied at a 5-fold dilution. Afterwards, 25 μL of each diluted sample were aliquoted into the assay plate per well in duplicates. Two controls were included by aliquoting 25 μL of assay buffer per well in duplicates to later account for 0% and 100% mAb binding. Afterwards, the mixture of microspheres and samples were pipetted up and down three times, which results in a 2-fold dilution of samples resulting in 10-, 20-, and 40-fold final dilutions. The plate was covered with a foil sealing sheet and incubated for 60 min (t 5 min) at room temperature on a plate shaker at 600 rpm. Meanwhile, an intermediary 50-fold (1:50) dilution of each example mAb (Anti-ZIKV #1 to 7) and Anti-PanDENV1-4 EDIII mAb was prepared by mixing 10 μL of the stock mAb solution with 490 μL of assay buffer. The 50-fold dilution was vortexed for 5 sec. Next, the 50-fold dilution was further diluted in assay buffer to reach the final mAb concentration, which corresponds to the EC₅₀ concentration of the mAb for ZIKV VLP-binding calculated under Example 2 after 10 min incubation with the ZIKV VLP coupled-microspheres (Table 5). The final mAb concentration solution was vortexed for 10 sec. After incubation of the samples with the microspheres no additional washing steps were carried out. 50 μL of the final mAb concentration solution were added to the wells. 50 μL of assay buffer were added to the wells corresponding to 0% mAb-binding. No additional mixing was performed. The plate was covered with a foil sealing sheet and incubated for 10 min at room temperature on a plate shaker at 600 rpm. After incubation, the assay plate was washed two times with PBS-T (0.05% (v/v) Tween-20 in PBS pH 7.4) in a magnetic plate washer (BioTek Instruments, Product Id. 400072). Afterwards, the plate was placed in a 96-well plate magnet (Life Technologies, Product Id. 32513) and incubated for 30 sec while covering the plate. Following the incubation step, the supernatant was removed. For detection of rabbit or mouse mAbs, respectively, a F(ab′)2-Goat anti-rabbit IgG (heavy and light chain) cross-adsorbed PE-conjugated (Invitrogen, Cat. No. 31846, Lot. No. TL2684941, 0.5 mg/mL) or a R-PE AffiniPure F(ab′)₂ fragment goat anti-mouse IgG (heavy and light chain; Jackson ImmunoResearch, Cat. No. 115-116-146, Lot. No. 143867, 0.5 mg/mL) secondary reporter Ab was applied. Secondary Abs were diluted 1:50 in assay buffer to achieve a final working concentration of 10 μg/mL by vortexing for 5 sec. 50 μL of the corresponding diluted detection Abs were added to each well. The plate was covered with a foil sealing sheet and incubation carried out for 30 min (t 2 min) at room temperature on a plate shaker at 600 rpm. The assay plate was washed two times with PBS-T in a magnetic plate washer. After the washing steps, the plate was placed in a 96-well plate magnet and incubated for 30 sec while covering the plate. Following the incubation step, the supernatant was removed. The microspheres were resuspended in 100 μL assay buffer per well. At this point, storage of the plate sealed with foil sealing sheet overnight at 4° C. was possible. Before sample read-out, the plate was allowed to re-equilibrate to room temperature for 20 min (t 5 min) if stored at 4° C. overnight. The plate was put on an orbital shaker at 600 rpm for at least 5 min in order to allow for complete resuspension of immunocomplexed microspheres. Finally, the plate was placed in the multiplexing MAGPIX® plate reader (Luminex Corporation, Austin, Texas). The program used was xPONENT® (Build 4.2.1705.0) and is set-up with sample volume: 50 μL per well; plate protocol: 96-well plate format; and microsphere protocol: Map, BP 50 regions, Type MagPlex. For an assay to be considered as valid, the MFI values from the blank controls were required to be very low compared to the MFI values resulting from the sample wells.

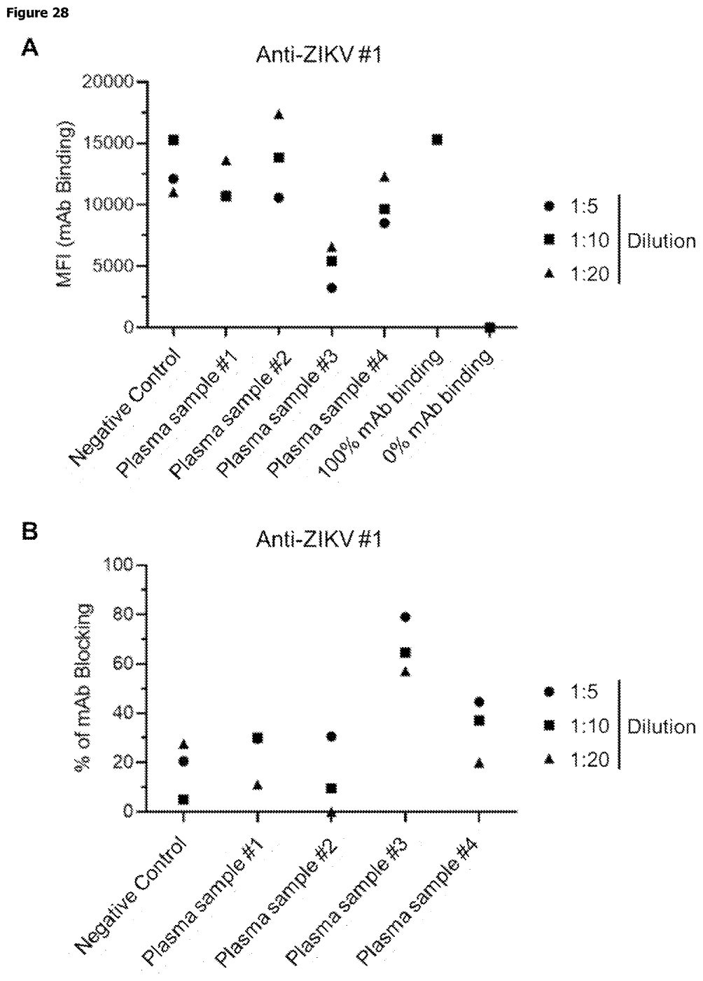

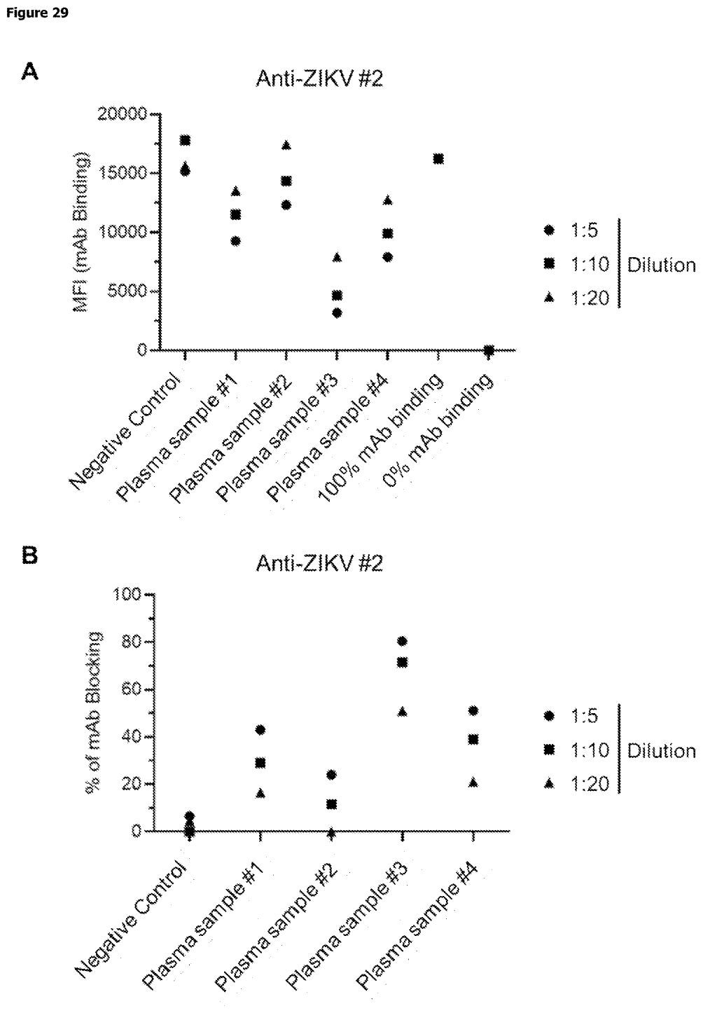

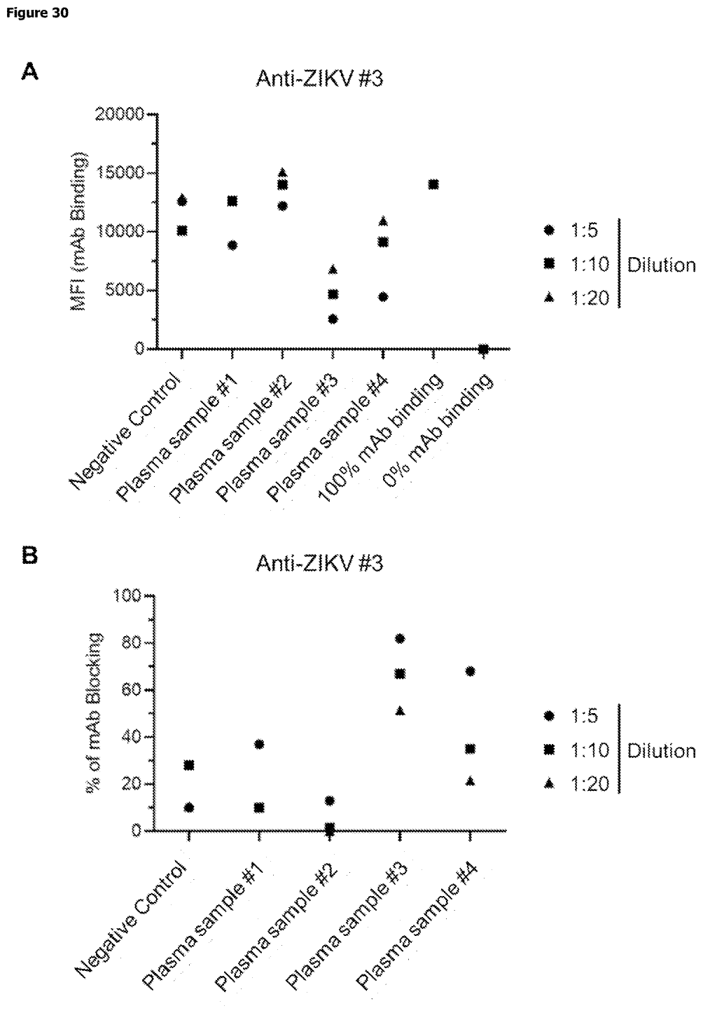

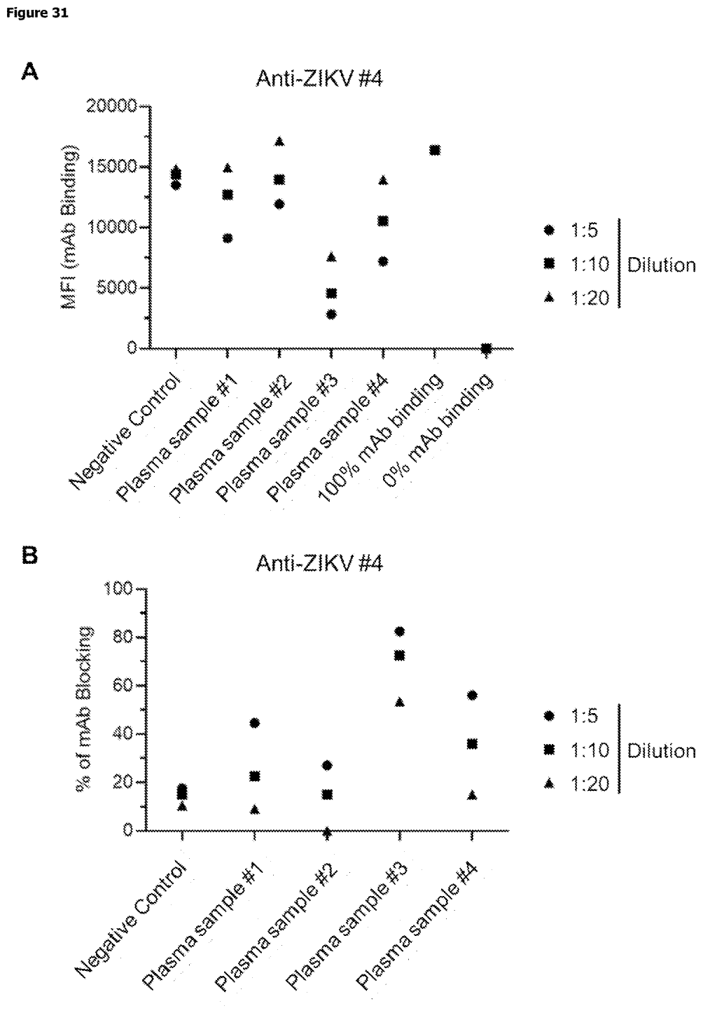

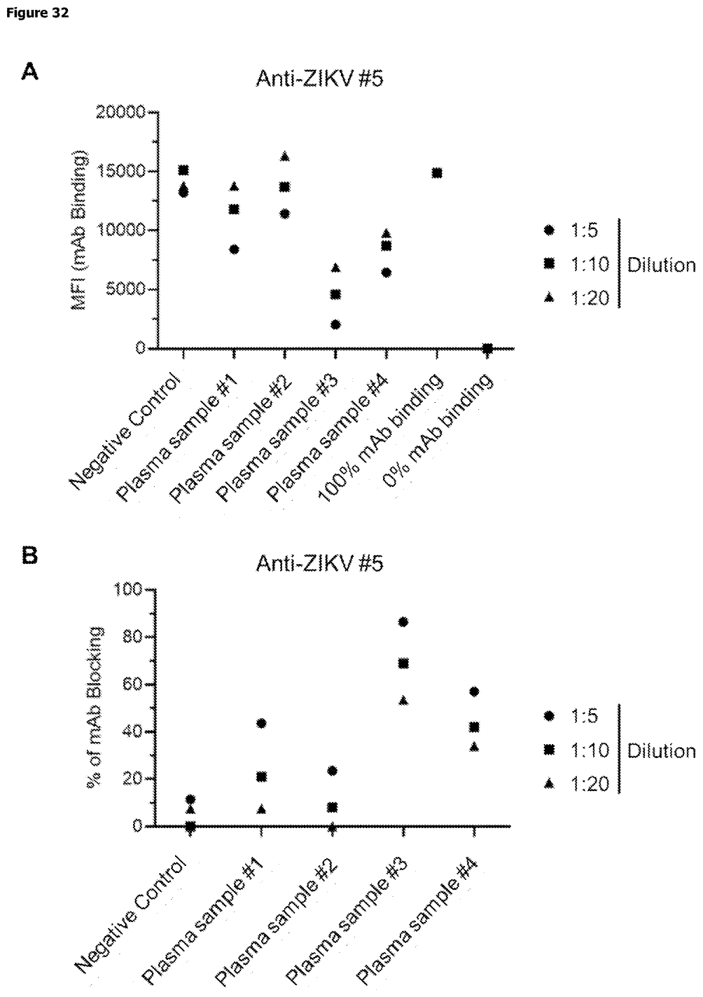

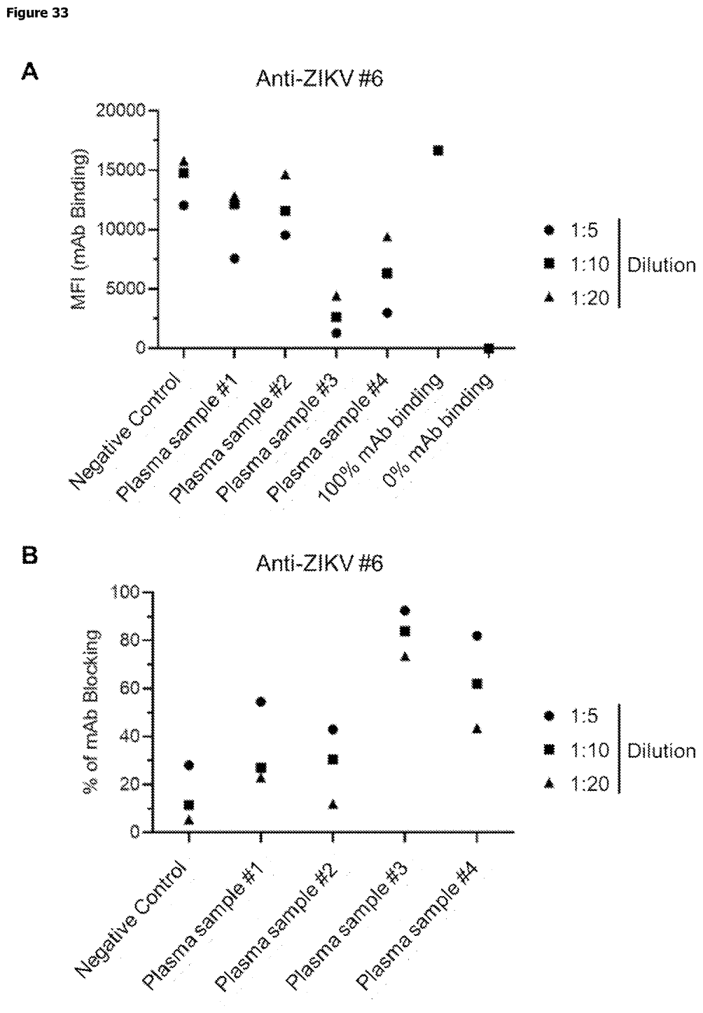

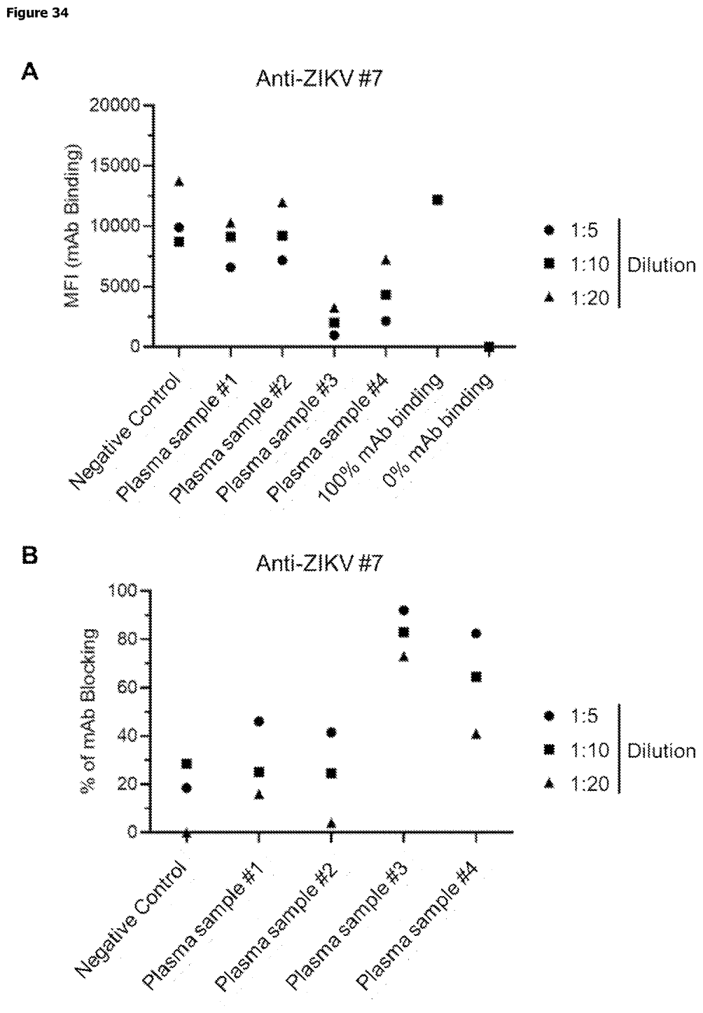

Data generated were analyzed and plotted using GraphPad Prism 8 version 8.1.0 (GraphPad Software, Inc.). MFI values reported by the MAGPIX® reader resulting from binding of Anti-ZIKV #1 to 7 and Anti-PanDENV1-4 EDIII mAbs are presented for the analyzed plasma samples and the negative control dependent on the plasma dilution (FIG. 28A-35A and FIG. 36). MFI values are indicative for the ability of each sample to block the binding of the mAbs to the ZIKV antigens coupled to the microspheres. High MFI values, which correspond to a high degree of mAb binding, were expected for the negative control, as no anti-ZIKV Abs were present within this sample. This was also expected for human plasma samples, which were reported to be ZIKV low-reactive and may contain cross-reactive Abs from previous flavivirus infections such as DENV, but no ZIKV specific Abs (plasma samples #1 and 2). In contrast, lower MFI values were expected for human plasma samples, which were reported to be ZIKV high-reactive (plasma samples #3 and 4), as ZIKV specific Abs were expected to be present in these samples. The MFI of each plasma sample dilution was divided by the mean MFI value of the signal of the 100% mAb binding control to result in the percentage of blocking of mAb binding (blockade-of-binding (BoB) values) for each sample dilution (FIG. 28B-35B).

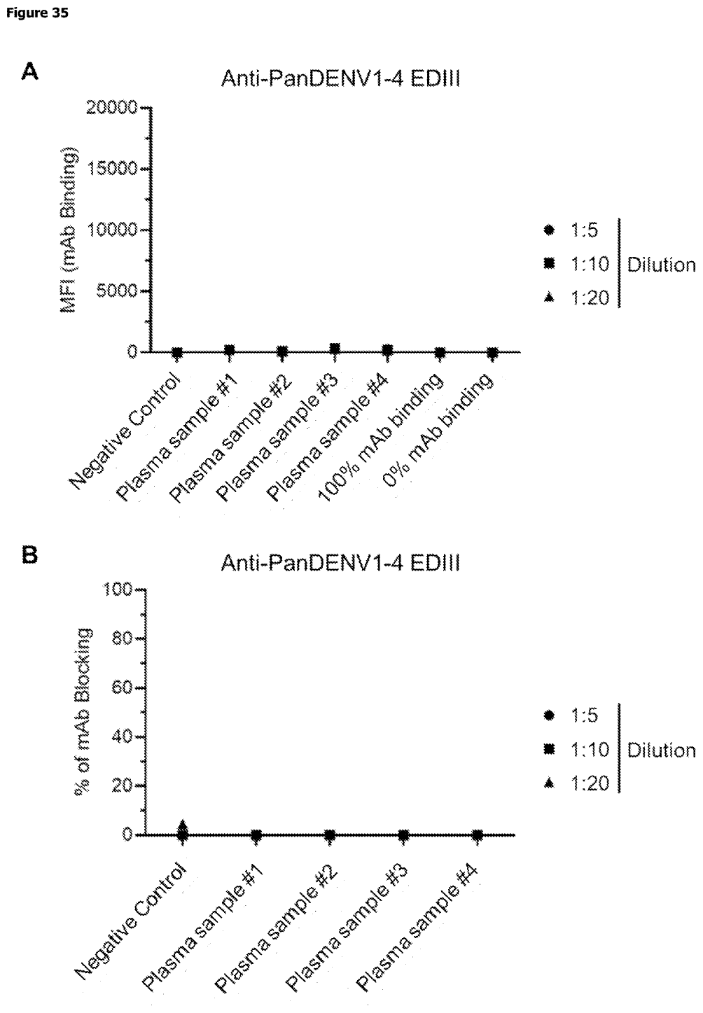

Satisfyingly, BoB values resulting from incubation of the negative control and human plasma samples #1 and 2, which are considered to be ZIKV low-reactive, with ZIKV VLPs as microsphere-coupled antigens were comparatively low for all anti-ZIKV #1 to 7 mAbs independent of the plasma dilution. On the other hand, higher BoB values were observed using the ZIKV high-reactive samples (human plasma samples #3 and 4; FIG. 28-34). Of note, no signal was observed using the Anti-PanDENV1-4 EDIII mAb (FIG. 35).

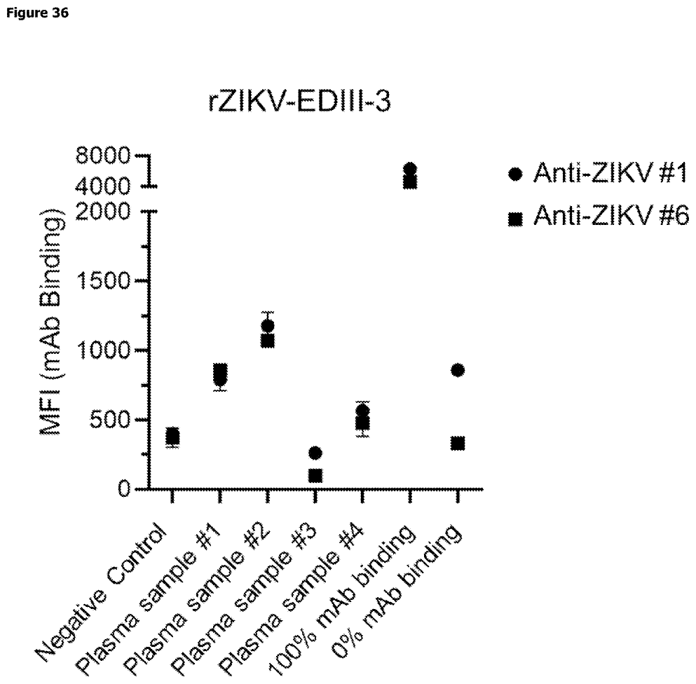

Contrarily, low MFI values were observed for human plasma samples #1 and 2 considered as ZIKV low-reactive when compared to the 100% mAb binding control using the rZIKV-EDIII-3 as antigen coupled to the microspheres, indicating unspecific blocking of mAb binding, as ZIKV specific Abs should be absent from these samples (FIG. 36). Moreover, a low MFI value comparable to human plasma samples #3 and 4, which are ZIKV high-reactive, was observed for the negative control, indicating unspecific binding of negative control components to the microsphere-coupled rZIKV-EDIII-3. In summary, these data suggest, that the rZIKV-EDIII-3 antigen was not suitable for the cMIA set-up.

### Example 5.2: Quantitative cMIA

### Example 5.2.1: Initial Quantitative cMIA Set-Up

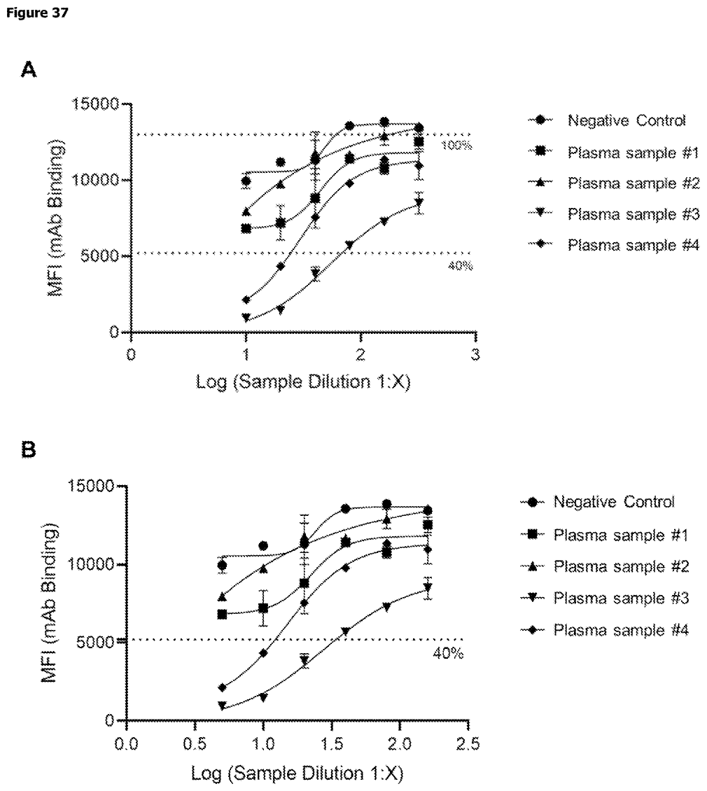

To evaluate the suitability of the cMIA set-up for a quantitative analysis of samples, an initial quantitative set-up was evaluated. Therefore, the cMIA was performed as described above in Example 5.1 using ZIKV VLP-coupled microspheres and final sample dilutions in the range of 10-, 40-, 80-, 160-, and 320-fold in the assay (cf. also FIG. 37A; FIG. 37B shows the same results as in FIG. 37A, however, MFI values are presented in dependency of the initial sample dilution prior to combination with the same volume of microspheres, i.e. sample dilutions of 5-160-fold).

For a quantitative analysis, MFI raw data obtained from the analysis in the MAGPIX Luminex reader were analyzed and plotted using GraphPad Prism 8 version 8.1.0 (GraphPad Software, Inc.). A mean value from the replicates of the 100% mAb binding control was calculated. From the mean value of the 100% mAb binding control signal, the value referring to 40% of the 100% binding control signal was calculated. The sample dilution values in normal scale (e.g. 10-, 20-, 40-fold dilution) were logarithmized (final sample dilution for FIG. 37A and initial sample dilution for FIG. 37B) and the MFI values in dependency of the logarithmized dilution were analyzed using a 4PL non-linear regression model [Sigmoidal, 4PL, X=Log (sample dilution)]. The logarithmized sample dilution resulting in an MFI signal referring to the 40% value of the 100% mAb binding signal calculated above was determined from the regression curves for each sample. The corresponding dilutions were de-logarithmized to result in the sample dilution in normal scale that is able to block 60% of the maximum mAb binding to the ZIKV VLPs (referred to as ZIKV-specific blockade titer). The blockade titers were considered to be valid if the R² value (coefficient of determination) is equal to or greater than 0.9000. A sample was considered as negative for ZIKV-specific Ab (no ZIKV-specific Abs present within the sample) if mAb binding could not be blocked to result in 40% or less mAb binding over the examined dilution range and/or if the R² value was less than 0.9000. Valid blockade titers can be used for the calculation of ZIKV specific Abs within the corresponding samples by comparison to a standard curve.

Plasma samples from Example 3 were analyzed with the initial quantitative set-up of the cMIA using anti-ZIKV #7. Human plasma samples #3 and 4, which were considered to be ZIKV high-reactive, showed a concentration dependent increase of blocking of mAb binding (indicated by the lower MFI values) to almost complete blockade of binding for the lowest sample dilution (FIG. 37). In contrast, the negative control as well as human plasma samples #1 and 2 did not show a high degree of blocking at comparable sample dilutions. Only the human plasma samples #3 and 4, which were considered as ZIKV high-reactive (presumptive ZIKV specific Ab positive) were able to block at least 60% or more of the binding of anti-ZIKV #7 by crossing the threshold of 40% mAb binding. For these two plasma samples, calculated ZIKV-specific blockade titers were valid (Table 7, R² values of greater than 0.900). Contrarily, ZIKV low-reactive (presumptive ZIKV negative) human plasma samples #1 and 2 were not capable to block the mAb binding efficiently. Therefore, the blockade titer could either not be calculated by the non-linear regression similar to the negative control for sample #1 or calculations did not result in an R² value of greater than 0.900 for sample #2.

TABLE 7
ZIKV-specific blockade titers for samples examined in the initial
quantitative cMIA using anti-ZIKV #7 calculated based on the
initial sample dilution (cf. FIG. 37B). Presented are the titers together
with the coefficient of determination (R² value) for the titers.
The titers refer to the dilution of the sample that inhibits 60%
of the maximal binding of the anti-ZIKV #7 to the ZIKV VLPs.

| | ZIKV-specific | |
|---|---|---|
| Sample | Blockade Titer | R² |
| Negative control | N/A | 0.77240 |
| Human plasma sample #1 | N/A | 0.66370 |
| Human plasma sample #2 | 4.10 (considered | 0.61510 |
| | invalid) | |
| Human plasma sample #3 | 32.78 (considered | 0.98620 |
| | valid) | |
| Human plasma sample #4 | 11.80 (considered | 0.99730 |
| | valid) | |

In summary, the initial quantitative cMIA set-up was able to distinguish between presumptive ZIKV high-reactive (ZIKV specific Ab containing samples) and ZIKV low-reactive samples independent of the presence of cross-reactive Abs resulting from other flavivirus infections as DENV infection.

### Example 5.2.2: Optimized Quantitative cMIA Set-Up

In a next step, the initial quantitiative cMIA set-up, which was shown to be capable to distinguish between ZIKV high- and low-reactive samples in Example 5.2.1, was further optimized as outlined below.

In the optimized quantitative cMIA set-up, samples were not heat-inactivated, as the MIA set-up was shown to work in a robust manner, independent of heat-inactivation (cf. Example 4). Samples were prepared by vortexing for 5 sec before transferring them into a deep-well 2 mL plate (ThermoFisher Scientific, Cat. No. 278752) for preparation of the serial sample dilutions. An initial 5-fold dilution per sample was performed using assay buffer (1% (w/v) BSA in 1-fold PBS, pH 7.4) in row A of the deep well plate. The initial dilution was mixed by pipetting up and down 10 times in row A. Then, 2-fold serial dilutions were performed by combining half volume of solution in row A with same volume of assay buffer in row B, and so on until row H. Mixing between dilution was performed by pipetting up and down 5 times. A total of eight 2-fold dilutions (5-fold to 640-fold) per sample (rows A-H; 5-, 10-, 20-, 40-, 80-, 160-, 320-, and 640-fold dilution, respectively) were performed. Deep well dilution plate was covered with a sealing sheet while a ZIKV VLP coupled-microspheres working mixture was prepared. ZIKV VLP coupled-microspheres prepared under Example 1 were vortexed for 20 sec. The coupled microsphere stock was diluted to a final concentration of 50 microspheres/μL in assay buffer and vortexed for 5 sec. The working mixture was kept at room temperature and 25 μL of the prepared working microsphere mixture were added per well of a black flat bottom 96-well assay plate (Corning Inc., Cat. No. 3915). Afterwards, 25 μL of each diluted sample were aliquoted into the assay plate per well in duplicates in a vertical plate layout (e.g. the 8 dilutions of the first sample were added in rows A-H and columns 1-2), resulting in a 2-fold dilution of each sample dilution (=final sample dilution; 10-, 20-, 40-, 80-, 160-, 320-, 640-, 1280-fold dilution, respectively) when combined with the 25 μL of the microsphere mixture per well. Two controls were included by aliquoting 25 μL of assay buffer per well in duplicates (columns 11-12) to later account for 0% and 100% mAb binding. The assay plate was covered with a foil sealing sheet and incubated for 60 min at room temperature on a plate shaker at 600 rpm. Meanwhile, an intermediary 50- or 1000-fold dilution of the mAb used (50-fold for Anti-ZIKV #6 to 7; 1000-fold for Anti-ZIKV #1 to 5) was prepared by mixing 5 μL of the stock mAb solution with 245 or 4995 μL of assay buffer, respectively. The intermediary dilution was vortexed for 5 sec. Next, the intermediary dilution was further diluted in assay buffer to reach the final mAb dilution, which corresponds to the EC₂₅ or EC₅₀ concentration (EC₅₀ for Anti-ZIKV #6 to 7; EC₂₅ for Anti-ZIKV #1 to 5) of the mAb for ZIKV VLP-binding calculated under Example 2 after 2 hr incubation with the ZIKV VLP coupled-microspheres (Table 5). The final mAb concentration solution was vortexed for 5 sec. After incubation of the samples with the microspheres, the assay plate was washed two times with PBS-T (0.05% (v/v) Tween-20 in PBS pH 7.4) in a magnetic plate washer (BioTek Instruments, Product Id. 400072). Afterwards, the plate was placed in a 96-well plate magnet (Life Technologies, Product Id. 32513) and incubated for 60 sec while covering the plate. Following the incubation step, the supernatant was removed. Then, 50 μL of the final mAb concentration solution were added to the sample-containing wells (columns 1-10) and 100% mAb binding (columns 11-12, rows E-H) wells and 50 μL of assay buffer were added to the wells corresponding to 0% mAb-binding (columns 11-12, rows A-D). The plate was covered with a foil sealing sheet and incubated for 2 hr at room temperature on a plate shaker at 600 rpm. After incubation with the mAb, the assay plate was washed two times with PBS-T (0.05% (v/v) Tween-20 in PBS pH 7.4) in a magnetic plate washer. Afterwards, the plate was placed in a 96-well plate magnet and incubated for 60 sec while covering the plate. Following the incubation step, the supernatant was removed. For detection of rabbit (Anti-ZIKV #1 to 5) or mouse (Anti-ZIKV #6 to 7) mAbs, respectively, a F(ab′)2-Goat anti-rabbit IgG (heavy and light chain) cross-adsorbed PE-conjugated (Invitrogen, Cat. No. 31846, Lot. No. TL2684941, 0.5 mg/mL) or a R-PE AffiniPure F(ab′)₂ fragment goat anti-mouse IgG (heavy and light chain; Jackson ImmunoResearch, Cat. No. 115-116-146, Lot. No. 143867, 0.5 mg/mL) secondary reporter Ab was applied. Secondary Abs were diluted 1:50 in assay buffer to achieve a final working concentration of 10 μg/mL by vortexing for 5 sec. 50 μL of the corresponding diluted secondary reporter Ab were added to all wells, including the 0% and 100% mAb binding control wells. The plate was covered with a foil sealing sheet and incubation carried out for 30 min at room temperature on a plate shaker at 600 rpm. After incubation of mAb, the assay plate was washed two times with PBS-T (0.05% (v/v) Tween-20 in PBS pH 7.4) in a magnetic plate washer. Afterwards, the plate was placed in a 96-well plate magnet and incubated for 60 sec while covering the plate. Following the incubation step, the supernatant was removed. After the washing steps, the microspheres were resuspended in 100 μL assay buffer per well. At this point, the plate can be stored with foil sealing sheet overnight at 4° C. Before sample read-out, the plate was allowed to re-equilibrate to room temperature for 20 min (t 5 min) if stored at 4° C. overnight. The plate was put on an orbital shaker at 600 rpm for at least 5 min in order to allow for complete resuspension of immunocomplexed microspheres. Finally, the plate was placed in the multiplexing MAGPIX® plate reader (Luminex Corporation, Austin, Texas). The program used was xPONENT® (Build 4.2.1705.0) and is set-up with sample volume: 50 μL per well; plate protocol: 96-well plate format; and microsphere protocol: Map, BP 50 regions, Type MagPlex.

The MFI data obtained from analysis in the MAGPIX Luminex reader were analyzed and plotted as described under Example 5.2.1 and ZIKV-specific blockade titers were calculated for each sample based on the final sample dilution.

### Example 5.2.3 the Quantitative cMIA is Able to Distinguish Samples Containing ZIKV-Specific Abs from Samples Containing Other Anti-Flaviviruses Abs

In a next step, it was evaluated whether the quantitative cMIA set-up is able to distinguish samples comprising ZIKV-specific Abs from samples comprising other anti-flavivirus Abs (including ZIKV cross-reactive Abs) in a robust manner. Therefore, human samples containing antibodies directed against ZIKV, DENV, Yellow Fever Virus (YFV), St. Louis Encephalitis Virus (SLEV), or West Nile Virus (WNV) as well as samples containing antibodies directed against both, ZIKV and DENV or WNV and DENV, as determined in corresponding immunoassays, were selected for analysis. In addition to human samples, also non-human primate samples from animals infected with ZIKV or DENV, or vaccinated with either YFV vaccine (STAMARIL; Sanofi Pasteur), Japanese Encephalitis virus (JEV) vaccine (IXIARO; Valneva Scotland Ltd.), ZIKV vaccine (purified inactivated vaccine, PIZV; see, for instance, WO 2019/090228), WNV vaccine (INNOVATOR; Fort Dodge Animal Health), or Tick-borne encephalitis virus (TBEV) vaccine (ENCEPUR; GlaxoSmithKline) were analyzed. The quantitative cMIA was carried out using anti-ZIKV #7 as described above under Example 5.2.2.

### Human Samples

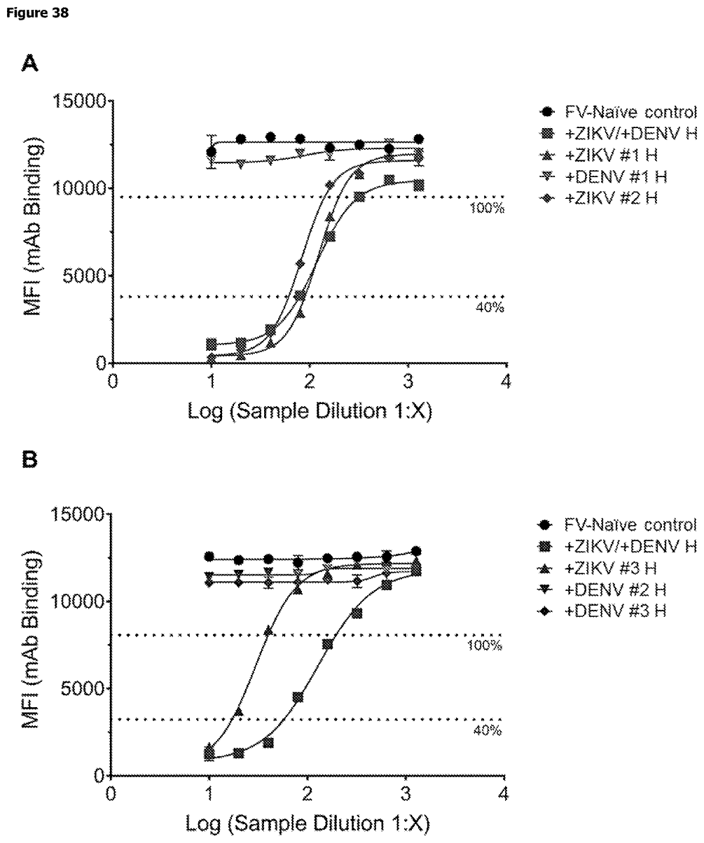

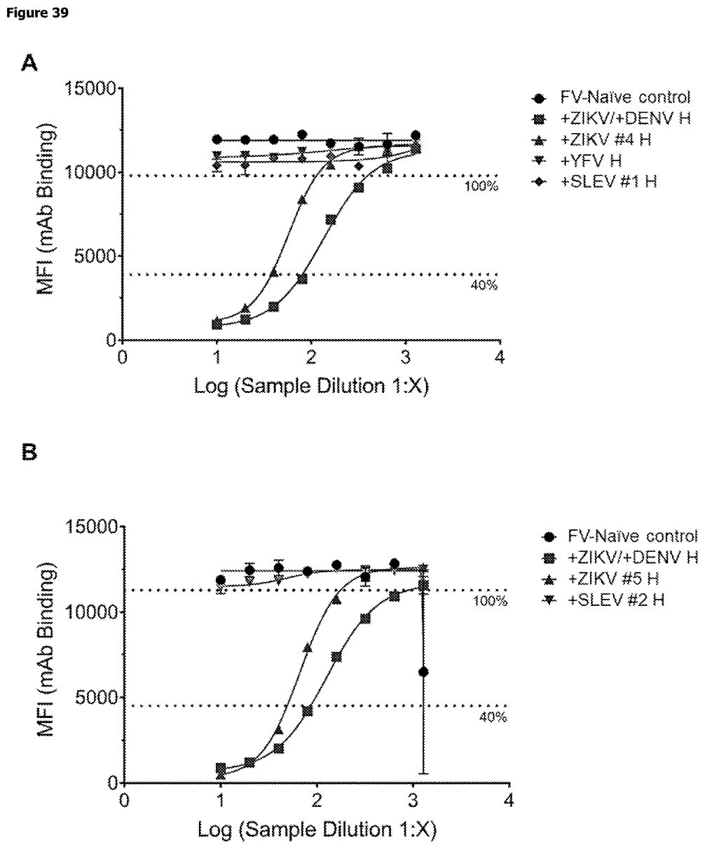

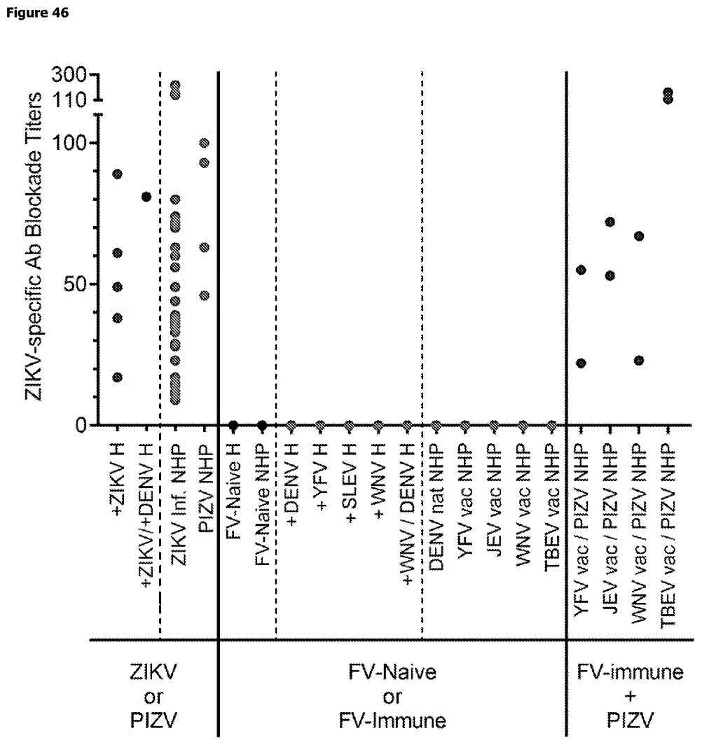

Human samples evaluted were serum samples, except for the negative control (which did not comprise anti-flavivirus Abs; “FV-Naïve control”) and the sample that contained both, anti-ZIKV and anti-DENV Abs, which were plasma samples. mAb binding was almost completely blocked by the five samples containing anti-ZIKV antibodies at lower sample dilutions (designated as “+ZIKV #1-5 H”; FIGS. 38 and 39). In addition, also the sample containing both, anti-ZIKV and anti-DENV Abs, was able to block mAb binding in a similar way (designated as “+ZIKV/+DENV H”; FIG. 38-45). In line with that, valid ZIKV-specific blockade titers could be determined for these samples (FIG. 46).

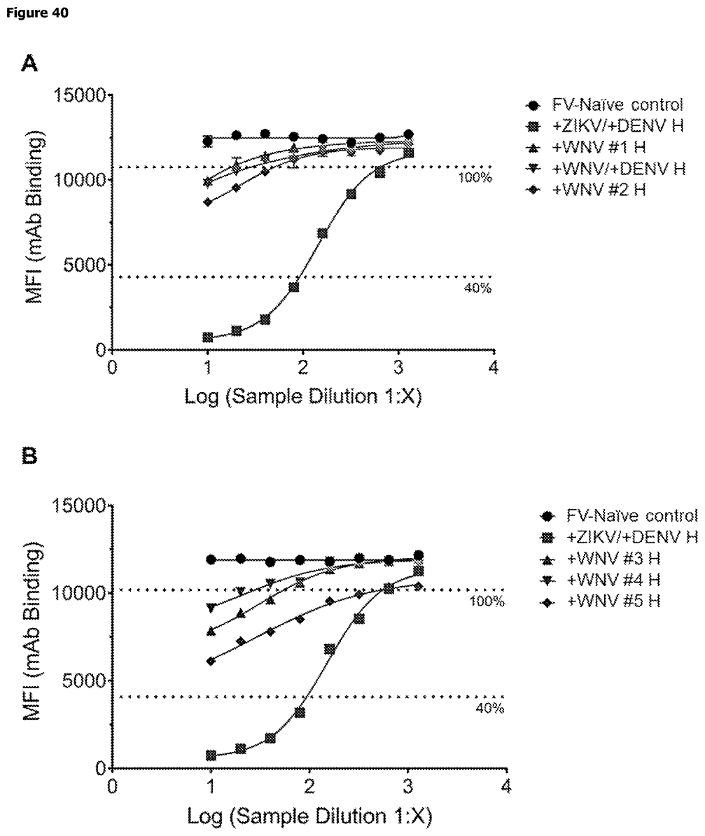

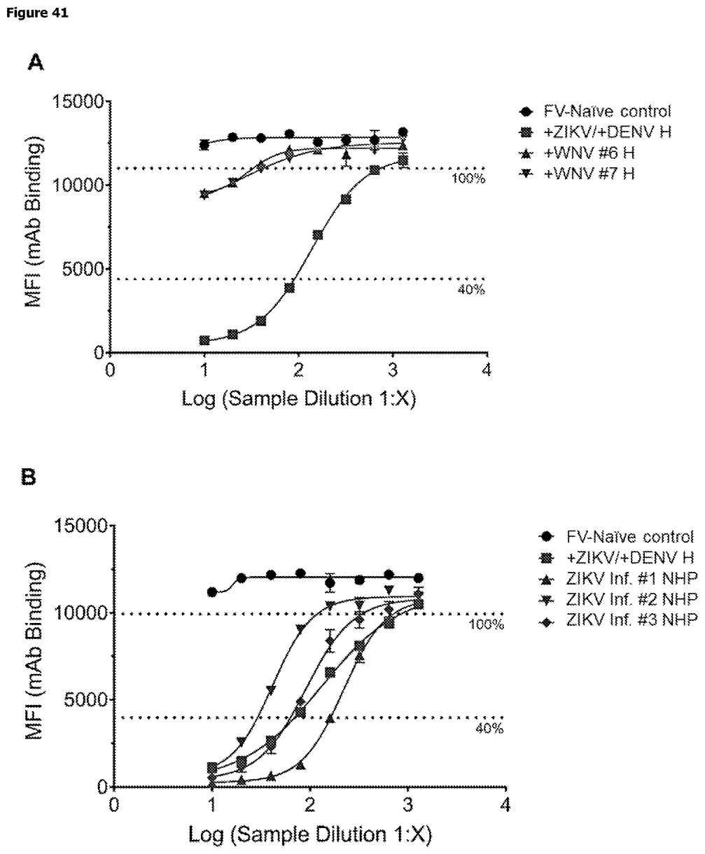

In contrast, mAb binding was not diminished over the sample dilution range similar to the negative control (“FV-Naïve control”) for the three samples containing anti-DENV Abs (designated as “+DENV #1-3 H”; FIG. 38), a sample containing anti-YFV Abs (designated as “+YFV H”; FIG. 39A), and two samples containing anti-SLEV Abs (designated as “+SLEV #1-2 H”; FIG. 39). Although samples containing anti-WNV Abs (designated as “+WNV #1-7 H”) and one sample containing both, anti-WNV and anti-DENV Abs (designated as “+WNV/+DENV”), showed a slight blocking at low sample dilutions, the threshold of 60% mAb blocking (resulting in less than 40% mAb binding) for considering a sample as ZIKV positive, was not reached for none of the samples (FIGS. 40 and 41A). In line with that, valid ZIKV-specific blockade titers could not be calculated for the samples comprising anti-DENV, YFV, SLEV, and WNV Abs (FIG. 46; designated as ZIKV-specific blockade titer of 0).

Consequently, the quantitative cMIA enables a robust differentiation between samples comprising ZIKV-specific antibodies, and samples comprising other flavivirus Abs (including ZIKV cross-reactive Abs) and thus enables characterization of the immune status of a human subject. No false-positives were detected.

### Non-Human Primate Samples

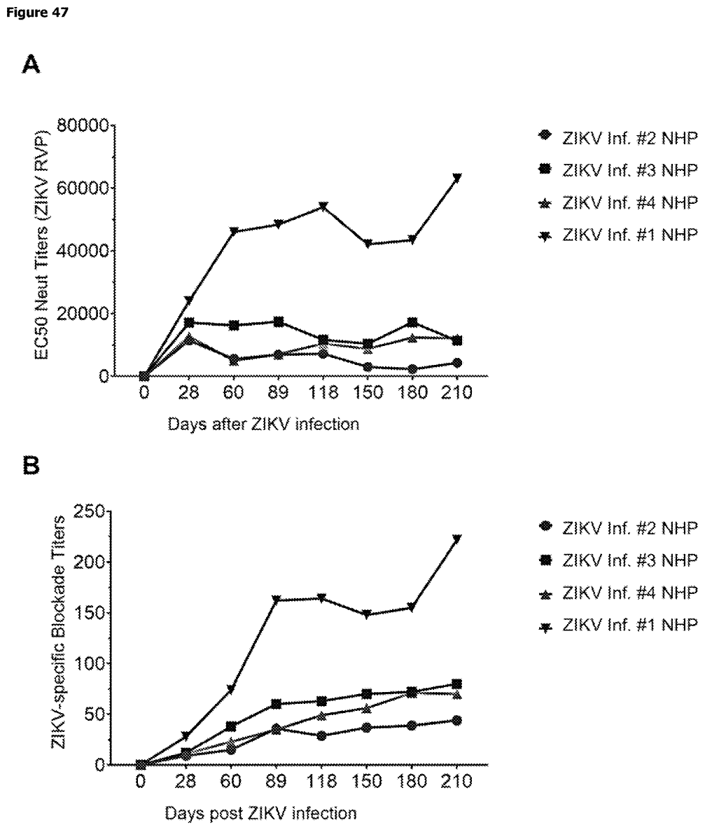

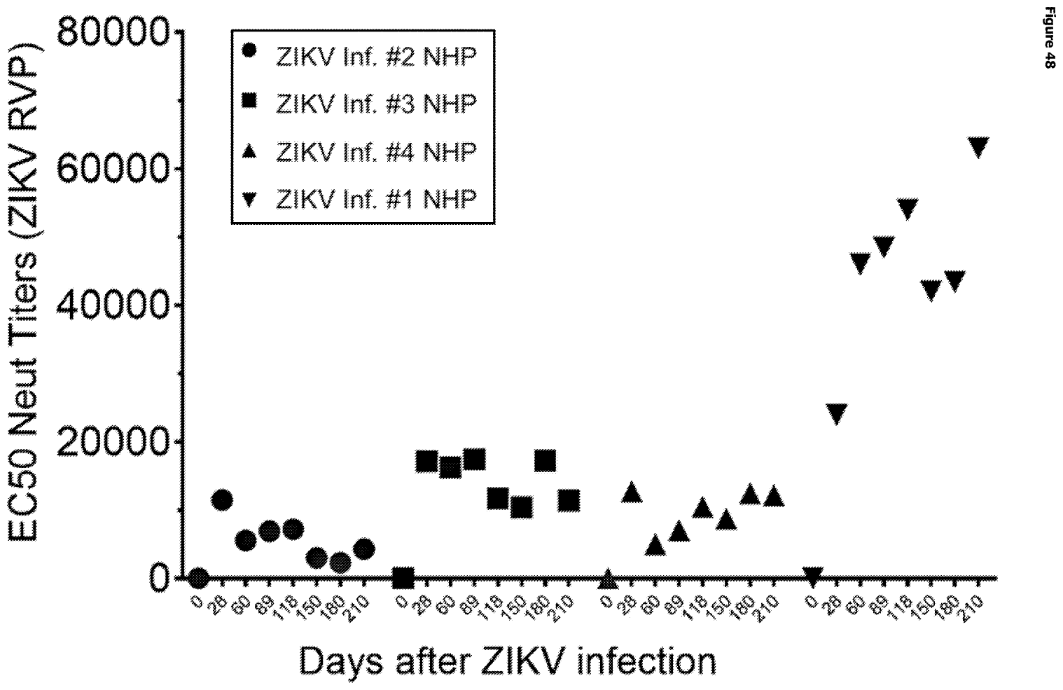

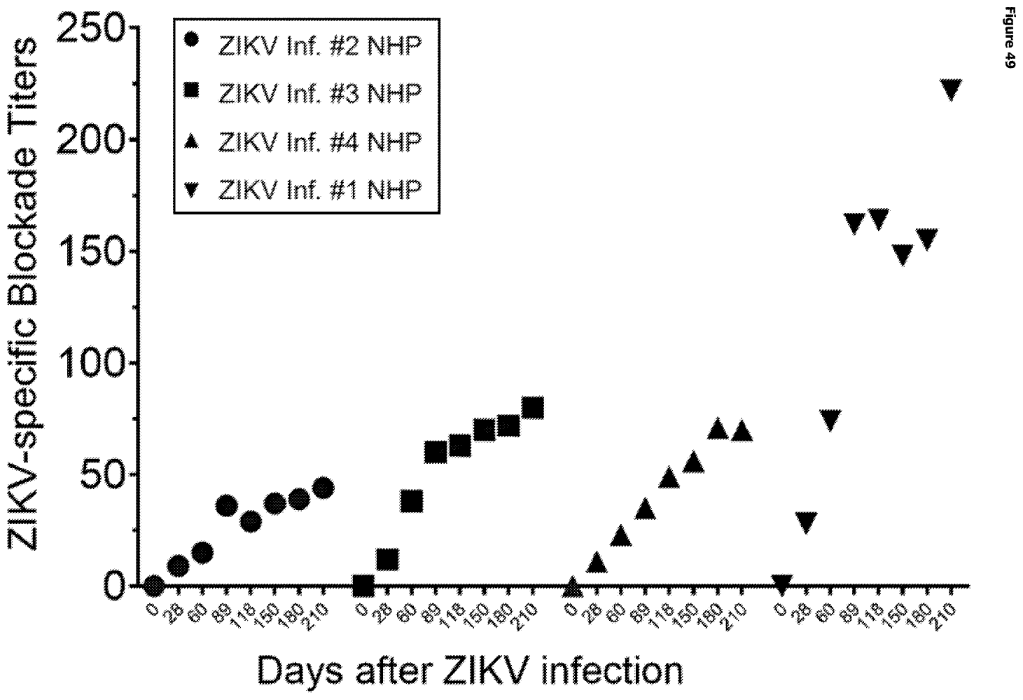

The assay set-up was further applied for analyzing serum samples from non-human primates (NHP). Therefore, serum samples from rhesus macaques after ZIKV primary infection were evaluated in the quantitative cMIA (three of them are depicted in FIG. 41B and are designated as “ZIKV Inf. #1-3 NHP”). Samples were collected 89 days post infection for one animal (“ZIKV Inf. #1 NHP” in FIG. 41B) and 118 days post infection for the other two animals depicted in FIG. 41B, respectively. The samples almost completely blocked mAb binding similar to the human samples comprising anti-ZIKV Abs (FIG. 41B). In line with that, valid ZIKV-specific blockade titers could be determined for all samples (FIG. 46). In contrast, valid ZIKV-specific blockade titers could not be determined using samples from animals with natural DENV infection (designated as ZIKV-specific blockade titer of 0 in FIG. 46), as mAb binding was not (sufficiently) blocked (samples designated as “DENV nat NHP” in FIG. 46). In addition, the development of ZIKV-specific blockade titers in dependency of the days post ZIKV infection correlated well with the development of neutralization titers in dependency of the days post ZIKV infection for the animals (exemplarily shown for four animals designated as “ZIKV Inf. #1-4 NHP”, wherein the numbering for animals #1-3 is the same as in FIG. 41B; FIG. 47). Neutralizing titers were determined with a ZIKV RVP assay carried out essentially as described in Example 3. In contrast to neutralizing titers that reached essentially a plateau after approximately 28 to 60 days post infection in animals #2-4, ZIKV-specific blockade titers continuously increased over the first 210 days post infection in these animals (FIGS. 48 and 49).

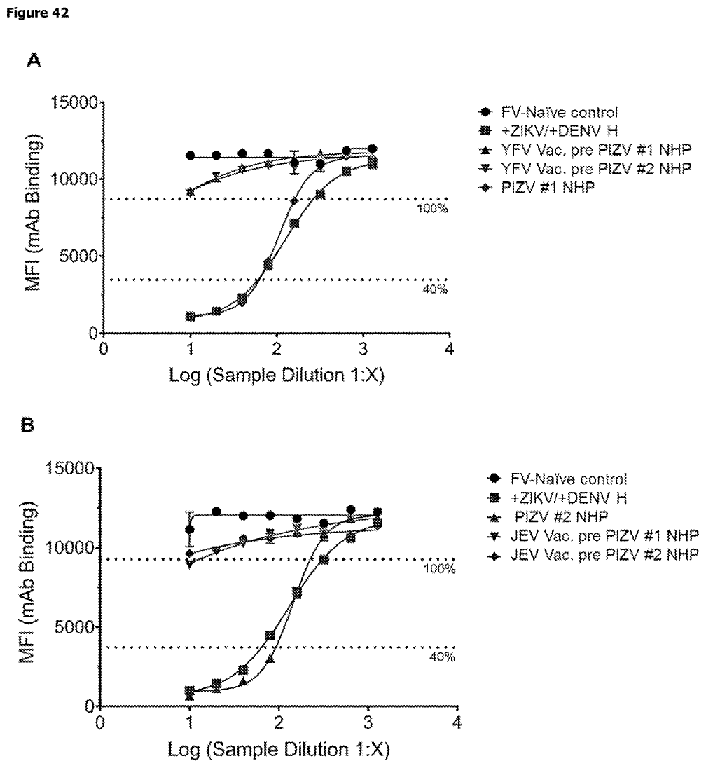

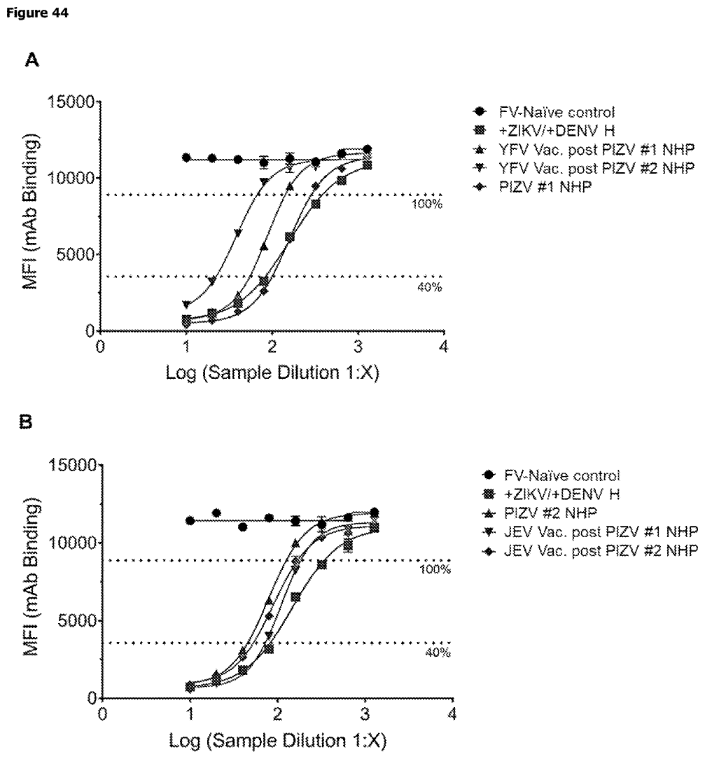

Moreover, similar results than with the ZIKV natural infected animals were observed for samples from four rhesus macaques each vaccinated with two doses of the purified inactivated zika vaccine (PIZV) on days 1 and 29. MFI values resulting from analysis of samples from two of those animals are depicted in FIGS. 42 and 44 (designated as “PIZV #1-2 NHP”), wherein in FIG. 42 the samples were taken 90 days post vaccination and in FIG. 44 the samples were taken 252 days post vaccination. In addition to the samples from animals with natural ZIKV infection, also for the samples from the vaccinated animals ZIKV-specific blockade titers were valid (FIG. 46).

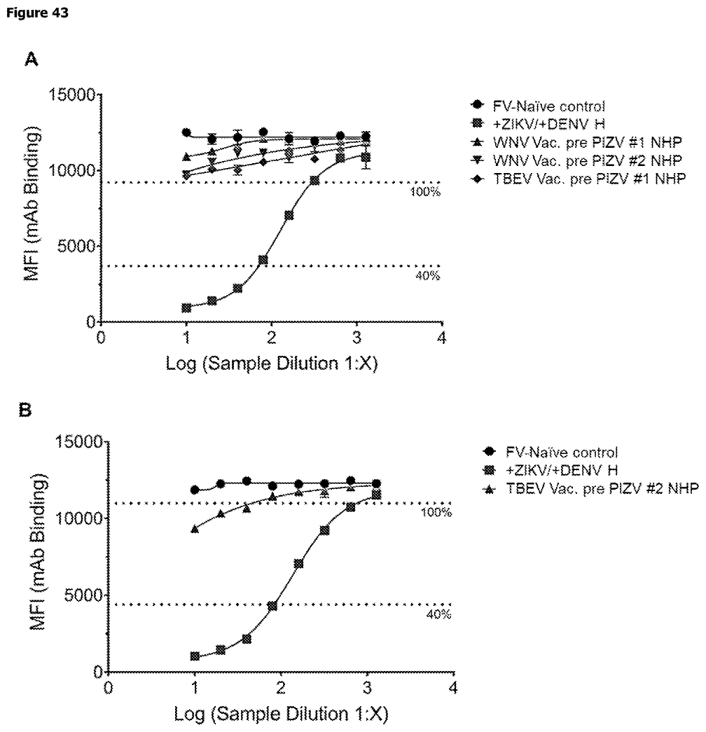

Contrarily, samples from rhesus macaques vaccinated with YFV, JEV, WNV, or TBEV vaccines were not capable of blocking mAb binding. Samples from two animals each vaccinated with two doses of a JEV vaccine (first dose at day 1, second dose at day 29; designated as “JEV Vac. pre PIZV #1-2 NHP”; FIG. 42B), from two animals each vaccinated with one dose of a YFV vaccine (designated as “YFV Vac. pre PIZV #1-2 NHP”; FIG. 42A), from two animals each vaccinated with two doses of a WNV vaccine (first dose at day 1, second dose at day 29; designated as “WNV Vac. pre PIZV #1-2 NHP”; FIG. 43A), and from two animals each vaccinated with two doses of a TBEV vaccine (first dose at day 1, second dose at day 29; designated as “TBEV Vac. pre PIZV #1-2 NHP”; FIG. 43) were analyzed. In line with the low blocking, valid ZIKV-specific blockade titers could not be calculated for these samples (FIG. 46).

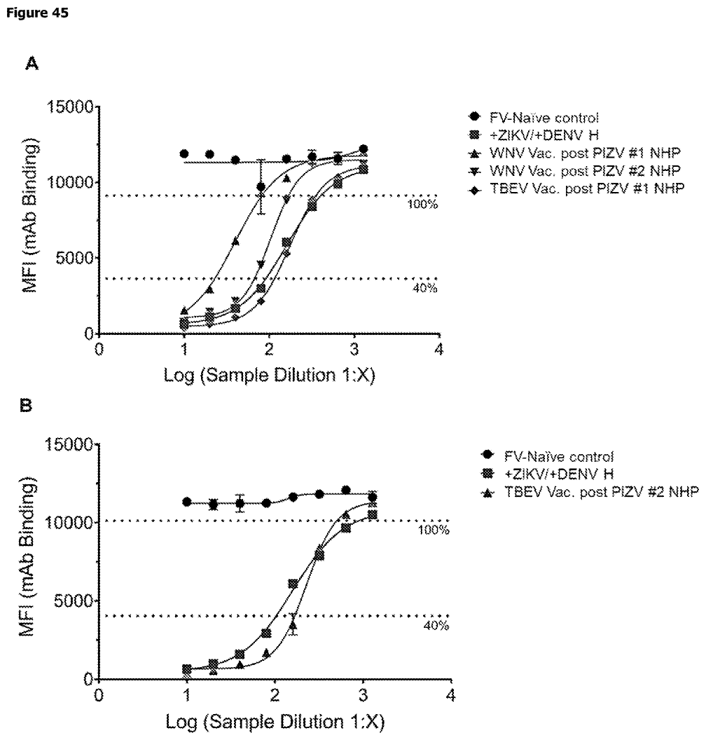

However, mAb binding was efficiently blocked by samples from the same vaccinated rhesus macaques described above, which were taken after an additional subsequent vaccination of those animals with two doses of PIZV 168 days after the last YFV, JEV, WNV, or TBEV vaccine dose, respectively (designated as “JEV Vac. post PIZV #1-2 NHP”, “YFV Vac. post PIZV #1-2 NHP”, “WNV Vac. post PIZV #1-2 NHP”, and “TBEV Vac. post PIZV #1-2 NHP”; FIGS. 44 and 45). Valid ZIKV-specific blockade titers were calculated for all animals after the subsequent vaccination with PIZV (FIG. 46).

### Example 5.2.4 Quantitative cMIA Using mAbs #1-5

To demonstrate that the quantitative cMIA is suitable to be performed with sevaral different mAbs, anti-ZIKV #1-5 were applied in the quantitative cMIA as described under Example 5.2.2. As described under Example 5.2.2., anti-ZIKV #1-5 were applied at final mAb concentrations corresponding to the EC₂₅ concentration of the mAb for ZIKV VLP-binding calculated under Example 2 after 2 hr incubation with the ZIKV VLP coupled-microspheres (Table 5). Anti-ZIKV #7 was applied at a final mAb concentration corresponding to the EC₅₀ concentration (as described above under Example 5.2.2 and applied under Example 5.2.3) and in addition also at a final mAb concentration corresponding to the EC₂₅ concentration of the mAb for ZIKV VLP-binding calculated under Example 2 after 2 hr incubation with the ZIKV VLP coupled-microspheres (Table 5).

Therefore, the human sample containing antibodies directed against both, ZIKV and DENV (designated as “+ZIKV/+DENV H”, as well as the negative control (which did not comprise anti-flavivirus Abs; designated as “FV-Naïve control”) described in Example 5.2.3 above were applied. In addition, the human samples containing antibodies directed against West Nile Virus (WNV) described in Example 5.2.3 above were pooled and analyzed (designated as “+WNV (human pool)”). Moreover, also the non-human primate samples from animals infected with DENV described in Example 5.2.3 were pooled for analysis (designated as “DENV nat NHP pool”). Finally, also the non-human primate samples from animals immunized with PIZV described in Example 5.2.3 were pooled for analysis (designated as “PIZV NHP pool”).

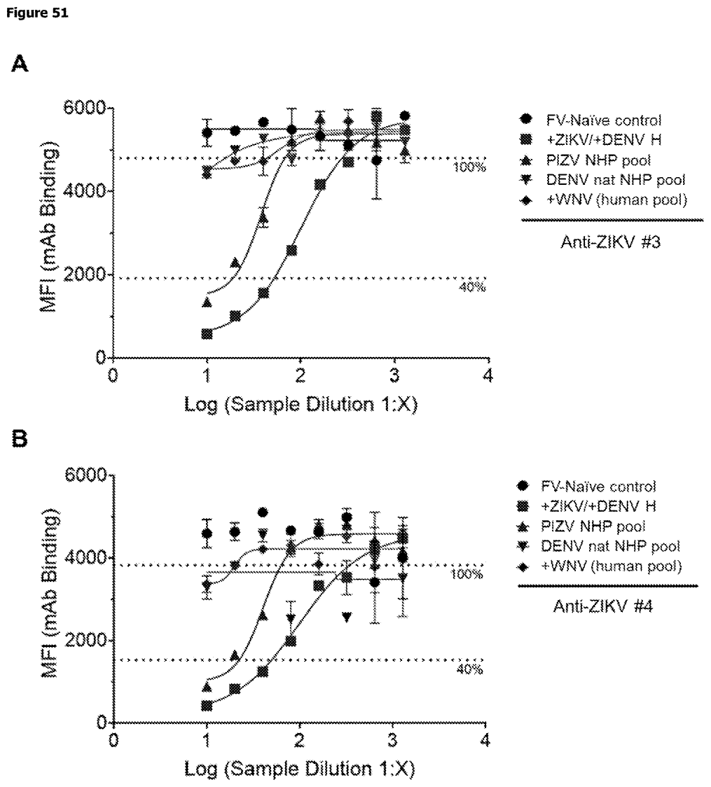

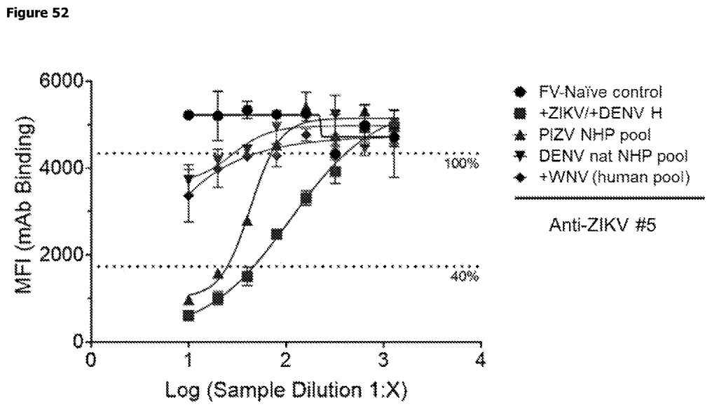

Each of anti-ZIKV #1-5 was able to differentiate the “PIZV NHP pool” and “+ZIKV/+DENV H” samples from the “DENV nat NHP pool” and the “+WNV (human pool)” samples, indicating a robust assay performance also with different mAbs (FIG. 50-52). In line with that, valid ZIKV-specific blockade titers could be calculated for the samples comprising anti-ZIKV Abs (“PIZV NHP pool” and “+ZIKV/+DENV H”), whereas valid ZIKV-specific blockade titers could not be calculated for samples which did not comprise ZIKV-specific antibodies (“+WNV (human pool)” and “DENV nat NHP pool”; Table 8; designated as ZIKV-specific blockade titer of 0) indicating that the quantitative cMIA is highly specific.

TABLE 8
ZIKV-specific blockade titers for samples examined in the quantitative cMIA using anti-
ZIKV #1-5 and #7 calculated based on the final sample dilution. Presented are
the titers for the different samples together with the final mAb concentration (EC₂₅ or
EC₅₀ concentrations, respectively). The titers refer to the dilution of the sample
that inhibits 60% of the maximal binding of the anti-ZIKV mAbs to the ZIKV VLPs.

| | mAb | FV-Naïve | DENV nat | +WNV | PIZV NHP | |
|---|---|---|---|---|---|---|
| Anti-ZIKV | concentration | control | NHP pool | (human pool) | pool | +ZIKV/+DENV H |
| #1 | EC₂₅ | 0 | 0 | 0 | 8 | 35 |
| #2 | EC₂₅ | 0 | 0 | 0 | 19 | 46 |
| #3 | EC₂₅ | 0 | 0 | 0 | 19 | 52 |
| #4 | EC₂₅ | 0 | 0 | 0 | 16 | 41 |
| #5 | EC₂₅ | 0 | 0 | 0 | 25 | 46 |
| #7 | EC₂₅ | 0 | 0 | 0 | 14 | 65 |
| | EC₅₀ | 0 | 0 | 0 | 19 | 46 |

### CONCLUSION

Although competitive ELISA set-ups have been used with ZIKV EDIII as plate-immobilized antigen (WO 2020/087038), our experimental data show that EDIII is not suitable for application as microsphere-coupled antigen. In particular, as used in WO 2020/087038, ZIKV EDIII C-terminally fused to a human IgG1 Fc tag (rZIKV-EDIII-3 in the present application) resulted in strong background signal in the total human IgG MIA when coupled to the microspheres (comp. Example 4). Moreover, rZIKV-EDIII-3 as microsphere coupled-antigen was not able to distinguish between ZIKV and other flavivirus infections in the cMIA. As rZIKV-EDIII-3, also other recombinant ZIKV EDIII tested herein were not suitable for the microsphere set-up. Solely ZIKV VLP showed satisfying and reliable results in both, the total IgG MIA, as well as the cMIA.

In summary, we could show that the cMIA set-up is able to reliably and efficiently detect and quantify anti-ZIKV Abs. With the use of different reporter Abs, we are able to detect both, strong-neutralizing, as well as weak-neutralizing Abs (i.e. by the use of Anti-ZIKV #5). With the application of a non-neutralizing reporter Ab, non-neutralizing anti-ZIKV Abs can be detected as well. Moreover, we could demonstrate that the reporter Abs used are highly selective for ZIKV VLPs, as the mAbs did not show binding to DENV1-4 VLPs within the same mAb concentration range applied for evaluation of binding to ZIKV VLPs. Therefore, our set-up is able to distinguish between ZIKV and other flavivirus infections by measuring ZIKV specific Abs instead of cross-reactive Abs, which underlines its suitability for use in diagnostics.

Compared to traditional assays such as competitive ELISA set-ups, a microsphere-based assay provides several advantages. This approach increases sensitivity and specificity, among other advantages such as the flexibility to single- or multi-plex antigens from different viruses in a single reaction (by the application of microspheres with different specific features), high-throughput (e.g. simplified washing procedures due to magnetic microspheres), cost-effectiveness (e.g. due to reduced sample volume, consumables, and labor), and short turnaround time.

Recently, it has been shown that results from competitive sample set-ups (i.e. set-ups using mAbs directed against ZIKV EDIII) measuring anti-ZIKV Ab titers correlate with protection in vivo (WO 2020/087038). This reinforces the fact that our cMIA is as well suitable for determining protection in subjects against ZIKV infection by measuring anti-ZIKV Abs. For instance, anti-ZIKV Abs may be induced by vaccination of the subjects or by natural ZIKV infections. Moreover, as the cMIA is selective for determination of anti-ZIKV Abs it is able to reliably distinguish between Abs induced by a ZIKV infection and cross-reactive Abs induced by any other flavivirus infection, such as a DENV, WNV, JEV, YFV, or SLEV infection.

In conclusion, ZIKV VLPs as antigens coupled to the microspheres in combination with all example mAbs (Anti-ZIKV #1-11) selected from Example 2 are well suitable for the cMIA. The results demonstrate the capability of the cMIA for reliable detection and quantification of ZIKV specific Abs in different samples from different origins (such as human or NHP) or in different sample types (such as serum or plasma). In addition, the assay is capable of distinguishing ZIKV specific Ab containing samples from ZIKV non- or low-reactive samples, independent of the presence of other (also ZIKV cross-reactive) anti-flavivirus Abs such as DENV, YFV, WNV, SLEV, or TBEV immunities, required by natural infection or vaccination.

Unless otherwise indicated, all numbers expressing quantities of ingredients, properties such as molecular weight, reaction conditions, and so forth used in the specification and claims are to be understood as being modified in all instances by the term “about”. As used herein the terms “about” and “approximately” means within 10 to 15% of the number, preferably within 5 to 10% of the number. Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

Numerous references have been made throughout this specification. Each of the above-cited references is individually incorporated herein by reference in its entirety. In case of conflict, the information in the present application prevail.

In the following, CDR sequences of anti-ZIKV Abs as listed in the sequence listing are additionally reproduced:

| | | SEQ | |
|---|---|---|---|
| | | ID NO | |
| | | in the | |
| | | sequence | |
| mAb | CDR | listing | CDR sequence |
| Anti-ZIKV #1 | VH-CDR1 | 7 | GFSFNSNYW |
| (Clone 102-1) | VH-CDR2 | 8 | FGGIHVT |
| | VH-CDR3 | 9 | IISTGGSHRFNL |
| | VL-CDR1 | 12 | ESIYTY |
| | VL-CDR2 | 13 | RAS |
| | VL-CDR3 | 14 | QATDVGGSGRGA |
| Anti-ZIKV #2 | VH-CDR1 | 21 | GFSFTDRHY |
| (Clone 242-3) | VH-CDR2 | 22 | YPGSSGST |
| | VH-CDR3 | 23 | ARSSYPDSSGYSYGMDL |
| | VL-CDR1 | 26 | QNINSN |
| | VL-CDR2 | 27 | LTS |
| | VL-CDR3 | 28 | QTYYDISNYGYA |
| Anti-ZIKV #3 | VH-CDR1 | 35 | GFSFTDRHY |
| (Clone 270- | VH-CDR2 | 36 | YPGSSGST |
| 12) | VH-CDR3 | 37 | ARSSYPDSSGYSYGMDL |
| | VL-CDR1 | 40 | QDINSN |
| | VL-CDR2 | 41 | LTS |
| | VL-CDR3 | 42 | QTYYDISNYGYA |
| Anti-ZIKV #4 | VH-CDR1 | 49 | GFSFSSGAY |
| (Clone 289-3) | VH-CDR2 | 50 | YTGDGRT |
| | VH-CDR3 | 51 | ARAIAVGAGYGVGNYFTL |
| | VL-CDR1 | 54 | ENIYGY |
| | VL-CDR2 | 55 | KAS |
| | VL-CDR3 | 56 | QSYYTSSSNADGSENA |
| Anti-ZIKV #5 | VH-CDR1 | 63 | GFDFSDRYY |
| (Clone 306-2) | VH-CDR2 | 64 | YVGSGDT |
| | VH-CDR3 | 65 | ARHPGTYF |
| | VL-CDR1 | 68 | QNIVNN |
| | VL-CDR2 | 69 | DTS |
| | VL-CDR3 | 70 | QTYYYYNKIING |
| Anti-ZIKV #6 | VH-CDR1 | 76 | GYTFTSY |
| (Clone ZV-67) | VH-CDR2 | 77 | YPRSGN |
| | VH-CDR3 | 78 | ENYGSVY |
| | VL-CDR1 | 80 | CKASQNVGTAVA |
| | VL-CDR2 | 81 | SASNRYT |
| | VL-CDR3 | 82 | QQFSSYPYT |
| Anti-ZIKV #7 | VH-CDR1 | 84 | GYTFTGYH |
| (Clone ZKA- | VH-CDR2 | 85 | INPNSGGT |
| 64) | VH-CDR3 | 86 | ARMSSSIWGFDH |
| | VL-CDR1 | 88 | QSVLIN |
| | VL-CDR2 | 89 | LIYGASSRA |
| | VL-CDR3 | 90 | QQYNDWPPIT |
| Anti-ZIKV #8 | VH-CDR1 | 95 | GFSFTDRHY |
| (Clone 260-2) | VH-CDR2 | 96 | YPGSSGST |
| | VH-CDR3 | 97 | ARSSYPDSSGYSYGMDL |
| | VL-CDR1 | 100 | QNINSN |
| | VL-CDR2 | 101 | LTS |
| | VL-CDR3 | 102 | QTYYDISNYGYA |
| Anti-ZIKV #9 | VH-CDR1 | 109 | GFSFTDRHY |
| and #10 | VH-CDR2 | 110 | YPGSSGST |
| (Clone 181- | VH-CDR3 | 111 | ARSSYPDSSGYSYGMDL |
| 4/329-2) | VL-CDR1 | 114 | QNINSN |
| | VL-CDR2 | 115 | LTS |
| | VL-CDR3 | 116 | QTYYDISNYGYA |
| Anti-ZIKV #11 | VH-CDR1 | 123 | GFSFSSRFY |
| (Clone 11-3) | VH-CDR2 | 124 | YGGSSGST |
| | VH-CDR3 | 125 | ARGGSTAAAGFNL |
| | VL-CDR1 | 128 | EDIYNL |
| | VL-CDR2 | 129 | YAS |
| | VL-CDR3 | 130 | QCNDYGGTYVPNA |
| Anti-ZIKV E | VH-CDR1 | 136 | GFTFSNYA |
| Protein | VH-CDR2 | 137 | IGRNGDSI |
| (Clone ZKA- | VH-CDR3 | 138 | VKDLAIPESYRIEADY |
| 78) | VL-CDR1 | 140 | QSVLYRSNNKNY |
| | VL-CDR2 | 141 | LIYWASTRE |
| | VL-CDR3 | 142 | QQYYSSPRT |
| Antibody | VH-CDR1 | 151 | GLDFSTNSY |
| Clone 78-2 | VH-CDR2 | 152 | IYVGDSSEI |
| | VH-CDR3 | 153 | ARDLPSFTAPYAGYLRL |
| | VL-CDR1 | 156 | TGYNVGDYP |
| | VL-CDR2 | 157 | YHTEEFKH |
| | VL-CDR3 | 158 | YTVHATESSLHYVF |
| Antibody | VH-CDR1 | 165 | EFDSSSNA |
| Clone 278-11 | VH-CDR2 | 166 | IYSGSGTI |
| | VH-CDR3 | 167 | ARYNTGGFYYDL |
| | VL-CDR1 | 170 | QRIGTN |
| | VL-CDR2 | 171 | KAS |
| | VL-CDR3 | 172 | QQGYSSNDADNT |

### SEQUENCE LISTING FREE TEXT

With respect to the requirements within WIPO Standard ST.25 concerning the presentation of nucleotide and amino acid sequence listings in patent applications, the free text as used in the sequence listing is repeated in the following:

| SEQ | | SEQ | | SEQ | |
|---|---|---|---|---|---|
| ID | | ID | | ID | |
| NO: | Free Text | NO: | Free Text | NO: | Free Text |
| 3 | ZIKV E-Protein | 34 | Anti-ZIKV #3 VH | 64 | Anti-ZIKV #5 VH-CDR2 |
| 4 | EDIII domain of Zika virus | 35 | Anti-ZIKV #3 VH-CDR1 | 65 | Anti-ZIKV #5 VH-CDR3 |
| 6 | Anti-ZIKV #1 VH | 36 | Anti-ZIKV #3 VH-CDR2 | 67 | Anti-ZIKV #5 VL |
| 7 | Anti-ZIKV #1 VH-CDR1 | 37 | Anti-ZIKV #3 VH-CDR3 | 68 | Anti-ZIKV #5 VL-CDR1 |
| 8 | Anti-ZIKV #1 VH-CDR2 | 39 | Anti-ZIKV #3 VL | 69 | Anti-ZIKV #5 VL-CDR2 |
| 9 | Anti-ZIKV #1 VH-CDR3 | 40 | Anti-ZIKV #3 VL-CDR1 | 70 | Anti-ZIKV #5 VL-CDR3 |
| 11 | Anti-ZIKV #1 VL | 41 | Anti-ZIKV #3 VL-CDR2 | 72 | Anti-ZIKV #5 VH DNA |
| 12 | Anti-ZIKV #1 VL-CDR1 | 42 | Anti-ZIKV #3 VL-CDR3 | 74 | Anti-ZIKV #5 VL DNA |
| 13 | Anti-ZIKV #1 VL-CDR2 | 44 | Anti-ZIKV #3 VH DNA | 75 | Anti-ZIKV #6 VH |
| 14 | Anti-ZIKV #1 VL-CDR3 | 46 | Anti-ZIKV #3 VL DNA | 76 | Anti-ZIKV #6 VH-CDR1 |
| 16 | Anti-ZIKV #1 VH DNA | 48 | Anti-ZIKV #4 VH | 77 | Anti-ZIKV #6 VH-CDR2 |
| 18 | Anti-ZIKV #1 VL DNA | 49 | Anti-ZIKV #4 VH-CDR1 | 78 | Anti-ZIKV #6 VH-CDR3 |
| 20 | Anti-ZIKV #2 VH | 50 | Anti-ZIKV #4 VH-CDR2 | 79 | Anti-ZIKV #6 VL |
| 21 | Anti-ZIKV #2 VH-CDR1 | 51 | Anti-ZIKV #4 VH-CDR3 | 80 | Anti-ZIKV #6 VL-CDR1 |
| 22 | Anti-ZIKV #2 VH-CDR2 | 53 | Anti-ZIKV #4 VL | 81 | Anti-ZIKV #6 VL-CDR2 |
| 23 | Anti-ZIKV #2 VH-CDR3 | 54 | Anti-ZIKV #4 VL-CDR1 | 82 | Anti-ZIKV #6 VL-CDR3 |
| 25 | Anti-ZIKV #2 VL | 55 | Anti-ZIKV #4 VL-CDR2 | 83 | Anti-ZIKV #7 VH |
| 26 | Anti-ZIKV #2 VL-CDR1 | 56 | Anti-ZIKV #4 VL-CDR3 | 84 | Anti-ZIKV #7 VH-CDR1 |
| 27 | Anti-ZIKV #2 VL-CDR2 | 58 | Anti-ZIKV #4 VH DNA | 85 | Anti-ZIKV #7 VH-CDR2 |
| 28 | Anti-ZIKV #2 VL-CDR3 | 60 | Anti-ZIKV #4 VL DNA | 86 | Anti-ZIKV #7 VH-CDR3 |
| 30 | Anti-ZIKV #2 VH DNA | 62 | Anti-ZIKV #5 VH | 87 | Anti-ZIKV #7 VL |
| 32 | Anti-ZIKV #2 VL DNA | 63 | Anti-ZIKV #5 VH-CDR1 | 88 | Anti-ZIKV #7 VL-CDR1 |
| 89 | Anti-ZIKV #7 VL-CDR2 | 102 | Anti-ZIKV #8 VL-CDR3 | 118 | Anti-ZIKV #9 and #10 VH |
| | | | | | DNA |
| 90 | Anti-ZIKV #7 VL-CDR3 | 104 | Anti-ZIKV #8 VH DNA | 120 | Anti-ZIKV #9 and #10 VL |
| | | | | | DNA |
| 91 | Anti-ZIKV #7 VH DNA | 106 | Anti-ZIKV #8 VL DNA | 122 | Anti-ZIKV #11 VH |
| 92 | Anti-ZIKV #7 VL DNA | 108 | Anti-ZIKV #9 and #10 VH | 123 | Anti-ZIKV #11 VH-CDR1 |
| 94 | Anti-ZIKV #8 VH | 109 | Anti-ZIKV #9 and #10 VH- | 124 | Anti-ZIKV #11 VH-CDR2 |
| | | | CDR1 | | |
| 95 | Anti-ZIKV #8 VH-CDR1 | 110 | Anti-ZIKV #9 and #10 VH- | 125 | Anti-ZIKV #11 VH-CDR3 |
| | | | CDR2 | | |
| 96 | Anti-ZIKV #8 VH-CDR2 | 111 | Anti-ZIKV #9 and #10 VH- | 127 | Anti-ZIKV #11 VL |
| | | | CDR3 | | |
| 97 | Anti-ZIKV #8 VH-CDR3 | 113 | Anti-ZIKV #9 and #10 VL | 128 | Anti-ZIKV #11 VL-CDR1 |
| 99 | Anti-ZIKV #8 VL | 114 | Anti-ZIKV #9 and #10 VL- | 129 | Anti-ZIKV #11 VL-CDR2 |
| | | | CDR1 | | |
| 100 | Anti-ZIKV #8 VL-CDR1 | 115 | Anti-ZIKV #9 and #10 VL- | 130 | Anti-ZIKV #11 VL-CDR3 |
| | | | CDR2 | | |
| 101 | Anti-ZIKV #8 VL-CDR2 | 116 | Anti-ZIKV #9 and #10 VL- | 132 | Anti-ZIKV #11 VH DNA |
| | | | CDR3 | | |
| 134 | Anti-ZIKV #11 VL DNA | 139 | Anti-ZIKV E Protein VL | 144 | Anti-ZIKV E Protein VL DNA |
| 135 | Anti-ZIKV E Protein VH | 140 | Anti-ZIKV E Protein VL-CDR1 | 150 | Antibody Clone 78-2 VH |
| 136 | Anti-ZIKV E Protein VH- | 141 | Anti-ZIKV E Protein VL-CDR2 | 151 | Antibody Clone 78-2 VH- |
| | CDR1 | | | | CDR1 |
| 137 | Anti-ZIKV E Protein VH- | 142 | Anti-ZIKV E Protein VL-CDR3 | 152 | Antibody Clone 78-2 VH- |
| | CDR2 | | | | CDR1 |
| 138 | Anti-ZIKV E Protein VH- | 143 | Anti-ZIKV E Protein VH DNA | 153 | Antibody Clone 78-2 VH- |
| | CDR3 | | | | CDR3 |
| 155 | Antibody Clone 78-2 VL | 164 | Antibody Clone 278-11 VH | 171 | Antibody Clone 278-11 VL- |
| | | | | | CDR2 |
| 156 | Antibody Clone 78-2 VL- | 165 | Antibody Clone 278-11 VH- | 172 | Antibody Clone 278-11 VL- |
| | CDR1 | | CDR1 | | CDR3 |
| 157 | Antibody Clone 78-2 VL- | 166 | Antibody Clone 278-11 VH- | 174 | Antibody Clone 278-11 VH |
| | CDR2 | | CDR2 | | DNA |
| 158 | Antibody Clone 78-2 VL- | 167 | Antibody Clone 278-11 VH- | 176 | Antibody Clone 278-11 VL |
| | CDR3 | | CDR3 | | DNA |
| 160 | Antibody Clone 78-2 VH | 169 | Antibody Clone 278-11 VL | | |
| | DNA | | | | |
| 162 | Antibody Clone 78-2 VL | 170 | Antibody Clone 278-11 VL- | | |
| | DNA | | CDR1 | | |

### Items of the Invention

### Microsphere Complex Comprising Microsphere Coupled to ZIKV VLP

1. A microsphere complex comprising a microsphere coupled to a zika virus like particle.
2. The microsphere complex of item 1, wherein the zika virus like particle is derived from zika virus strain Z1106033 characterized by SEQ ID NO: 1 and/or SEQ ID NO: 2.
3. The microsphere complex of item 1, wherein the zika virus like particle comprises structural proteins of zika virus strain Z1106033 characterized by SEQ ID NO: 1 and/or SEQ ID NO: 2.
4. The microsphere complex of item 1, wherein the zika virus like particle comprises the envelope glycoprotein, membrane protein, and/or pre-membrane protein which are at least 70%, or at least 75%, or least 80%, or at least 85%, or at least 90%, or at least 95% or 100% identical to corresponding parts of SEQ ID NO: 2.
5. The microsphere complex of any one of items 1 to 4, wherein the zika virus like particle is produced in human embryonic kidney (HEK293) cells.
6. The microsphere complex of any one of items 1 to 5, wherein the microsphere is a polystyrene microsphere.
7. The microsphere complex of any one of items 1 to 6, wherein the microsphere is magnetic.
8. The microsphere complex of any one of items 1 to 7, wherein the microsphere has a diameter in the range from about 0.01 to about 100 μm, preferably in the range from about 1 to 10 μm.
9. The microsphere complex of any one of items 1 to 8, wherein the microsphere contains carboxylate groups at the microsphere surface.
10. The microsphere complex of item 9, wherein coupling of the microsphere to the zika virus like particle occurs by formation of an amide bond between a carboxylate group of the microsphere and an amine group of the zika virus like particle.
11. The microsphere complex of any of items 1 to 10, wherein the microsphere can be identified by a specific feature.
12. The microsphere complex of item 11, wherein the specific feature is that the microsphere comprises one or more fluorescent dyes having a specific emission spectrum.
13. The microsphere complex of item 12, wherein the one or more fluorescent dyes are selected from the group consisting of squaraine, phthalocyanine, naphthalocyanine, and any derivative thereof.

### Kit

14. A kit comprising:
   an amount of the microsphere complex of any one of items 1 to 13, and
   an amount of a reporter antibody that binds to the zika virus like particle of the microsphere complex.
15. The kit of item 14, wherein the reporter antibody is a zika virus neutralizing antibody.
16. The kit of item 14 or 15, wherein the reporter antibody does not cross-react with antigens from other flaviviruses, such as dengue virus, West Nile virus, Japanese encephalitis virus, Yellow Fever Virus, St. Louis Encephalitis virus, and Tick Borne Encephalitis virus.
17. The kit of item 16, wherein the reporter antibody does not cross-react with dengue virus antigens, such as dengue virus like particles.
18. The kit of any one of items 14 to 17, wherein the reporter antibody is a monoclonal antibody.
19. The kit of any one of items 14 to 18, wherein the reporter antibody is derived from a non-human origin.
20. The kit of any of items 14 to 19, wherein the reporter antibody is attached to at least one detectable label, preferably by the heavy chain constant region of the reporter antibody.
21. The kit of item 20, wherein the reporter antibody is directly attached to the at least one detectable label, preferably by the heavy chain constant region of the reporter antibody.
22. The kit of item 20, wherein the reporter antibody is indirectly attached to the at least one detectable label, preferably by the heavy chain constant region of the reporter antibody, wherein the reporter antibody reacts with a secondary reporter antibody directly attached to at least one detectable label.
23. The kit of item 22, wherein the secondary reporter antibody is directly attached to the at least one detectable label, preferably by the heavy chain constant region of the secondary reporter antibody.
24. The kit of any of items 20 to 23, wherein the at least one detectable label is a fluorescence label, such as xanthene, fluorescein isothiocyanate, rhodamine, phycoerythrin, cyanine, coumarin, and any derivative thereof.
25. The kit of item 24, wherein the at least one detectable label is phycoerythrin.
26. The kit of any of items 14 to 25, wherein the reporter antibody provides an EC₅₀ value towards the zika virus like particle coupled to the microsphere within the microsphere complex of less than 0.5 μg/mL, or less than 0.4 μg/mL or less than 0.3 μg/mL or less than 0.2 μg/mL or less than 0.15 μg/mL or less than 0.1 μg/mL or less than 0.09 μg/mL or less than 0.08 μg/mL or less than 0.07 μg/mL or less than 0.05 μg/mL or less than 0.04 μg/mL or less than 0.03 μg/mL or less than 0.01 μg/mL.
27. The kit of any of items 14 to 25, wherein the reporter antibody is a zika virus specific reporter antibody, wherein the reporter antibody provides an EC₅₀ value towards the zika virus like particle coupled to the microsphere within the microsphere complex which is lower than each EC₅₀ value which said reporter antibody provides when tested in binding towards other microsphere complexes comprising a microsphere coupled to a dengue virus like particle.
28. The kit of the item 27, wherein the dengue virus like particle is a dengue serotype 1 virus like particle and/or a dengue serotype 2 virus like particle and/or a dengue serotype 3 virus like particle and/or a dengue serotype 4 virus like particle.
29. The kit of any of items 27 or 28, wherein the EC₅₀ value towards the zika virus like particle is less than 0.5 μg/mL, or less than 0.4 μg/mL or less than 0.3 μg/mL or less than 0.2 μg/mL or less than 0.15 μg/mL or less than 0.1 μg/mL or less than 0.09 μg/mL or less than 0.08 μg/mL or less than 0.07 μg/mL or less than 0.05 μg/mL or less than 0.04 μg/mL or less than 0.03 μg/mL or less than 0.01 μg/mL and the EC₅₀ towards the dengue virus like particle is at least 1 μg/mL or at least 1.1 μg/mL or at least 1.2 μg/mL or at least 1.3 μg/mL or at least 1.4 μg/mL.
30. The kit of any one of items 14 to 29, wherein the reporter antibody binds to the zika virus envelope glycoprotein domain III of the envelope glycoprotein encoded by SEQ ID NO: 3.
31. The kit of item 30, wherein the reporter antibody binds to amino acids T309 and G337 of SEQ ID NO: 3.
32. The kit of item 30, wherein the reporter antibody binds to amino acid E370 of SEQ ID NO: 3.
33. The kit of item 30, wherein the reporter antibody binds to amino acids T397 and H398 of SEQ ID NO: 3.
34. The kit of item 30, wherein the reporter antibody binds to amino acids E162, G181, G182, and K301 of SEQ ID NO: 3.
35. The kit of any one of items 14 to 30, wherein the reporter antibody comprises
   a heavy chain variable region (VH) comprising a heavy chain complementary determining region 1 (VH-CDR1) amino acid sequence of SEQ ID NO: 7, a heavy chain complementary determining region 2 (VH-CDR2) amino acid sequence of SEQ ID NO: 8, and a heavy chain complementary determining region 3 (VH-CDR3) amino acid sequence of SEQ ID NO: 9, and
   a light chain variable region (VL) comprising a light chain complementary determining region 1 (VL-CDR1) amino acid sequence of SEQ ID NO: 12, a light chain complementary determining region 2 (VL-CDR2) amino acid sequence of SEQ ID NO: 13, and a light chain complementary determining region 3 (VL-CDR3) amino acid sequence of SEQ ID NO: 14.
36. The kit of any one of items 14 to 30, wherein the reporter antibody comprises
   a heavy chain variable region (VH) comprising a heavy chain complementary determining region 1 (VH-CDR1) amino acid sequence of SEQ ID NO: 21, a heavy chain complementary determining region 2 (VH-CDR2) amino acid sequence of SEQ ID NO: 22, and a heavy chain complementary determining region 3 (VH-CDR3) amino acid sequence of SEQ ID NO: 23, and
   a light chain variable region (VL) comprising a light chain complementary determining region 1 (VL-CDR1) amino acid sequence of SEQ ID NO: 26, a light chain complementary determining region 2 (VL-CDR2) amino acid sequence of SEQ ID NO: 27, and a light chain complementary determining region 3 (VL-CDR3) amino acid sequence of SEQ ID NO: 28.
37. The kit of any one of items 14 to 30, wherein the reporter antibody comprises
   a heavy chain variable region (VH) comprising a heavy chain complementary determining region 1 (VH-CDR1) amino acid sequence of SEQ ID NO: 35, a heavy chain complementary determining region 2 (VH-CDR2) amino acid sequence of SEQ ID NO: 36, and a heavy chain complementary determining region 3 (VH-CDR3) amino acid sequence of SEQ ID NO: 37, and
   a light chain variable region (VL) comprising a light chain complementary determining region 1 (VL-CDR1) amino acid sequence of SEQ ID NO: 40, a light chain complementary determining region 2 (VL-CDR2) amino acid sequence of SEQ ID NO: 41, and a light chain complementary determining region 3 (VL-CDR3) amino acid sequence of SEQ ID NO: 42.
38. The kit of any one of items 14 to 30, wherein the reporter antibody comprises
   a heavy chain variable region (VH) comprising a heavy chain complementary determining region 1 (VH-CDR1) amino acid sequence of SEQ ID NO: 49, a heavy chain complementary determining region 2 (VH-CDR2) amino acid sequence of SEQ ID NO: 50, and a heavy chain complementary determining region 3 (VH-CDR3) amino acid sequence of SEQ ID NO: 51, and
   a light chain variable region (VL) comprising a light chain complementary determining region 1 (VL-CDR1) amino acid sequence of SEQ ID NO: 54, a light chain complementary determining region 2 (VL-CDR2) amino acid sequence of SEQ ID NO: 55, and a light chain complementary determining region 3 (VL-CDR3) amino acid sequence of SEQ ID NO: 56.
39. The kit of any one of items 14 to 30, wherein the reporter antibody comprises
   a heavy chain variable region (VH) comprising a heavy chain complementary determining region 1 (VH-CDR1) amino acid sequence of SEQ ID NO: 63, a heavy chain complementary determining region 2 (VH-CDR2) amino acid sequence of SEQ ID NO: 64, and a heavy chain complementary determining region 3 (VH-CDR3) amino acid sequence of SEQ ID NO: 65, and
   a light chain variable region (VL) comprising a light chain complementary determining region 1 (VL-CDR1) amino acid sequence of SEQ ID NO: 68, a light chain complementary determining region 2 (VL-CDR2) amino acid sequence of SEQ ID NO: 69, and a light chain complementary determining region 3 (VL-CDR3) amino acid sequence of SEQ ID NO: 70.
40. The kit of any one of items 14 to 30, wherein the reporter antibody comprises
   a heavy chain variable region (VH) comprising a heavy chain complementary determining region 1 (VH-CDR1) amino acid sequence of SEQ ID NO: 76, a heavy chain complementary determining region 2 (VH-CDR2) amino acid sequence of SEQ ID NO: 77, and a heavy chain complementary determining region 3 (VH-CDR3) amino acid sequence of SEQ ID NO: 78, and
   a light chain variable region (VL) comprising a light chain complementary determining region 1 (VL-CDR1) amino acid sequence of SEQ ID NO: 80, a light chain complementary determining region 2 (VL-CDR2) amino acid sequence of SEQ ID NO: 81, and a light chain complementary determining region 3 (VL-CDR3) amino acid sequence of SEQ ID NO: 82.
41. The kit of any one of items 14 to 30, wherein the reporter antibody comprises
   a heavy chain variable region (VH) comprising a heavy chain complementary determining region 1 (VH-CDR1) amino acid sequence of SEQ ID NO: 84, a heavy chain complementary determining region 2 (VH-CDR2) amino acid sequence of SEQ ID NO: 85, and a heavy chain complementary determining region 3 (VH-CDR3) amino acid sequence of SEQ ID NO: 86, and
   a light chain variable region (VL) comprising a light chain complementary determining region 1 (VL-CDR1) amino acid sequence of SEQ ID NO: 88, a light chain complementary determining region 2 (VL-CDR2) amino acid sequence of SEQ ID NO: 89, and a light chain complementary determining region 3 (VL-CDR3) amino acid sequence of SEQ ID NO: 90.
42. The kit of any one of items 14 to 30, wherein the reporter antibody comprises
   a heavy chain variable region (VH) comprising a heavy chain complementary determining region 1 (VH-CDR1) amino acid sequence of SEQ ID NO: 95, a heavy chain complementary determining region 2 (VH-CDR2) amino acid sequence of SEQ ID NO: 96, and a heavy chain complementary determining region 3 (VH-CDR3) amino acid sequence of SEQ ID NO: 97, and
   a light chain variable region (VL) comprising a light chain complementary determining region 1 (VL-CDR1) amino acid sequence of SEQ ID NO: 100, a light chain complementary determining region 2 (VL-CDR2) amino acid sequence of SEQ ID NO: 101, and a light chain complementary determining region 3 (VL-CDR3) amino acid sequence of SEQ ID NO: 102.
43. The kit of any one of items 14 to 30, wherein the reporter antibody comprises
   a heavy chain variable region (VH) comprising a heavy chain complementary determining region 1 (VH-CDR1) amino acid sequence of SEQ ID NO: 109, a heavy chain complementary determining region 2 (VH-CDR2) amino acid sequence of SEQ ID NO: 110, and a heavy chain complementary determining region 3 (VH-CDR3) amino acid sequence of SEQ ID NO: 111, and
   a light chain variable region (VL) comprising a light chain complementary determining region 1 (VL-CDR1) amino acid sequence of SEQ ID NO: 114, a light chain complementary determining region 2 (VL-CDR2) amino acid sequence of SEQ ID NO: 115, and a light chain complementary determining region 3 (VL-CDR3) amino acid sequence of SEQ ID NO: 116.
44. The kit of any one of items 14 to 30, wherein the reporter antibody comprises
   a heavy chain variable region (VH) comprising a heavy chain complementary determining region 1 (VH-CDR1) amino acid sequence of SEQ ID NO: 123, a heavy chain complementary determining region 2 (VH-CDR2) amino acid sequence of SEQ ID NO: 124, and a heavy chain complementary determining region 3 (VH-CDR3) amino acid sequence of SEQ ID NO: 125, and
   a light chain variable region (VL) comprising a light chain complementary determining region 1 (VL-CDR1) amino acid sequence of SEQ ID NO: 128, a light chain complementary determining region 2 (VL-CDR2) amino acid sequence of SEQ ID NO: 129, and a light chain complementary determining region 3 (VL-CDR3) amino acid sequence of SEQ ID NO: 130.
45. The kit of any one of items 14 to 30, wherein the reporter antibody comprises a heavy chain variable region (VH) amino acid sequence of SEQ ID NO: 6, and a light chain variable region (VL) amino acid sequence of SEQ ID NO: 11.
46. The kit of any one of items 14 to 30, wherein the reporter antibody comprises a heavy chain variable region (VH) amino acid sequence of SEQ ID NO: 20, and a light chain variable region (VL) amino acid sequence of SEQ ID NO: 25.
47. The kit of any one of items 14 to 30, wherein the reporter antibody comprises a heavy chain variable region (VH) amino acid sequence of SEQ ID NO: 34, and a light chain variable region (VL) amino acid sequence of SEQ ID NO: 39.
48. The kit of any one of items 14 to 30, wherein the reporter antibody comprises a heavy chain variable region (VH) amino acid sequence of SEQ ID NO: 48, and a light chain variable region (VL) amino acid sequence of SEQ ID NO: 53.
49. The kit of any one of items 14 to 30, wherein the reporter antibody comprises a heavy chain variable region (VH) amino acid sequence of SEQ ID NO: 62, and a light chain variable region (VL) amino acid sequence of SEQ ID NO: 67.
50. The kit of any one of items 14 to 30, wherein the reporter antibody comprises a heavy chain variable region (VH) amino acid sequence of SEQ ID NO: 75, and a light chain variable region (VL) amino acid sequence of SEQ ID NO: 79.
51. The kit of any one of items 14 to 30, wherein the reporter antibody comprises a heavy chain variable region (VH) amino acid sequence of SEQ ID NO: 83, and a light chain variable region (VL) amino acid sequence of SEQ ID NO: 87.
52. The kit of any one of items 14 to 30, wherein the reporter antibody comprises a heavy chain variable region (VH) amino acid sequence of SEQ ID NO: 94, and a light chain variable region (VL) amino acid sequence of SEQ ID NO: 99.
53. The kit of any one of items 14 to 30, wherein the reporter antibody comprises a heavy chain variable region (VH) amino acid sequence of SEQ ID NO: 108, and a light chain variable region (VL) amino acid sequence of SEQ ID NO: 113.
54. The kit of any one of items 14 to 30, wherein the reporter antibody comprises a heavy chain variable region (VH) amino acid sequence of SEQ ID NO: 122, and a light chain variable region (VL) amino acid sequence of SEQ ID NO: 127.
55. The kit of any one of items 14 to 30, wherein the reporter antibody comprises a heavy chain (H) amino acid sequence of SEQ ID NO: 5, and a light chain (V) amino acid sequence of SEQ ID NO: 10.
56. The kit of any one of items 14 to 30, wherein the reporter antibody comprises a heavy chain (H) amino acid sequence of SEQ ID NO: 19, and a light chain (V) amino acid sequence of SEQ ID NO: 24.
57. The kit of any one of items 14 to 30, wherein the reporter antibody comprises a heavy chain (H) amino acid sequence of SEQ ID NO: 33, and a light chain (V) amino acid sequence of SEQ ID NO: 38.
58. The kit of any one of items 14 to 30, wherein the reporter antibody comprises a heavy chain (H) amino acid sequence of SEQ ID NO: 47, and a light chain (V) amino acid sequence of SEQ ID NO: 52.
59. The kit of any one of items 14 to 30, wherein the reporter antibody comprises a heavy chain (H) amino acid sequence of SEQ ID NO: 61, and a light chain (V) amino acid sequence of SEQ ID NO: 66.
60. The kit of any one of items 14 to 30, wherein the reporter antibody comprises a heavy chain (H) amino acid sequence of SEQ ID NO: 93, and a light chain (V) amino acid sequence of SEQ ID NO: 98.
61. The kit of any one of items 14 to 30, wherein the reporter antibody comprises a heavy chain (H) amino acid sequence of SEQ ID NO: 107, and a light chain (V) amino acid sequence of SEQ ID NO: 112.
62. The kit of any one of items 14 to 30, wherein the reporter antibody comprises a heavy chain (H) amino acid sequence of SEQ ID NO: 121, and a light chain (V) amino acid sequence of SEQ ID NO: 126.

### Method for Detecting Anti-Zika Virus Antibodies

63. A method for detecting a signal from a reporter antibody indicative for the presence and/or amount of anti-zika virus antibodies in a sample from a subject comprising the steps of:
   Step 1: providing a kit according to any one of items 14 to 62, including an amount of said microsphere complex and an amount of said reporter antibody,
   Step 2: contacting the amount of said microsphere complex and the amount of said reporter antibody of step 1 with the sample to allow binding of the anti-zika virus antibodies in the sample to the zika virus like particles coupled to the microspheres in the microsphere complex while competing with the reporter antibody, and
   Step 3: detecting a signal from the reporter antibody bound to the zika virus like particles coupled to the microspheres in the microsphere complex in step 2.
64. The method of item 63, wherein in step 2 the amount of said microsphere complex and the amount of said reporter antibody of step 1 are concomitantly contacted with the sample.
65. The method of item 63, comprising the steps of:
   Step 1: providing a kit according to any of items 14 to 62, including an amount of said microsphere complex and an amount of said reporter antibody,
   Step 2.1: contacting the amount of said microsphere complex of step 1 with the sample to allow binding of the anti-zika virus antibodies in the sample to the zika virus like particles coupled to the microspheres in the microsphere complex,
   Step 2.2: contacting said amount of reporter antibody with said microsphere complex and the sample of step 2.1 to allow binding of the reporter antibody to the zika virus like particles coupled to the microspheres in the microsphere complex, and
   Step 3: detecting a signal from the reporter antibody bound to the zika virus like particles coupled to the microspheres in the microsphere complex in step 2.2.
66. The method of item 63, comprising the steps of:
   Step 1: providing a kit according to any of items 14 to 62, including an amount of said microsphere complex and an amount of said reporter antibody,
   Step 2.1: contacting the amount of said microsphere complex of step 1 with the sample to allow binding of anti-zika virus antibodies in the sample to the zika virus like particles coupled to the microspheres in the microsphere complex,
   Step 2.2: contacting said amount of reporter antibody with said microsphere complex and the sample of step 2.1 to allow binding of the reporter antibody to the zika virus like particles coupled to the microspheres,
   Step 2.3: contacting said amount of reporter antibody, said amount of microsphere complex, and the sample of step 2.2 with an amount of a secondary reporter antibody to allow binding of the secondary reporter antibody to the constant region of the reporter antibody, and
   Step 3: detecting a signal from the secondary reporter antibody bound to the reporter antibody in step 2.3, wherein the reporter antibody is bound to the zika virus like particles coupled to the microspheres in the microsphere complex in step 2.2.
67. The method of item 63, wherein contacting in step 2 is carried out for about 10 to about 250 min.
68. The method of item 65, wherein contacting in step 2.1 is carried out for about 60 min and contacting in step 2.2 is carried out for about 10 min or for about 120 min.
69. The method of item 66, wherein contacting in step 2.1 is carried out for about 60 min and contacting in step 2.2 is carried out for about 10 min or for about 120 min and contacting in step 2.3 is carried out for about 30 min.
70. The method of any of items 63 to 69, wherein the signal in step 3 is resulting from the at least one detectable label.
71. The method of any of items 63 to 70, wherein the signal in step 3 is a fluorescence signal.
72. The method of item 71, wherein the signal in step 3 is a fluorescence signal resulting from phycoerythrin.
73. The method of any of items 63 to 72 for detecting the presence and/or amount of anti-zika virus antibodies in a sample from a subject comprising the further steps of:
   Step 4: determining the presence and/or the amount of the reporter antibody bound to the zika virus like particles coupled to the microspheres in the microsphere complex from the signal of step 3, and
   Step 5: determining the presence and/or the amount of anti-zika virus antibodies in the sample based on the presence and/or the amount of the reporter antibody determined in step 4.
74. The method of any of items 63 to 73, wherein the sample is a sample from the group consisting of blood, urine, serum, blood plasma, cerebrospinal fluid, and lymph fluid.
75. The method of item 74, wherein the sample is a blood plasma sample or serum sample.
76. The method of any of items 63 to 75, wherein the anti-zika virus antibodies from the sample of the subject are zika virus neutralizing antibodies.
77. The method of any of items 63 to 76, wherein the subject is a subject from the group consisting of mouse, primate, non-human primate, human, rabbit, cat, rat, horse, and sheep.
78. The method of item 77, wherein the subject is a non-human primate.
79. The method of item 77, wherein the subject is a human.

### Method for Determining an Antibody Correlate of Protection Against Zika Virus Infection

80. A method for determining an antibody correlate of protection against zika virus infection for a zika virus vaccine in a type of non-human subjects comprising the steps of:
   Step 1: selecting a group of said subjects which are zika virus naive,
   Step 2: dividing the group of subjects into at least two subgroups, wherein one subgroup functions as control group and at least one subgroup functions as inoculation group,
   Step 3: inoculating said at least one inoculation group with a dose of the zika virus vaccine,
   Step 4: challenging all subjects with an infectious amount of the zika virus,
   Step 5: determining the amount of anti-zika virus antibodies for each subject according to items 63 to
76 at least after inoculation with the zika virus vaccine and before challenging with the infectious amount of the zika virus,
   Step 6: determining presence or absence of viremia in all subjects after challenging with the infectious amount of the zika virus,
   Step 7: repeating steps 3 to 6 with further inoculation groups with increasing vaccine doses until absence of viremia is determined in all subjects of one inoculation group in step 6, and
   Step 8: determining the amount of anti-zika virus antibodies after inoculation with the zika virus vaccine and before challenging with the infectious amount of the zika virus associated with absence of viremia after challenging with the infectious amount of zika virus as antibody correlate of protection.
81. The method for determining an antibody correlate of protection according to item 80, wherein the zika virus vaccine in step 3 is a purified inactivated zika virus vaccine.
82. The method for determining an antibody correlate of protection according to any of items 80 or 81, wherein the zika virus in step 4 is zika virus strain PRVABC59.
83. The method for determining an antibody correlate of protection according to any of items 80 to 82, wherein the type of non-human subjects is selected from the group consisting of mice, primates, non-human primates, rabbits, cats, rats, horses, or sheep.
84. The method for determining an antibody correlate of protection according to item 83, wherein the type of non-human subjects is non-human primates.
85. A method for determining an antibody correlate of protection against zika virus infection in human subjects by mathematically modeling the correlate of protection of a non-human subject as determined according to any one of items 80 to 84 to fit human subjects.

### Method for Diagnosing the Protection of a Subject Against a Zika Virus Infection

86. A method for diagnosing the protection of a human subject against a zika virus infection comprising the steps of:
   Step 1: providing a sample from the human subject outside the human body,
   Step 2: determining the amount of anti-zika virus antibodies in the sample from the human subject according to items 63 to 76, and
   Step 3: determining protection by comparing the amount of anti-zika virus antibodies determined in step 2 to the antibody correlate of protection against zika virus infection in human subjects, which optionally has been determined according to item 85.
87. A method for diagnosing the protection of a non-human subject against a zika virus infection comprising the steps of:
   Step 1: providing a sample from the non-human subject outside the non-human body,
   Step 2: determining the amount of anti-zika virus antibodies in the sample from the non-human subject according to items 63 to 76, and
   Step 3: determining protection by comparing the amount of anti-zika virus antibodies determined in step 2 to the antibody correlate of protection determined according to items 80 to 84 in this type of non-human subjects.

### Method for Diagnosing a Zika Virus Infection

88. A method for diagnosing a zika virus infection in a subject comprising the steps of:
   Step 1: providing a sample from the subject outside the subject body,
   Step 2: determining the amount of anti-zika virus antibodies in the sample according to items 63 to 79, and
   Step 3: determining infection by comparing said amount of anti-zika virus antibodies to established amounts of anti-zika virus antibodies in zika virus infected subjects.
89. The method for diagnosing a zika virus infection according to item 88, wherein the subject is a human.
90. The method of any of items 88 or 89, wherein the zika virus infection is acute.
91. The method of any of items 88 or 89, wherein the zika virus infection is convalescent.

### Method for Detecting Total Anti-Zika Antibodies

92. A method for detecting a signal from a detection antibody indicative for the presence and/or amount of anti-zika virus antibodies in a sample from a subject comprising the steps of:
   Step 1: contacting an amount of a microsphere complex according to any one of items 1 to 13 with the sample to allow binding of anti-zika virus antibodies in the sample to the zika virus like particles coupled to the microspheres in the microsphere complex,
   Step 2: contacting an amount of a detection antibody with the microsphere complex and the sample of step 1 to allow binding of the detection antibody to the heavy chain constant region of the anti-zika virus antibodies bound to the zika virus like particles coupled to the microspheres in the microsphere complex, wherein the detection antibody binds to the anti-zika virus antibodies with the variable region of the detection antibody and wherein the detection antibody is attached to at least one detectable label, and
   Step 3: detecting a signal from the detection antibody bound to the anti-zika virus antibodies in step 2.
93. The method according to item 92 for determining the presence and/or amount of anti-zika virus antibodies in a sample from a subject, wherein the method comprises the further steps of:
   Step 4: determining the presence and/or amount of the detection antibody bound to the anti-zika virus antibodies from the signal of step 3, and
   Step 5: determining the presence and/or amount of anti-zika virus antibodies in the sample from the presence and/or amount of the detection antibody determined in step 4.
94. The method according to items 92 or 93, wherein contacting in step 1 is carried out for about 60 min and contacting in step 2 is carried out for about 30 min.
95. The method according to any one of items 92 to 94, wherein the detection antibody is attached to the at least one detectable label by the heavy chain constant region.
96. The method according to any one of items 92 to 95, wherein the at least one detectable label is a fluorescence label, such as phycoerythrin.
97. The method according to any one of items 92 to 96, wherein the signal in step 3 is resulting from the at least one detectable label, preferably the signal is a fluorescence signal.
98. The method according to any one of items 92 to 97, wherein the sample is a sample from the group consisting of blood, urine, serum, blood plasma, cerebrospinal fluid, and lymph fluid, in particular the sample is a serum or blood plasma sample.
99. The method according to any one of items 92 to 98, wherein the subject is from the group consisting of mouse, primate, non-human primate, human, rabbit, cat, rat, horse, and sheep, in particular the subject is a human.
100. The method for determining an antibody correlate of protection against zika virus infection according to any one of items 80 to 85, wherein the amount of anti-zika virus antibodies for each subject in step 5 is determined according to the method of any one of items 92 to 99.
101. The method for diagnosing the protection of a human subject against a zika virus infection according to item 86,
   wherein the amount of anti-zika virus antibodies in the sample in step 2 is determined according to the method of any one of items 92 to 98, and
   wherein protection in step 3 is determined by comparing the amount of anti-zika virus antibodies to the antibody correlate of protection determined in human subjects according to item 100.
102. The method for diagnosing the protection of a non-human subject against a zika virus infection according to item 87,
   wherein the amount of anti-zika virus antibodies in the sample in step 2 is determined according to the method of any one of items 92 to 98, and
   wherein protection in step 3 is determined by comparing the amount of anti-zika virus antibodies to the antibody correlate of protection determined in this type of non-human subjects according to item 100.

### Microsphere Complex Comprising a Microsphere Coupled to a DENV VLP and Corresponding Kit

103. A microsphere complex comprising a microsphere coupled to a dengue virus like particle.
104. The microsphere complex of item 103, wherein the microsphere is coupled to a dengue-1 virus like particle.
105. The microsphere complex of item 104, wherein the dengue-1 virus like particle is derived from dengue-1 virus strain Puerto Rico/US/BID-V853/1998 characterized by SEQ ID NO: 179 and/or SEQ ID NO: 180.
106. The microsphere complex of item 104, wherein the dengue-1 virus like particle comprises structural proteins of dengue-1 virus strain Puerto Rico/US/BID-V853/1998 characterized by SEQ ID NO: 179 and/or SEQ ID NO: 180.
107. The microsphere complex of item 104, wherein the dengue-1 virus like particle comprises the envelope protein, the membrane protein, and/or the pre-membrane protein which are at least 70%, or at least 75%, or at least 80%, or at least 85%, or at least 90%, or at least 95% or 100% identical to corresponding parts of SEQ ID NO: 180.
108. The microsphere complex of item 103, wherein the microsphere is coupled to a dengue-2 virus like particle.
109. The microsphere complex of item 108, wherein the dengue-2 virus like particle is derived from dengue-2 virus strain Thailand/16681/84 characterized by SEQ ID NO: 181 and/or SEQ ID NO: 182.
110. The microsphere complex of item 108, wherein the dengue-2 virus like particle comprises structural proteins of dengue-2 virus strain Thailand/16681/84 characterized by SEQ ID NO: 181 and/or SEQ ID NO: 182.
111. The microsphere complex of item 108, wherein the dengue-2 virus like particle comprises the envelope protein, the membrane protein, and/or the pre-membrane protein which are at least 70%, or at least 75%, or at least 80%, or at least 85%, or at least 90%, or at least 95% or 100% identical to corresponding parts of SEQ ID NO: 182.
112. The microsphere complex of item 103, wherein the microsphere is coupled to a dengue-3 virus like particle.
113. The microsphere complex of item 112, wherein the dengue-3 virus like particle is derived from dengue-3 virus strain Sri Lanka D3/H/IMTSSA-SRI/2000/1266 characterized by SEQ ID NO: 183 and/or SEQ ID NO: 184.
114. The microsphere complex of item 112, wherein the dengue-3 virus like particle comprises structural proteins of dengue-3 virus strain Sri Lanka D3/H/IMTSSA-SRI/2000/1266 characterized by SEQ ID NO: 183 and/or SEQ ID NO: 184.
115. The microsphere complex of item 112, wherein the dengue-3 virus like particle comprises the envelope protein, the membrane protein, and/or the pre-membrane protein which are at least 70%, or at least 75%, or at least 80%, or at least 85%, or at least 90%, or at least 95% or 100% identical to corresponding parts of SEQ ID NO: 184.
116. The microsphere complex of item 103, wherein the microsphere is coupled to a dengue-4 virus like particle.
117. The microsphere complex of item 116, wherein the dengue-4 virus like particle is derived from dengue-4 virus strain Dominica/814669/1981 characterized by SEQ ID NO: 185 and/or SEQ ID NO: 186.
118. The microsphere complex of item 116, wherein the dengue-4 virus like particle comprises structural proteins of dengue-4 virus strain Dominica/814669/1981 characterized by SEQ ID NO: 185 and/or SEQ ID NO: 186.
119. The microsphere complex of item 116, wherein the dengue-4 virus like particle comprises the envelope protein, the membrane protein, and/or the pre-membrane protein which are at least 70%, or at least 75%, or at least 80%, or at least 85%, or at least 90%, or at least 95% or 100% identical to corresponding parts of SEQ ID NO: 186.
120. The microsphere complex of any one of items 103 to 119, wherein the dengue virus like particle is produced in human embryonic kidney (HEK293) cells.

### Method for Preventing Zika Disease

121. A method for preventing zika disease in a human subject comprising the steps of:
   Step 1: obtaining a sample from the human subject,
   Step 2: determining the amount of anti-zika virus antibodies in the sample from the human subject according to items 63-76,
   Step 3: determining whether the human subject has an amount of anti-zika virus antibodies to confer protection by comparing the amount of anti-zika virus antibodies determined in step 2 to the antibody correlate of protection against zika virus infection in human subjects, which optionally has been determined according to item 85, and
   Step 4: administering to the human subject a zika virus vaccine if the human subject has an amount of anti-zika antibodies that is lower than the antibody correlate of protection against zika virus infection in human subjects, which optionally has been determined according to item 85.
122. A zika virus vaccine for use in a method for preventing zika disease in a human subject, the method comprising the steps of:
   Step 1: providing a sample from the human subject outside the human body,
   Step 2: determining the amount of anti-zika virus antibodies in the sample from the human subject according to items 63-76,
   Step 3: determining whether the human subject has an amount of anti-zika virus antibodies to confer protection by comparing the amount of anti-zika virus antibodies determined in step 2 to the antibody correlate of protection against zika virus infection in human subjects, which optionally has been determined according to item 85, and
   Step 4: administering to the human subject the zika virus vaccine if the human subject has an amount of anti-zika antibodies that is lower than the antibody correlate of protection against zika virus infection in human subjects, which optionally has been determined according to item 85.
123. The method according to item 121, wherein the zika virus vaccine is a purified inactivated zika virus vaccine.
124. The method according to item 121 or 123, wherein the human subject is a woman.
125. The zika virus vaccine for use according to item 122, wherein the zika virus vaccine is a purified inactivated zika virus vaccine.
126. The zika virus vaccine for use according to item 122 or 125, wherein the human subject is a woman.

### Method for Assaying the Presence of a Zika Virus Infection

127. A method for assaying the presence of a zika virus infection in a subject comprising the steps of:
   Step 1: obtaining a sample from the subject,
   Step 2: determining the amount of anti-zika virus antibodies in the sample according to items 63-79, and
   Step 3: determining the presence of a zika virus infection by comparing said amount of anti-zika virus antibodies to established amounts of anti-zika virus antibodies in zika virus infected subjects.
128. The method for assaying the presence of a zika virus infection according to item 127, wherein the subject is a human.
129. The method of any of items 127 or 128, wherein the zika virus infection is acute.
130. The method of any of items 127 or 128, wherein the zika virus infection is convalescent.

1. A microsphere complex comprising a microsphere coupled to a zika virus like particle.

2. The microsphere complex of claim 1, wherein the zika virus like particle comprises the envelope glycoprotein, membrane protein, and/or pre-membrane protein which are at least 70%, or at least 75%, or least 80%, or at least 85%, or at least 90%, or at least 95% or 100% identical to corresponding parts of SEQ ID NO: 2.

3. A kit comprising:
an amount of the microsphere complex of claim 1, and
an amount of a reporter antibody that binds to the zika virus like particle of the microsphere complex, preferably wherein the reporter antibody is a zika virus neutralizing antibody, and/or does not cross-react with antigens from other flaviviruses, such as dengue virus, West Nile virus, Japanese encephalitis virus, Yellow Fever Virus, St. Louis Encephalitis virus, and Tick Borne Encephalitis virus.

4.-5. (canceled)

6. The kit of claim 3, wherein the reporter antibody is attached to at least one detectable label, optionally by the heavy chain constant region of the reporter antibody.

7. The kit of claim 6, wherein the at least one detectable label is a fluorescence label, such as xanthene, fluorescein isothiocyanate, rhodamine, phycoerythrin, cyanine, coumarin, and any derivative thereof.

8. The kit of claim 3, wherein the reporter antibody provides an EC₅₀ value towards the zika virus like particle coupled to the microsphere within the microsphere complex of less than 0.5 μg/mL, or less than 0.4 μg/mL or less than 0.3 μg/mL or less than 0.2 μg/mL or less than 0.15 μg/mL or less than 0.1 μg/mL or less than 0.09 μg/mL or less than 0.08 μg/mL or less than 0.07 μg/mL or less than 0.05 μg/mL or less than 0.04 μg/mL or less than 0.03 μg/mL or less than 0.01 μg/mL.

9. The kit of claim 3, wherein the reporter antibody comprises
a heavy chain variable region (VH) comprising a heavy chain complementary determining region 1 (VH-CDR1) amino acid sequence of SEQ ID NO: 7, a heavy chain complementary determining region 2 (VH-CDR2) amino acid sequence of SEQ ID NO: 8, and a heavy chain complementary determining region 3 (VH-CDR3) amino acid sequence of SEQ ID NO: 9, and
a light chain variable region (VL) comprising a light chain complementary determining region 1 (VL-CDR1) amino acid sequence of SEQ ID NO: 12, a light chain complementary determining region 2 (VL-CDR2) amino acid sequence of SEQ ID NO: 13, and a light chain complementary determining region 3 (VL-CDR3) amino acid sequence of SEQ ID NO: 14,
or
a heavy chain variable region (VH) comprising a heavy chain complementary determining region 1 (VH-CDR1) amino acid sequence of SEQ ID NO: 21, a heavy chain complementary determining region 2 (VH-CDR2) amino acid sequence of SEQ ID NO: 22, and a heavy chain complementary determining region 3 (VH-CDR3) amino acid sequence of SEQ ID NO: 23, and
a light chain variable region (VL) comprising a light chain complementary determining region 1 (VL-CDR1) amino acid sequence of SEQ ID NO: 26, a light chain complementary determining region 2 (VL-CDR2) amino acid sequence of SEQ ID NO: 27, and a light chain complementary determining region 3 (VL-CDR3) amino acid sequence of SEQ ID NO: 28;
or
a heavy chain variable region (VH) comprising a heavy chain complementary determining region 1 (VH-CDR1) amino acid sequence of SEQ ID NO: 35, a heavy chain complementary determining region 2 (VH-CDR2) amino acid sequence of SEQ ID NO: 36, and a heavy chain complementary determining region 3 (VH-CDR3) amino acid sequence of SEQ ID NO: 37, and
a light chain variable region (VL) comprising a light chain complementary determining region 1 (VL-CDR1) amino acid sequence of SEQ ID NO: 40, a light chain complementary determining region 2 (VL-CDR2) amino acid sequence of SEQ ID NO: 41, and a light chain complementary determining region 3 (VL-CDR3) amino acid sequence of SEQ ID NO: 42,
or
a heavy chain variable region (VH) comprising a heavy chain complementary determining region 1 (VH-CDR1) amino acid sequence of SEQ ID NO: 49, a heavy chain complementary determining region 2 (VH-CDR2) amino acid sequence of SEQ ID NO: 50, and a heavy chain complementary determining region 3 (VH-CDR3) amino acid sequence of SEQ ID NO: 51, and
a light chain variable region (VL) comprising a light chain complementary determining region 1 (VL-CDR1) amino acid sequence of SEQ ID NO: 54, a light chain complementary determining region 2 (VL-CDR2) amino acid sequence of SEQ ID NO: 55, and a light chain complementary determining region 3 (VL-CDR3) amino acid sequence of SEQ ID NO: 56,
or
a heavy chain variable region (VH) comprising a heavy chain complementary determining region 1 (VH-CDR1) amino acid sequence of SEQ ID NO: 63, a heavy chain complementary determining region 2 (VH-CDR2) amino acid sequence of SEQ ID NO: 64, and a heavy chain complementary determining region 3 (VH-CDR3) amino acid sequence of SEQ ID NO: 65, and
a light chain variable region (VL) comprising a light chain complementary determining region 1 (VL-CDR1) amino acid sequence of SEQ ID NO: 68, a light chain complementary determining region 2 (VL-CDR2) amino acid sequence of SEQ ID NO: 69, and a light chain complementary determining region 3 (VL-CDR3) amino acid sequence of SEQ ID NO: 70,
or
a heavy chain variable region (VH) comprising a heavy chain complementary determining region 1 (VH-CDR1) amino acid sequence of SEQ ID NO: 76, a heavy chain complementary determining region 2 (VH-CDR2) amino acid sequence of SEQ ID NO: 77, and a heavy chain complementary determining region 3 (VH-CDR3) amino acid sequence of SEQ ID NO: 78, and
a light chain variable region (VL) comprising a light chain complementary determining region 1 (VL-CDR1) amino acid sequence of SEQ ID NO: 80, a light chain complementary determining region 2 (VL-CDR2) amino acid sequence of SEQ ID NO: 81, and a light chain complementary determining region 3 (VL-CDR3) amino acid sequence of SEQ ID NO: 82;
or
a heavy chain variable region (VH) comprising a heavy chain complementary determining region 1 (VH-CDR1) amino acid sequence of SEQ ID NO: 84, a heavy chain complementary determining region 2 (VH-CDR2) amino acid sequence of SEQ ID NO: 85, and a heavy chain complementary determining region 3 (VH-CDR3) amino acid sequence of SEQ ID NO: 86, and
a light chain variable region (VL) comprising a light chain complementary determining region 1 (VL-CDR1) amino acid sequence of SEQ ID NO: 88, a light chain complementary determining region 2 (VL-CDR2) amino acid sequence of SEQ ID NO: 89, and a light chain complementary determining region 3 (VL-CDR3) amino acid sequence of SEQ ID NO: 90,
or
a heavy chain variable region (VH) comprising a heavy chain complementary determining region 1 (VH-CDR1) amino acid sequence of SEQ ID NO: 95, a heavy chain complementary determining region 2 (VH-CDR2) amino acid sequence of SEQ ID NO: 96, and a heavy chain complementary determining region 3 (VH-CDR3) amino acid sequence of SEQ ID NO: 97, and
a light chain variable region (VL) comprising a light chain complementary determining region 1 (VL-CDR1) amino acid sequence of SEQ ID NO: 100, a light chain complementary determining region 2 (VL-CDR2) amino acid sequence of SEQ ID NO: 101, and a light chain complementary determining region 3 (VL-CDR3) amino acid sequence of SEQ ID NO: 102,
or
a heavy chain variable region (VH) comprising a heavy chain complementary determining region 1 (VH-CDR1) amino acid sequence of SEQ ID NO: 109, a heavy chain complementary determining region 2 (VH-CDR2) amino acid sequence of SEQ ID NO: 110, and a heavy chain complementary determining region 3 (VH-CDR3) amino acid sequence of SEQ ID NO: 111, and
a light chain variable region (VL) comprising a light chain complementary determining region 1 (VL-CDR1) amino acid sequence of SEQ ID NO: 114, a light chain complementary determining region 2 (VL-CDR2) amino acid sequence of SEQ ID NO: 115, and a light chain complementary determining region 3 (VL-CDR3) amino acid sequence of SEQ ID NO: 116,
or
a heavy chain variable region (VH) comprising a heavy chain complementary determining region 1 (VH-CDR1) amino acid sequence of SEQ ID NO: 123, a heavy chain complementary determining region 2 (VH-CDR2) amino acid sequence of SEQ ID NO: 124, and a heavy chain complementary determining region 3 (VH-CDR3) amino acid sequence of SEQ ID NO: 125, and
a light chain variable region (VL) comprising a light chain complementary determining region 1 (VL-CDR1) amino acid sequence of SEQ ID NO: 128, a light chain complementary determining region 2 (VL-CDR2) amino acid sequence of SEQ ID NO: 129, and a light chain complementary determining region 3 (VL-CDR3) amino acid sequence of SEQ ID NO: 130.

10.-18. (canceled)

19. A method for detecting a signal from a reporter antibody indicative for the presence and/or amount of anti-zika virus antibodies in a sample from a subject comprising the steps of:
Step 1: providing a kit according to claim 3, including an amount of said microsphere complex and an amount of said reporter antibody,
Step 2: contacting the amount of said microsphere complex and the amount of said reporter antibody of step 1 with the sample to allow binding of the anti-zika virus antibodies in the sample to the zika virus like particles coupled to the microspheres in the microsphere complex while competing with the reporter antibody, and
Step 3: detecting a signal from the reporter antibody bound to the zika virus like particles coupled to the microspheres in the microsphere complex in step 2

20. The method of claim 19, comprising the steps of:
Step 1: providing a kit according to claim 3, including an amount of said microsphere complex and an amount of said reporter antibody,
Step 2.1: contacting the amount of said microsphere complex of step 1 with the sample to allow binding of the anti-zika virus antibodies in the sample to the zika virus like particles coupled to the microspheres in the microsphere complex,
Step 2.2: contacting said amount of reporter antibody with said microsphere complex and the sample of step 2.1 to allow binding of the reporter antibody to the zika virus like particles coupled to the microspheres in the microsphere complex, and
Step 3: detecting a signal from the reporter antibody bound to the zika virus like particles coupled to the microspheres in the microsphere complex in step 2.2.

21. The method of claim 19, comprising the steps of:
Step 1: providing a kit according to claim 3, including an amount of said microsphere complex and an amount of said reporter antibody,
Step 2.1: contacting the amount of said microsphere complex of step 1 with the sample to allow binding of anti-zika virus antibodies in the sample to the zika virus like particles coupled to the microspheres in the microsphere complex,
Step 2.2: contacting said amount of reporter antibody with said microsphere complex and the sample of step 2.1 to allow binding of the reporter antibody to the zika virus like particles coupled to the microspheres,
Step 2.3: contacting said amount of reporter antibody, said amount of microsphere complex, and the sample of step 2.2 with an amount of a secondary reporter antibody to allow binding of the secondary reporter antibody to the constant region of the reporter antibody, and
Step 3: detecting a signal from the secondary reporter antibody bound to the reporter antibody in step 2.3, wherein the reporter antibody is bound to the zika virus like particles coupled to the microspheres in the microsphere complex in step 2.2.

22. The method of claim 19 for detecting the presence and/or amount of anti-zika virus antibodies in a sample from a subject comprising the further steps of:
Step 4: determining the presence and/or the amount of the reporter antibody bound to the zika virus like particles coupled to the microspheres in the microsphere complex from the signal of step 3, and
Step 5: determining the presence and/or the amount of anti-zika virus antibodies in the sample based on the presence and/or the amount of the reporter antibody determined in step 4.

23. The method of claim 19, wherein the sample is a sample from the group consisting of blood, urine, serum, blood plasma, cerebrospinal fluid, and lymph fluid.

24. The method of claim 19, wherein the subject is a subject from the group consisting of mouse, primate, non-human primate, human, rabbit, cat, rat, horse, and sheep.

25. A method for determining an antibody correlate of protection against zika virus infection for a zika virus vaccine in a type of non-human subjects comprising the steps of:
Step 1: selecting a group of said subjects which are zika virus naive,
Step 2: dividing the group of subjects into at least two subgroups, wherein one subgroup functions as control group and at least one subgroup functions as inoculation group,
Step 3: inoculating said at least one inoculation group with a dose of the zika virus vaccine,
Step 4: challenging all subjects with an infectious amount of the zika virus,
Step 5: determining the amount of anti-zika virus antibodies for each subject according to claim 19 at least after inoculation with the zika virus vaccine and before challenging with the infectious amount of the zika virus,
Step 6: determining presence or absence of viremia in all subjects after challenging with the infectious amount of the zika virus,
Step 7: repeating steps 3 to 6 with further inoculation groups with increasing vaccine doses until absence of viremia is determined in all subjects of one inoculation group in step 6, and
Step 8: determining the amount of anti-zika virus antibodies after inoculation with the zika virus vaccine and before challenging with the infectious amount of the zika virus associated with absence of viremia after challenging with the infectious amount of zika virus as antibody correlate of protection.

26. A method for determining an antibody correlate of protection against zika virus infection in human subjects by mathematically modeling the correlate of protection of a non-human subject as determined according to claim 25 to fit human subjects.

27. A method for diagnosing the protection of a human subject against a zika virus infection comprising the steps of:
Step 1: providing a sample from the human subject outside the human body,
Step 2: determining the amount of anti-zika virus antibodies in the sample from the human subject according to claim 19, and
Step 3: determining protection by comparing the amount of anti-zika virus antibodies determined in step 2 to the antibody correlate of protection against zika virus infection in human subjects.

28. A method for diagnosing the protection of a non-human subject against a zika virus infection comprising the steps of:
Step 1: providing a sample from the non-human subject outside the non-human body,
Step 2: determining the amount of anti-zika virus antibodies in the sample from the non-human subject according to claim 19, and
Step 3: determining protection by comparing the amount of anti-zika virus antibodies determined in step 2 to the antibody correlate of protection.

29. A method for diagnosing a zika virus infection in a subject comprising the steps of:
Step 1: providing a sample from the subject outside the subject body,
Step 2: determining the amount of anti-zika virus antibodies in the sample according to claim 19, and
Step 3: determining infection by comparing said amount of anti-zika virus antibodies to established amounts of anti-zika virus antibodies in zika virus infected subjects.

30. The method for diagnosing a zika virus infection according to claim 29, wherein the subject is a human.

31. The method of claim 29, wherein the zika virus infection is acute.

32. The method of claim 29, wherein the zika virus infection is convalescent.

33. A method for detecting a signal from a detection antibody indicative for the presence and/or amount of anti-zika virus antibodies in a sample from a subject comprising the steps of:
Step 1: contacting an amount of a microsphere complex according to claim 1 with the sample to allow binding of anti-zika virus antibodies in the sample to the zika virus like particles coupled to the microspheres in the microsphere complex,
Step 2: contacting an amount of a detection antibody with the microsphere complex and the sample of step 1 to allow binding of the detection antibody to the heavy chain constant region of the anti-zika virus antibodies bound to the zika virus like particles coupled to the microspheres in the microsphere complex, wherein the detection antibody binds to the anti-zika virus antibodies with the variable region of the detection antibody and wherein the detection antibody is attached to at least one detectable label, and
Step 3: detecting a signal from the detection antibody bound to the anti-zika virus antibodies in step 2.

34. The method according to claim 33 for determining the presence and/or amount of anti-zika virus antibodies in a sample from a subject, wherein the method comprises the further steps of:
Step 4: determining the presence and/or amount of the detection antibody bound to the anti-zika virus antibodies from the signal of step 3, and
Step 5: determining the presence and/or amount of anti-zika virus antibodies in the sample from the presence and/or amount of the detection antibody determined in step 4.

35. A method for preventing zika disease in a human subject comprising the steps of:
Step 1: obtaining a sample from the human subject,
Step 2: determining the amount of anti-zika virus antibodies in the sample from the human subject according to claim 19,
Step 3: determining whether the human subject has an amount of anti-zika virus antibodies to confer protection by comparing the amount of anti-zika virus antibodies determined in step 2 to the antibody correlate of protection against zika virus infection in human subjects, and
Step 4: administering to the human subject a zika virus vaccine if the human subject has an amount of anti-zika antibodies that is lower than the antibody correlate of protection against zika virus infection in human subjects.


SEQUENCE LISTING (186 sequences)

SEQ ID NO: 1 - DNA, 10374 bp, Zika virus
acaggttttattttggatttggaaacgagagtttctggtcatgaaaaacccaaaaaagaaatccggaggattccggattgtcaatatgctaaaacgcggagtagcccgtgtgagcccctttgggggcttgaagaggctgccagccggacttctgctgggtcatgggcccatcaggatggtcttggcgattctagcctttttgagattcacggcaatcaagccatcactgggtctcatcaatagatggggttcagtggggaaaaaagaggctatggaaataataaagaagttcaagaaagatctggctgccatgctgagaataatcaatgctaggaaggagaagaagagacgaggcgcagatactagtgtcggaattgttggcctcctgctgaccacagctatggcagcggaggtcactagacgtgggagtgcatactatatgtacttggacagaaacgatgctggggaggccatatcttttccaaccacattggggatgaataagtgttatatacagatcatggatcttggacacacgtgtgatgccaccatgagctatgaatgccctatgctggatgagggggtggaaccagatgacgtcgattgttggtgcaacacgacgtcaacttgggttgtgtacggaacctgccatcacaaaaaaggtgaagcacggagatctagaagagctgtgacgctcccctcccattccactaggaagctgcaaacgcggtcgcaaacctggttggaatcaagagaatacacaaagcacttgattagagtcgaaaattggatattcaggaaccctggcttcgcgttagcagcagctgccatcgcttggcttttgggaagctcaacgagccaaaaagtcatatacttggtcatgatactgctgattgccccggcatacagcatcaggtgcataggagtcagcaatagggactttgtggaaggtatgtcaggtgggacttgggttgatgttgtcttggaacatggaggttgtgtcactgtaatggcacaggacaaaccgactgtcgacatagagctggttacaacaacagtcagcaacatggcggaggtaagatcctactgctatgaggcatcaatatcagacatggcttcggacagccgctgcccaacacaaggtgaagcctaccttgacaagcaatcagacactcaatatgtctgcaaaagaacgttagtggacagaggctggggaaatggatgtggactttttggcaaagggagcctggtgacatgcgctaagtttgcatgctccaagaaaatgaccgggaagagcatccagccagagaatctggagtaccggataatgctgtcagttcatggctcccagcacagtgggatgatcgttaatgacacaggacatgaaactgatgagaatagagcgaaagttgagataacgcccaattcaccaagagccgaagccaccctgggggggtttggaagcctaggacttgattgtgaaccgaggacaggccttgacttttcagatttgtattacttgactatgaataacaagcactggctggttcacaaggagtggttccacgacattccattaccttggcacgctggggcagacaccggaactccacactggaacaacaaagaagcactggtagagttcaaggacgcacatgccaaaaggcaaactgtcgtggttctagggagtcaagaaggagcagttcacacggcccttgctggagctctggaggctgagatggatggtgcaaagggaaggctgtcctctggccacttgaaatgtcgcctgaaaatggataaacttagattgaagggcgtgtcatactccttgtgtactgcagcgttcacattcaccaagatcccggctgaaacactgcacgggacagtcacagtggaggtacagtacgcagggacagatggaccttgcaaggttccagctcagatggcggtggacatgcaaactctgaccccagttgggaggttgataaccgctaaccccgtaatcactgaaagcactgagaactctaagatgatgctggaacttgatccaccatttggggactcttacattgtcataggagtcggggagaagaagatcacccaccactggcacaggagtggcagcaccattggaaaagcatttgaagccactgtgagaggtgccaagagaatggcagtcttgggagacacagcctgggactttggatcagttggaggcgctctcaactcattgggcaagggcatccatcaaatctttggagcagctttcaaatcattgtttggaggaatgtcctggttctcacaaattctcattggaacgttgctgatgtggttgggtctgaacgcaaagaatggatctatttcccttatgtgcttggccttagggggagtgttgatcttcttatccacagccgtctctgctgatgtggggtgctcggtggacttctcaaagaaggagacgagatgcggtacaggggtgttcgtctataacgacgttgaagcctggagggacaggtacaagtaccatcctgactccccccgtagattggcagcagcagtcaagcaagcctgggaagatggtatctgcgggatctcctctgtttcaagaatggaaaacatcatgtggagatcagtagaaggggagctcaacgcaatcctggaagagaatggagttcaactgacggtcgttgtgggatctgtaaaaaaccccatgtggagaggtccacagagattgcccgtgcctgtgaacgagctgccccacggctggaaggcttgggggaaatcgtacttcgtcagagcagcaaagacaaataacagctttgtcgtggatggtgacacactgaaggaatgcccactcaaacatagagcatggaacagctttcttgtggaggatcatgggttcggggtatttcacactagtgtctggctcaaggttagagaagattattcattagagtgtgatccagccgttattggaacagctgttaagggaaaggaggctgtacacagtgatctaggctactggattgagagtgagaagaatgacacatggaggctgaagagggcccatctgatcgagatgaaaacatgtgaatggccaaagtcccacacattgtggacagatggaatagaagagagtgatctgatcatacccaagtctttagctgggccactcagccatcacaataccagagagggctacaggacccaaatgaaagggccatggcacagtgaagagcttgaaattcggtttgaggaatgcccaggcactaaggtccacgtggaggaaacatgtggaacgagaggaccatctctgagatcaaccactgcaagcggaagggtgatcgaggaatggtgctgcagggagtgcacaatgcccccactgtcgttccgggctaaagatggctgttggtatggaatggagataaggcccaggaaagaaccagaaagcaacttagtaaggtcaatggtgactgcaggatcaactgatcacatggaccacttctcccttggagtgcttgtgattctgctcatggtgcaggaagggttgaagaagagaatgaccacaaagatcatcataagcacatcaatggcagtgctggtagctatgatcctgggaggattttcaatgagtgacctggctaagcttgcaattttgatgggtgccaccttcgcggaaatgaacactggaggagatgtagctcatctggcgctgatagcggcattcaaagtcagaccagcgttgctggtatctttcatcttcagagctaattggacaccccgtgaaagcatgctgctggccttggcctcgtgtcttttgcaaactgcgatctccgccttggaaggcgacctgatggttctcatcaatggttttgctttggcctggttggcaatacgagcgatggttgttccacgcactgataacatcaccttggcaatcctggctgctctgacaccactggcccggggcacactgcttgtggcgtggagagcaggccttgctacttgcggggggtttatgctcctctctctgaagggaaaaggcagtgtgaagaagaacttaccatttgtcatggccctgggactaaccgctgtgaggctggtcgaccccatcaacgtggtgggactgctgttgctcacaaggagtgggaagcggagctggccccctagcgaagtactcacagctgttggcctgatatgcgcattggctggagggttcgccaaggcagatatagagatggctgggcccatggccgcggtcggtctgctaattgtcagttacgtggtctcaggaaagagtgtggacatgtacattgaaagagcaggtgacatcacatgggaaaaagatgcggaagtcactggaaacagtccccggctcgatgtggcgctagatgagagtggtgatttctccctggtggaggatgacggtccccccatgagagagatcatactcaaggtggtcctgatgaccatctgtggcatgaacccaatagccataccctttgcagctggagcgtggtacgtatacgtgaagactggaaaaaggagtggtgctctatgggatgtgcctgctcccaaggaagtaaaaaagggggagaccacagatggagtgtacagagtaatgactcgtagactgctaggttcaacacaagttggagtgggagttatgcaagagggggtctttcacactatgtggcacgtcacaaaaggatccgcgctgagaagcggtgaagggagacttgatccatactggggagatgtcaagcaggatctggtgtcatactgtggtccatggaagctagatgccgcctgggacgggcacagcgaggtgcagctcttggccgtgccccccggagagagagcgaggaacatccagactctgcccggaatatttaagacaaaggatggggacattggagcggttgcgctggattacccagcaggaacttcaggatctcctatcctagacaagtgtgggagagtgataggactttatggcaatggggtcgtgatcaaaaatgggagttatgttagtgccatcacccaagggaggagggaggaagagactcctgttgagtgcttcgagccttcgatgctgaagaagaagcagctaactgtcttagacttgcatcctggagctgggaaaaccaggagagttcttcctgaaatagtccgtgaagccataaaaacaagactccgtactgtgatcttagctccaaccagggttgtcgctgctgaaatggaggaggcccttagagggcttccagtgcgttatatgacaacagcagtcaatgtcacccactctggaacagaaatcgtcgacttaatgtgccatgccaccttcacttcgcgtctactacagccaatcagagtccccaactataatctgtatattatggatgaggcccacttcacagatccctcaagtatagcagcaagaggatacatttcaacaagggttgagatgggcgaggcggccgccatcttcatgaccgccacgccaccaggaacccgtgacgcatttccggactccaactcaccaattatggacaccgaagtggaagtcccagagagagcctggagctcaggctttgattgggtgacggatcattctggaaaaacagtttggtttgttccaagcgtgaggaacggcaatgagatcgcagcttgtctgacaaaggctggaaaacgggtcatacagctcagcagaaagacttttgagacagagttccagaaaacaaaacatcaagagtgggactttgtcgtgacaactgacatttcagagatgggcgccaactttaaagctgaccgtgtcatagattccaggagatgcctaaagccggtcatacttgatggcgagagagtcattctggctggacccatgcctgtcacacatgccagcgctgcccagaggagggggcgcataggcaggaatcccaacaaacctggagatgagtatctgtatggaggtgggtgcgcagagactgacgaagaccatgcacactggcttgaagcaagaatgctccttgacaatatttacctccaagatggcctcatagcctcgctctatcgacctgaggccgacaaagtagcagccattgagggagagttcaagcttaggacggagcaaaggaagacctttgtggaactcatgaaaagaggagatcttcctgtttggctggcctatcaggttgcatctgccggaataacctacacagatagaagatggtgctttgatggcacgaccaacaacaccataatggaagacagtgtgccggcagaagtgtggaccagacacggagagaaaagagtgctcaaaccgaggtggatggacgccagagtttgttcagatcatgcggccctgaagtcattcaaggagtttgccgctgggaaaagaggagcggcttttggagtgatggaagccctgggaacactgccaggacacatgacagagagattccaggaagccattgacaacctcgctgtgctcatgcgggcagagactggaagcaggccttacaaagccgcggcggcccaattgccggagaccctagagaccattatgcttttggggttgctgggaacagtctcgctgggaatcttcttcgtcttgatgaggaacaagggcatagggaagatgggctttggaatggtgactcttggggccagcgcatggctcatgtggctctcggaaattgagccagccagaattgcatgtgtcctcattgttgtgttcctattgctggtggtgctcatacctgagccagaaaagcaaagatctccccaggacaaccaaatggcaatcatcatcatggtagcagtaggtcttctgggcttgattaccgccaatgaactcggatggttggagagaacaaagagtgacctaagccatctaatgggaaggagagaggagggggcaaccataggattctcaatggacattgacctgcggccagcctcagcttgggccatctatgctgccttgacaactttcattaccccagccgtccaacatgcagtgaccacctcatacaacaactactccttaatggcgatggccacgcaagctggagtgttgtttggtatgggcaaagggatgccattctacgcatgggactttggagtcccgctgctaatgataggttgctactcacaattaacacccctgaccctaatagtggccatcattttgctcgtggcgcactacatgtacttgatcccagggctgcaggcagcagctgcgcgtgctgcccagaagagaacggcagctggcatcatgaagaaccctgttgtggatggaatagtggtgactgacattgacacaatgacaattgacccccaagtggagaaaaagatgggacaggtgctactcatagcagtagccgtctccagcgccatactgtcgcggaccgcctgggggtggggggaggctggggccctgatcacagccgcaacttccactttgtgggaaggctctccgaacaagtactggaactcctctacagccacttcactgtgtaacatttttaggggaagttacttggctggagcttctctaatctacacagtaacaagaaacgctggcttggtcaagagacgtgggggtggaacaggagagaccctgggagagaaatggaaggcccgcttgaaccagatgtcggccctggagttctactcctacaaaaagtcaggcatcaccgaggtgtgcagagaagaggcccgccgcgccctcaaggacggtgtggcaacgggaggccatgctgtgtcccgaggaagtgcaaagctgagatggttggtggagcggggatacctgcagccctatggaaaggtcattgatcttggatgtggcagagggggctggagttactacgccgccaccatccgcaaagttcaagaagtgaaaggatacacaaaaggaggccctggtcatgaagaacccgtgttggtgcaaagctatgggtggaacatagtccgtcttaagagtggggtggacgtctttcatatggcggctgagccgtgtgacacgttgctgtgtgacataggtgagtcatcatctagtcctgaagtggaagaagcacggacgctcagagtcctctccatggtgggggattggcttgaaaaaagaccaggagccttttgtataaaagtgttgtgcccatacaccagcactatgatggaaaccctggagcgactgcagcgtaggtatgggggaggactggtcagagtgccactctcccgcaactctacacatgagatgtactgggtctctggagcgaaaagcaacaccataaaaagtgtgtccaccacgagccagctcctcttggggcgcatggacgggcctaggaggccagtgaaatatgaggaggatgtgaatctcggctctggcacgcgggctgtggtaagctgcgctgaagctcccaacatgaagatcattggtaaccgcattgaaaggatccgcagtgagcacgcggaaacgtggttctttgacgagaaccacccatataggacatgggcttaccatggaagctatgaggcccccacacaagggtcagcgtcctctctaataaacggggttgtcaggctcctgtcaaaaccctgggatgtggtgactggagtcacaggaatagccatgaccgacaccacaccgtatggtcagcaaagagttttcaaggaaaaagtggacactagggtgccagacccccaagaaggcactcgtcaggttatgagcatggtctcttcctggttgtggaaagagctaggcaaacacaaacggccacgagtctgtaccaaagaagagttcatcaacaaggttcgtagcaatgcagcattaggggcaatatttgaagaggaaaaagagtggaagactgcagtggaagctgtgaacgatccaaggttctgggctctagtggacaaggaaagagagcaccacctgagaggagagtgccagagttgtgtgtacaacatgatgggaaaaagagaaaagaaacaaggggaatttggaaaggccaagggcagccgcgccatctggtatatgtggctaggggctagatttctagagttcgaagcccttggattcttgaacgaggatcactggatggggagagagaactcaggaggtggtgttgaagggctgggattacaaagactcggatatgtcctagaagagatgagtcgtataccaggaggaaggatgtatgcagatgacactgctggctgggacacccgcattagcaggtttgatctggagaatgaagctctaatcaccaaccaaatggagaaagggcacagggccttggcattggccataatcaagtacacataccaaaacaaagtggtaaaggtccttagaccagctgaaaaagggaaaacagttatggacattatttcgagacaagaccaaagggggagcggacaagttgtcacttacgctcttaacacatttaccaacctagtggtgcaactcattcggaatatggaggctgaggaagttctagagatgcaagacttgtggctgctgcggaggtcagagaaagtgactaactggttgcagagcaacggatgggataggctcaaacgaatggcagtcagtggagatgattgcgttgtgaagccaattgatgataggtttgcacatgccctcaggttcttgaatgatatgggaaaagttaggaaggacacacaagagtggaaaccctcaactggatgggacaactgggaagaagttccgttttgctcccaccacttcaacaagctccatctcaaggacgggaggtccattgtggttccctgccgccaccaagatgaactgattggccgggcccgcgtctctccaggggcgggatggagcatccgggagactgcttgcctagcaaaatcatatgcgcaaatgtggcagctcctttatttccacagaagggacctccgactgatggccaatgccatttgttcatctgtgccagttgactgggttccaactgggagaactacctggtcaatccatggaaagggagaatggatgaccactgaagacatgcttgtggtgtggaacagagtgtggattgaggagaacgaccacatggaagacaagaccccagttacgaaatggacagacattccctatttgggaaaaagggaagacttgtggtgtggatctctcatagggcacagaccgcgcaccacctgggctgagaacattaaaaacacagtcaacatggtgcgcaggatcataggtgatgaagaaaagtacatggactacctatccacccaagttcgctacttgggtgaagaagggtctacacctggagtgctgtaagcaccaatcttaatgttgtcaggcctgctagtcagccacagcttggggaaagctgtgcagcc

SEQ ID NO: 2 - protein, 3423 aa, Zika virus
MKNPKKKSGGFRIVNMLKRGVARVSPFGGLKRLPAGLLLGHGPIRMVLAILAFLRFTAIKPSLGLINRWGSVGKKEAMEIIKKFKKDLAAMLRIINARKEKKRRGADTSVGIVGLLLTTAMAAEVTRRGSAYYMYLDRNDAGEAISFPTTLGMNKCYIQIMDLGHTCDATMSYECPMLDEGVEPDDVDCWCNTTSTWVVYGTCHHKKGEARRSRRAVTLPSHSTRKLQTRSQTWLESREYTKHLIRVENWIFRNPGFALAAAAIAWLLGSSTSQKVIYLVMILLIAPAYSIRCIGVSNRDFVEGMSGGTWVDVVLEHGGCVTVMAQDKPTVDIELVTTTVSNMAEVRSYCYEASISDMASDSRCPTQGEAYLDKQSDTQYVCKRTLVDRGWGNGCGLFGKGSLVTCAKFACSKKMTGKSIQPENLEYRIMLSVHGSQHSGMIVNDTGHETDENRAKVEITPNSPRAEATLGGFGSLGLDCEPRTGLDFSDLYYLTMNNKHWLVHKEWFHDIPLPWHAGADTGTPHWNNKEALVEFKDAHAKRQTVVVLGSQEGAVHTALAGALEAEMDGAKGRLSSGHLKCRLKMDKLRLKGVSYSLCTAAFTFTKIPAETLHGTVTVEVQYAGTDGPCKVPAQMAVDMQTLTPVGRLITANPVITESTENSKMMLELDPPFGDSYIVIGVGEKKITHHWHRSGSTIGKAFEATVRGAKRMAVLGDTAWDFGSVGGALNSLGKGIHQIFGAAFKSLFGGMSWFSQILIGTLLMWLGLNAKNGSISLMCLALGGVLIFLSTAVSADVGCSVDFSKKETRCGTGVFVYNDVEAWRDRYKYHPDSPRRLAAAVKQAWEDGICGISSVSRMENIMWRSVEGELNAILEENGVQLTVVVGSVKNPMWRGPQRLPVPVNELPHGWKAWGKSYFVRAAKTNNSFVVDGDTLKECPLKHRAWNSFLVEDHGFGVFHTSVWLKVREDYSLECDPAVIGTAVKGKEAVHSDLGYWIESEKNDTWRLKRAHLIEMKTCEWPKSHTLWTDGIEESDLIIPKSLAGPLSHHNTREGYRTQMKGPWHSEELEIRFEECPGTKVHVEETCGTRGPSLRSTTASGRVIEEWCCRECTMPPLSFRAKDGCWYGMEIRPRKEPESNLVRSMVTAGSTDHMDHFSLGVLVILLMVQEGLKKRMTTKIIISTSMAVLVAMILGGFSMSDLAKLAILMGATFAEMNTGGDVAHLALIAAFKVRPALLVSFIFRANWTPRESMLLALASCLLQTAISALEGDLMVLINGFALAWLAIRAMVVPRTDNITLAILAALTPLARGTLLVAWRAGLATCGGFMLLSLKGKGSVKKNLPFVMALGLTAVRLVDPINVVGLLLLTRSGKRSWPPSEVLTAVGLICALAGGFAKADIEMAGPMAAVGLLIVSYVVSGKSVDMYIERAGDITWEKDAEVTGNSPRLDVALDESGDFSLVEDDGPPMREIILKVVLMTICGMNPIAIPFAAGAWYVYVKTGKRSGALWDVPAPKEVKKGETTDGVYRVMTRRLLGSTQVGVGVMQEGVFHTMWHVTKGSALRSGEGRLDPYWGDVKQDLVSYCGPWKLDAAWDGHSEVQLLAVPPGERARNIQTLPGIFKTKDGDIGAVALDYPAGTSGSPILDKCGRVIGLYGNGVVIKNGSYVSAITQGRREEETPVECFEPSMLKKKQLTVLDLHPGAGKTRRVLPEIVREAIKTRLRTVILAPTRVVAAEMEEALRGLPVRYMTTAVNVTHSGTEIVDLMCHATFTSRLLQPIRVPNYNLYIMDEAHFTDPSSIAARGYISTRVEMGEAAAIFMTATPPGTRDAFPDSNSPIMDTEVEVPERAWSSGFDWVTDHSGKTVWFVPSVRNGNEIAACLTKAGKRVIQLSRKTFETEFQKTKHQEWDFVVTTDISEMGANFKADRVIDSRRCLKPVILDGERVILAGPMPVTHASAAQRRGRIGRNPNKPGDEYLYGGGCAETDEDHAHWLEARMLLDNIYLQDGLIASLYRPEADKVAAIEGEFKLRTEQRKTFVELMKRGDLPVWLAYQVASAGITYTDRRWCFDGTTNNTIMEDSVPAEVWTRHGEKRVLKPRWMDARVCSDHAALKSFKEFAAGKRGAAFGVMEALGTLPGHMTERFQEAIDNLAVLMRAETGSRPYKAAAAQLPETLETIMLLGLLGTVSLGIFFVLMRNKGIGKMGFGMVTLGASAWLMWLSEIEPARIACVLIVVFLLLVVLIPEPEKQRSPQDNQMAIIIMVAVGLLGLITANELGWLERTKSDLSHLMGRREEGATIGFSMDIDLRPASAWAIYAALTTFITPAVQHAVTTSYNNYSLMAMATQAGVLFGMGKGMPFYAWDFGVPLLMIGCYSQLTPLTLIVAIILLVAHYMYLIPGLQAAAARAAQKRTAAGIMKNPVVDGIVVTDIDTMTIDPQVEKKMGQVLLIAVAVSSAILSRTAWGWGEAGALITAATSTLWEGSPNKYWNSSTATSLCNIFRGSYLAGASLIYTVTRNAGLVKRRGGGTGETLGEKWKARLNQMSALEFYSYKKSGITEVCREEARRALKDGVATGGHAVSRGSAKLRWLVERGYLQPYGKVIDLGCGRGGWSYYAATIRKVQEVKGYTKGGPGHEEPVLVQSYGWNIVRLKSGVDVFHMAAEPCDTLLCDIGESSSSPEVEEARTLRVLSMVGDWLEKRPGAFCIKVLCPYTSTMMETLERLQRRYGGGLVRVPLSRNSTHEMYWVSGAKSNTIKSVSTTSQLLLGRMDGPRRPVKYEEDVNLGSGTRAVVSCAEAPNMKIIGNRIERIRSEHAETWFFDENHPYRTWAYHGSYEAPTQGSASSLINGVVRLLSKPWDVVTGVTGIAMTDTTPYGQQRVFKEKVDTRVPDPQEGTRQVMSMVSSWLWKELGKHKRPRVCTKEEFINKVRSNAALGAIFEEEKEWKTAVEAVNDPRFWALVDKEREHHLRGECQSCVYNMMGKREKKQGEFGKAKGSRAIWYMWLGARFLEFEALGFLNEDHWMGRENSGGGVEGLGLQRLGYVLEEMSRIPGGRMYADDTAGWDTRISRFDLENEALITNQMEKGHRALALAIIKYTYQNKVVKVLRPAEKGKTVMDIISRQDQRGSGQVVTYALNTFTNLVVQLIRNMEAEEVLEMQDLWLLRRSEKVTNWLQSNGWDRLKRMAVSGDDCVVKPIDDRFAHALRFLNDMGKVRKDTQEWKPSTGWDNWEEVPFCSHHFNKLHLKDGRSIVVPCRHQDELIGRARVSPGAGWSIRETACLAKSYAQMWQLLYFHRRDLRLMANAICSSVPVDWVPTGRTTWSIHGKGEWMTTEDMLVVWNRVWIEENDHMEDKTPVTKWTDIPYLGKREDLWCGSLIGHRPRTTWAENIKNTVNMVRRIIGDEEKYMDYLSTQVRYLGEEGSTPGVL

SEQ ID NO: 3 - protein, 504 aa, Artificial Sequence
  ZIKV E-Protein
IRCIGVSNRDFVEGMSGGTWVDVVLEHGGCVTVMAQDKPTVDIELVTTTVSNMAEVRSYCYEASISDMASDSRCPTQGEAYLDKQSDTQYVCKRTLVDRGWGNGCGLFGKGSLVTCAKFACSKKMTGKSIQPENLEYRIMLSVHGSQHSGMIVNDTGHETDENRAKVEITPNSPRAEATLGGFGSLGLDCEPRTGLDFSDLYYLTMNNKHWLVHKEWFHDIPLPWHAGADTGTPHWNNKEALVEFKDAHAKRQTVVVLGSQEGAVHTALAGALEAEMDGAKGRLSSGHLKCRLKMDKLRLKGVSYSLCTAAFTFTKIPAETLHGTVTVEVQYAGTDGPCKVPAQMAVDMQTLTPVGRLITANPVITESTENSKMMLELDPPFGDSYIVIGVGEKKITHHWHRSGSTIGKAFEATVRGAKRMAVLGDTAWDFGSVGGALNSLGKGIHQIFGAAFKSLFGGMSWFSQILIGTLLMWLGLNTKNGSISLMCLALGGVLIFLSTAVSA

SEQ ID NO: 4 - DNA, 330 bp, Artificial Sequence
  EDIII domain of Zika virus
agattgaagggcgtgtcatactccctgtgcaccgccgcattcacctttaccaaaattccggcggagactctgcacggtacggtgacggttgaagtccagtatgcgggcaccgacggtccgtgtaaagtgccggctcaaatggcagtcgacatgcagaccctgacgccggtgggtcgtctgattacggcgaatccagtcatcaccgagagcaccgaaaacagcaagatgatgctggaactggatcctccgttcggcgatagctacatcgttattggcgttggtgagaaaaagatcacccatcactggcacaggagtggcagcaccatttaa

SEQ ID NO: 5 - protein, 441 aa, Oryctolagus cuniculus
QSLEESGGDLVKPGASLTLTSKVSGFSFNSNYWICWVRQAPGKGLEWIGCVFGGIHVTYYANWVKGRFTISKPSSTTVTLEMTSLTAADTATYFCIISTGGSHRFNLWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 6 - protein, 118 aa, Artificial sequence
  Anti-ZIKV #1 VH
QSLEESGGDLVKPGASLTLTSKVSGFSFNSNYWICWVRQAPGKGLEWIGCVFGGIHVTYYANWVKGRFTISKPSSTTVTLEMTSLTAADTATYFCIISTGGSHRFNLWGPGTLVTVSS

SEQ ID NO: 7 - protein, 9 aa, Artificial Sequence
  Anti-ZIKV #1 VH-CDR1
GFSFNSNYW

SEQ ID NO: 8 - protein, 7 aa, Artificial sequence
  Anti-ZIKV #1 VH-CDR2
FGGIHVT

SEQ ID NO: 9 - protein, 12 aa, Artificial sequence
  Anti-ZIKV #1 VH-CDR3
IISTGGSHRFNL

SEQ ID NO: 10 - protein, 214 aa, Oryctolagus cuniculus
DVVMTQTPASVEAAVGGTVTIKCQASESIYTYLSWYQQKPGQPPKLLIYRASTLDSGVPSRFSGSGSGTEFTLTISDLDCADAASYYCQATDVGGSGRGAFGGGTEVVVEGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 11 - protein, 110 aa, Artificial Sequence
  Anti-ZIKV #1 VL
DVVMTQTPASVEAAVGGTVTIKCQASESIYTYLSWYQQKPGQPPKLLIYRASTLDSGVPSRFSGSGSGTEFTLTISDLDCADAASYYCQATDVGGSGRGAFGGGTEVVVE

SEQ ID NO: 12 - protein, 6 aa, Artificial Sequence
  Anti-ZIKV #1 VL-CDR1
ESIYTY

SEQ ID NO: 13 - protein, 3 aa, Artificial Sequence
  Anti-ZIKV #1 VL-CDR2
RAS

SEQ ID NO: 14 - protein, 12 aa, Artificial Sequence
  Anti-ZIKV #1 VL-CDR3
QATDVGGSGRGA

SEQ ID NO: 15 - DNA, 1383 bp, Oryctolagus cuniculus
atggagactgggctgcgctggcttctcctggtcgctgtgctcaaaggtgtccaatgtcagtcgttggaggagtccgggggagacctggtcaagcctggggcatccctgacactcacctccaaagtctctggattctccttcaatagcaactattggatatgctgggtccgccaggctccagggaaggggctggagtggatcggatgcgtttttggtggtattcatgtcacatattacgcgaattgggtgaaaggccgattcaccatctccaaaccctcgtcgaccacggtgactctggaaatgaccagtctgacagccgcggacacggccacttatttctgtattattagtactggcggtagtcacagatttaatttgtggggcccaggcaccctggtcaccgtctcctcagggcaacctaaggctccatcagtcttcccactggccccctgctgcggggacacacccagctccacggtgaccctgggctgcctggtcaaagggtacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccacgtgcccaccccctgaactcctggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccggccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaatga

SEQ ID NO: 16 - DNA, 411 bp, Artificial Sequence
  Anti-ZIKV #1 VH DNA
atggagactgggctgcgctggcttctcctggtcgctgtgctcaaaggtgtccaatgtcagtcgttggaggagtccgggggagacctggtcaagcctggggcatccctgacactcacctccaaagtctctggattctccttcaatagcaactattggatatgctgggtccgccaggctccagggaaggggctggagtggatcggatgcgtttttggtggtattcatgtcacatattacgcgaattgggtgaaaggccgattcaccatctccaaaccctcgtcgaccacggtgactctggaaatgaccagtctgacagccgcggacacggccacttatttctgtattattagtactggcggtagtcacagatttaatttgtggggcccaggcaccctggtcaccgtctcctca

SEQ ID NO: 17 - DNA, 711 bp, Oryctolagus cuniculus
atggacacgagggcccccactcagctgctggggctcctgctgctctggctcccaggtgccagatgtgatgttgtgatgacccagactccagcctccgtggaggcagctgtgggaggcacagtcaccatcaagtgccaggccagtgagagcatttatacctacttatcctggtatcagcagaaaccagggcagcctcccaaactcctgatctatagggcatccactctggattctggggtcccatcgcggttcagcggcagtggatctgggacagagttcactctcaccatcagcgacctggactgtgccgatgctgccagttattattgtcaggctactgatgttggtggtagtggtaggggtgctttcggcggagggaccgaggtggtggtcgaaggtgatccagttgcacctactgtcctcatcttcccaccagctgctgatcaggtggcaactggaacagtcaccatcgtgtgtgtggcgaataaatactttcccgatgtcaccgtcacctgggaggtggatggcaccacccaaacaactggcatcgagaacagtaaaacaccgcagaattctgcagattgtacctacaacctcagcagcactctgacactgaccagcacacagtacaacagccacaaagagtacacctgcaaggtgacccagggcacgacctcagtcgtccagagcttcaataggggtgactgttag

SEQ ID NO: 18 - DNA, 396 bp, Artificial Sequence
  Anti-ZIKV #1 VL DNA
atggacacgagggcccccactcagctgctggggctcctgctgctctggctcccaggtgccagatgtgatgttgtgatgacccagactccagcctccgtggaggcagctgtgggaggcacagtcaccatcaagtgccaggccagtgagagcatttatacctacttatcctggtatcagcagaaaccagggcagcctcccaaactcctgatctatagggcatccactctggattctggggtcccatcgcggttcagcggcagtggatctgggacagagttcactctcaccatcagcgacctggactgtgccgatgctgccagttattattgtcaggctactgatgttggtggtagtggtaggggtgctttcggcggagggaccgaggtggtggtcgaa

SEQ ID NO: 19 - protein, 448 aa, Oryctolagus cuniculus
QAQLEESGGDLVKPEGSLTLTCTASGFSFTDRHYMCWIRQAPGKGLEWIACVYPGSSGSTYYASWARGRFTISKTSSTTVTLRMTSLTAADTATYFCARSSYPDSSGYSYGMDLWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 20 - protein, 125 aa, Artificial sequence
  Anti-ZIKV #2 VH
QAQLEESGGDLVKPEGSLTLTCTASGFSFTDRHYMCWIRQAPGKGLEWIACVYPGSSGSTYYASWARGRFTISKTSSTTVTLRMTSLTAADTATYFCARSSYPDSSGYSYGMDLWGPGTLVTVSS

SEQ ID NO: 21 - protein, 9 aa, Artificial sequence
  Anti-ZIKV #2 VH-CDR1
GFSFTDRHY

SEQ ID NO: 22 - protein, 8 aa, Artificial Sequence
  Anti-ZIKV #2 VH-CDR2
YPGSSGST

SEQ ID NO: 23 - protein, 17 aa, Artificial Sequence
  Anti-ZIKV #2 VH-CDR3
ARSSYPDSSGYSYGMDL

SEQ ID NO: 24 - protein, 214 aa, Oryctolagus cuniculus
DIVMTQTPASVEAAVGGTVTIKCQASQNINSNLAWYQQKPGQRPKFLMYLTSKLASGVPSRFRGSGSGTQFTLTISDLECADAATYYCQTYYDISNYGYAFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 25 - protein, 110 aa, Artificial Sequence
  Anti-ZIKV #2 VL
DIVMTQTPASVEAAVGGTVTIKCQASQNINSNLAWYQQKPGQRPKFLMYLTSKLASGVPSRFRGSGSGTQFTLTISDLECADAATYYCQTYYDISNYGYAFGGGTEVVVK

SEQ ID NO: 26 - protein, 6 aa, Artificial sequence
  Anti-ZIKV #2 VL-CDR1
QNINSN

SEQ ID NO: 27 - protein, 3 aa, Artificial Sequence
  Anti-ZIKV #2 VL-CDR2
LTS

SEQ ID NO: 28 - protein, 12 aa, Artificial sequence
  Anti-ZIKV #2 VL-CDR3
QTYYDISNYGYA

SEQ ID NO: 29 - DNA, 1404 bp, Oryctolagus cuniculus
atggagactgggctgcgctggcttctcctggtcgctgtgctcaaaggtgtccagtgtcaggcgcagctggaggagtccgggggagacctggtcaagcctgagggatccctgacactcacctgcacagcctctggattctccttcactgacaggcattacatgtgctggatccgccaggctccggggaaggggctggagtggatcgcatgcgtttatcctggtagtagtggtagcacttactacgcgagctgggcgagaggccgattcaccatctccaaaacctcgtcgaccacggtgactctgcgcatgaccagtctgacagccgcggacacggccacctatttctgtgcgagatcctcatatcctgatagtagtggctatagttatggcatggacctctggggccctgggaccctcgtcaccgtctcttcagggcaacctaaggctccatcagtcttcccactggccccctgctgcggggacacacccagctccacggtgaccctgggctgcctggtcaaagggtacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccacgtgcccaccccctgaactcctggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccggccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaatga

SEQ ID NO: 30 - DNA, 432 bp, Artificial Sequence
  Anti-ZIKV #2 VH DNA
atggagactgggctgcgctggcttctcctggtcgctgtgctcaaaggtgtccagtgtcaggcgcagctggaggagtccgggggagacctggtcaagcctgagggatccctgacactcacctgcacagcctctggattctccttcactgacaggcattacatgtgctggatccgccaggctccggggaaggggctggagtggatcgcatgcgtttatcctggtagtagtggtagcacttactacgcgagctgggcgagaggccgattcaccatctccaaaacctcgtcgaccacggtgactctgcgcatgaccagtctgacagccgcggacacggccacctatttctgtgcgagatcctcatatcctgatagtagtggctatagttatggcatggacctctggggccctgggaccctcgtcaccgtctcttca

SEQ ID NO: 31 - DNA, 714 bp, Oryctolagus cuniculus
atggacacgagggcccccactcagctgctggggctcctgttgctctggctcccaggtgccagatgtgctgacattgtgatgacccagactccagcctccgtggaggcagctgtgggaggcacagtcaccatcaagtgccaggccagtcagaacattaacagcaatttagcctggtatcagcagaaaccagggcagcgtcccaagttcctgatgtatctgacatccaaactggcatctggggtcccatcgcggttcagaggcagtggatctgggacacagttcactctcaccatcagcgacctggagtgtgccgatgctgccacttactactgtcaaacctattatgatattagtaattatggatatgctttcggcggagggaccgaggtggtggtcaaaggtgatccagttgcacctactgtcctcatcttcccaccagctgctgatcaggtggcaactggaacagtcaccatcgtgtgtgtggcgaataaatactttcccgatgtcaccgtcacctgggaggtggatggcaccacccaaacaactggcatcgagaacagtaaaacaccgcagaattctgcagattgtacctacaacctcagcagcactctgacactgaccagcacacagtacaacagccacaaagagtacacctgcaaggtgacccagggcacgacctcagtcgtccagagcttcaataggggtgactgttag

SEQ ID NO: 32 - DNA, 399 bp, Artificial sequence
  Anti-ZIKV #2 VL DNA
atggacacgagggcccccactcagctgctggggctcctgttgctctggctcccaggtgccagatgtgctgacattgtgatgacccagactccagcctccgtggaggcagctgtgggaggcacagtcaccatcaagtgccaggccagtcagaacattaacagcaatttagcctggtatcagcagaaaccagggcagcgtcccaagttcctgatgtatctgacatccaaactggcatctggggtcccatcgcggttcagaggcagtggatctgggacacagttcactctcaccatcagcgacctggagtgtgccgatgctgccacttactactgtcaaacctattatgatattagtaattatggatatgctttcggcggagggaccgaggtggtggtcaaa

SEQ ID NO: 33 - protein, 448 aa, Oryctolagus cuniculus
QAQLEESGGDLVKPEGSLTLTCTASGFSFTDRHYMCWVRQAPGKGLEWIACVYPGSSGSTYYASWARGRFTISKTSSTTVTLRMTSLTAADTATYFCARSSYPDSSGYSYGMDLWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 34 - protein, 125 aa, Artificial Sequence
  Anti-ZIKV #3 VH
QAQLEESGGDLVKPEGSLTLTCTASGFSFTDRHYMCWVRQAPGKGLEWIACVYPGSSGSTYYASWARGRFTISKTSSTTVTLRMTSLTAADTATYFCARSSYPDSSGYSYGMDLWGPGTLVTVSS

SEQ ID NO: 35 - protein, 9 aa, Artificial sequence
  Anti-ZIKV #3 VH-CDR1
GFSFTDRHY

SEQ ID NO: 36 - protein, 8 aa, Artificial Sequence
  Anti-ZIKV #3 VH-CDR2
YPGSSGST

SEQ ID NO: 37 - protein, 17 aa, Artificial Sequence
  Anti-ZIKV #3 VH-CDR3
ARSSYPDSSGYSYGMDL

SEQ ID NO: 38 - protein, 214 aa, Oryctolagus cuniculus
DIVMTQTPASVEAAVGGTVTIKCQASQDINSNLAWYQQKPGQRPKFLMYLTSKLASGVPSRFRGSGSGTQFTLTISDLECADAATYYCQTYYDISNYGYAFGGGTEVVVKGDPVAPTVLIFPPAADQVATGTVTIVCVANKYFPDVTVTWEVDGTTRTTGIENSKTPQNSADCTYNLSSTLTLTSTQYNSHKEYTCKVTQGTTSVVQSFNRGDC

SEQ ID NO: 39 - protein, 110 aa, Artificial Sequence
  Anti-ZIKV #3 VL
DIVMTQTPASVEAAVGGTVTIKCQASQDINSNLAWYQQKPGQRPKFLMYLTSKLASGVPSRFRGSGSGTQFTLTISDLECADAATYYCQTYYDISNYGYAFGGGTEVVVK

SEQ ID NO: 40 - protein, 6 aa, Artificial Sequence
  Anti-ZIKV #3 VL-CDR1
QDINSN

SEQ ID NO: 41 - protein, 3 aa, Artificial Sequence
  Anti-ZIKV #3 VL-CDR2
LTS

SEQ ID NO: 42 - protein, 12 aa, Artificial Sequence
  Anti-ZIKV #3 VL-CDR3
QTYYDISNYGYA

SEQ ID NO: 43 - DNA, 1404 bp, Oryctolagus cuniculus
atggagactgggctgcgctggcttctcctggtcgctgtgctcaaaggtgtccagtgtcaggcgcagctggaggagtccgggggagacctggtcaagcctgagggatccctgacactcacctgcacagcctctggattctccttcactgacaggcattacatgtgctgggtccgccaggctccggggaaggggctggagtggatcgcatgcgtttatcctggtagtagtggtagcacttactacgcgagctgggcgagaggccgattcaccatctccaaaacctcgtcgaccacggtgactctgcgcatgaccagtctgacagccgcggacacggccacctatttctgtgcgagatcctcatatcctgatagtagtggctatagttatggcatggacctctggggccctgggaccctcgtcaccgtctcttcagggcaacctaaggctccatcagtcttcccactggccccctgctgcggggacacacccagctccacggtgaccctgggctgcctggtcaaagggtacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccacgtgcccaccccctgaactcctggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccggccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaatga

SEQ ID NO: 44 - DNA, 432 bp, Artificial Sequence
  Anti-ZIKV #3 VH DNA
atggagactgggctgcgctggcttctcctggtcgctgtgctcaaaggtgtccagtgtcaggcgcagctggaggagtccgggggagacctggtcaagcctgagggatccctgacactcacctgcacagcctctggattctccttcactgacaggcattacatgtgctgggtccgccaggctccggggaaggggctggagtggatcgcatgcgtttatcctggtagtagtggtagcacttactacgcgagctgggcgagaggccgattcaccatctccaaaacctcgtcgaccacggtgactctgcgcatgaccagtctgacagccgcggacacggccacctatttctgtgcgagatcctcatatcctgatagtagtggctatagttatggcatggacctctggggccctgggaccctcgtcaccgtctcttca

SEQ ID NO: 45 - DNA, 714 bp, Oryctolagus cuniculus
atggacacgagggcccccactcagctgctggggctcctgttgctctggctcccaggtgccagatgtgctgacattgtgatgacccagactccagcctccgtggaggcagctgtgggaggcacagtcaccatcaagtgccaggccagtcaggacattaacagcaatttagcctggtatcagcagaaaccagggcagcgtcccaagttcctgatgtatctgacatccaaactggcatctggggtcccatcgcggttcagaggcagtggatctgggacacagttcactctcaccatcagcgacctggagtgtgccgatgctgccacttactactgtcaaacctattatgatattagtaattatggatatgctttcggcggagggaccgaggtggtggtcaaaggtgatccagttgcacctactgtcctcatcttcccaccagctgctgatcaggtggcaactggaacagtcaccatcgtgtgtgtggcgaataaatactttcccgatgtcaccgtcacctgggaggtggatggcaccacccgaacaactggcatcgagaacagtaaaacaccgcagaattctgcagattgtacctacaacctcagcagcactctgacactgaccagcacacagtacaacagccacaaagagtacacctgcaaggtgacccagggcacgacctcagtcgtccagagcttcaataggggtgactgttag

SEQ ID NO: 46 - DNA, 398 bp, Artificial Sequence
  Anti-ZIKV #3 VL DNA
atggacacgagggcccccactcagctgctggggctcctgttgctctggctcccaggtgccagatgtgctgacattgtgatgacccagactccagcctccgtggaggcagctgtgggaggcacagtcaccatcaagtgccaggccagtcaggacattaacagcaatttagcctggtatcagcagaaaccagggcagcgtcccaagttcctgatgtatctgacatccaaactggcatctggggtcccatcgcggttcagaggcagtggatctgggacacagttcactctcaccatcagcgacctggagtgtgccgatgctgccacttactactgtcaaacctattatgatattagtaattatggatatgctttcggcggagggaccgaggtggtggtcaa

SEQ ID NO: 47 - protein, 448 aa, Oryctolagus cuniculus
QEELEESGGDLVKPGASLTLTCKASGFSFSSGAYICWVRQAPGKGPEWIACIYTGDGRTYYANWAKRRFTISSSSSTTVALEMPSLTDADTATYFCARAIAVGAGYGVGNYFTLWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 48 - protein, 125 aa, Artificial sequence
  Anti-ZIKV #4 VH
QEELEESGGDLVKPGASLTLTCKASGFSFSSGAYICWVRQAPGKGPEWIACIYTGDGRTYYANWAKRRFTISSSSSTTVALEMPSLTDADTATYFCARAIAVGAGYGVGNYFTLWGPGTLVTVSS

SEQ ID NO: 49 - protein, 9 aa, Artificial Sequence
  Anti-ZIKV #4 VH-CDR1
GFSFSSGAY

SEQ ID NO: 50 - protein, 7 aa, Artificial Sequence
  Anti-ZIKV #4 VH-CDR2
YTGDGRT

SEQ ID NO: 51 - protein, 18 aa, Artificial Sequence
  Anti-ZIKV #4 VH-CDR3
ARAIAVGAGYGVGNYFTL

SEQ ID NO: 52 - protein, 218 aa, Oryctolagus cuniculus
DIVMTQTPSSVSAAVGGTVTINCHASENIYGYLSWYQQKPGQPPKLLIYKASTLASGVPSRFKGSGAGTQFTLTITALECADAATYYCQSYYTSSSNADGSENAFGGGTEVVVEGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 53 - protein, 114 aa, Artificial Sequence
  Anti-ZIKV #4 VL
DIVMTQTPSSVSAAVGGTVTINCHASENIYGYLSWYQQKPGQPPKLLIYKASTLASGVPSRFKGSGAGTQFTLTITALECADAATYYCQSYYTSSSNADGSENAFGGGTEVVVE

SEQ ID NO: 54 - protein, 6 aa, Artificial Sequence
  Anti-ZIKV #4 VL-CDR1
ENIYGY

SEQ ID NO: 55 - protein, 3 aa, Artificial Sequence
  Anti-ZIKV #4 VL-CDR2
KAS

SEQ ID NO: 56 - protein, 16 aa, Artificial Sequence
  Anti-ZIKV #4 VL-CDR3
QSYYTSSSNADGSENA

SEQ ID NO: 57 - DNA, 1404 bp, Oryctolagus cuniculus
atggagactgggctgcgctggcttctcctggtcgctgtgctcaaaggtgtccagtgtcaggaggagctggaggagtccgggggagacctggtcaagcctggggcatccctgacactcacctgcaaagcctctggattctccttcagtagtggcgcctacatatgctgggtccgccaggctccagggaaggggcctgagtggatcgcgtgtatttatactggtgatggccgcacatactacgcgaactgggcgaagcgccgcttcaccatctccagttcctcgtcgaccacggtggctctggaaatgcccagtctgacagacgcggacacggcgacctatttctgtgcgcgagcaattgctgttggcgctggttatggtgttggaaactactttaccttgtggggcccaggcaccctggtcaccgtctcctcagggcaacctaaggctccatcagtcttcccactggccccctgctgcggggacacacccagctccacggtgaccctgggctgcctggtcaaagggtacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccacgtgcccaccccctgaactcctggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccggccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaatga

SEQ ID NO: 58 - DNA, 432 bp, Artificial Sequence
  Anti-ZIKV #4 VH DNA
atggagactgggctgcgctggcttctcctggtcgctgtgctcaaaggtgtccagtgtcaggaggagctggaggagtccgggggagacctggtcaagcctggggcatccctgacactcacctgcaaagcctctggattctccttcagtagtggcgcctacatatgctgggtccgccaggctccagggaaggggcctgagtggatcgcgtgtatttatactggtgatggccgcacatactacgcgaactgggcgaagcgccgcttcaccatctccagttcctcgtcgaccacggtggctctggaaatgcccagtctgacagacgcggacacggcgacctatttctgtgcgcgagcaattgctgttggcgctggttatggtgttggaaactactttaccttgtggggcccaggcaccctggtcaccgtctcctca

SEQ ID NO: 59 - DNA, 723 bp, Oryctolagus cuniculus
atggacacgagggcccccactcagctgctggggctcctgctgctctggctcccaggtgccatatgtgacattgtgatgacccagactccatcttccgtgtctgcagctgtgggaggcacagtcaccatcaattgccacgccagtgagaacatttacggctacttatcctggtatcagcagaaaccagggcagcctcccaagctcctgatctacaaggcttccactctggcatctggggtcccatcgcggttcaaaggcagtggagctgggacacagttcactctcaccatcaccgccctggagtgtgccgatgctgccacttactactgtcaaagttattatactagtagtagtaatgctgatggttcggagaatgcttttggcggagggaccgaggtggtggtcgaaggtgatccagttgcacctactgtcctcatcttcccaccagctgctgatcaggtggcaactggaacagtcaccatcgtgtgtgtggcgaataaatactttcccgatgtcaccgtcacctgggaggtggatggcaccacccaaacaactggcatcgagaacagtaaaacaccgcagaattctgcagattgtacctacaacctcagcagcactctgacactgaccagcacacagtacaacagccacaaagagtacacctgcaaggtgacccagggcacgacctcagtcgtccagagcttcaataggggtgactgttag

SEQ ID NO: 60 - DNA, 408 bp, Artificial Sequence
  Anti-ZIKV #4 VL DNA
atggacacgagggcccccactcagctgctggggctcctgctgctctggctcccaggtgccatatgtgacattgtgatgacccagactccatcttccgtgtctgcagctgtgggaggcacagtcaccatcaattgccacgccagtgagaacatttacggctacttatcctggtatcagcagaaaccagggcagcctcccaagctcctgatctacaaggcttccactctggcatctggggtcccatcgcggttcaaaggcagtggagctgggacacagttcactctcaccatcaccgccctggagtgtgccgatgctgccacttactactgtcaaagttattatactagtagtagtaatgctgatggttcggagaatgcttttggcggagggaccgaggtggtggtcgaa

SEQ ID NO: 61 - protein, 439 aa, Oryctolagus cuniculus
QSLEESGGDLVKPGASLTLTCTASGFDFSDRYYMCWVRQAPGKGLEWIGCIYVGSGDTYYASWAKGRFTISRTSSTTVTLQMTRLTAADTATYFCARHPGTYFTLWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 62 - protein, 116 aa, Artificial Sequence
  Anti-ZIKV #5 VH
QSLEESGGDLVKPGASLTLTCTASGFDFSDRYYMCWVRQAPGKGLEWIGCIYVGSGDTYYASWAKGRFTISRTSSTTVTLQMTRLTAADTATYFCARHPGTYFTLWGPGTLVTVSS

SEQ ID NO: 63 - protein, 9 aa, Artificial Sequence
  Anti-ZIKV #5 VH-CDR1
GFDFSDRYY

SEQ ID NO: 64 - protein, 7 aa, Artificial Sequence
  Anti-ZIKV #5 VH-CDR2
YVGSGDT

SEQ ID NO: 65 - protein, 8 aa, Artificial Sequence
  Anti-ZIKV #5 VH-CDR3
ARHPGTYF

SEQ ID NO: 66 - protein, 214 aa, Oryctolagus cuniculus
DVVMTQTPSSVSAAVGGTVTINCQASQNIVNNLAWYQQRPGQPPKLLIYDTSTLASGVPSRFKGSGSGTEFTLTISDLECADAATYYCQTYYYYNKIINGFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 67 - protein, 110 aa, Artificial Sequence
  Anti-ZIKV #5 VL
DVVMTQTPSSVSAAVGGTVTINCQASQNIVNNLAWYQQRPGQPPKLLIYDTSTLASGVPSRFKGSGSGTEFTLTISDLECADAATYYCQTYYYYNKIINGFGGGTEVVVK

SEQ ID NO: 68 - protein, 6 aa, Artificial Sequence
  Anti-ZIKV #5 VL-CDR1
QNIVNN

SEQ ID NO: 69 - protein, 3 aa, Artificial Sequence
  Anti-ZIKV #5 VL-CDR2
DTS

SEQ ID NO: 70 - protein, 12 aa, Artificial Sequence
  Anti-ZIKV #5 VL-CDR3
QTYYYYNKIING

SEQ ID NO: 71 - DNA, 1377 bp, Oryctolagus cuniculus
atggagactgggctgcgctggcttctcctggtcgctgtgctcaaaggtgtccagtgtcagtcgttggaggagtccgggggagacctggtcaagcctggggcatccctgacactcacctgcacagcctctggattcgacttcagtgacagatattacatgtgttgggtccgccaggctccagggaaggggctggagtggatcggatgcatttatgttggtagtggtgacacttattatgcgagctgggcgaaaggccgattcaccatctccagaacctcgtcgaccacggtgactctgcaaatgacccgtctgacagccgcggacacggccacctatttctgtgcgagacatcctggtacttactttactttatggggcccaggcaccctggtcaccgtctcctcagggcaacctaaggctccatcagtcttcccactggccccctgctgcggggacacacccagctccacggtgaccctgggctgcctggtcaaagggtacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccacgtgcccaccccctgaactcctggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccggccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaatga

SEQ ID NO: 72 - DNA, 405 bp, Artificial Sequence
  Anti-ZIKV #5 VH DNA
atggagactgggctgcgctggcttctcctggtcgctgtgctcaaaggtgtccagtgtcagtcgttggaggagtccgggggagacctggtcaagcctggggcatccctgacactcacctgcacagcctctggattcgacttcagtgacagatattacatgtgttgggtccgccaggctccagggaaggggctggagtggatcggatgcatttatgttggtagtggtgacacttattatgcgagctgggcgaaaggccgattcaccatctccagaacctcgtcgaccacggtgactctgcaaatgacccgtctgacagccgcggacacggccacctatttctgtgcgagacatcctggtacttactttactttatggggcccaggcaccctggtcaccgtctcctca

SEQ ID NO: 73 - DNA, 711 bp, Oryctolagus cuniculus
atggacacgagggcccccactcagctgctggggctcctgctgctctggctcccaggtgccagatgtgatgttgtgatgacccagactccatcctccgtgtctgcagctgtgggaggcacagtcaccatcaattgccaggccagtcagaacattgtcaacaatttagcctggtatcagcagagaccagggcagcctcccaaactcctgatctatgatacatccactctggcatctggggtcccatcgcggttcaaaggcagtggatctgggacagagttcactctcaccatcagcgacctggagtgtgccgatgctgccacttactactgtcaaacctattattattataataaaattataaatggtttcggcggagggaccgaggtggtggtcaaaggtgatccagttgcacctactgtcctcatcttcccaccagctgctgatcaggtggcaactggaacagtcaccatcgtgtgtgtggcgaataaatactttcccgatgtcaccgtcacctgggaggtggatggcaccacccaaacaactggcatcgagaacagtaaaacaccgcagaattctgcagattgtacctacaacctcagcagcactctgacactgaccagcacacagtacaacagccacaaagagtacacctgcaaggtgacccagggcacgacctcagtcgtccagagcttcaataggggtgactgttag

SEQ ID NO: 74 - DNA, 396 bp, Artificial Sequence
  Anti-ZIKV #5 VL DNA
atggacacgagggcccccactcagctgctggggctcctgctgctctggctcccaggtgccagatgtgatgttgtgatgacccagactccatcctccgtgtctgcagctgtgggaggcacagtcaccatcaattgccaggccagtcagaacattgtcaacaatttagcctggtatcagcagagaccagggcagcctcccaaactcctgatctatgatacatccactctggcatctggggtcccatcgcggttcaaaggcagtggatctgggacagagttcactctcaccatcagcgacctggagtgtgccgatgctgccacttactactgtcaaacctattattattataataaaattataaatggtttcggcggagggaccgaggtggtggtcaaa

SEQ ID NO: 75 - protein, 116 aa, Artificial Sequence
  Anti-ZIKV #6 VH
QAQLQQSGTGLARPGASVKLSCKASGYTFTSYGISWVTQRAGQGLEWIGVIYPRSGNTYYNEKFRGKATLTADKSSSSAYMELRGLTAEDSAVYFCARENYGSVYWGQGTTLTVSS

SEQ ID NO: 76 - protein, 7 aa, Artificial Sequence
  Anti-ZIKV #6 VH-CDR1
GYTFTSY

SEQ ID NO: 77 - protein, 6 aa, Artificial Sequence
  Anti-ZIKV #6 VH-CDR2
YPRSGN

SEQ ID NO: 78 - protein, 7 aa, Artificial Sequence
  Anti-ZIKV #6 VH-CDR3
ENYGSVY

SEQ ID NO: 79 - protein, 107 aa, Artificial Sequence
  Anti-ZIKV #6 VL
DIVMTQSQKFMSTSVGDRVSITCKASQNVGTAVAWYQQKPGQSPKLLIYSASNRYTGVPDRFTGSGSGTDFTLTISNMQSEDLADYFCQQFSSYPYTFGGGTKLEIK

SEQ ID NO: 80 - protein, 12 aa, Artificial Sequence
  Anti-ZIKV #6 VL-CDR1
CKASQNVGTAVA

SEQ ID NO: 81 - protein, 7 aa, Artificial Sequence
  Anti-ZIKV #6 VL-CDR2
SASNRYT

SEQ ID NO: 82 - protein, 9 aa, Artificial Sequence
  Anti-ZIKV #6 VL-CDR3
QQFSSYPYT

SEQ ID NO: 83 - protein, 119 aa, Artificial Sequence
  Anti-ZIKV #7 VH
QVQLVQSGAEVKKPGASVKVSCKASGYTFTGYHIDWVRQARGQGLEWMGRINPNSGGTNYAQKFQGRVTMTRDTSISTAYMQLSRLRSDDSAVYYCARMSSSIWGFDHWGQGTLVTVSS

SEQ ID NO: 84 - protein, 8 aa, Artificial Sequence
  Anti-ZIKV #7 VH-CDR1
GYTFTGYH

SEQ ID NO: 85 - protein, 8 aa, Artificial Sequence
  Anti-ZIKV #7 VH-CDR2
INPNSGGT

SEQ ID NO: 86 - protein, 12 aa, Artificial Sequence
  Anti-ZIKV #7 VH-CDR3
ARMSSSIWGFDH

SEQ ID NO: 87 - protein, 108 aa, Artificial Sequence
  Anti-ZIKV #7 VL
EIVMTQSPATLSVSPGERATLSCRASQSVLINLAWYQQKPGQAPRLLIYGASSRATGIPARFSGSGSGTEFTLTISSLQSEDFAVYYCQQYNDWPPITFGQGTRLEIK

SEQ ID NO: 88 - protein, 6 aa, Artificial Sequence
  Anti-ZIKV #7 VL-CDR1
QSVLIN

SEQ ID NO: 89 - protein, 9 aa, Artificial Sequence
  Anti-ZIKV #7 VL-CDR2
LIYGASSRA

SEQ ID NO: 90 - protein, 10 aa, Artificial Sequence
  Anti-ZIKV #7 VL-CDR3
QQYNDWPPIT

SEQ ID NO: 91 - DNA, 358 bp, Artificial Sequence
  Anti-ZIKV #7 VH DNA
caggtgcagctggtccagagcggagcagaggtgaagaaacccggcgcctcagtgaaggtcagctgcaaagcttccggctacaccttcacagggtatcacatcgactgggtgaggcaggcaagaggacagggactggaatggatgggacggattaaccctaattctggcgggaccaactacgcccagaagtttcagggccgagtgactatgaccagagacaccagcatctccacagcttatatgcagctgtcccggctgagatctgacgatagtgccgtctactattgtgctcggatgagctcctctatttggggcttcgatcattgggggcagggaacactggtgactgtcagttcag

SEQ ID NO: 92 - DNA, 325 bp, Artificial Sequence
  Anti-ZIKV #7 VL DNA
gagatcgtgatgactcagtctccagccaccctgtcagtcagcccaggagaacgggcaaccctgtcttgcagagcctcccagtctgtgctgattaacctggcttggtaccagcagaagccaggccaggcaccccgactgctgatctatggagcatcctccagggctaccggcattcctgcacgcttcagtggatcaggaagcggaacagagtttaccctgacaatctctagtctgcagtccgaagacttcgctgtctactattgtcagcagtacaatgattggccccctatcacatttggccaggggactagactggagatcaagc

SEQ ID NO: 93 - protein, 448 aa, Oryctolagus cuniculus
QAQLEESGGDLVKPEGSLTLTCTASGFSFTDRHYMCWVRQAPGKGLEWIACVYPGSSGSTYYASWARGRFTISKTSSTTVTLRMTSLTAADTATYFCARSSYPDSSGYSYGMDLWGPGTLVTVPSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 94 - protein, 125 aa, Artificial Sequence
  Anti-ZIKV #8 VH
QAQLEESGGDLVKPEGSLTLTCTASGFSFTDRHYMCWVRQAPGKGLEWIACVYPGSSGSTYYASWARGRFTISKTSSTTVTLRMTSLTAADTATYFCARSSYPDSSGYSYGMDLWGPGTLVTVPS

SEQ ID NO: 95 - protein, 9 aa, Artificial Sequence
  Anti-ZIKV #8 VH-CDR1
GFSFTDRHY

SEQ ID NO: 96 - protein, 8 aa, Artificial Sequence
  Anti-ZIKV #8 VH-CDR2
YPGSSGST

SEQ ID NO: 97 - protein, 17 aa, Artificial Sequence
  Anti-ZIKV #8 VH-CDR3
ARSSYPDSSGYSYGMDL

SEQ ID NO: 98 - protein, 214 aa, Oryctolagus cuniculus; identical to SEQ ID NO: 24

SEQ ID NO: 99 - protein, 110 aa, Artificial Sequence; identical to SEQ ID NO: 25
  Anti-ZIKV #8 VL

SEQ ID NO: 100 - protein, 6 aa, Artificial Sequence
  Anti-ZIKV #8 VL-CDR1
QNINSN

SEQ ID NO: 101 - protein, 3 aa, Artificial Sequence
  Anti-ZIKV #8 VL-CDR2
LTS

SEQ ID NO: 102 - protein, 12 aa, Artificial Sequence
  Anti-ZIKV #8 VL-CDR3
QTYYDISNYGYA

SEQ ID NO: 103 - DNA, 1404 bp, Oryctolagus cuniculus
atggagactgggctgcgctggcttctcctggtcgctgtgctcaaaggtgtccagtgtcaggcgcagctggaggagtccgggggagacctggtcaagcctgagggatccctgacactcacctgcacagcctctggattctccttcactgacaggcattacatgtgctgggtccgccaggctccggggaaggggctggagtggatcgcatgcgtttatcctggtagtagtggtagcacttactacgcgagctgggcgagaggccgattcaccatctccaaaacctcgtcgaccacggtgactctgcgcatgaccagtctgacagccgcggacacggccacctatttctgtgcgagatcctcatatcctgatagtagtggctatagttatggcatggacctctggggccctgggaccctcgtcaccgtcccttcagggcaacctaaggctccatcagtcttcccactggccccctgctgcggggacacacccagctccacggtgaccctgggctgcctggtcaaagggtacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccacgtgcccaccccctgaactcctggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccggccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaatga

SEQ ID NO: 104 - DNA, 432 bp, Artificial Sequence
  Anti-ZIKV #8 VH DNA
atggagactgggctgcgctggcttctcctggtcgctgtgctcaaaggtgtccagtgtcaggcgcagctggaggagtccgggggagacctggtcaagcctgagggatccctgacactcacctgcacagcctctggattctccttcactgacaggcattacatgtgctgggtccgccaggctccggggaaggggctggagtggatcgcatgcgtttatcctggtagtagtggtagcacttactacgcgagctgggcgagaggccgattcaccatctccaaaacctcgtcgaccacggtgactctgcgcatgaccagtctgacagccgcggacacggccacctatttctgtgcgagatcctcatatcctgatagtagtggctatagttatggcatggacctctggggccctgggaccctcgtcaccgtcccttca

SEQ ID NO: 105 - DNA, 714 bp, Oryctolagus cuniculus; identical to SEQ ID NO: 31

SEQ ID NO: 106 - DNA, 399 bp, Artificial Sequence; identical to SEQ ID NO: 32
  Anti-ZIKV #8 VL DNA

SEQ ID NO: 107 - protein, 448 aa, Oryctolagus cuniculus; identical to SEQ ID NO: 33

SEQ ID NO: 108 - protein, 125 aa, Artificial Sequence; identical to SEQ ID NO: 34
  Anti-ZIKV #9 and #10 VH

SEQ ID NO: 109 - protein, 9 aa, Artificial Sequence
  Anti-ZIKV #9 and #10 VH-CDR1
GFSFTDRHY

SEQ ID NO: 110 - protein, 8 aa, Artificial Sequence
  Anti-ZIKV #9 and #10 VH-CDR2
YPGSSGST

SEQ ID NO: 111 - protein, 17 aa, Artificial Sequence
  Anti-ZIKV #9 and #10 VH-CDR3
ARSSYPDSSGYSYGMDL

SEQ ID NO: 112 - protein, 214 aa, Oryctolagus cuniculus; identical to SEQ ID NO: 24

SEQ ID NO: 113 - protein, 110 aa, Artificial Sequence; identical to SEQ ID NO: 25
  Anti-ZIKV #9 and #10 VL

SEQ ID NO: 114 - protein, 6 aa, Artificial Sequence
  Anti-ZIKV #9 and #10 VL-CDR1
QNINSN

SEQ ID NO: 115 - protein, 3 aa, Artificial Sequence
  Anti-ZIKV #9 and #10 VL-CDR2
LTS

SEQ ID NO: 116 - protein, 12 aa, Artificial Sequence
  Anti-ZIKV #9 and #10 VL-CDR3
QTYYDISNYGYA

SEQ ID NO: 117 - DNA, 1404 bp, Oryctolagus cuniculus; identical to SEQ ID NO: 43

SEQ ID NO: 118 - DNA, 432 bp, Artificial Sequence; identical to SEQ ID NO: 44
  Anti-ZIKV #9 and #10 VH DNA

SEQ ID NO: 119 - DNA, 714 bp, Oryctolagus cuniculus; identical to SEQ ID NO: 31

SEQ ID NO: 120 - DNA, 399 bp, Artificial Sequence; identical to SEQ ID NO: 32
  Anti-ZIKV #9 and #10 VL DNA

SEQ ID NO: 121 - protein, 444 aa, Oryctolagus cuniculus
QEQLVESGGGLVQPEGSLTITCTGSGFSFSSRFYMCWVRQAPGKGLEWIACIYGGSSGSTYYASWAKGRFTISKTSSTTVTLQISSLTAADTATYFCARGGSTAAAGFNLWGPGTLLTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 122 - protein, 121 aa, Artificial Sequence
  Anti-ZIKV #11 VH
QEQLVESGGGLVQPEGSLTITCTGSGFSFSSRFYMCWVRQAPGKGLEWIACIYGGSSGSTYYASWAKGRFTISKTSSTTVTLQISSLTAADTATYFCARGGSTAAAGFNLWGPGTLLTVSS

SEQ ID NO: 123 - protein, 9 aa, Artificial Sequence
  Anti-ZIKV #11 VH-CDR1
GFSFSSRFY

SEQ ID NO: 124 - protein, 8 aa, Artificial Sequence
  Anti-ZIKV #11 VH-CDR2
YGGSSGST

SEQ ID NO: 125 - protein, 13 aa, Artificial Sequence
  Anti-ZIKV #11 VH-CDR3
ARGGSTAAAGFNL

SEQ ID NO: 126 - protein, 215 aa, Oryctolagus cuniculus
DVVMTQTPASVSATVGGTVTIKCQASEDIYNLLAWYQQKPGQPPKLLIYYASTLASGVPSRFKGSGSGTEFTLTISDLECADAATYYCQCNDYGGTYVPNAFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 127 - protein, 111 aa, Artificial Sequence
  Anti-ZIKV #11 VL
DVVMTQTPASVSATVGGTVTIKCQASEDIYNLLAWYQQKPGQPPKLLIYYASTLASGVPSRFKGSGSGTEFTLTISDLECADAATYYCQCNDYGGTYVPNAFGGGTEVVVK

SEQ ID NO: 128 - protein, 6 aa, Artificial Sequence
  Anti-ZIKV #11 VL-CDR1
EDIYNL

SEQ ID NO: 129 - protein, 3 aa, Artificial Sequence
  Anti-ZIKV #11 VL-CDR2
YAS

SEQ ID NO: 130 - protein, 13 aa, Artificial Sequence
  Anti-ZIKV #11 VL-CDR3
QCNDYGGTYVPNA

SEQ ID NO: 131 - DNA, 1392 bp, Oryctolagus cuniculus
atggagactgggctgcgctggcttctcctggtcgctgtgctcaaaggtgtccagtgtcaggagcagctggtggagtccgggggaggcctggtccagcctgagggatccctgacaatcacctgcacaggttctggattctccttcagtagcaggttctacatgtgctgggtccgccaggctccagggaaggggctggagtggatcgcatgtatttatggtggtagtagtggtagcacttactacgcgagctgggcgaaaggccgattcaccatctccaaaacctcgtcgaccacggtgactctgcaaatttccagtctgacagccgcggacacggccacctatttctgtgccagaggtggtagtactgctgctgcgggctttaacttgtggggcccaggcaccctgctcaccgtctcctcagggcaacctaaggctccatcagtcttcccactggccccctgctgcggggacacacccagctccacggtgaccctgggctgcctggtcaaagggtacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccacgtgcccaccccctgaactcctggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccggccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaatga

SEQ ID NO: 132 - DNA, 420 bp, Artificial Sequence
  Anti-ZIKV #11 VH DNA
atggagactgggctgcgctggcttctcctggtcgctgtgctcaaaggtgtccagtgtcaggagcagctggtggagtccgggggaggcctggtccagcctgagggatccctgacaatcacctgcacaggttctggattctccttcagtagcaggttctacatgtgctgggtccgccaggctccagggaaggggctggagtggatcgcatgtatttatggtggtagtagtggtagcacttactacgcgagctgggcgaaaggccgattcaccatctccaaaacctcgtcgaccacggtgactctgcaaatttccagtctgacagccgcggacacggccacctatttctgtgccagaggtggtagtactgctgctgcgggctttaacttgtggggcccaggcaccctgctcaccgtctcctca

SEQ ID NO: 133 - DNA, 714 bp, Oryctolagus cuniculus
atggacacgagggcccccactcagctgctggggctcctgctgctctggctcccaggtgccagatgtgatgttgtgatgacccagactccagcctccgtgtctgcaactgtgggaggcacagtcaccatcaagtgccaggccagtgaggacatttataacttattggcctggtatcagcagaaaccagggcagcctcccaaactcctgatctattatgcatccactctggcatctggggtcccatcacggttcaaaggcagtggatctgggacagagttcactctcaccatcagcgacctggagtgtgccgatgctgccacttactactgtcaatgtaatgattatggtggtacttatgtccccaatgctttcggcggagggaccgaggtggtggtcaagggtgatccagttgcacctactgtcctcatcttcccaccagctgctgatcaggtggcaactggaacagtcaccatcgtgtgtgtggcgaataaatactttcccgatgtcaccgtcacctgggaggtggatggcaccacccaaacaactggcatcgagaacagtaaaacaccgcagaattctgcagattgtacctacaacctcagcagcactctgacactgaccagcacacagtacaacagccacaaagagtacacctgcaaggtgacccagggcacgacctcagtcgtccagagcttcaataggggtgactgttag

SEQ ID NO: 134 - DNA, 399 bp, Artificial Sequence
  Anti-ZIKV #11 VL DNA
atggacacgagggcccccactcagctgctggggctcctgctgctctggctcccaggtgccagatgtgatgttgtgatgacccagactccagcctccgtgtctgcaactgtgggaggcacagtcaccatcaagtgccaggccagtgaggacatttataacttattggcctggtatcagcagaaaccagggcagcctcccaaactcctgatctattatgcatccactctggcatctggggtcccatcacggttcaaaggcagtggatctgggacagagttcactctcaccatcagcgacctggagtgtgccgatgctgccacttactactgtcaatgtaatgattatggtggtacttatgtccccaatgctttcggcggagggaccgaggtggtggtcaag

SEQ ID NO: 135 - protein, 123 aa, Artificial Sequence
  Anti-ZIKV E Protein VH
EVQLAESGGGLVQPGGSLTLSCSGSGFTFSNYAMVWARQAPGKGLEYVSGIGRNGDSIYYTDSVKGRFTISRDNSKSMVYLQMSSLRTEDTAVYYCVKDLAIPESYRIEADYWGQGTLVIVSA

SEQ ID NO: 136 - protein, 8 aa, Artificial Sequence
  Anti-ZIKV E Protein VH-CDR1
GFTFSNYA

SEQ ID NO: 137 - protein, 8 aa, Artificial Sequence
  Anti-ZIKV E Protein VH-CDR2
IGRNGDSI

SEQ ID NO: 138 - protein, 16 aa, Artificial Sequence
  Anti-ZIKV E Protein VH-CDR3
VKDLAIPESYRIEADY

SEQ ID NO: 139 - protein, 113 aa, Artificial Sequence
  Anti-ZIKV E Protein VL
DIVMTQSPDSLAVSLGERATINCKSSQSVLYRSNNKNYLSWYQQKPGQPPKLLIYWASTRESGVPDRFSGSGSGTDFTLTISPLQAEDVAVYYCQQYYSSPRTFGQGTKVEIK

SEQ ID NO: 140 - protein, 12 aa, Artificial Sequence
  Anti-ZIKV E Protein VL-CDR1
QSVLYRSNNKNY

SEQ ID NO: 141 - protein, 9 aa, Artificial Sequence
  Anti-ZIKV E Protein VL-CDR2
LIYWASTRE

SEQ ID NO: 142 - protein, 9 aa, Artificial Sequence
  Anti-ZIKV E Protein VL-CDR3
QQYYSSPRT

SEQ ID NO: 143 - DNA, 370 bp, Artificial Sequence
  Anti-ZIKV E Protein VH DNA
gaggtgcagctggcagaatcaggcgggggactggtccagcctggcggcagcctgacactgtcttgcagtggatcaggcttcacttttagtaactatgcaatggtgtgggcaaggcaggctcctgggaagggactggagtatgtctctggcatcgggcgcaacggggactctatctactatactgatagtgtgaagggccggttcaccatcagcagagacaatagcaaatccatggtgtacctgcagatgagctccctgcgaaccgaagacacagcagtgtactattgcgtgaaagatctggccatccccgagtcctacagaattgaagctgattattggggacagggcaccctggtcatcgtgagcgccg

SEQ ID NO: 144 - DNA, 340 bp, Artificial Sequence
  Anti-ZIKV E Protein VL DNA
gacatcgtgatgacacagtctccagatagtctggcagtcagtctgggggagagggccactattaactgcaagagctcccagtccgtgctgtaccgctctaacaacaagaattacctgtcttggtatcagcagaagcccggacagccccctaaactgctgatctattgggcttcaacccgggaaagcggcgtcccagacagattctcaggcagcgggtccggaacagacttcaccctgacaattagccccctgcaggcagaggacgtggctgtctactattgtcagcagtactattctagtcctcgaactttcggccaggggaccaaggtggaaatcaaac

SEQ ID NO: 145 - protein, 468 aa, Mus musculus
MRWSWIFLFLLSGTAGVLSEVQLQQSGPELVKPGTSVKISCKTSGYTFTEYTIHWVKQSHGKSLAWIGGIDPNSGGTNYSPNFKGKATLTVDKSSSTAYMDLRSLSSEDSAVYFCARIYHYDGYFDVWGAGTAVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 146 - protein, 236 aa, Mus musculus
MDMRTPAQFLGILLLWFPGIKCDIKMTQSPSSMYASLGERVTITCKASQDINSYLTWFQQKPGKSPKTLIYRANRLIDGVPSRFSGSGSGQDYSLTISSLDYEDMGIYYCLQYDEFPPTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 147 - DNA, 1407 bp, Mus musculus
atgagatggagctggatctttctctttctcctgtcaggaactgcaggtgtcctctctgaggtccagctgcaacagtctggacctgagctggtgaagcctgggacttcagtgaagatatcctgcaagacttctggatacacattcactgaatataccatacactgggtgaagcagagccatggaaagagccttgcgtggattggaggtattgatcctaacagtggtggtactaactacagcccgaacttcaagggcaaggccacattgactgttgacaagtcctccagcacagcctacatggacctccgcagcctgtcatctgaggattctgcagtctacttctgtgcaaggatctatcattacgacggatacttcgatgtctggggcgcagggaccgcggtcaccgtctcctcagccaaaacaacagccccatcggtctatccactggcccctgtgtgtggagatacaactggctcctcggtgactctaggatgcctggtcaagggttatttccctgagccagtgaccttgacctggaactctggatccctgtccagtggtgtgcacaccttcccagctgtcctgcagtctgacctctacaccctcagcagctcagtgactgtaacctcgagcacctggcccagccagtccatcacctgcaatgtggcccacccggcaagcagcaccaaggtggacaagaaaattgagcccagagggcccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacagacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaatga

SEQ ID NO: 148 - DNA, 711 bp, Mus musculus
atggacatgaggacccctgctcagtttcttggaatcttgttgctctggtttccaggtatcaaatgtgacatcaagatgacccagtctccatcttcaatgtatgcatctctaggagagagagtcactatcacttgcaaggcgagtcaggacattaatagctatttaacctggttccagcagaaaccagggaaatctcccaagaccctgatctatcgtgcaaacagattgatagatggggtcccatcaaggttcagtggcagtggatctgggcaagattattctctcaccatcagcagcctggactatgaagatatgggaatttattattgtctacagtatgatgagtttcctcccacgttcggaggggggaccaagctggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttag

SEQ ID NO: 149 - protein, 448 aa, Oryctolagus cuniculus
QQQLVESGGGLVKPGASLTLNCKASGLDFSTNSYMCWVRQAPGKGLEWIACIYVGDSSEIYYASWAKGRFTISKTSSTTVTLQMTSLTAADTATYFCARDLPSFTAPYAGYLRLWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 150 - protein, 125 aa, Artificial Sequence
  Antibody Clone 78-2 VH
QQQLVESGGGLVKPGASLTLNCKASGLDFSTNSYMCWVRQAPGKGLEWIACIYVGDSSEIYYASWAKGRFTISKTSSTTVTLQMTSLTAADTATYFCARDLPSFTAPYAGYLRLWGPGTLVTVSS

SEQ ID NO: 151 - protein, 9 aa, Artificial Sequence
  Antibody Clone 78-2 VH-CDR1
GLDFSTNSY

SEQ ID NO: 152 - protein, 9 aa, Artificial Sequence
  Antibody Clone 78-2 VH-CDR1
IYVGDSSEI

SEQ ID NO: 153 - protein, 17 aa, Artificial Sequence
  Antibody Clone 78-2 VH-CDR3
ARDLPSFTAPYAGYLRL

SEQ ID NO: 154 - protein, 225 aa, Oryctolagus cuniculus
QPVLTQPPSLSASLGTTARLTCTLRTGYNVGDYPLLWLQRVPGRPPRYLLTYHTEEFKHQGSGVHSRFSGSKDASENAGVLSISGLQPEDEANYYCYTVHATESSLHYVFGGGTQLTVTGQPAVTPSVILFPPSSEELKDNKATLVCLINDFYPGTVKVNWKADGTPVTQGVDTTQPSKQSNSKYAASSFLSLSANQWKSYQSVTCQVTHEGHTVEKSLAPAECS

SEQ ID NO: 155 - protein, 119 aa, Artificial Sequence
  Antibody Clone 78-2 VL
QPVLTQPPSLSASLGTTARLTCTLRTGYNVGDYPLLWLQRVPGRPPRYLLTYHTEEFKHQGSGVHSRFSGSKDASENAGVLSISGLQPEDEANYYCYTVHATESSLHYVFGGGTQLTVT

SEQ ID NO: 156 - protein, 9 aa, Artificial Sequence
  Antibody Clone 78-2 VL-CDR1
TGYNVGDYP

SEQ ID NO: 157 - protein, 8 aa, Artificial Sequence
  Antibody Clone 78-2 VL-CDR2
YHTEEFKH

SEQ ID NO: 158 - protein, 14 aa, Artificial Sequence
  Antibody Clone 78-2 VL-CDR3
YTVHATESSLHYVF

SEQ ID NO: 159 - DNA, 1404 bp, Oryctolagus cuniculus
atggagactgggctgcgctggcttctcctggtcgctgtgctcaaaggtgtccagtgtcagcagcagctggtggagtccgggggaggcctggtcaagcctggggcatccctgacactcaactgcaaagcctctggactcgacttcagtaccaactcctacatgtgctgggtccgccaggctccagggaagggcctggagtggatcgcatgcatttatgttggtgatagtagtgagatttattacgcgagctgggcgaaaggccgattcaccatctccaaaacctcgtcgaccacggtgactctacaaatgaccagtctgacagccgcggacacggccacctatttctgtgcgagagatctcccttcctttactgctccttatgctggttatcttcgcttgtggggcccaggcaccctggtcaccgtctcctcagggcaacctaaggctccatcagtcttcccactggccccctgctgcggggacacacccagctccacggtgaccctgggctgcctggtcaaagggtacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccacgtgcccaccccctgaactcctggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccggccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaatga

SEQ ID NO: 160 - DNA, 432 bp, Artificial Sequence
  Antibody Clone 78-2 VH DNA
atggagactgggctgcgctggcttctcctggtcgctgtgctcaaaggtgtccagtgtcagcagcagctggtggagtccgggggaggcctggtcaagcctggggcatccctgacactcaactgcaaagcctctggactcgacttcagtaccaactcctacatgtgctgggtccgccaggctccagggaagggcctggagtggatcgcatgcatttatgttggtgatagtagtgagatttattacgcgagctgggcgaaaggccgattcaccatctccaaaacctcgtcgaccacggtgactctacaaatgaccagtctgacagccgcggacacggccacctatttctgtgcgagagatctcccttcctttactgctccttatgctggttatcttcgcttgtggggcccaggcaccctggtcaccgtctcctca

SEQ ID NO: 161 - DNA, 732 bp, Oryctolagus cuniculus
atggcctgggctcctctgctcctgctgctcctctcccactgcacaggttccctctcccagcctgtgctgactcagccgccctccctgtctgcgtctctgggcacaacagccagactcacctgcaccctgaggactggctacaatgttggcgactaccctttactgtggctccagcgggtgccagggaggcctcccaggtatctcctgacctaccacacagaagaatttaaacatcagggctccggggtgcacagccgcttctctggatccaaagacgcctcagagaatgccggtgtcctgagcatctctgggctgcagcccgaggacgaggccaactattattgttatactgttcatgctactgagagcagcctccattatgtgttcggcggagggacacaactgaccgtcacaggtcagcccgcggtgaccccctcggtcattttgttcccgccctcctctgaggaactcaaggacaacaaggccaccttggtgtgtctgatcaatgacttctacccgggcaccgtgaaggtgaactggaaggccgatggtactcccgtcacccagggcgtggacaccacccagccctccaaacagagcaacagcaagtatgcggccagcagcttcctcagcctgtcagccaatcaatggaaatcctaccagagtgtcacctgccaggtcacgcatgagggccacacggtggagaagagcctggcccctgcagaatgctcc

SEQ ID NO: 162 - DNA, 414 bp, Artificial Sequence
  Antibody Clone 78-2 VL DNA
atggcctgggctcctctgctcctgctgctcctctcccactgcacaggttccctctcccagcctgtgctgactcagccgccctccctgtctgcgtctctgggcacaacagccagactcacctgcaccctgaggactggctacaatgttggcgactaccctttactgtggctccagcgggtgccagggaggcctcccaggtatctcctgacctaccacacagaagaatttaaacatcagggctccggggtgcacagccgcttctctggatccaaagacgcctcagagaatgccggtgtcctgagcatctctgggctgcagcccgaggacgaggccaactattattgttatactgttcatgctactgagagcagcctccattatgtgttcggcggagggacacaactgaccgtcaca

SEQ ID NO: 163 - protein, 442 aa, Oryctolagus cuniculus
QCQSLEESGGGLVQPEGSLTLACTASEFDSSSNAMCWVRQAPGKGLEWIARIYSGSGTIYYANWARGRFTISKTSSTTVTLQMTSLTDADTATYFCARYNTGGFYYDLWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 164 - protein, 119 aa, Artificial Sequence
  Antibody Clone 278-11 VH
QCQSLEESGGGLVQPEGSLTLACTASEFDSSSNAMCWVRQAPGKGLEWIARIYSGSGTIYYANWARGRFTISKTSSTTVTLQMTSLTDADTATYFCARYNTGGFYYDLWGPGTLVTVSS

SEQ ID NO: 165 - protein, 8 aa, Artificial Sequence
  Antibody Clone 278-11 VH-CDR1
EFDSSSNA

SEQ ID NO: 166 - protein, 8 aa, Artificial Sequence
  Antibody Clone 278-11 VH-CDR2
IYSGSGTI

SEQ ID NO: 167 - protein, 12 aa, Artificial Sequence
  Antibody Clone 278-11 VH-CDR3
ARYNTGGFYYDL

SEQ ID NO: 168 - protein, 214 aa, Oryctolagus cuniculus
ALVMTQTPASVEAAVGGTVTINCQASQRIGTNLAWYQQKPGQPPKVLIYKASTLASGVSSRFIGSGSGTDYTLTISGVECDDAATYYCQQGYSSNDADNTFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 169 - protein, 110 aa, Artificial Sequence
  Antibody Clone 278-11 VL
ALVMTQTPASVEAAVGGTVTINCQASQRIGTNLAWYQQKPGQPPKVLIYKASTLASGVSSRFIGSGSGTDYTLTISGVECDDAATYYCQQGYSSNDADNTFGGGTEVVVK

SEQ ID NO: 170 - protein, 6 aa, Artificial Sequence
  Antibody Clone 278-11 VL-CDR1
QRIGTN

SEQ ID NO: 171 - protein, 3 aa, Artificial Sequence
  Antibody Clone 278-11 VL-CDR2
KAS

SEQ ID NO: 172 - protein, 12 aa, Artificial Sequence
  Antibody Clone 278-11 VL-CDR3
QQGYSSNDADNT

SEQ ID NO: 173 - DNA, 1370 bp, Oryctolagus cuniculus
atggagactgggctgcgctggcttctcctggtcgctgtgctcaaaggtgtccagtgtcagtcgttggaggagtccgggggaggcctggtccagcctgagggatccctgacactcgcctgcacagctttgaattcgactccagtagcaatgcaatgtgctgggtccgccaggctccagggaagggactggagtggatcgcacgcatctatagtggtagtggtactatttactacgctaactgggcgagaggccgatcaccatctccaagacctcgtcgaccacggtgactctgcaaatgaccagtctgacagacgcggacacggccacctatttctgtgcgagatacaatactggtgggttctattatgacttgtggggcccggcaccctggtcaccgtctcctcagggcaacctaaggctccatcagtcttcccactggccccctgctgcggggacacacccagctccacggtgaccctgggctgcctggtcaaagggtacctcccggagccagtgaccgtgacctggaactgggcaccctcaccaatggggtacgcaccttcccgtccgtccggcagtcctcaggcctctactcgctgagcagcgtggtgagcgtgacctcaagcagccagcccgtcacctgcaacgtggcccacccaccaccaacaccaaagtggacaagaccgttgcgccctcgacatgcagcaagcccacgtgcccaccccctgaactcctggggggaccgtctgtcttcatcttccccccaaaacccaaggacaccctcatatctcacgcacccccgaggtcacatgcgtggtggtggacgtgagccaggatgaccccgaggtgcagttcacatggtacataaacaacgagcaggtgcgcaccgcccggccgccgctacgggagcagcgttcaacagcacgatccgcgtggtcagcaccctccccatcgcgcaccaggactggctgaggggcaaggagttcaagtgcaaagtccacaacaaggcactcccggcccccatcgagaaaaccatctccaagccagagggcagcccctggagccgaaggtctacaccatgggccctccccgggaggagctgagcagcaggtcggtcagcctgacctgcatgatcaacggcttctacccttccgacatctcggtggatgggagaagaacgggaaggcagaggacaactacaagaccacgccggccgtgctggacagcgacggctcctacttcctctacagcaagctctcagtgcccacgagtgagtggcagcggggcgacgtctcacctgctccgtgatgcacgaggccttgcacaaccactacacgcagaagtccatctcccgctctccgggtaaatga

SEQ ID NO: 174 - DNA, 405 bp, Artificial Sequence
  Antibody Clone 278-11 VH DNA
atggagactgggctgcgctggcttctcctggtcgctgtgctcaaaggtgtccagtgtcagtcgttggaggagtccgggggaggcctggtccagcctgagggatccctgacactcgcctgcacagctttgaattcgactccagtagcaatgcaatgtgctgggtccgccaggctccagggaagggactggagtggatcgcacgcatctatagtggtagtggtactatttactacgctaactgggcgagaggccgatcaccatctccaagacctcgtcgaccacggtgactctgcaaatgaccagtctgacagacgcggacacggccacctatttctgtgcgagatacaatactggtgggttctattatgacttgtggggcccggcaccctggtcaccgtctcctca

SEQ ID NO: 175 - DNA, 706 bp, Oryctolagus cuniculus
atggacacgagggcccccactcagctgctggggctcctgctgctctggctcccaggtgccagatgtgcccttgtgatgacccagactccagcctccgtggaggcagctgtgggaggcacagtcaccacaattgccaggccagtcagagaattggtactaatttagcctggtatcagcagaaaccagggcagcctcccaaggtcctgatctacaaggcatccactctggcatctggggtctcatcgcggttcatagcagtggatctgggacagactatactctcaccatcagcggcgtggagtgtgacgatgctgccacttactactgtcaacagggttatagtagtaatgatgccgataatactttcggcggagggaccgagtggtggtcaaaggtgatccagttgcacctactgtcctcatcttcccaccagctgctgatcaggtggcaactggaacagtcaccatcgtgtgtgtggcgaataaatactttcccgatgtcaccgtcacctgggaggtggtggcaccacccaaacaactggcatcgagaacagtaaaacaccgcagaattctgcagattgtacctacaacctcagcagcactctgacactgaccagcacacagtacaacagccacaaagagtacaccgcaaggtgacccagggcacgacctcagtcgtccagagcttcaataggggtgactgttag

SEQ ID NO: 176 - DNA, 393 bp, Artificial Sequence
  Antibody Clone 278-11 VL DNA
atggacacgagggcccccactcagctgctggggctcctgctgctctggctcccaggtgccagatgtgcccttgtgatgacccagactccagcctccgtggaggcagctgtgggaggcacagtcaccacaattgccaggccagtcagagaattggtactaatttagcctggtatcagcagaaaccagggcagcctcccaaggtcctgatctacaaggcatccactctggcatctggggtctcatcgcggttcatagcagtggatctgggacagactatactctcaccatcagcggcgtggagtgtgacgatgctgccacttactactgtcaacagggttatagtagtaatgatgccgataatactttcggcggagggaccgagtggtggtcaaa

SEQ ID NO: 177 - DNA, 10969 bp, Japanese Encephalitis Virus Strain SA-14
agaagtttatctgtgtgaacttcttggcttagtatcgttgagaagaatcgagagattagtgcagtttaaacagttttttagaacggaagataaccatgactaaaaaaccaggagggcccggtaaaaaccgggctatcaatatgctgaaacgcggcctaccccgcgtattcccactagtgggagtgaagagggtagtaatgagcttgttggacggcagagggccagtacgtttcgtgctggctcttatcacgttcttcaagtttacagcattagccccgaccaaggcgcttttaggccgatggaaagcagtggaaaagagtgtggcaatgaaacatcttactagtttcaaacgagaacttggaacactcattgacgccgtgaacaagcggggcagaaagcaaaacaaaagaggaggaaatgaaggctcaatcatgtggctcgcgagcttggcagttgtcatagcttgtgcaggagccatgaagttgtcgaatttccaggggaagcttttgatgaccatcaacaacacggacattgcagacgttatcgtgattcccacctcaaaaggagagaacagatgctgggtccgggcaatcgacgtcggctacatgtgtgaggacactatcacgtacgaatgtcctaagcttaccatgggcaatgatccagaggatgtggattgctggtgtgacaaccaagaagtctacgtccaatatggacggtgcacgcggaccaggcattccaagcgaagcaggagatccgtgtcggtccaaacacatggggagagttcactagtgaataaaaaagaggcttggctggattcaacgaaagccacacgatatctcatgaaaactgagaactggatcataaggaatcctggctatgctttcctggcggcggtacttggctggatgcttggcagtaacaacggtcaacgcgtggtatttaccatcctcctgctgttggtcgctccggcttacagttttaattgtctgggaatgggcaatcgtgacttcatagaaggagccagtggagccacttgggtggacttggtgctagaaggagatagctgcttgacaatcatggcaaacgacaaaccaacattggacgtccgcatgattaacatcgaagctagccaacttgctgaggtcagaagttactgctatcatgcttcagtcactgacatctcgacggtggctcggtgccccacgactggagaagcccacaacgagaagcgagctgatagtagctatgtgtgcaaacaaggcttcactgaccgtgggtggggcaacggatgtggacttttcgggaagggaagcattgacacatgtgcaaaattctcctgcaccagtaaagcgattgggagaacaatccagccagaaaacatcaaatacgaagttggcatttttgtgcatggaaccaccacttcggaaaaccatgggaattattcagcgcaagttggggcgtcccaggcggcaaagtttacagtaacacccaatgctccttcgataaccctcaaacttggtgactacggagaagtcacactggactgtgagccaaggagtggactgaacactgaagcgttttacgtcatgaccgtggggtcaaagtcatttctggtccatagggagtggtttcatgacctcgctctcccctggacgtccccttcgagcacagcgtggagaaacagagaactcctcatggaatttgaaggggcgcacgccacaaaacagtccgttgttgctcttgggtcacaggaaggaggcctccatcaggcgttggcaggagccatcgtggtggagtactcaagctcagtgaagttaacatcaggccacctgaaatgtaggctgaaaatggacaaactggctctgaaaggcacaacctatggcatgtgtacagaaaaattctcgttcgcgaaaaatccggtggacactggtcacggaacagttgtcattgaactctcctactctgggagtgatggcccctgcaaaattccgattgtttccgttgcgagcctcaatgacatgacccccgttgggcggctggtgacagtgaaccccttcgtcgcgacttccagtgccaactcaaaggtgctggtcgagatggaaccccccttcggagactcctacatcgtagttggaaggggagacaagcagatcaaccaccattggcacaaagctggaagcacgctgggcaaggccttttcaacaactttgaagggagctcaaagactggcagcgttgggcgacacagcctgggactttggctctattggaggggtcttcaactccataggaagagccgttcaccaagtgtttggtggtgccttcagaacactctttgggggaatgtcttggatcacacaagggctaatgggtgccctactgctctggatgggcgtcaacgcacgagaccgatcaattgctttggccttcttagccacaggaggtgtgctcgtgttcttagcgaccaatgtgcatgctgacactggatgtgccattgacatcacaagaaaagagatgagatgtggaagtggcatcttcgtgcacaacgacgtggaagcctgggtggataggtataaatatttgccagaaacgcccagatccctagcgaagatcgtccacaaagcgcacaaggaaggcgtgtgcggagtcagatctgtcactagactggagcaccaaatgtgggaagccgtaagggacgaattgaacgtcctgctcaaagagaatgcagtggacctcagtgtggttgtgaacaagcccgtgggaagatatcgctcagcccctaaacgcctatccatgacgcaagagaagtttgaaatgggctggaaagcatggggaaaaagcatcctctttgccccggaattggctaactccacatttgtcgtagatggacctgagacaaaggaatgccctgatgagcacagagcttggaacagcatgcaaatcgaagacttcggctttggcatcacatcaacccgtgtgtggctgaaaattagagaggagagcactgacgagtgtgatggagcgatcataggcacggctgtcaaaggacatgtggcagtccatagtgacttgtcgtactggattgagagtcgctacaacgacacatggaaacttgagagggcagtctttggagaggtcaaatcttgcacttggccagagacacacaccctttggggagatgatgttgaggaaagtgaactcatcattccgcacaccatagccggaccaaaaagcaagcacaatcggagggaagggtataagacacaaaaccagggaccttgggatgagaatggcatagtcttggactttgattattgcccagggacaaaagtcaccattacagaggattgtagcaagagaggcccttcggtcagaaccactactgacagtggaaagttgatcactgactggtgctgtcgcagttgctcccttccgcccctacgattccggacagaaaatggctgctggtacggaatggaaatcagacctgttatgcatgatgaaacaacactcgtcagatcacaggttgatgctttcaaaggtgaaatggttgacccttttcagctgggccttctggtgatgtttctggccacccaggaagtccttcgcaagaggtggacggccagattgaccattcctgcggttttgggggtcctacttgtgctgatgcttgggggtatcacttacactgatttggcgaggtatgtggtgctagtcgctgctgctttcgcagaggccaacagtggaggagacgtcctgcaccttgctttgattgccgtttttaagatccaaccagcatttctagtgatgaacatgcttagcacgagatggacgaaccaagaaaacgtgattctggtcctaggggctgcctttttccaattggcctcagtagatctgcaaataggagtccacggaatcctgaatgccgccgctatagcatggatgattgtccgagcgatcaccttccccacaacctcctccgtcaccatgccagtcttagcgcttctaactccggggatgagggctctatacctagacacttacagaatcatcctcctcgtcatagggatttgctccctgctgcacgagaggaaaaagaccatggcaaaaaagaaaggagctgtactcttgggcttagcgctcacatccactggatggttctcgcccaccactatagctgccggactaatggtctgcaacccaaacaagaagagagggtggccagctactgagtttttgtcggcagttggattgatgtttgccatcgtaggtggtttggccgagttggatattgaatccatgtcaatacccttcatgctggcaggtctcatggcagtgtcctacgtggtgtcaggaaaagcaacagatatgtggcttgaacgggccgccgacatcagctgggagatggatgctgcaatcacaggaagcagtcggaggctggatgtgaaactggatgatgacggagattttcacttgattgatgatcccggtgttccatggaaggtctgggtcctgcgcatgtcttgcattggcttagccgccctcacgccttgggccatcgttcccgccgctttcggttattggctcactttaaaaacaacaaaaagagggggcgtgttttgggacacgccatccccaaaaccttgctcaaaaggagacaccactacaggagtctaccgaattatggctagagggattcttggcacttaccaggccggcgtcggagtcatgtacgagaatgttttccacacactatggcacacaactagaggagcagccattatgagtggagaaggaaaattgacgccatactggggtagtgtgagagaagaccgcatagcttacggaggcccatggaggtttgaccgaaaatggaatggaacagatgacgtgcaagtgatcgtggtagaaccggggaaggctgcagtaaacatccagacaaaaccaggagtgtttcggactcccttcggggaggttggggctgttagtctggattacccgcgaggaacatccggctcacccattctggattccaatggagacattataggcctatacggcaatggagttgagcttggcgatggctcatacgtcagcgccatcgtgcagggtgaccgtcaggaggaaccagtcccagaagcttacaccccaaacatgttgagaaagagacagatgactgtgctagatttgcaccctggttcagggaaaaccaggaaaattctgccacaaataattaaggacgctatccagcagcgcctaagaacagctgtgttggcaccgacgcgggtggtagcagcagaaatggcagaagctttgagagggctcccagtacgatatcaaacttcagcagtgcagagagagcaccaagggaatgaaatagtggatgtgatgtgccacgccactctgacccatagactgatgtcaccgaacagagtgcccaactacaacctatttgtcatggatgaagctcatttcaccgacccagccagtatagccgcacgaggatacattgctaccaaggtggaattaggggaggcagcagccatctttatgacagcgaccccgcctggaaccacggatccttttcctgactcaaatgccccaatccatgatttgcaagatgagataccagacagggcatggagcagtggatacgaatggatcacagaatatgcgggtaaaaccgtgtggtttgtggcgagcgtaaaaatggggaatgagattgcaatgtgcctccaaagagcggggaaaaaggtcatccaactcaaccgcaagtcctatgacacagaatacccaaaatgtaagaatggagactgggattttgtcattaccaccgacatctctgaaatgggggccaacttcggtgcgagcagggtcatcgactgtagaaagagcgtgaaacccaccatcttagaagagggagaaggcagagtcatcctcggaaacccatctcccataaccagtgcaagcgcagctcaacggaggggcagagtaggcagaaaccccaaccaagttggagatgaataccactatgggggggctaccagtgaagatgacagtaacctagcccattggacagaggcaaagatcatgttagacaacatacacatgcccaatggactggtggcccagctctatggaccagagagggaaaaggctttcacaatggatggcgaataccgtctcagaggtgaagaaaagaaaaacttcttagagctgcttaggacggctgacctcccggtgtggctggcctacaaggtggcgtccaatggcattcagtacaccgacagaaagtggtgttttgatgggccgcgtacgaatgccatactggaggacaacaccgaggtagagatagtcacccggatgggtgagaggaaaatcctcaagccgagatggcttgatgcaagagtttatgcagatcaccaagccctcaagtggttcaaagactttgcagcagggaagagatcagccgttagcttcatagaggtgctcggtcgcatgcctgagcatttcatgggaaagacgcgggaagctttagacaccatgtacttggttgcaacggctgagaaaggtgggaaagcacaccgaatggctctcgaagagctgccagatgcactggaaaccatcacacttattgtcgccattactgtgatgacaggaggattcttcctactaatgatgcagcgaaagggtatagggaagatgggtcttggagctctagtgctcacgctagctaccttcttcctgtgggcggcagaggttcctggaaccaaaatagcagggaccctgctgatcgccctgctgctgatggtggttctcatcccagaaccggaaaaacagaggtcacagacagataaccaactggcggtgtttctcatctgtgtcttgaccgtggttggagtggtggcagcaaacgagtacgggatgctagaaaaaaccaaagcagatctcaagagcatgtttggcggaaagacgcaggcatcaggactgactggattgccaagcatggcactggacctgcgtccagccacagcctgggcactgtatggggggagcacagtcgtgctaacccctcttctgaagcacctgatcacgtcggaatacgtcaccacatcgctagcctcaattaactcacaagctggctcattattcgtcttgccacgaggcgtgccttttaccgacctagacttgaccgttggcctcgtcttccttggctgttggggtcaaatcaccctcacaacgtttctgacagccatggttctggcgacacttcactatgggtacatgctccctggatggcaagcagaagcactcagggctgcccagagaaggacagcggctggaataatgaagaatgccgttgttgacggaatggtcgccactgatgtgcctgaactggaaaggactactcctctgatgcaaaagaaagtcggacaggtgctcctcataggggtaagcgtggcagcgttcctcgtcaaccctaatgtcaccactgtgagagaagcaggggtgttggtgacggcggctacgcttactttgtgggacaatggagccagtgccgtttggaattccaccacagccacgggactctgccatgtcatgcgaggtagctacctggctggaggctccattgcttggactctcatcaagaacgctgataagccctccttgaaaaggggaaggcctgggggcaggacgctaggggagcagtggaaggaaaaactaaatgccatgagcagagaagagttttttaaataccggagagaggccataatcgaggtggaccgcactgaagcacgcagggctagacgtgaaaataacatagtgggaggacatccggtttcgcgaggctcagcaaaactccgttggcttgtggagaaaggatttgtctcgccaataggaaaagtcattgatctagggtgtgggcgtggaggatggagctactacgcagcaaccctgaagaaggtccaggaagtcagaggatacacgaaaggtggggcgggacatgaagaaccgatgctcatgcagagctacggctggaacctggtctccctgaagagtggagtggacgtgttttacaaaccttcagagcccagtgacaccctgttctgtgacataggggaatcctccccaagtccagaagtagaagaacaacgcacactacgcgtcctagagatgacatctgactggttgcaccgaggacctagagagttctgcattaaagttctctgcccttacatgcccaaggttatagaaaaaatggaagttctgcagcgccgcttcggaggtgggctagtgcgtctccccctgtcccgaaactccaatcacgagatgtattgggttagtggagccgctggcaatgtggtgcacgctgtgaacatgaccagccaggtactactggggcgaatggatcgcacagtgtggagagggccaaagtatgaggaagatgtcaacctagggagcggaacaagagccgtgggaaagggagaagtccatagcaatcaggagaaaatcaagaagagaatccagaagcttaaagaagaattcgccacaacgtggcacaaagaccctgagcatccataccgcacttggacataccacggaagctatgaagtgaaggctactggctcagccagctctctcgtcaacggagtggtggagctcatgagcaaaccttgggacgccattgccaacgtcaccaccatggccatgactgacaccaccccttttggacagcaaagagttttcaaggagaaagttgacacgaaggctcctgagccaccagctggagccaaggaagtgctcaacgagaccaccaactggctgtgggcccacttgtcacgggaaaaaagaccccgcttgtgcaccaaggaagaattcataaagaaagtcaacagcaacgcggctcttggagcagtgttcgctgaacagaatcaatggagcacggcgcgtgaggctgtggatgacccgcggttttgggagatggttgatgaagagagggaaaaccatctgcgaggagagtgtcacacatgtatctacaacatgatgggaaaaagagagaagaagcctggagagtttggaaaagctaaaggaagcagggccatttggttcatgtggcttggagcacggtatctagagtttgaagctttggggttcctgaatgaagaccattggctgagccgagagaattcaggaggtggagtggaaggctcaggcgtccaaaagctgggatacatcctccgtgacatagcaggaaagcaaggagggaaaatgtacgctgatgataccgccgggtgggacactagaattaccagaactgatttagaaaatgaagctaaggtactggagctcctagacggtgaacaccgcatgctcgcccgagccataattgaactgacttacaggcacaaagtggtcaaggtcatgagacctgcagcagaaggaaagaccgtgatggacgtgatatcaagagaagatcaaagggggagtggacaggtggtcacttatgctcttaacactttcacgaacatcgctgtccagctcgtcaggctgatggaggctgagggggtcattggaccacaacacttggaacagctacctagaaaaaccaagatagctgtcaggacctggctctttgagaatggagaggagagagtgaccaggatggcgatcagcggagacgactgtgtcgtcaagccgctggacgacagattcgccacagccctccacttcctcaacgcaatgtcaaaggtcagaaaagacatccaggaatggaagccttcgcatggctggcacgattggcagcaagttcccttctgctctaaccattttcaggagattgtgatgaaagatggaaggagtatagttgtcccgtgcagaggacaggatgagctgataggcagggctcgcatctctccaggagctggatggaatgtgaaggacacagcttgcctggccaaagcatatgcacagatgtggctactcctatacttccatcgcagggacttgcgtctcatggcaaatgcgatttgctcagcagtgccagtggattgggtgcccacaggcaggacatcctggtcaatacactcgaaaggagagtggatgaccacggaagacatgctgcaggtctggaacagagtctggattgaagaaaatgaatggatgatggacaagactccaatcacaagctggacagacgttccgtatgtgggaaagcgtgaggacatctggtgtggcagcctcatcggaacgcgatccagagcaacctgggctgagaacatctatgcggcgataaaccaggttagagctgtcattgggaaagaaaattatgttgactacatgacctcactcaggagatacgaagacgtcttgatccaggaagacagggtcatctagtgtgatttaaggtagaaaagtagactatgtaaataatgtaaatgagaaaatgcatgcatatggagtcaggccagcaaaagctgccaccggatactgggtagacggtgctgcctgcgtctcagtcccaggaggactgggttaacaaatctgacaacagaaagtgagaaagccctcagaaccgtctcggaagtaggtccctgctcactggaagttgaaagaccaacgtcaggccacaaatttgtgccactccgctagggagtgcggcctgcgcagccccaggaggactgggttaccaaagccgttgagcccccacggcccaagcctcgtctaggatgcaatagacgaggtgtaaggactagaggttagaggagaccccgtggaaacaacaacatgcggcccaagccccctcgaagctgtagaggaggtggaaggactagaggttagaggagaccccgcatttgcatcaaacagcatattgacacctgggaatagactgggagatcttctgctctatctcaacatcagctactaggcacagagcgccgaagtatgtagctggtggtgaggaagaacac

SEQ ID NO: 178 - protein, 3432 aa, Japanese Encephalitis Virus Strain SA-14
MTKKPGGPGKNRAINMLKRGLPRVFPLVGVKRVVMSLLDGRGPVRFVLALITFFKFTALAPTKALLGRWKAVEKSVAMKHLTSFKRELGTLIDAVNKRGRKQNKRGGNEGSIMWLASLAVVIACAGAMKLSNFQGKLLMTINNTDIADVIVIPTSKGENRCWVRAIDVGYMCEDTITYECPKLTMGNDPEDVDCWCDNQEVYVQYGRCTRTRHSKRSRRSVSVQTHGESSLVNKKEAWLDSTKATRYLMKTENWIIRNPGYAFLAAVLGWMLGSNNGQRVVFTILLLLVAPAYSFNCLGMGNRDFIEGASGATWVDLVLEGDSCLTIMANDKPTLDVRMINIEASQLAEVRSYCYHASVTDISTVARCPTTGEAHNEKRADSSYVCKQGFTDRGWGNGCGLFGKGSIDTCAKFSCTSKAIGRTIQPENIKYEVGIFVHGTTTSENHGNYSAQVGASQAAKFTVTPNAPSITLKLGDYGEVTLDCEPRSGLNTEAFYVMTVGSKSFLVHREWFHDLALPWTSPSSTAWRNRELLMEFEGAHATKQSVVALGSQEGGLHQALAGAIVVEYSSSVKLTSGHLKCRLKMDKLALKGTTYGMCTEKFSFAKNPVDTGHGTVVIELSYSGSDGPCKIPIVSVASLNDMTPVGRLVTVNPFVATSSANSKVLVEMEPPFGDSYIVVGRGDKQINHHWHKAGSTLGKAFSTTLKGAQRLAALGDTAWDFGSIGGVFNSIGRAVHQVFGGAFRTLFGGMSWITQGLMGALLLWMGVNARDRSIALAFLATGGVLVFLATNVHADTGCAIDITRKEMRCGSGIFVHNDVEAWVDRYKYLPETPRSLAKIVHKAHKEGVCGVRSVTRLEHQMWEAVRDELNVLLKENAVDLSVVVNKPVGRYRSAPKRLSMTQEKFEMGWKAWGKSILFAPELANSTFVVDGPETKECPDEHRAWNSMQIEDFGFGITSTRVWLKIREESTDECDGAIIGTAVKGHVAVHSDLSYWIESRYNDTWKLERAVFGEVKSCTWPETHTLWGDDVEESELIIPHTIAGPKSKHNRREGYKTQNQGPWDENGIVLDFDYCPGTKVTITEDCSKRGPSVRTTTDSGKLITDWCCRSCSLPPLRFRTENGCWYGMEIRPVMHDETTLVRSQVDAFKGEMVDPFQLGLLVMFLATQEVLRKRWTARLTIPAVLGVLLVLMLGGITYTDLARYVVLVAAAFAEANSGGDVLHLALIAVFKIQPAFLVMNMLSTRWTNQENVILVLGAAFFQLASVDLQIGVHGILNAAAIAWMIVRAITFPTTSSVTMPVLALLTPGMRALYLDTYRIILLVIGICSLLHERKKTMAKKKGAVLLGLALTSTGWFSPTTIAAGLMVCNPNKKRGWPATEFLSAVGLMFAIVGGLAELDIESMSIPFMLAGLMAVSYVVSGKATDMWLERAADISWEMDAAITGSSRRLDVKLDDDGDFHLIDDPGVPWKVWVLRMSCIGLAALTPWAIVPAAFGYWLTLKTTKRGGVFWDTPSPKPCSKGDTTTGVYRIMARGILGTYQAGVGVMYENVFHTLWHTTRGAAIMSGEGKLTPYWGSVREDRIAYGGPWRFDRKWNGTDDVQVIVVEPGKAAVNIQTKPGVFRTPFGEVGAVSLDYPRGTSGSPILDSNGDIIGLYGNGVELGDGSYVSAIVQGDRQEEPVPEAYTPNMLRKRQMTVLDLHPGSGKTRKILPQIIKDAIQQRLRTAVLAPTRVVAAEMAEALRGLPVRYQTSAVQREHQGNEIVDVMCHATLTHRLMSPNRVPNYNLFVMDEAHFTDPASIAARGYIATKVELGEAAAIFMTATPPGTTDPFPDSNAPIHDLQDEIPDRAWSSGYEWITEYAGKTVWFVASVKMGNEIAMCLQRAGKKVIQLNRKSYDTEYPKCKNGDWDFVITTDISEMGANFGASRVIDCRKSVKPTILEEGEGRVILGNPSPITSASAAQRRGRVGRNPNQVGDEYHYGGATSEDDSNLAHWTEAKIMLDNIHMPNGLVAQLYGPEREKAFTMDGEYRLRGEEKKNFLELLRTADLPVWLAYKVASNGIQYTDRKWCFDGPRTNAILEDNTEVEIVTRMGERKILKPRWLDARVYADHQALKWFKDFAAGKRSAVSFIEVLGRMPEHFMGKTREALDTMYLVATAEKGGKAHRMALEELPDALETITLIVAITVMTGGFFLLMMQRKGIGKMGLGALVLTLATFFLWAAEVPGTKIAGTLLIALLLMVVLIPEPEKQRSQTDNQLAVFLICVLTVVGVVAANEYGMLEKTKADLKSMFGGKTQASGLTGLPSMALDLRPATAWALYGGSTVVLTPLLKHLITSEYVTTSLASINSQAGSLFVLPRGVPFTDLDLTVGLVFLGCWGQITLTTFLTAMVLATLHYGYMLPGWQAEALRAAQRRTAAGIMKNAVVDGMVATDVPELERTTPLMQKKVGQVLLIGVSVAAFLVNPNVTTVREAGVLVTAATLTLWDNGASAVWNSTTATGLCHVMRGSYLAGGSIAWTLIKNADKPSLKRGRPGGRTLGEQWKEKLNAMSREEFFKYRREAIIEVDRTEARRARRENNIVGGHPVSRGSAKLRWLVEKGFVSPIGKVIDLGCGRGGWSYYAATLKKVQEVRGYTKGGAGHEEPMLMQSYGWNLVSLKSGVDVFYKPSEPSDTLFCDIGESSPSPEVEEQRTLRVLEMTSDWLHRGPREFCIKVLCPYMPKVIEKMEVLQRRFGGGLVRLPLSRNSNHEMYWVSGAAGNVVHAVNMTSQVLLGRMDRTVWRGPKYEEDVNLGSGTRAVGKGEVHSNQEKIKKRIQKLKEEFATTWHKDPEHPYRTWTYHGSYEVKATGSASSLVNGVVELMSKPWDAIANVTTMAMTDTTPFGQQRVFKEKVDTKAPEPPAGAKEVLNETTNWLWAHLSREKRPRLCTKEEFIKKVNSNAALGAVFAEQNQWSTAREAVDDPRFWEMVDEERENHLRGECHTCIYNMMGKREKKPGEFGKAKGSRAIWFMWLGARYLEFEALGFLNEDHWLSRENSGGGVEGSGVQKLGYILRDIAGKQGGKMYADDTAGWDTRITRTDLENEAKVLELLDGEHRMLARAIIELTYRHKVVKVMRPAAEGKTVMDVISREDQRGSGQVVTYALNTFTNIAVQLVRLMEAEGVIGPQHLEQLPRKTKIAVRTWLFENGEERVTRMAISGDDCVVKPLDDRFATALHFLNAMSKVRKDIQEWKPSHGWHDWQQVPFCSNHFQEIVMKDGRSIVVPCRGQDELIGRARISPGAGWNVKDTACLAKAYAQMWLLLYFHRRDLRLMANAICSAVPVDWVPTGRTSWSIHSKGEWMTTEDMLQVWNRVWIEENEWMMDKTPITSWTDVPYVGKREDIWCGSLIGTRSRATWAENIYAAINQVRAVIGKENYVDYMTSLRRYEDVLIQEDRVI

SEQ ID NO: 179 - DNA, 10690 bp, Dengue-1 virus
acaagaacagtttcgaatcggaagcttgcttaacgtagttctaacagttttttattagagagcagatctctgatgaacaaccaacggaaaaagacgggtcgaccgtctttcaatatgctgaaacgcgcgagaaaccgcgtgtcaactggttcacagttggcgaagagattctcaaaaggattgctttcgggccaaggacccatgaaattggtgatggcttttatagcatttctaagatttctagccatacccccaacagcaggaattttggctagatggagctcattcaagaagaatggagcgatcaaagtgttacggggtttcaaaaaagagatctcaagcatgttgaatattatgaacaggaggaaaaaatctgtgaccatgctcctcatgctgctgcccacagccctggcgttccatttgactacacgagggggagagccacacatgatagttagtaagcaggaaagaggaaagtcactgttgtttaagacctctgcaggcatcaatatgtgcactctcattgcgatggatctgggagagttatgcgaggacacaatgacctacaaatgcccccggatcactgaggcggaaccagatgacgttgactgctggtgcaatgccacagacacatgggtgacctatgggacatgttctcaaaccggcgaacaccgacgagacaaacgttccgtggcactggccccacacgtgggacttggtctagaaacaagaaccgaaacatggatgtcctctgaaggtgcctggaaacaaatacaaaaagtggagacttgggctttgagacacccaggattcacggtgacagccctttttttagcacatgccataggaacatccattactcagaaagggatcattttcattttgctgatgctagtaacaccatcaatggccatgcgatgtgtgggaataggcaacagggacttcgttgaaggactgtcaggagcaacgtgggtggacgtagtattggagcatggaagctgcgtcaccaccatggcaaaagataaaccaacattggacattgaactcttgaagacggaggtcacaaaccctgccgtcttgcgcaaactgtgcattgaagctaaaatatcaaacaccaccaccgattcaaggtgtccaacacaaggagaggccacactggtggaagaacaagactcgaactttgtgtgtcgacgaacgtttgtggacagaggctggggtaatggctgcggactatttggaaaaggaagcctactgacgtgtgctaagttcaagtgtgtgacaaaactagaagggaagatagttcaatatgaaaacttaaaatattcagtgatagtcactgtccacactggggaccagcaccaggtgggaaacgagactacagaacatggaacaattgcaactataacacctcaagctcctacgtcggaaatacagctgactgactacggagccctcacattggactgctcacctagaacagggctggactttaatgagatggttctattgacaatgaaagaaaaatcatggcttgtccacaaacaatggtttctagacttaccactgccttggacttcaggagcttcaacatctcaagagacttggaacagacaagatttgctggtcacattcaagacagctcatgcaaagaaacaggaagtagtcgtactgggatcacaggaaggagcaatgcacactgcgttgactggggcgacagaaatccagacgtcaggaacgacaacgatctttgcaggacacctgaaatgcagactaaaaatggataaactgactttaaaagggatgtcatatgtaatgtgcacaggctcatttaagctagagaaggaagtggctgagacccagcatggaactgttctagtgcaggttaaatacgaaggaacagatgcaccatgcaagatccctttttcggcccaagatgagaaaggagtgacccagaatgggagattgataacagccaaccccatagtcactgacaaagaaaaaccagtcaacattgagacagaaccaccttttggtgagagctacatcgtggtaggggcaggtgaaaaagctttgaaactgagctggttcaagaaagggagcagcatagggaaaatgttcgaagcaactgcccgaggagcacgaaggatggctatcctgggagacaccgcatgggactttggctctataggaggagtgttcacatcagtgggaaaattggtacaccaggtttttggagccgcatatggggttctgttcagcggtgtttcttggaccatgaaaataggaatagggattctgctgacatggttgggattaaattcaaggagcacgtcactttcgatgacgtgcattgcggttggcatggtcacactgtacctaggagtcatggttcaagcggactcgggatgtgtaatcaactggaagggcagagaactcaaatgtggaagtggtatttttgtcactaatgaagtccacacttggacagagcaatacaaattccaggctgactccccaaaaagactgtcagcagccattgggaaggcatgggaagagggcgtgtgtggaattcgatcagccacgcgtcttgagaacatcatgtggaagcaaatatcaaatgaattgaaccacattctacttgaaaatgacatgaaattcacagtggttgtaggagatgctaatggaattttggcccaagggaaaaaaatgattaggccacaacccatggaacatagatactcatggaaaagctggggaaaagccaaaatcataggagcagacatacagaatagcaccttcatcatcgacggcccagatactccagaatgccccgatgaccaaagagcgtggaacatttgggaagttgaagactatgggtttggaattttcacgacaaacatatggctgaaattgcgcgactcctacacccaaatgtgtgaccaccggctaatgtccgctgccatcaaagacagcaaggcagtccatgctgacatggggtactggatagaaagtgaaaagaacgagacctggaagctagcgagagcctccttcatagaagtcaagacatgtatctggccaaaatcccacactctatggagtaatggagttttggaaagtgaaatgataatcccaaagatatatggaggaccaatatctcagcacaactacagaccagggtatttcacacaaacagcagggccatggcacctaggcaagttggaattggatttcgacttgtgtgaaggtaccacagttgttgtggatgaacattgtggaaatcgaggcccatctctcagaactacaacagtcacaggaaagataatccatgaatggtgttgcagatcctgcacgttaccccccttacgcttcagaggagaagacggatgttggtatggcatggaaatcagaccagttaaggagaaggaggaaaacctagttaggtcaatggtctctgcagggtcaggagaagtggacagtttttcactagggatactatgcgtatcaataatgattgaagaagtgatgagatccagatggagtagaaagatgctgatgactggaacattggctgttttcctccttcttataatgggacaactgacatggaatgacctgattaggttatgcatcatggttggagctaatgcttcagacaggatggggatgggaacaacgtacctggccttgatggctactttcaaaatgagaccaatgttcgctgttgggctactatttcgcagactaacatccagagaagttcttcttctcacgattggattaagcctggtggcatccgtggagttaccaaattctttggaggagctaggggatggacttgcaatgggtatcatgatgttaaaattattgactgaatttcagccacaccagttatggaccaccttactgtctttgacatttatcaaaacaactctttcattggattatgcatggaggacaatggctatggtgctgtcaatcgtatctctctttcctttatgtctgtctacgacctcccaaaaaacaacatggcttccggtgctgttaggatcttttggatgcaaaccattaaccatgtttcttataacagaaaacaaaatctggggaaggaaaagttggcccctcaatgaaggaattatggctattggaatagtcagcattctactaagttcactccttaaaaatgatgtgccgttggccggcccactaatagctggaggcatgctaatagcatgttatgtcatatccggaagctcagccgatttatcattggagaaagcggctgaggtatcctgggaagaagaagcagaacactctggtacctcacacaacatattagtagaggtccaagatgatggaactatgaaaataaaagatgaagagagggatgacacactcaccatactcctcaaagcaactctgctggcagtctcaggagtgtacccaatgtcaataccagcaactcttttcgtgtggtatttttggcagaaaaagaaacagagatcaggagtgttatgggacacacccagccctccggaagtggaaagagcagttcttgatgatggcatctatagaatcctgcaaagaggactgttgggcaggtcccaagtgggagtgggagttttccaggacggcgtgttccacacaatgtggcacgtcaccaggggagctgtccttatgtaccaagggaagaggttggaaccaagctgggccagtgtcaaaaaggacttgatctcatatggaggaggttggagatttcaaggatcatggaacacgggagaagaggtgcaggtgatagccgttgaaccaggaaaaaatcctaaaaatgtacagacaacgccgggtaccttcaagactcccgaaggcgaagttggagccatagctctagattttaaacccggcacatctggatctcccatcgtgaacagagagggaaaaatagtaggtctttatggaaatggagtggtgacaacaagtggaacctacgtcagtgccattgcccaagctaaaacatcacaagaagggcctctaccagagattgaggaggaggtgtttaagaaaaggaacttaacaataatggacctgcatccaggatcgggaaaaacaagaagatatcttccagccatagtccgtgaggccataaaaaggaaactgcgcacgttaatcttggctcccacaagagttgtcgcctctgaaatggcagaggcgctcaagggaatgccaataagatatcagacaacagcagtgaagagtgaacacacaggaagggagatagtagacctcatgtgccacgccacttttaccatgcgtctcttatctccagtgagagttcccaattacaacatgattatcatggatgaagcacattttaccgatccagccagcatagcggccagagggtacatctcaactcgagtgggtatgggtgaagcagctgcgatctttatgacagccactcccccaggatcggtggaggcctttccacaaagtaatgcagttatccaagatgaggaaagagacattcctgagagatcatggaactcaggctacgactggatcactgattttccaggtaaaacagtctggtttgttccaagcattaagtcaggaaatgacattgctaactgtttaagaaagaacggaaaacgggtgatccaattgagcagaaaaacctttgacactgagtaccagaaaacaagaaacaatgactgggactatgttgtcacaacagacatttccgaaatgggggcaaatttccgggccgacagggtaatagacccaaggcggtgcttgaaaccagtgatactaaaagatggtccagagcgtgtcattctagccggaccgatgccagtgactgtggccagtgctgcccagagaagaggaagaattggaaggaaccaaaacaaggaaggtgatcagtacgtttatatgggacagcctttaaataatgatgaggatcacgctcattggacagaagcaaaaatgctccttgacaatataaacactccagaagggatcattccagccctctttgagccagagagagaaaagagtgcagcaatagatggagagtacagactgcggggagaagcaaggaaaacgttcgtggagctcatgagaagaggagatctaccagtttggctatcctacaaagttgcctcagaaggtttccagtactccgacagaaggtggtgctttgacggggaaaggaacaaccaggtgttggaggagaacatggacgtggagatctggacaaaggaaggagaaaggaagaaattgcgaccccgctggttggatgccagaacatactctgatccactggcccttcgtgagttcaaagagtttgcagcaggaagaagaagtgtctcaggtgacctaatattggaaatagggaaacttccacaacatttgacgctaagagcccagaatgctctggataacttggtcatgttgcacaattccgaacaaggaggaaaagcctatagacatgctatggaggaactaccagacaccatagaaacattgatgctcctagctttgatagctgtgctgactggtggagtgacgctgttcttcctatcaggaaaaggtctagggaaaacatccattggcctgctctgtgtgatggcctcaagcgcactgttatggatggccaatgtggagccacattggatagcggcctccatcatactagagttctttctgatggtgctgctcattccagagccagacagacagcgcactccacaggacaaccagctagcatacgtggtgataggtttgttattcatgatactgacagtggcagccaatgagatgggattattggaaaccacaaagaaagacctggggattggctatgtagccgccgaaaaccaacaccatgccacgatgctggacgtagacctacgcccagcttcagcctggaccctctatgcagtagccacaacagttatcactcccatgatgagacacacaattgaaaacacaacagcaaacatttccctgacagccattgcaaaccaggcagctatattgatgggacttgacaagggatggccaatatcgaagatggacataggagttccacttctcgccttagggtgctattcccaggtgaacccattgacactgacagcggcggtgttgatgttagtggctcattatgccataattggaccaggactgcaagcaaaggccactagagaagctcaaaaaaggacagcggccggaataatgaaaaatccaaccgtagacgggattgttgcaatagacttggatcctgtggtttatgatgcaaaatttgaaaaacagttaggccaaataatgttactgatactttgtacatcacagattctcttgatgcggaccacatgggccctgtgtgaatccatcacactggctactggacccttgaccactctctgggagggatctccaggaaaattctggaataccacgatagcagtgtccatggcaaacatcttcaggggaagttatctagcaggagcaggtctggccttctcgttaatgaaatctttgggaggaggcaggagaggtacgggggctcaaggagaaacactgggagagaaatggaaaagacagttaaaccaactgagcaagtcagaattcaacacctacaaaaggagtgggattatggaggtggacagatccgaagccaaagagggattaaaaagaggagaaacaaccaaacatgcagtgtcgagaggaacagccaaactgaggtggtttgtggagaggaacctcgtgaaaccagaagggaaagtcatagacctcggttgtggaagaggtggctggtcatattattgtgctgggctgaagaaagtcactgaagtcaagggatacacaaaaggaggacctggacatgaggaacctatcccaatggcgacctatggatggaacctagtaaagctatactctggaaaagatgtattttttacaccacctgagaaatgtgatacccttctgtgtgatataggagagtcctctccgaatccaactatagaagaaggaagaacgttacgtgttctaaagatggtagaaccatggctcagaggaaaccaattctgcataaaaattctaaatccttacatgccaagtgtggtagaaactctggagcaaatgcaaagaaaacatggagggatgctagtgcgaaacccactctcaagaaattccacccatgaaatgtactgggtttcatgtggaacaggaaacattgtgtcggcagtgaacatgacatccagaatgttactgaatcgattcacaatgactcacaggaaaccaacatatgaaagagacgtggacttaggcgctggaacaagacatgtggcagtggaaccagaggtagccaacctagatattattggccagaggatagagaatataaaaaatgaacacaagtcaacatggtattatgatgaagacaatccatacaaaacatgggcctatcatggatcatatgaggtcaaaccatcaggatcagcctcatctatggtgaatggcgtggtgagattgctcacaaaaccatgggatgtcatccccatggtcacacaaatagccatgaccgataccacacccttcggacaacagagagtgtttaaagagaaagtagatacgcgcacgccgagagcaaaacgaggcacagcacaaattatggaggtgacagccaagtggctatggggtttcctttccagaaacaaaaaacccagaatctgcacaagagaggagttcacaagaaaggttaggtcaaacgcagcaataggagcagtgttcgttgatgaaaaccaatggaactcagcaaaagaagcagtggaagacgaaaggttttgggatctcgtgcacagagagagggaacttcataaacagggaaaatgtgccacgtgtgtctacaacatgatggggaagagagagaaaaaattgggagagtttggaaaggcaaaaggaagtcgtgcaatatggtacatgtggctgggagcacgctttctagagttcgaagcccttggtttcatgaatgaagatcactggttcagtagagagaattcattcagtggagtggaaggagaaggactgcacaaacttggatacatactcagagacatatcaaagatcccggggggaaacatgtatgcagatgacacagccggatgggacacaagaataacagaggatgatcttcagaatgaggccaaaatcactgacatcatggagcctgaacacgctctattagctacgtcaatttttaagctgacctaccaaaacaaggtggtaagggtgcaaagaccagcaaaaaatggaaccgtgatggatgttatatccagacgagaccagagaggaagtgggcaggtcggaacctatggcttaaatactttcaccaatatggaggtccaactaataagacaaatggagtctgaaggaatctttttacccagcgaattggaaacccccaatctagctgagagagttcttaactggttggaaaaacatggcactgaaaggctgaaaagaatggcaattagcggagatgattgtgtggtgaaaccagttgatgacaggtttgcaacagccttaatagctctgaatgacatgggaaaagtaagaaaagacataccgcagtgggaaccttcaaaaggatggaatgactggcagcaagtgcctttctgttcacaccatttccaccagctgatcatgaaggatgggagggaaatagtggtgccatgccgcaaccaagatgaacttgtgggcagggctagagtatcacaaggcgccggatggagcctgagagaaactgcttgcctaggcaagtcatatgcacaaatgtggcagctgatgtacttccacaggagagacctaagactagcggctaatgctatctgttcagctgtcccagttgactgggttccaaccagccgcacaacctggtcaatccatgctcaccatcaatggatgacaacagaagatatgttatcagtgtggaatagggtttggatagaggaaaacccatggatggaggataaaactcatgtgtccagctgggaagaagtcccatacttagggaaaagggaagatcaatggtgtggatccctgataggcttgacagcgagggccacttgggccactaacatacaagtggccataaaccaagtgagaaggctcatcggaaatgagaattatctagattacatgacatcaatgaagagattcaagaatgagagtgatcccgaaggggcactctggtaagtcaacacactcatgaaataaaggaaaatggaagatcaaataaagtaagaagtcaggccagattaagccatagtacggaaaaagctatgctgcctgtgagccccgtccaaggacgtaaaatgaagtcaggccgaaagccacggcttgagcaagccgtgctgcctgtggctccatcgtggggatgtaaaaacccgggaggctgcaacccatggaagctgtacgcatggggtagcagactagtggttagaggagacccctcccaagacacaacgcagcagcggggcccaacaccaggggaagctgtaccctggtggtaaggactagaggttagaggagaccccccgcacaacaacaaacagcatattgacgctgggagagaccagagatcctgctgtctctacagcatcattccaggcacagaacgccagaaaatggaatg

SEQ ID NO: 180 - protein, 3392 aa, Dengue-1 virus
MNNQRKKTGRPSFNMLKRARNRVSTGSQLAKRFSKGLLSGQGPMKLVMAFIAFLRFLAIPPTAGILARWSSFKKNGAIKVLRGFKKEISSMLNIMNRRKKSVTMLLMLLPTALAFHLTTRGGEPHMIVSKQERGKSLLFKTSAGINMCTLIAMDLGELCEDTMTYKCPRITEAEPDDVDCWCNATDTWVTYGTCSQTGEHRRDKRSVALAPHVGLGLETRTETWMSSEGAWKQIQKVETWALRHPGFTVTALFLAHAIGTSITQKGIIFILLMLVTPSMAMRCVGIGNRDFVEGLSGATWVDVVLEHGSCVTTMAKDKPTLDIELLKTEVTNPAVLRKLCIEAKISNTTTDSRCPTQGEATLVEEQDSNFVCRRTFVDRGWGNGCGLFGKGSLLTCAKFKCVTKLEGKIVQYENLKYSVIVTVHTGDQHQVGNETTEHGTIATITPQAPTSEIQLTDYGALTLDCSPRTGLDFNEMVLLTMKEKSWLVHKQWFLDLPLPWTSGASTSQETWNRQDLLVTFKTAHAKKQEVVVLGSQEGAMHTALTGATEIQTSGTTTIFAGHLKCRLKMDKLTLKGMSYVMCTGSFKLEKEVAETQHGTVLVQVKYEGTDAPCKIPFSAQDEKGVTQNGRLITANPIVTDKEKPVNIETEPPFGESYIVVGAGEKALKLSWFKKGSSIGKMFEATARGARRMAILGDTAWDFGSIGGVFTSVGKLVHQVFGAAYGVLFSGVSWTMKIGIGILLTWLGLNSRSTSLSMTCIAVGMVTLYLGVMVQADSGCVINWKGRELKCGSGIFVTNEVHTWTEQYKFQADSPKRLSAAIGKAWEEGVCGIRSATRLENIMWKQISNELNHILLENDMKFTVVVGDANGILAQGKKMIRPQPMEHRYSWKSWGKAKIIGADIQNSTFIIDGPDTPECPDDQRAWNIWEVEDYGFGIFTTNIWLKLRDSYTQMCDHRLMSAAIKDSKAVHADMGYWIESEKNETWKLARASFIEVKTCIWPKSHTLWSNGVLESEMIIPKIYGGPISQHNYRPGYFTQTAGPWHLGKLELDFDLCEGTTVVVDEHCGNRGPSLRTTTVTGKIIHEWCCRSCTLPPLRFRGEDGCWYGMEIRPVKEKEENLVRSMVSAGSGEVDSFSLGILCVSIMIEEVMRSRWSRKMLMTGTLAVFLLLIMGQLTWNDLIRLCIMVGANASDRMGMGTTYLALMATFKMRPMFAVGLLFRRLTSREVLLLTIGLSLVASVELPNSLEELGDGLAMGIMMLKLLTEFQPHQLWTTLLSLTFIKTTLSLDYAWRTMAMVLSIVSLFPLCLSTTSQKTTWLPVLLGSFGCKPLTMFLITENKIWGRKSWPLNEGIMAIGIVSILLSSLLKNDVPLAGPLIAGGMLIACYVISGSSADLSLEKAAEVSWEEEAEHSGTSHNILVEVQDDGTMKIKDEERDDTLTILLKATLLAVSGVYPMSIPATLFVWYFWQKKKQRSGVLWDTPSPPEVERAVLDDGIYRILQRGLLGRSQVGVGVFQDGVFHTMWHVTRGAVLMYQGKRLEPSWASVKKDLISYGGGWRFQGSWNTGEEVQVIAVEPGKNPKNVQTTPGTFKTPEGEVGAIALDFKPGTSGSPIVNREGKIVGLYGNGVVTTSGTYVSAIAQAKTSQEGPLPEIEEEVFKKRNLTIMDLHPGSGKTRRYLPAIVREAIKRKLRTLILAPTRVVASEMAEALKGMPIRYQTTAVKSEHTGREIVDLMCHATFTMRLLSPVRVPNYNMIIMDEAHFTDPASIAARGYISTRVGMGEAAAIFMTATPPGSVEAFPQSNAVIQDEERDIPERSWNSGYDWITDFPGKTVWFVPSIKSGNDIANCLRKNGKRVIQLSRKTFDTEYQKTRNNDWDYVVTTDISEMGANFRADRVIDPRRCLKPVILKDGPERVILAGPMPVTVASAAQRRGRIGRNQNKEGDQYVYMGQPLNNDEDHAHWTEAKMLLDNINTPEGIIPALFEPEREKSAAIDGEYRLRGEARKTFVELMRRGDLPVWLSYKVASEGFQYSDRRWCFDGERNNQVLEENMDVEIWTKEGERKKLRPRWLDARTYSDPLALREFKEFAAGRRSVSGDLILEIGKLPQHLTLRAQNALDNLVMLHNSEQGGKAYRHAMEELPDTIETLMLLALIAVLTGGVTLFFLSGKGLGKTSIGLLCVMASSALLWMANVEPHWIAASIILEFFLMVLLIPEPDRQRTPQDNQLAYVVIGLLFMILTVAANEMGLLETTKKDLGIGYVAAENQHHATMLDVDLRPASAWTLYAVATTVITPMMRHTIENTTANISLTAIANQAAILMGLDKGWPISKMDIGVPLLALGCYSQVNPLTLTAAVLMLVAHYAIIGPGLQAKATREAQKRTAAGIMKNPTVDGIVAIDLDPVVYDAKFEKQLGQIMLLILCTSQILLMRTTWALCESITLATGPLTTLWEGSPGKFWNTTIAVSMANIFRGSYLAGAGLAFSLMKSLGGGRRGTGAQGETLGEKWKRQLNQLSKSEFNTYKRSGIMEVDRSEAKEGLKRGETTKHAVSRGTAKLRWFVERNLVKPEGKVIDLGCGRGGWSYYCAGLKKVTEVKGYTKGGPGHEEPIPMATYGWNLVKLYSGKDVFFTPPEKCDTLLCDIGESSPNPTIEEGRTLRVLKMVEPWLRGNQFCIKILNPYMPSVVETLEQMQRKHGGMLVRNPLSRNSTHEMYWVSCGTGNIVSAVNMTSRMLLNRFTMTHRKPTYERDVDLGAGTRHVAVEPEVANLDIIGQRIENIKNEHKSTWYYDEDNPYKTWAYHGSYEVKPSGSASSMVNGVVRLLTKPWDVIPMVTQIAMTDTTPFGQQRVFKEKVDTRTPRAKRGTAQIMEVTAKWLWGFLSRNKKPRICTREEFTRKVRSNAAIGAVFVDENQWNSAKEAVEDERFWDLVHRERELHKQGKCATCVYNMMGKREKKLGEFGKAKGSRAIWYMWLGARFLEFEALGFMNEDHWFSRENSFSGVEGEGLHKLGYILRDISKIPGGNMYADDTAGWDTRITEDDLQNEAKITDIMEPEHALLATSIFKLTYQNKVVRVQRPAKNGTVMDVISRRDQRGSGQVGTYGLNTFTNMEVQLIRQMESEGIFLPSELETPNLAERVLNWLEKHGTERLKRMAISGDDCVVKPVDDRFATALIALNDMGKVRKDIPQWEPSKGWNDWQQVPFCSHHFHQLIMKDGREIVVPCRNQDELVGRARVSQGAGWSLRETACLGKSYAQMWQLMYFHRRDLRLAANAICSAVPVDWVPTSRTTWSIHAHHQWMTTEDMLSVWNRVWIEENPWMEDKTHVSSWEEVPYLGKREDQWCGSLIGLTARATWATNIQVAINQVRRLIGNENYLDYMTSMKRFKNESDPEGALW

SEQ ID NO: 181 - DNA, 10723 bp, Dengue-2 virus
agttgttagtctacgtggaccgacaaagacagattctttgagggagctaagctcaacgtagttctaacagttttttaattagagagcagatctctgatgaataaccaacggaaaaaggcgaaaaacacgcctttcaatatgctgaaacgcgagagaaaccgcgtgtcgactgtgcaacagctgacaaagagattctcacttggaatgctgcagggacgaggaccattaaaactgttcatggccctggtggcgttccttcgtttcctaacaatcccaccaacagcagggatattgaagagatggggaacaattaaaaaatcaaaagctattaatgttttgagagggttcaggaaagagattggaaggatgctgaacatcttgaataggagacgcagatctgcaggcatgatcattatgctgattccaacagtgatggcgttccatttaaccacacgtaacggagaaccacacatgatcgtcagcagacaagagaaagggaaaagtcttctgtttaaaacagaggatggcgtgaacatgtgtaccctcatggccatggaccttggtgaattgtgtgaagacacaatcacgtacaagtgtccccttctcaggcagaatgagccagaagacatagactgttggtgcaactctacgtccacgtgggtaacttatgggacgtgtaccaccatgggagaacatagaagagaaaaaagatcagtggcactcgttccacatgtgggaatgggactggagacacgaactgaaacatggatgtcatcagaaggggcctggaaacatgtccagagaattgaaacttggatcttgagacatccaggcttcaccatgatggcagcaatcctggcatacaccataggaacgacacatttccaaagagccctgattttcatcttactgacagctgtcactccttcaatgacaatgcgttgcataggaatgtcaaatagagactttgtggaaggggtttcaggaggaagctgggttgacatagtcttagaacatggaagctgtgtgacgacgatggcaaaaaacaaaccaacattggattttgaactgataaaaacagaagccaaacagcctgccaccctaaggaagtactgtatagaggcaaagctaaccaacacaacaacagaatctcgctgcccaacacaaggggaacccagcctaaatgaagagcaggacaaaaggttcgtctgcaaacactccatggtagacagaggatggggaaatggatgtggactatttggaaagggaggcattgtgacctgtgctatgttcagatgcaaaaagaacatggaaggaaaagttgtgcaaccagaaaacttggaatacaccattgtgataacacctcactcaggggaagagcatgcagtcggaaatgacacaggaaaacatggcaaggaaatcaaaataacaccacagagttccatcacagaagcagaattgacaggttatggcactgtcacaatggagtgctctccaagaacgggcctcgacttcaatgagatggtgttgctgcagatggaaaataaagcttggctggtgcacaggcaatggttcctagacctgccgttaccatggttgcccggagcggacacacaagggtcaaattggatacagaaagagacattggtcactttcaaaaatccccatgcgaagaaacaggatgttgttgttttaggatcccaagaaggggccatgcacacagcacttacaggggccacagaaatccaaatgtcatcaggaaacttactcttcacaggacatctcaagtgcaggctgagaatggacaagctacagctcaaaggaatgtcatactctatgtgcacaggaaagtttaaagttgtgaaggaaatagcagaaacacaacatggaacaatagttatcagagtgcaatatgaaggggacggctctccatgcaagatcccttttgagataatggatttggaaaaaagacatgtcttaggtcgcctgattacagtcaacccaattgtgacagaaaaagatagcccagtcaacatagaagcagaacctccattcggagacagctacatcatcataggagtagagccgggacaactgaagctcaactggtttaagaaaggaagttctatcggccaaatgtttgagacaacaatgaggggggcgaagagaatggccattttaggtgacacagcctgggattttggatccttgggaggagtgtttacatctataggaaaggctctccaccaagtctttggagcaatctatggagctgccttcagtggggtttcatggactatgaaaatcctcataggagtcattatcacatggataggaatgaattcacgcagcacctcactgtctgtgacactagtattggtgggaattgtgacactgtatttgggagtcatggtgcaggccgatagtggttgcgttgtgagctggaaaaacaaagaactgaaatgtggcagtgggattttcatcacagacaacgtgcacacatggacagaacaatacaagttccaaccagaatccccttcaaaactagcttcagctatccagaaagcccatgaagagggcatttgtggaatccgctcagtaacaagactggagaatctgatgtggaaacaaataacaccagaattgaatcacattctatcagaaaatgaggtgaagttaactattatgacaggagacatcaaaggaatcatgcaggcaggaaaacgatctctgcggcctcagcccactgagctgaagtattcatggaaaacatggggcaaagcaaaaatgctctctacagagtctcataaccagacctttctcattgatggccccgaaacagcagaatgccccaacacaaatagagcttggaattcgttggaagttgaagactatggctttggagtattcaccaccaatatatggctaaaattgaaagaaaaacaggatgtattctgcgactcaaaactcatgtcagcggccataaaagacaacagagccgtccatgccgatatgggttattggatagaaagtgcactcaatgacacatggaagatagagaaagcctctttcattgaagttaaaaactgccactggccaaaatcacacaccctctggagcaatggagtgctagaaagtgagatgataattccaaagaatctcgctggaccagtgtctcaacacaactatagaccaggctaccatacacaaataacaggaccatggcatctaggtaagcttgagatggactttgatttctgtgatggaacaacagtggtagtgactgaggactgcggaaatagaggaccctctttgagaacaaccactgcctctggaaaactcataacagaatggtgctgccgatcttgcacattaccaccgctaagatacagaggtgaggatgggtgctggtacgggatggaaatcagaccattgaaggagaaagaagagaatttggtcaactccttggtcacagctggacatgggcaggtcgacaacttttcactaggagtcttgggaatggcattgttcctggaggaaatgcttaggacccgagtaggaacgaaacatgcaatactactagttgcagtttcttttgtgacattgatcacagggaacatgtcctttagagacctgggaagagtgatggttatggtaggcgccactatgacggatgacataggtatgggcgtgacttatcttgccctactagcagccttcaaagtcagaccaacttttgcagctggactactcttgagaaagctgacctccaaggaattgatgatgactactataggaattgtactcctctcccagagcaccataccagagaccattcttgagttgactgatgcgttagccttaggcatgatggtcctcaaaatggtgagaaatatggaaaagtatcaattggcagtgactatcatggctatcttgtgcgtcccaaacgcagtgatattacaaaacgcatggaaagtgagttgcacaatattggcagtggtgtccgtttccccactgctcttaacatcctcacagcaaaaaacagattggataccattagcattgacgatcaaaggtctcaatccaacagctatttttctaacaaccctctcaagaaccagcaagaaaaggagctggccattaaatgaggctatcatggcagtcgggatggtgagcattttagccagttctctcctaaaaaatgatattcccatgacaggaccattagtggctggagggctcctcactgtgtgctacgtgctcactggacgatcggccgatttggaactggagagagcagccgatgtcaaatgggaagaccaggcagagatatcaggaagcagtccaatcctgtcaataacaatatcagaagatggtagcatgtcgataaaaaatgaagaggaagaacaaacactgaccatactcattagaacaggattgctggtgatctcaggactttttcctgtatcaataccaatcacggcagcagcatggtacctgtgggaagtgaagaaacaacgggccggagtattgtgggatgttccttcacccccacccatgggaaaggctgaactggaagatggagcctatagaattaagcaaaaagggattcttggatattcccagatcggagccggagtttacaaagaaggaacattccatacaatgtggcatgtcacacgtggcgctgttctaatgcataaaggaaagaggattgaaccatcatgggcggacgtcaagaaagacctaatatcatatggaggaggctggaagttagaaggagaatggaaggaaggagaagaagtccaggtattggcactggagcctggaaaaaatccaagagccgtccaaacgaaacctggtcttttcaaaaccaacgccggaacaataggtgctgtatctctggacttttctcctggaacgtcaggatctccaattatcgacaaaaaaggaaaagttgtgggtctttatggtaatggtgttgttacaaggagtggagcatatgtgagtgctatagcccagactgaaaaaagcattgaagacaacccagagatcgaagatgacattttccgaaagagaagactgaccatcatggacctccacccaggagcgggaaagacgaagagataccttccggccatagtcagagaagctataaaacggggtttgagaacattaatcttggcccccactagagttgtggcagctgaaatggaggaagcccttagaggacttccaataagataccagaccccagccatcagagctgagcacaccgggcgggagattgtggacctaatgtgtcatgccacatttaccatgaggctgctatcaccagttagagtgccaaactacaacctgattatcatggacgaagcccatttcacagacccagcaagtatagcagctagaggatacatctcaactcgagtggagatgggtgaggcagctgggatttttatgacagccactcccccgggaagcagagacccatttcctcagagcaatgcaccaatcatagatgaagaaagagaaatccctgaacgttcgtggaattccggacatgaatgggtcacggattttaaagggaagactgtttggttcgttccaagtataaaagcaggaaatgatatagcagcttgcctgaggaaaaatggaaagaaagtgatacaactcagtaggaagacctttgattctgagtatgtcaagactagaaccaatgattgggacttcgtggttacaactgacatttcagaaatgggtgccaatttcaaggctgagagggttatagaccccagacgctgcatgaaaccagtcatactaacagatggtgaagagcgggtgattctggcaggacctatgccagtgacccactctagtgcagcacaaagaagagggagaataggaagaaatccaaaaaatgagaatgaccagtacatatacatgggggaacctctggaaaatgatgaagactgtgcacactggaaagaagctaaaatgctcctagataacatcaacacgccagaaggaatcattcctagcatgttcgaaccagagcgtgaaaaggtggatgccattgatggcgaataccgcttgagaggagaagcaaggaaaacctttgtagacttaatgagaagaggagacctaccagtctggttggcctacagagtggcagctgaaggcatcaactacgcagacagaaggtggtgttttgatggagtcaagaacaaccaaatcctagaagaaaacgtggaagttgaaatctggacaaaagaaggggaaaggaagaaattgaaacccagatggttggatgctaggatctattctgacccactggcgctaaaagaatttaaggaatttgcagccggaagaaagtctctgaccctgaacctaatcacagaaatgggtaggctcccaaccttcatgactcagaaggcaagagacgcactggacaacttagcagtgctgcacacggctgaggcaggtggaagggcgtacaaccatgctctcagtgaactgccggagaccctggagacattgcttttactgacacttctggctacagtcacgggagggatctttttattcttgatgagcggaaggggcatagggaagatgaccctgggaatgtgctgcataatcacggctagcatcctcctatggtacgcacaaatacagccacactggatagcagcttcaataatactggagttttttctcatagttttgcttattccagaacctgaaaaacagagaacaccccaagacaaccaactgacctacgttgtcatagccatcctcacagtggtggccgcaaccatggcaaacgagatgggtttcctagaaaaaacgaagaaagatctcggattgggaagcattgcaacccagcaacccgagagcaacatcctggacatagatctacgtcctgcatcagcatggacgctgtatgccgtggccacaacatttgttacaccaatgttgagacatagcattgaaaattcctcagtgaatgtgtccctaacagctatagccaaccaagccacagtgttaatgggtctcgggaaaggatggccattgtcaaagatggacatcggagttccccttctcgccattggatgctactcacaagtcaaccccataactctcacagcagctcttttcttattggtagcacattatgccatcatagggccaggactccaagcaaaagcaaccagagaagctcagaaaagagcagcggcgggcatcatgaaaaacccaactgtcgatggaataacagtgattgacctagatccaataccttatgatccaaagtttgaaaagcagttgggacaagtaatgctcctagtcctctgcgtgactcaagtattgatgatgaggactacatgggctctgtgtgaggctttaaccttagctaccgggcccatctccacattgtgggaaggaaatccagggaggttttggaacactaccattgcggtgtcaatggctaacatttttagagggagttacttggccggagctggacttctcttttctattatgaagaacacaaccaacacaagaaggggaactggcaacataggagagacgcttggagagaaatggaaaagccgattgaacgcattgggaaaaagtgaattccagatctacaagaaaagtggaatccaggaagtggatagaaccttagcaaaagaaggcattaaaagaggagaaacggaccatcacgctgtgtcgcgaggctcagcaaaactgagatggttcgttgagagaaacatggtcacaccagaagggaaagtagtggacctcggttgtggcagaggaggctggtcatactattgtggaggactaaagaatgtaagagaagtcaaaggcctaacaaaaggaggaccaggacacgaagaacccatccccatgtcaacatatgggtggaatctagtgcgtcttcaaagtggagttgacgttttcttcatcccgccagaaaagtgtgacacattattgtgtgacataggggagtcatcaccaaatcccacagtggaagcaggacgaacactcagagtccttaacttagtagaaaattggttgaacaacaacactcaattttgcataaaggttctcaacccatatatgccctcagtcatagaaaaaatggaagcactacaaaggaaatatggaggagccttagtgaggaatccactctcacgaaactccacacatgagatgtactgggtatccaatgcttccgggaacatagtgtcatcagtgaacatgatttcaaggatgttgatcaacagatttacaatgagatacaagaaagccacttacgagccggatgttgacctcggaagcggaacccgtaacatcgggattgaaagtgagataccaaacctagatataattgggaaaagaatagaaaaaataaagcaagagcatgaaacatcatggcactatgaccaagaccacccatacaaaacgtgggcataccatggtagctatgaaacaaaacagactggatcagcatcatccatggtcaacggagtggtcaggctgctgacaaaaccttgggacgtcgtccccatggtgacacagatggcaatgacagacacgactccatttggacaacagcgcgtttttaaagagaaagtggacacgagaacccaagaaccgaaagaaggcacgaagaaactaatgaaaataacagcagagtggctttggaaagaattagggaagaaaaagacacccaggatgtgcaccagagaagaattcacaagaaaggtgagaagcaatgcagccttgggggccatattcactgatgagaacaagtggaagtcggcacgtgaggctgttgaagatagtaggttttgggagctggttgacaaggaaaggaatctccatcttgaaggaaagtgtgaaacatgtgtgtacaacatgatgggaaaaagagagaagaagctaggggaattcggcaaggcaaaaggcagcagagccatatggtacatgtggcttggagcacgcttcttagagtttgaagccctaggattcttaaatgaagatcactggttctccagagagaactccctgagtggagtggaaggagaagggctgcacaagctaggttacattctaagagacgtgagcaagaaagagggaggagcaatgtatgccgatgacaccgcaggatgggatacaagaatcacactagaagacctaaaaaatgaagaaatggtaacaaaccacatggaaggagaacacaagaaactagccgaggccattttcaaactaacgtaccaaaacaaggtggtgcgtgtgcaaagaccaacaccaagaggcacagtaatggacatcatatcgagaagagaccaaagaggtagtggacaagttggcacctatggactcaatactttcaccaatatggaagcccaactaatcagacagatggagggagaaggagtctttaaaagcattcagcacctaacaatcacagaagaaatcgctgtgcaaaactggttagcaagagtggggcgcgaaaggttatcaagaatggccatcagtggagatgattgtgttgtgaaacctttagatgacaggttcgcaagcgctttaacagctctaaatgacatgggaaagattaggaaagacatacaacaatgggaaccttcaagaggatggaatgattggacacaagtgcccttctgttcacaccatttccatgagttaatcatgaaagacggtcgcgtactcgttgttccatgtagaaaccaagatgaactgattggcagagcccgaatctcccaaggagcagggtggtctttgcgggagacggcctgtttggggaagtcttacgcccaaatgtggagcttgatgtacttccacagacgcgacctcaggctggcggcaaatgctatttgctcggcagtaccatcacattgggttccaacaagtcgaacaacctggtccatacatgctaaacatgaatggatgacaacggaagacatgctgacagtctggaacagggtgtggattcaagaaaacccatggatggaagacaaaactccagtggaatcatgggaggaaatcccatacttggggaaaagagaagaccaatggtgcggctcattgattgggttaacaagcagggccacctgggcaaagaacatccaagcagcaataaatcaagttagatcccttataggcaatgaagaatacacagattacatgccatccatgaaaagattcagaagagaagaggaagaagcaggagttctgtggtagaaagcaaaactaacatgaaacaaggctagaagtcaggtcggattaagccatagtacggaaaaaactatgctacctgtgagccccgtccaaggacgttaaaagaagtcaggccatcataaatgccatagcttgagtaaactatgcagcctgtagctccacctgagaaggtgtaaaaaatccgggaggccacaaaccatggaagctgtacgcatggcgtagtggactagcggttagaggagacccctcccttacaaatcgcagcaacaatgggggcccaaggcgagatgaagctgtagtctcgctggaaggactagaggttagaggagacccccccgaaacaaaaaacagcatattgacgctgggaaagaccagagatcctgctgtctcctcagcatcattccaggcacagaacgccagaaaatggaatggtgctgttgaatcaacaggttct

SEQ ID NO: 182 - protein, 3391 aa, Dengue-2 virus
MNNQRKKAKNTPFNMLKRERNRVSTVQQLTKRFSLGMLQGRGPLKLFMALVAFLRFLTIPPTAGILKRWGTIKKSKAINVLRGFRKEIGRMLNILNRRRRSAGMIIMLIPTVMAFHLTTRNGEPHMIVSRQEKGKSLLFKTEDGVNMCTLMAMDLGELCEDTITYKCPLLRQNEPEDIDCWCNSTSTWVTYGTCTTMGEHRREKRSVALVPHVGMGLETRTETWMSSEGAWKHVQRIETWILRHPGFTMMAAILAYTIGTTHFQRALIFILLTAVTPSMTMRCIGMSNRDFVEGVSGGSWVDIVLEHGSCVTTMAKNKPTLDFELIKTEAKQPATLRKYCIEAKLTNTTTESRCPTQGEPSLNEEQDKRFVCKHSMVDRGWGNGCGLFGKGGIVTCAMFRCKKNMEGKVVQPENLEYTIVITPHSGEEHAVGNDTGKHGKEIKITPQSSITEAELTGYGTVTMECSPRTGLDFNEMVLLQMENKAWLVHRQWFLDLPLPWLPGADTQGSNWIQKETLVTFKNPHAKKQDVVVLGSQEGAMHTALTGATEIQMSSGNLLFTGHLKCRLRMDKLQLKGMSYSMCTGKFKVVKEIAETQHGTIVIRVQYEGDGSPCKIPFEIMDLEKRHVLGRLITVNPIVTEKDSPVNIEAEPPFGDSYIIIGVEPGQLKLNWFKKGSSIGQMFETTMRGAKRMAILGDTAWDFGSLGGVFTSIGKALHQVFGAIYGAAFSGVSWTMKILIGVIITWIGMNSRSTSLSVTLVLVGIVTLYLGVMVQADSGCVVSWKNKELKCGSGIFITDNVHTWTEQYKFQPESPSKLASAIQKAHEEGICGIRSVTRLENLMWKQITPELNHILSENEVKLTIMTGDIKGIMQAGKRSLRPQPTELKYSWKTWGKAKMLSTESHNQTFLIDGPETAECPNTNRAWNSLEVEDYGFGVFTTNIWLKLKEKQDVFCDSKLMSAAIKDNRAVHADMGYWIESALNDTWKIEKASFIEVKNCHWPKSHTLWSNGVLESEMIIPKNLAGPVSQHNYRPGYHTQITGPWHLGKLEMDFDFCDGTTVVVTEDCGNRGPSLRTTTASGKLITEWCCRSCTLPPLRYRGEDGCWYGMEIRPLKEKEENLVNSLVTAGHGQVDNFSLGVLGMALFLEEMLRTRVGTKHAILLVAVSFVTLITGNMSFRDLGRVMVMVGATMTDDIGMGVTYLALLAAFKVRPTFAAGLLLRKLTSKELMMTTIGIVLLSQSTIPETILELTDALALGMMVLKMVRNMEKYQLAVTIMAILCVPNAVILQNAWKVSCTILAVVSVSPLLLTSSQQKTDWIPLALTIKGLNPTAIFLTTLSRTSKKRSWPLNEAIMAVGMVSILASSLLKNDIPMTGPLVAGGLLTVCYVLTGRSADLELERAADVKWEDQAEISGSSPILSITISEDGSMSIKNEEEEQTLTILIRTGLLVISGLFPVSIPITAAAWYLWEVKKQRAGVLWDVPSPPPMGKAELEDGAYRIKQKGILGYSQIGAGVYKEGTFHTMWHVTRGAVLMHKGKRIEPSWADVKKDLISYGGGWKLEGEWKEGEEVQVLALEPGKNPRAVQTKPGLFKTNAGTIGAVSLDFSPGTSGSPIIDKKGKVVGLYGNGVVTRSGAYVSAIAQTEKSIEDNPEIEDDIFRKRRLTIMDLHPGAGKTKRYLPAIVREAIKRGLRTLILAPTRVVAAEMEEALRGLPIRYQTPAIRAEHTGREIVDLMCHATFTMRLLSPVRVPNYNLIIMDEAHFTDPASIAARGYISTRVEMGEAAGIFMTATPPGSRDPFPQSNAPIIDEEREIPERSWNSGHEWVTDFKGKTVWFVPSIKAGNDIAACLRKNGKKVIQLSRKTFDSEYVKTRTNDWDFVVTTDISEMGANFKAERVIDPRRCMKPVILTDGEERVILAGPMPVTHSSAAQRRGRIGRNPKNENDQYIYMGEPLENDEDCAHWKEAKMLLDNINTPEGIIPSMFEPEREKVDAIDGEYRLRGEARKTFVDLMRRGDLPVWLAYRVAAEGINYADRRWCFDGVKNNQILEENVEVEIWTKEGERKKLKPRWLDARIYSDPLALKEFKEFAAGRKSLTLNLITEMGRLPTFMTQKARDALDNLAVLHTAEAGGRAYNHALSELPETLETLLLLTLLATVTGGIFLFLMSGRGIGKMTLGMCCIITASILLWYAQIQPHWIAASIILEFFLIVLLIPEPEKQRTPQDNQLTYVVIAILTVVAATMANEMGFLEKTKKDLGLGSIATQQPESNILDIDLRPASAWTLYAVATTFVTPMLRHSIENSSVNVSLTAIANQATVLMGLGKGWPLSKMDIGVPLLAIGCYSQVNPITLTAALFLLVAHYAIIGPGLQAKATREAQKRAAAGIMKNPTVDGITVIDLDPIPYDPKFEKQLGQVMLLVLCVTQVLMMRTTWALCEALTLATGPISTLWEGNPGRFWNTTIAVSMANIFRGSYLAGAGLLFSIMKNTTNTRRGTGNIGETLGEKWKSRLNALGKSEFQIYKKSGIQEVDRTLAKEGIKRGETDHHAVSRGSAKLRWFVERNMVTPEGKVVDLGCGRGGWSYYCGGLKNVREVKGLTKGGPGHEEPIPMSTYGWNLVRLQSGVDVFFIPPEKCDTLLCDIGESSPNPTVEAGRTLRVLNLVENWLNNNTQFCIKVLNPYMPSVIEKMEALQRKYGGALVRNPLSRNSTHEMYWVSNASGNIVSSVNMISRMLINRFTMRYKKATYEPDVDLGSGTRNIGIESEIPNLDIIGKRIEKIKQEHETSWHYDQDHPYKTWAYHGSYETKQTGSASSMVNGVVRLLTKPWDVVPMVTQMAMTDTTPFGQQRVFKEKVDTRTQEPKEGTKKLMKITAEWLWKELGKKKTPRMCTREEFTRKVRSNAALGAIFTDENKWKSAREAVEDSRFWELVDKERNLHLEGKCETCVYNMMGKREKKLGEFGKAKGSRAIWYMWLGARFLEFEALGFLNEDHWFSRENSLSGVEGEGLHKLGYILRDVSKKEGGAMYADDTAGWDTRITLEDLKNEEMVTNHMEGEHKKLAEAIFKLTYQNKVVRVQRPTPRGTVMDIISRRDQRGSGQVGTYGLNTFTNMEAQLIRQMEGEGVFKSIQHLTITEEIAVQNWLARVGRERLSRMAISGDDCVVKPLDDRFASALTALNDMGKIRKDIQQWEPSRGWNDWTQVPFCSHHFHELIMKDGRVLVVPCRNQDELIGRARISQGAGWSLRETACLGKSYAQMWSLMYFHRRDLRLAANAICSAVPSHWVPTSRTTWSIHAKHEWMTTEDMLTVWNRVWIQENPWMEDKTPVESWEEIPYLGKREDQWCGSLIGLTSRATWAKNIQAAINQVRSLIGNEEYTDYMPSMKRFRREEEEAGVLW

SEQ ID NO: 183 - DNA, 10707 bp, Dengue-3 virus
agttgttagtctacgtggaccgacaagaacagtttcgactcggaagcttgcttaacgtagtgctgacagttttttattagagagcagatctctgatgaacaaccaacggaagaagacgggaaaaccgtctatcaatatgctgaaacgcgtgagaaaccgtgtgtcaactggatcacagttggcgaagagattctcaaaaggactgctgaacggccagggaccaatgaaattggttatggcgttcatagctttcctcagatttctagccattccaccaacagcaggagtcttggctagatggggaaccttcaagaagtcgggggccattaaggtcctgaaaggcttcaagaaggagatctcaaacatgctgagcataatcaaccaacggaaaaagacatcgctctgtctcatgatgatattgccagcagcacttgctttccacttgacttcacgagatggagagccgcgcatgattgtggggaagaatgaaagaggtaaatccctactttttaagacagcctctggaatcaacatgtgcacactcatagccatggatttgggagagatgtgtgatgacacggtcacttacaaatgcccccacattaccgaagtggaacctgaagacattgactgctggtgcaaccttacatcaacatgggtgacttatggaacgtgcaatcaagctggagagcatagacgcgacaagagatcagtggcgttagctccccatgtcggcatgggactggacacacgcacccaaacctggatgtcggctgaaggagcttggagacaagtcgagaaggtagagacatgggcccttaggcacccagggttcaccatactagccctatttctcgcccattacataggcacttccctgacccagaaggtggttattttcatattattaatgctggtcaccccatccatgacaatgagatgtgtgggagtaggaaacagagattttgtggaagggctatcaggagctacgtgggttgacgtggtgctcgagcacggggggtgtgtgactaccatggctaagaacaagcccacgctggatatagagcttcagaagaccgaggccacccaactggcgaccctaaggaagctatgcattgaggggaaaattaccaacataacaactgactcaagatgtcctacccaaggggaagcggttttgcctgaggagcaggaccagaactacgtgtgtaagcatacatacgtagacagaggttgggggaacggttgtggtttgtttggcaaaggaagcttggtaacatgtgcgaaatttcaatgcctggaaccaatagagggaaaagtggtgcaatatgagaacctcaaatacaccgtcatcattacagtgcacacaggagaccaacaccaggtgggaaatgaaacgcaaggagtcacggctgagataacacctcaggcatcaaccactgaagccatcttgcctgaatatggaacccttgggctagaatgctcaccacggacaggtttggatttcaatgaaatgatcttactaacaatgaagaacaaagcatggatggtacatagacaatggttctttgacctacctctaccatgggcatcaggagctacaacagaaacaccaacctggaacaggaaggagcttcttgtgacattcaaaaacgcacatgcgaaaaaacaagaagtagttgtccttggatcgcaagagggagcaatgcataccgcactgacaggagctacagaaatccaaaactcaggaggcacaagcattttcgcggggcacttaaaatgtagacttaagatggacaaattggaactcaaggggatgagctatgcaatgtgcacgaatacctttgtgttgaagaaagaagtctcagaaacgcagcacgggacaatactcattaaggttgagtacaaaggggaagatgcaccttgcaagattcccttttccacagaggatggacaagggaaagctcataatggcagactgatcacagccaaccctgtggtgactaagaaggaggagcctgtcaatattgaggctgaacctccttttggggaaagcaatatagtaattggaattggagacaacgccttgaaaatcaactggtacaagaaggggagctcgattgggaagatgttcgaggccactgaaaggggtgcaaggcgcatggccatcttgggagacacagcttgggactttggatcagtgggtggtgttctgaactcattaggcaaaatggtgcaccaaatatttggaagtgcttatacagccctgttcagtggagtctcttgggtgatgaaaattggaataggtgtcctcttgacttggatagggttgaattcaaaaaacacatccatgtcattttcatgcattgcgataggaatcattacactctatctgggagctgtggtacaagctgacatggggtgtgtcataaactggaagggcaaagaactcaaatgtggaagcggaattttcgtcaccaatgaggtccatacctggacagagcaatacaaattccaagcagactccccaaaaagattggcaacagccattgcaggcgcctgggagaatggagtgtgtggaattaggtcaacaaccagaatggagaatctcttgtggaagcaaatagccaatgaactgaactacatattatgggaaaacaatatcaaattaacggtagttgtgggcgatacacttggggtcttagagcaagggaaaagaacactaacaccacaacccatggagctaaaatactcatggaaaacgtggggaaaggcaaaaatagtgacagctgaaacacaaaattcctctttcataatagacgggccaaacacaccggagtgtccaagtgcctcaagagcatggaatgtgtgggaggtggaagattacgggttcggagtcttcacaaccaacatatggctgaaactccgagaggtctacacccaactatgtgaccataggctaatgtcggcagctgtcaaggatgagagggccgtgcatgccgacatgggctactggatagaaagccaaaagaatggaagttggaagctagaaaaagcatccctcatagaggtaaaaacctgcacatggccaaaatcacacactctctggactaatggtgtgctagagagtgacatgatcatcccaaagagtctagctggtcctatctcacaacacaactacaggcccgggtaccacacccaaacggcaggaccctggcacttaggaaaattggagctggacttcaactactgtgaaggaacaacagttgtcatcacagaaagctgtgggacaagaggcccatcattgagaacaacaacagtgtcagggaagttgatacacgaatggtgttgccgctcgtgcacacttccccccctgcgatacatgggagaagacggctgctggtatggcatggaaatcagacccatcagtgagaaagaagagaacatggtaaagtctttagtctcagcgggaagtggaaaggtggacaacttcacaatgggtgtcttgtgtttggcaatcctctttgaagaggtgttgagaggaaaatttgggaagaaacacatgattgcaggggttttctttacgtttgtgctccttctctcagggcaaataacatggagagacatggcgcacacactaataatgatcgggtccaacgcctctgacaggatgggaatgggcgtcacctacctagctctaattgcaacatttaaaatccagccattcttggctttgggatttttcctaagaaagctgacatctagagaaaatttattgttaggagttgggttggccatggcaacaacgttacaactgccagaggacattgaacaaatggcaaatggagtcgctctggggctcatggctcttaaactgataacacaatttgaaacataccaattgtggacggcattagtctccttaacgtgttcaaacacaatttttacgttgactgttgcctggagaacagccactctgattttggccggagtttcgcttttaccagtgtgccagtcttcaagcatgaggaaaacagattggctcccaatgacagtggcagctatgggagttccaccccttccactttttatttttagcttgaaagacacactcaaaaggagaagctggccactgaatgaaggggtgatggctgttgggcttgtgagcattctggccagttctctccttagaaatgatgtgcccatggctggaccattagtggccgggggcttgctgatagcgtgctacgtcataactggcacgtcagcggacctcactgtagaaaaagccccagatgtaacatgggaggaagaggctgagcagacaggagtgtcccacaacttaatgatcacagttgatgatgatggaacaatgagaataaaagatgatgagactgagaacatcctaacagtgcttttaaaaacagcattactaatagtatcaggcatttttccatactccatacccgcaacattgttggtctggcacacttggcaaaaacaaacccaaagatccggcgttttatgggacgtacccagccccccagagacacagaaagcagaactggaagaaggggtttataggatcaaacagcaaggaatttttgggaaaacccaagtaggggttggagtacagaaagaaggagtcttccacaccatgtggcacgtcacaagaggggcagtgttgacacataatgggaaaagactggaaccaaactgggctagtgtgaaaaaagatctgatttcatatggaggaggatggagactgagcgcacaatggcaaaagggggaggaggtgcaggttattgccgtagagccagggaagaacccaaagaactttcaaaccacgccaggcactttccagactactacaggggaaataggagcaattgcactggatttcaagcctggaacttcaggatctcctatcataaatagagagggaaaggtagtgggactgtatggcaatggagtggttacaaagaatggtggctatgtcagcggaatagcgcaaacaaatgcagaaccagatggaccgacaccagagttggaagaagagatgttcaaaaagcgaaacctgaccataatggatcttcatcctgggtcaggaaagacacggaaataccttccagctattgtcagagaggcaatcaagagacgtttaagaaccttaattttggcaccgacaagggtggttgcagctgagatggaagaagcattgaaagggctcccaataaggtaccaaacaacagcaacaaaatctgaacacacaggaagagagattgttgatctaatgtgccacgcaacgttcacaatgcgtttgctgtcaccagttagggttccaaattacaacttgataataatggatgaggcccatttcacagacccagccagtatagcggctagagggtacatatcaactcgtgttggaatgggagaggcagccgcaatcttcatgacagcaacaccccctggaacagctgatgcctttcctcagagcaacgctccaattcaagatgaagaaagggacataccagaacgctcatggaattcaggcaatgaatggattaccgacttcgctgggaaaacggtgtggtttgtccctagcattaaagccggaaatgacatagcaaactgcttgcgaaaaaacgggaaaaaagtcattcaacttagtaggaagacttttgacacagaatatcagaagactaaactgaatgattgggactttgtggtgacaactgacatttcagaaatgggggccaatttcaaagcagatagagtgatcgacccaagaagatgtctcaaaccagtgatcttgacagatggaccagagcgggtgatcctggccggaccaatgccagtcaccgcggcgagtgctgcgcaaaggagagggagagttggcaggaacccacaaaaagagaatgaccagtacatattcacgggccagcctctcaacaatgatgaagaccatgctcactggacagaagcaaaaatgctgctggacaacatcaacacaccagaagggattataccagctctctttgaaccagaaagggagaagtcagccgccatagacggtgagtatcgcctgaagggtgagtccaggaagactttcgtggaactcatgaggaggggtgaccttccagtttggttagcccataaagtagcatcagaaggaatcaaatacacagatagaaaatggtgctttgatgggcaacgcaataatcaaattttagaggagaacatggatgtggaaatttggacaaaggaaggagaaaagaaaaaattgagacctaggtggcttgatgcccgcacttattcagatccattggcactcaaggaattcaaggactttgcggctggcagaaagtcaatcgcccttgatcttgtgacagaaataggaagagtgccttcacatctagcccacagaacaagaaacgctctggacaatctggtgatgctgcatacgtcagaagatggcggtagggcttacaggcatgcggtggaggaactaccagaaacaatggaaacactcctactcttgggactaatgatcttgttgacaggtggagcaatgcttttcttgatatcaggtaaagggattggaaagacttcaataggactcatttgtgtaatcgcttccagcggcatgttgtggatggccgaagttccactccaatggatcgcgtcggctatagtcctggagttttttatgatggtgttgctcataccagaaccagaaaagcagagaaccccccaagacaaccaactcgcatatgtcgtgataggcatacttacattggctgcaacaatagcagccaatgaaatgggactgctggaaaccacaaagagagacttaggaatgtctaaggagccaggtgttgtttctccaaccagctatttggatgtggacttgcacccagcatcagcctggacattgtacgccgtggccactacagtaataacaccaatgttaagacataccatagagaattctacagcaaatgtgtccctggcagctatagccaaccaggcagtggtcctgatgggtttggacaaaggatggccaatatcaaaaatggacttaggcgtgccactactggcactgggttgctattcacaagtgaacccactgactctaactgcggcagtacttttgctaatcacacattatgctatcataggtccaggattgcaagcaaaagccacccgtgaagctcagaaaaggacagctgctggaataatgaagaatccaacagtggatgggataatgacaatagacctagattctgtaatatttgattcaaaatttgaaaaacaactgggacaggttatgctcctggttttgtgcgcagtccaactcttgctaatgagaacatcatgggccttgtgtgaagctttaactctagctacaggaccaataacaacactctgggaaggatcacctggtaagttctggaacaccacgatagctgtttccatggcgaacatttttagagggagctatttagcaggagctgggcttgctttttctattatgaaatcagttggaacaggaaaaagaggaacaggctcacaaggtgaaactttaggagaaaaatggaaaaagaaattaaatcaattatcccggaaagagtttgacctttacaagaaatctggaatcactgaagtggatagaacagaagccaaagaagggttgaaaagaggagagacaacacatcatgccgtgtcccgaggtagcgcaaaacttcaatggtttgtggaaagaaacatggtcgttcccgaaggaagagtcatagacttgggctgtggaagaggaggctggtcatattactgtgcaggactgaaaaaagtcacagaagtgcgaggatacacaaaaggcggtccaggacacgaagaaccagtacctatgtctacatatggatggaacatagttaagttaatgagcggaaaggatgtgttctatctcccacctgaaaagtgtgataccctgttgtgtgacattggagaatcttcaccaagcccaacagtggaagagagcagaactataagagttttgaagatggttgaaccatggctaaaaaacaaccagttttgcattaaagttttgaacccttacatgccaactgtgattgagcacctagaaagactacaaaggaaacatggaggaatgcttgtgagaaatccactttcacgaaactccacgcacgaaatgtactggatatctaatggcacaggtaacattgtctcttcagtcaacatggtgtctagattgctactgaacaggttcacgatgacacacaggagacccaccatagagaaagatgtggatttaggagcaggaactcgacatgttaatgcggaaccagaaacacccaacatggatgtcattggggaaagaataaaaaggatcaaggaggagcataattcaacatggcactatgatgacgaaaacccctacaaaacgtgggcttaccatggatcctatgaagtcaaagccacaggctcagcctcctccatgataaatggagtcgtgaaactcctcaccaaaccatgggatgtggtgcccatggtgacacagatggcaatgacagacacaactccatttggccagcagagagtctttaaagagaaagtggacaccaggacgcccaggcccatgccagggacaagaaaggctatggagatcacagcggagtggctctggagaaccctgggaaggaacaaaagacccagattatgcacaagggaagagtttacaaaaaaggtcagaactaacgcagccatgggcgccgttttcacagaggagaaccaatgggacagtgcgaaagctgctgttgaggatgaagaattttggaaacttgtggacagagaacgtgaactccacaaattgggcaaatgtggaagctgcgtttataacatgatgggcaagagagagaaaaaacttggagagtttggcaaagcaaaaggcagtagagctatatggtacatgtggttgggagccaggtaccttgagttcgaagcccttggattcttaaatgaagaccactggttctcgcgtgaaaactcttacagtggagtagaaggagaaggactgcacaagctaggctacatattaagggacatttccaagatacccggaggagccatgtatgctgatgacacagctggttgggacacaagaataacagaagatgacctgcacaatgaggaaaagatcatacagcaaatggaccctgaacacaggcagttagcgaacgctatattcaagctcacataccaaaacaaagtggtcaaagttcaacgaccgactccaacgggcacggtaatggatattatatctaggaaagaccaaaggggcagtggacaactgggaacttatggcctgaatacattcaccaacatggaagcccagttagtcagacaaatggaaggagaaggtgtgctgacaaaggcagacctcgagaaccctcatctgctagagaagaaaatcacacaatggttggaaaccaaaggagtggagaggttaaaaagaatggccattagcggggatgattgcgtggtgaaaccaatcgatgacaggttcgctaatgccctgcttgctttgaacgatatgggaaaggttcggaaagacatacctcaatggcagccatcaaagggatggcatgattggcaacaggttcctttctgctcccaccactttcatgaattgatcatgaaagatggaagaaagttggtggttccctgcagaccccaggacgaactaataggaagagcaagaatctctcaaggagcgggatggagccttagagaaactgcatgtctggggaaagcctacgcccaaatgtggagtctcatgtattttcacagaagagatctcagattagcatccaacgccatatgttcagcagtaccagtccactgggttcccacaagtagaacgacatggtctattcatgctcaccatcagtggatgactacagaagacatgcttactgtttggaacagggtgtggatagaggaaaatccatggatggaagacaaaactccagttacaacttgggaaaatgttccatatctaggaaagagagaagaccaatggtgtggatcacttattggtctcacttccagagcaacctgggcccagaacatacccacagcaattcaacaggtgagaagccttataggcaatgaagagttcctggactacatgccttcaatgaagagattcaggaaggaagaggagtcggagggagccatttggtaaacgtaggaagtggaaaagaggctaactgtcaggccaccttaagccacagtacggaagaagctgtgctgcctgtgagccccgtccaaggacgttaaaagaagaagtcaggccccaaagccacggtttgagcaaaccgtgctgcctgtagctccgtcgtggggacgtaaaacctgggaggctgcaaactgtggaagctgtacgcacggtgtagcagactagcggttagaggagacccctcccatgacacaacgcagcagcggggcccgagcactgagggaagctgtacctccttgcaaaggactagaggttagaggagaccccccgcaaataaaaacagcatattgacgctgggagagaccagagatcctgctgtctcctcagcatcattccaggcacagaacgccagaaaatggaatggtgctgttgaatcaacaggttct

SEQ ID NO: 184 - protein, 3390 aa, Dengue-3 virus
MNNQRKKTGKPSINMLKRVRNRVSTGSQLAKRFSKGLLNGQGPMKLVMAFIAFLRFLAIPPTAGVLARWGTFKKSGAIKVLKGFKKEISNMLSIINQRKKTSLCLMMILPAALAFHLTSRDGEPRMIVGKNERGKSLLFKTASGINMCTLIAMDLGEMCDDTVTYKCPHITEVEPEDIDCWCNLTSTWVTYGTCNQAGEHRRDKRSVALAPHVGMGLDTRTQTWMSAEGAWRQVEKVETWALRHPGFTILALFLAHYIGTSLTQKVVIFILLMLVTPSMTMRCVGVGNRDFVEGLSGATWVDVVLEHGGCVTTMAKNKPTLDIELQKTEATQLATLRKLCIEGKITNITTDSRCPTQGEAVLPEEQDQNYVCKHTYVDRGWGNGCGLFGKGSLVTCAKFQCLEPIEGKVVQYENLKYTVIITVHTGDQHQVGNETQGVTAEITPQASTTEAILPEYGTLGLECSPRTGLDFNEMILLTMKNKAWMVHRQWFFDLPLPWASGATTETPTWNRKELLVTFKNAHAKKQEVVVLGSQEGAMHTALTGATEIQNSGGTSIFAGHLKCRLKMDKLELKGMSYAMCTNTFVLKKEVSETQHGTILIKVEYKGEDAPCKIPFSTEDGQGKAHNGRLITANPVVTKKEEPVNIEAEPPFGESNIVIGIGDNALKINWYKKGSSIGKMFEATERGARRMAILGDTAWDFGSVGGVLNSLGKMVHQIFGSAYTALFSGVSWVMKIGIGVLLTWIGLNSKNTSMSFSCIAIGIITLYLGAVVQADMGCVINWKGKELKCGSGIFVTNEVHTWTEQYKFQADSPKRLATAIAGAWENGVCGIRSTTRMENLLWKQIANELNYILWENNIKLTVVVGDTLGVLEQGKRTLTPQPMELKYSWKTWGKAKIVTAETQNSSFIIDGPNTPECPSASRAWNVWEVEDYGFGVFTTNIWLKLREVYTQLCDHRLMSAAVKDERAVHADMGYWIESQKNGSWKLEKASLIEVKTCTWPKSHTLWTNGVLESDMIIPKSLAGPISQHNYRPGYHTQTAGPWHLGKLELDFNYCEGTTVVITESCGTRGPSLRTTTVSGKLIHEWCCRSCTLPPLRYMGEDGCWYGMEIRPISEKEENMVKSLVSAGSGKVDNFTMGVLCLAILFEEVLRGKFGKKHMIAGVFFTFVLLLSGQITWRDMAHTLIMIGSNASDRMGMGVTYLALIATFKIQPFLALGFFLRKLTSRENLLLGVGLAMATTLQLPEDIEQMANGVALGLMALKLITQFETYQLWTALVSLTCSNTIFTLTVAWRTATLILAGVSLLPVCQSSSMRKTDWLPMTVAAMGVPPLPLFIFSLKDTLKRRSWPLNEGVMAVGLVSILASSLLRNDVPMAGPLVAGGLLIACYVITGTSADLTVEKAPDVTWEEEAEQTGVSHNLMITVDDDGTMRIKDDETENILTVLLKTALLIVSGIFPYSIPATLLVWHTWQKQTQRSGVLWDVPSPPETQKAELEEGVYRIKQQGIFGKTQVGVGVQKEGVFHTMWHVTRGAVLTHNGKRLEPNWASVKKDLISYGGGWRLSAQWQKGEEVQVIAVEPGKNPKNFQTTPGTFQTTTGEIGAIALDFKPGTSGSPIINREGKVVGLYGNGVVTKNGGYVSGIAQTNAEPDGPTPELEEEMFKKRNLTIMDLHPGSGKTRKYLPAIVREAIKRRLRTLILAPTRVVAAEMEEALKGLPIRYQTTATKSEHTGREIVDLMCHATFTMRLLSPVRVPNYNLIIMDEAHFTDPASIAARGYISTRVGMGEAAAIFMTATPPGTADAFPQSNAPIQDEERDIPERSWNSGNEWITDFAGKTVWFVPSIKAGNDIANCLRKNGKKVIQLSRKTFDTEYQKTKLNDWDFVVTTDISEMGANFKADRVIDPRRCLKPVILTDGPERVILAGPMPVTAASAAQRRGRVGRNPQKENDQYIFTGQPLNNDEDHAHWTEAKMLLDNINTPEGIIPALFEPEREKSAAIDGEYRLKGESRKTFVELMRRGDLPVWLAHKVASEGIKYTDRKWCFDGQRNNQILEENMDVEIWTKEGEKKKLRPRWLDARTYSDPLALKEFKDFAAGRKSIALDLVTEIGRVPSHLAHRTRNALDNLVMLHTSEDGGRAYRHAVEELPETMETLLLLGLMILLTGGAMLFLISGKGIGKTSIGLICVIASSGMLWMAEVPLQWIASAIVLEFFMMVLLIPEPEKQRTPQDNQLAYVVIGILTLAATIAANEMGLLETTKRDLGMSKEPGVVSPTSYLDVDLHPASAWTLYAVATTVITPMLRHTIENSTANVSLAAIANQAVVLMGLDKGWPISKMDLGVPLLALGCYSQVNPLTLTAAVLLLITHYAIIGPGLQAKATREAQKRTAAGIMKNPTVDGIMTIDLDSVIFDSKFEKQLGQVMLLVLCAVQLLLMRTSWALCEALTLATGPITTLWEGSPGKFWNTTIAVSMANIFRGSYLAGAGLAFSIMKSVGTGKRGTGSQGETLGEKWKKKLNQLSRKEFDLYKKSGITEVDRTEAKEGLKRGETTHHAVSRGSAKLQWFVERNMVVPEGRVIDLGCGRGGWSYYCAGLKKVTEVRGYTKGGPGHEEPVPMSTYGWNIVKLMSGKDVFYLPPEKCDTLLCDIGESSPSPTVEESRTIRVLKMVEPWLKNNQFCIKVLNPYMPTVIEHLERLQRKHGGMLVRNPLSRNSTHEMYWISNGTGNIVSSVNMVSRLLLNRFTMTHRRPTIEKDVDLGAGTRHVNAEPETPNMDVIGERIKRIKEEHNSTWHYDDENPYKTWAYHGSYEVKATGSASSMINGVVKLLTKPWDVVPMVTQMAMTDTTPFGQQRVFKEKVDTRTPRPMPGTRKAMEITAEWLWRTLGRNKRPRLCTREEFTKKVRTNAAMGAVFTEENQWDSAKAAVEDEEFWKLVDRERELHKLGKCGSCVYNMMGKREKKLGEFGKAKGSRAIWYMWLGARYLEFEALGFLNEDHWFSRENSYSGVEGEGLHKLGYILRDISKIPGGAMYADDTAGWDTRITEDDLHNEEKIIQQMDPEHRQLANAIFKLTYQNKVVKVQRPTPTGTVMDIISRKDQRGSGQLGTYGLNTFTNMEAQLVRQMEGEGVLTKADLENPHLLEKKITQWLETKGVERLKRMAISGDDCVVKPIDDRFANALLALNDMGKVRKDIPQWQPSKGWHDWQQVPFCSHHFHELIMKDGRKLVVPCRPQDELIGRARISQGAGWSLRETACLGKAYAQMWSLMYFHRRDLRLASNAICSAVPVHWVPTSRTTWSIHAHHQWMTTEDMLTVWNRVWIEENPWMEDKTPVTTWENVPYLGKREDQWCGSLIGLTSRATWAQNIPTAIQQVRSLIGNEEFLDYMPSMKRFRKEEESEGAIW

SEQ ID NO: 185 - DNA, 10649 bp, Dengue-4 virus
agttgttagtctgtgtggaccgacaaggacagttccaaatcggaagcttgcttaacacagttctaacagtttgtttgaatagagagcagatctctggaaaaatgaaccaacgaaaaaaggtggttagaccacctttcaatatgctgaaacgcgagagaaaccgcgtatcaacccctcaagggttggtgaagagattctcaaccggacttttttctgggaaaggacccttacggatggtgctagcattcatcacgtttttgcgagtcctttccatcccaccaacagcagggattctgaagagatggggacagttgaagaaaaataaggccatcaagatactgattggattcaggaaggagataggccgcatgctgaacatcttgaacgggagaaaaaggtcaacgataacattgctgtgcttgattcccaccgtaatggcgttttccctcagcacaagagatggcgaacccctcatgatagtggcaaaacatgaaagggggagacctctcttgtttaagacaacagaggggatcaacaaatgcactctcattgccatggacttgggtgaaatgtgtgaggacactgtcacgtataaatgccccctactggtcaataccgaacctgaagacattgattgctggtgcaacctcacgtctacctgggtcatgtatgggacatgcacccagagcggagaacggagacgagagaagcgctcagtagctttaacaccacattcaggaatgggattggaaacaagagctgagacatggatgtcatcggaaggggcttggaagcatgctcagagagtagagagctggatactcagaaacccaggattcgcgctcttggcaggatttatggcttatatgattgggcaaacaggaatccagcgaactgtcttctttgtcctaatgatgctggtcgccccatcctacggaatgcgatgcgtaggagtaggaaacagagactttgtggaaggagtctcaggtggagcatgggtcgacctggtgctagaacatggaggatgcgtcacaaccatggcccagggaaaaccaaccttggattttgaactgactaagacaacagccaaggaagtggctctgttaagaacctattgcattgaagcctcaatatcaaacataactacggcaacaagatgtccaacgcaaggagagccttatctgaaagaggaacaggaccaacagtacatttgccggagagatgtggtagacagagggtggggcaatggctgtggcttgtttggaaaaggaggagttgtgacatgtgcgaagttttcatgttcggggaagataacaggcaatttggtccaaattgagaaccttgaatacacagtggttgtaacagtccacaatggagacacccatgcagtaggaaatgacacatccaatcatggagttacagccatgataactcccaggtcaccatcggtggaagtcaaattgccggactatggagaactaacactcgattgtgaacccaggtctggaattgactttaatgagatgattctgatgaaaatgaaaaagaaaacatggctcgtgcataagcaatggtttttggatctgcctcttccatggacagcaggagcagacacatcagaggttcactggaattacaaagagagaatggtgacatttaaggttcctcatgccaagagacaggatgtgacagtgctgggatctcaggaaggagccatgcattctgccctcgctggagccacagaagtggactccggtgatggaaatcacatgtttgcaggacatcttaagtgcaaagtccgtatggagaaattgagaatcaagggaatgtcatacacgatgtgttcaggaaagttttcaattgacaaagagatggcagaaacacagcatgggacaacagtggtgaaagtcaagtatgaaggtgctggagctccgtgtaaagtccccatagagataagagatgtaaacaaggaaaaagtggttgggcgtatcatctcatccacccctttggctgagaataccaacagtgtaaccaacatagaattagaacccccctttggggacagctacatagtgataggtgttggaaacagcgcattaacactccattggttcaggaaagggagttccattggcaagatgtttgagtccacatacagaggtgcaaaacgaatggccattctaggtgaaacagcttgggattttggttccgttggtggactgttcacatcattgggaaaggctgtgcaccaggtttttggaagtgtgtatacaaccatgtttggaggagtctcatggatgattagaatcctaattgggttcttagtgttgtggattggcacgaactcgaggaacacttcaatggctatgacgtgcatagctgttggaggaatcactctgtttctgggcttcacagttcaagcagacatgggttgtgtggcgtcatggagtgggaaagaattgaagtgtggaagcggaatttttgtggttgacaacgtgcacacttggacagaacagtacaaatttcaaccagagtccccagcgagactagcgtctgcaatattaaatgcccacaaagatggggtctgtggaattagatcaaccacgaggctggaaaatgtcatgtggaagcaaataaccaacgagctaaactatgttctctgggaaggaggacatgacctcactgtagtggctggggatgtgaagggggtgttgaccaaaggcaagagagcactcacacccccagtgagtgatctgaaatattcatggaagacatggggaaaagcaaaaatcttcaccccagaagcaagaaatagcacatttttaatagacggaccagacacctctgaatgccccaatgaacgaagagcatggaactctcttgaggtggaagactatggatttggcatgttcacgaccaacatatggatgaaattccgagaaggaagttcagaagtgtgtgaccacaggttaatgtcagctgcaattaaagatcagaaagctgtgcatgctgacatgggttattggatagagagctcaaaaaaccagacctggcagatagagaaagcatctcttattgaagtgaaaacatgtctgtggcccaagacccacacactgtggagcaatggagtgctggaaagccagatgctcattccaaaatcatatgcgggccctttttcacagcacaattaccgccagggctatgccacgcaaaccgtgggcccatggcacttaggcaaattagagatagactttggagaatgccccggaacaacagtcacaattcaggaggattgtgaccatagaggcccatctttgaggaccaccactgcatctggaaaactagtcacgcaatggtgctgccgctcctgcacgatgcctcccttaaggttcttgggagaagatgggtgctggtatgggatggagattaggcccttgagtgaaaaagaagagaacatggtcaaatcacaggtgacggccggacagggcacatcagaaactttttctatgggtctgttgtgcctgaccttgtttgtggaagaatgcttgaggagaagagtcactaggaaacacatgatattagttgtggtgatcactctttgtgctatcatcctgggaggcctcacatggatggacttactacgagccctcatcatgttgggggacactatgtctggtagaataggaggacagatccacctagccatcatggcagtgttcaagatgtcaccaggatacgtgctgggtgtgtttttaaggaaactcacttcaagagagacagcactaatggtaataggaatggccatgacaacggtgctttcaattccacatgaccttatggaactcattgatggaatatcactgggactaattttgctaaaaatagtaacacagtttgacaacacccaagtgggaaccttagctctttccttgactttcataagatcaacaatgccattggtcatggcttggaggaccattatggctgtgttgtttgtggtcacactcattcctttgtgcaggacaagctgtcttcaaaaacagtctcattgggtagaaataacagcactcatcctaggagcccaagctctgccagtgtacctaatgactcttatgaaaggagcctcaagaagatcttggcctcttaacgagggcataatggctgtgggtttggttagtctcttaggaagcgctcttttaaagaatgatgtccctttagctggcccaatggtggcaggaggcttacttctggcggcttacgtgatgagtggtagctcagcagatctgtcactagagaaggccgccaacgtgcagtgggatgaaatggcagacataacaggctcaagcccaatcgtagaagtgaagcaggatgaagatggctctttctccatacgggacgtcgaggaaaccaatatgataacccttttggtgaaactggcactgataacagtgtcaggtctctaccccttggcaattccagtcacaatgaccttatggtacatgtggcaagtgaaaacacaaagatcaggagccctgtgggacgtcccctcacccgctgccactaaaaaagccgcactgtctgaaggagtgtacaggatcatgcaaagagggttattcgggaaaactcaggttggagtagggatacacatggaaggtgtatttcacacaatgtggcatgtaacaagaggatcagtgatctgccacgagactgggagattggagccatcttgggctgacgtcaggaatgacatgatatcatacggtgggggatggaggcttggagacaaatgggacaaagaagaagacgttcaggtcctcgccatagaaccaggaaaaaatcctaaacatgtccaaacgaaacctggccttttcaagaccctaactggagaaattggagcagtaacattagatttcaaacccggaacgtctggttctcccatcatcaacaggaaaggaaaagtcatcggactctatggaaatggagtagttaccaaatcaggtgattacgtcagtgccataacgcaagccgaaagaattggagagccagattatgaagtggatgaggacatttttcgaaagaaaagattaactataatggacttacaccccggagctggaaagacaaaaagaattcttccatcaatagtgagagaagccttaaaaaggaggctacgaactttgattttagctcccacgagagtggtggcggccgagatggaagaggccctacgtggactgccaatccgttatcagaccccagctgtgaaatcagaacacacaggaagagagattgtagacctcatgtgtcatgcaaccttcacaacaagacttttgtcatcaaccagggttccaaattacaaccttatagtgatggatgaagcacatttcaccgatccttctagtgtcgcggctagaggatacatctcgaccagggtggaaatgggagaggcagcagccatcttcatgaccgcaacccctcccggagcgacagatccctttccccagagcaacagcccaatagaagacatcgagagggaaattccggaaaggtcatggaacacagggttcgactggataacagactaccaagggaaaactgtgtggtttgttcccagcataaaagctggaaatgacattgcaaattgtttgagaaagtcgggaaagaaagttatccagttgagtaggaaaacctttgatacagagtatccaaaaacgaaactcacggactgggactttgtggtcactacagacatatctgaaatgggggccaattttagagccgggagagtgatagaccctagaagatgcctcaagccagttatcctaccagatgggccagagagagtcattttagcaggtcctattccagtgactccagcaagcgctgctcagagaagagggcgaataggaaggaacccagcacaagaagacgaccaatacgttttctccggagacccactaaaaaatgatgaagatcatgcccactggacagaagcaaagatgctgcttgacaatatctacaccccagaagggatcattccaacattgtttggtccggaaagggaaaaaacccaagccattgatggagagtttcgcctcagaggggaacaaaggaagacttttgtggaattaatgaggagaggagaccttccggtgtggctgagctataaggtagcttctgctggcatttcttacgaagatcgggaatggtgcttcacaggggaaagaaataaccaaattttagaagaaaacatggaggttgaaatttggactagagagggagaaaagaaaaagctaaggccaagatggttagatgcacgtgtatacgctgaccccatggctttgaaggatttcaaggagtttgccagtggaaggaagagtataactctcgacatcctaacagagattgccagtttgccaacttacctttcctctagggccaagctcgcccttgataacatagtcatgctccacacaacagaaagaggagggagggcctatcaacacgccctgaacgaacttccggagtcactggaaacactcatgcttgtagctttactaggtgctatgacagcaggcatcttcctgtttttcatgcaagggaaaggaatagggaaattgtcaatgggtttgataaccattgcggtggctagtggcttgctctgggtagcagaaattcaaccccagtggatagcggcctcaatcatactagagttttttctcatggtactgttgataccggaaccagaaaaacaaaggaccccacaagacaatcaattgatctacgtcatattgaccattctcaccatcattggtctaatagcagccaacgagatggggctgattgaaaaaacaaaaacggattttgggttttaccaggtaaaaacagaaaccaccatcctcgatgtggacttgagaccagcttcagcatggacgctctatgcagtagccaccacaattctgactcccatgctgagacacaccatagaaaacacgtcggccaacctatctctagcagccattgccaaccaggcagccgtcctaatggggcttggaaaaggatggccgctccacagaatggacctcggtgtgccgctgttagcaatgggatgctattctcaagtgaacccaacaaccttgacagcatccttagtcatgcttttagtccattatgcaataataggcccaggattgcaggcaaaagccacaagagaggcccagaaaaggacagctgctgggatcatgaaaaatcccacagtggacgggataacagtaatagatctagaaccaatatcctatgacccaaaatttgaaaagcaattagggcaggtcatgctactagtcttgtgtgctggacaactactcttgatgagaacaacatgggctttctgtgaagtcttgactttggccacaggaccaatcttgaccttgtgggagggcaacccgggaaggttttggaacacgaccatagccgtatccaccgccaacattttcaggggaagttacttggcgggagctggactggctttttcactcataaagaatgcacaaacccctaggaggggaactgggaccacaggagagacactgggagagaagtggaagagacagctaaactcattagacagaaaagagtttgaagagtataaaagaagtggaatactagaagtggacaggactgaagccaagtctgccctgaaagatgggtctaaaatcaagcatgcagtatcaagagggtccagtaagatcagatggattgttgagagagggatggtaaagccaaaagggaaagttgtagatcttggctgtgggagaggaggatggtcttattacatggcgacactcaagaacgtgactgaagtgaaagggtatacaaaaggaggtccaggacatgaagaaccgattcccatggctacttatggttggaatttggtcaaactccattcaggggttgacgtgttctacaaacccacagagcaagtggacaccctgctctgtgatattggggagtcatcttctaatccaacaatagaggaaggaagaacattaagagttttgaagatggtggagccatggctctcttcaaaacctgaattctgcatcaaagtccttaacccctacatgccaacagtcatagaagagctggagaaactgcagagaaaacatggtgggaaccttgtcagatgcccgctgtccaggaactccacccatgagatgtattgggtgtcaggagcgtcgggaaacattgtgagctctgtgaacacaacatcaaagatgttgttgaacaggttcacaacaaggcataggaaacccacttatgagaaggacgtagatcttggggcaggaacgagaagtgtctccactgaaacagaaaaaccagacatgacaatcattgggagaaggcttcagcgattgcaagaagagcacaaagaaacctggcattatgatcaggaaaacccatacagaacctgggcgtatcatggaagctatgaagctccttcgacaggctctgcatcctccatggtgaacggggtggtaaaactgctaacaaaaccctgggatgtgattccaatggtgactcagttagccatgacagatacaaccccttttgggcaacaaagagtgttcaaagagaaggtggataccagaacaccacaaccaaaacccggtacacgaatggttatgaccacgacagccaattggctgtgggccctccttggaaagaagaaaaatcccagactgtgcacaagggaagagttcatctcaaaagttagatcaaacgcagccataggcgcagtctttcaggaagaacagggatggacatcagccagtgaagctgtgaatgacagccggttttgggaactggttgacaaagaaagggccctacaccaggaagggaaatgtgaatcgtgtgtctataacatgatgggaaaacgtgagaaaaagttaggagagtttggcagagccaagggaagccgagcaatctggtacatgtggctgggagcgcggtttctggaatttgaagccctgggttttttgaatgaagatcactggtttggcagagaaaattcatggagtggagtggaaggggaaggtctgcacagattgggatatatcctggaggagatagacaagaaggatggagacctaatgtatgctgatgacacagcaggctgggacacaagaatcactgaggatgaccttcaaaatgaggaactgatcacggaacagatggctccccaccacaagatcctagccaaagccattttcaaactaacctatcaaaacaaagtggtgaaagtcctcagacccacaccgcggggagcggtgatggatatcatatccaggaaagaccaaagaggtagtggacaagttggaacatatggtttgaacacattcaccaacatggaagttcaactcatccgccaaatggaagctgaaggagtcatcacacaagatgacatgcagaacccaaaagggttgaaagaaagagttgagaaatggctgaaagagtgtggtgtcgacaggttaaagaggatggcaatcagtggagacgattgcgtggtgaagcccctagatgagaggtttggcacttccctcctcttcttgaacgacatgggaaaggtgaggaaagacattccgcagtgggaaccatctaagggatggaaaaactggcaagaggttcctttttgctcccaccactttcacaagatctttatgaaggatggccgctcactagttgttccatgtagaaaccaggatgaactgatagggagagccagaatctcgcagggagctggatggagcttaagagaaacagcctgcctgggcaaagcttacgcccagatgtggtcgcttatgtacttccacagaagggatctgcgtttagcctccatggccatatgctcagcagttccaacggaatggtttccaacaagcagaacaacatggtcaatccacgctcatcaccagtggatgaccactgaagatatgctcaaagtgtggaacagagtgtggatagaagacaaccctaatatgactgacaagactccagtccattcgtgggaagatataccttacctagggaaaagagaggatttgtggtgtggatccctgattggactttcttccagagccacctgggcgaagaacattcatacggccataacccaggtcaggaacctgatcggaaaagaggaatacgtggattacatgccagtaatgaaaagatacagtgctccttcagagagtgaaggagttctgtaattaccaacaacaaacaccaaaggctattgaagtcaggccacttgtgccacggtttgagcaaaccgtgctgcctgtagctccgccaataatgggaggcgtaataatccccagggaggccatgcgccacggaagctgtacgcgtggcatattggactagcggttagaggagacccctcccatcactgataaaacgcagcaaaagggggcccgaagccaggaggaagctgtactcctggtggaaggactagaggttagaggagacccccccaacacaaaaacagcatattgacgctgggaaagaccagagatcctgctgtctctgcaacatcaatccaggcacagagcgccgcaagatggattggtgttgttgatccaacaggttct

SEQ ID NO: 186 - protein, 3387 aa, Dengue-4 virus
MNQRKKVVRPPFNMLKRERNRVSTPQGLVKRFSTGLFSGKGPLRMVLAFITFLRVLSIPPTAGILKRWGQLKKNKAIKILIGFRKEIGRMLNILNGRKRSTITLLCLIPTVMAFSLSTRDGEPLMIVAKHERGRPLLFKTTEGINKCTLIAMDLGEMCEDTVTYKCPLLVNTEPEDIDCWCNLTSTWVMYGTCTQSGERRREKRSVALTPHSGMGLETRAETWMSSEGAWKHAQRVESWILRNPGFALLAGFMAYMIGQTGIQRTVFFVLMMLVAPSYGMRCVGVGNRDFVEGVSGGAWVDLVLEHGGCVTTMAQGKPTLDFELTKTTAKEVALLRTYCIEASISNITTATRCPTQGEPYLKEEQDQQYICRRDVVDRGWGNGCGLFGKGGVVTCAKFSCSGKITGNLVQIENLEYTVVVTVHNGDTHAVGNDTSNHGVTAMITPRSPSVEVKLPDYGELTLDCEPRSGIDFNEMILMKMKKKTWLVHKQWFLDLPLPWTAGADTSEVHWNYKERMVTFKVPHAKRQDVTVLGSQEGAMHSALAGATEVDSGDGNHMFAGHLKCKVRMEKLRIKGMSYTMCSGKFSIDKEMAETQHGTTVVKVKYEGAGAPCKVPIEIRDVNKEKVVGRIISSTPLAENTNSVTNIELEPPFGDSYIVIGVGNSALTLHWFRKGSSIGKMFESTYRGAKRMAILGETAWDFGSVGGLFTSLGKAVHQVFGSVYTTMFGGVSWMIRILIGFLVLWIGTNSRNTSMAMTCIAVGGITLFLGFTVQADMGCVASWSGKELKCGSGIFVVDNVHTWTEQYKFQPESPARLASAILNAHKDGVCGIRSTTRLENVMWKQITNELNYVLWEGGHDLTVVAGDVKGVLTKGKRALTPPVSDLKYSWKTWGKAKIFTPEARNSTFLIDGPDTSECPNERRAWNSLEVEDYGFGMFTTNIWMKFREGSSEVCDHRLMSAAIKDQKAVHADMGYWIESSKNQTWQIEKASLIEVKTCLWPKTHTLWSNGVLESQMLIPKSYAGPFSQHNYRQGYATQTVGPWHLGKLEIDFGECPGTTVTIQEDCDHRGPSLRTTTASGKLVTQWCCRSCTMPPLRFLGEDGCWYGMEIRPLSEKEENMVKSQVTAGQGTSETFSMGLLCLTLFVEECLRRRVTRKHMILVVVITLCAIILGGLTWMDLLRALIMLGDTMSGRIGGQIHLAIMAVFKMSPGYVLGVFLRKLTSRETALMVIGMAMTTVLSIPHDLMELIDGISLGLILLKIVTQFDNTQVGTLALSLTFIRSTMPLVMAWRTIMAVLFVVTLIPLCRTSCLQKQSHWVEITALILGAQALPVYLMTLMKGASRRSWPLNEGIMAVGLVSLLGSALLKNDVPLAGPMVAGGLLLAAYVMSGSSADLSLEKAANVQWDEMADITGSSPIVEVKQDEDGSFSIRDVEETNMITLLVKLALITVSGLYPLAIPVTMTLWYMWQVKTQRSGALWDVPSPAATKKAALSEGVYRIMQRGLFGKTQVGVGIHMEGVFHTMWHVTRGSVICHETGRLEPSWADVRNDMISYGGGWRLGDKWDKEEDVQVLAIEPGKNPKHVQTKPGLFKTLTGEIGAVTLDFKPGTSGSPIINRKGKVIGLYGNGVVTKSGDYVSAITQAERIGEPDYEVDEDIFRKKRLTIMDLHPGAGKTKRILPSIVREALKRRLRTLILAPTRVVAAEMEEALRGLPIRYQTPAVKSEHTGREIVDLMCHATFTTRLLSSTRVPNYNLIVMDEAHFTDPSSVAARGYISTRVEMGEAAAIFMTATPPGATDPFPQSNSPIEDIEREIPERSWNTGFDWITDYQGKTVWFVPSIKAGNDIANCLRKSGKKVIQLSRKTFDTEYPKTKLTDWDFVVTTDISEMGANFRAGRVIDPRRCLKPVILPDGPERVILAGPIPVTPASAAQRRGRIGRNPAQEDDQYVFSGDPLKNDEDHAHWTEAKMLLDNIYTPEGIIPTLFGPEREKTQAIDGEFRLRGEQRKTFVELMRRGDLPVWLSYKVASAGISYEDREWCFTGERNNQILEENMEVEIWTREGEKKKLRPRWLDARVYADPMALKDFKEFASGRKSITLDILTEIASLPTYLSSRAKLALDNIVMLHTTERGGRAYQHALNELPESLETLMLVALLGAMTAGIFLFFMQGKGIGKLSMGLITIAVASGLLWVAEIQPQWIAASIILEFFLMVLLIPEPEKQRTPQDNQLIYVILTILTIIGLIAANEMGLIEKTKTDFGFYQVKTETTILDVDLRPASAWTLYAVATTILTPMLRHTIENTSANLSLAAIANQAAVLMGLGKGWPLHRMDLGVPLLAMGCYSQVNPTTLTASLVMLLVHYAIIGPGLQAKATREAQKRTAAGIMKNPTVDGITVIDLEPISYDPKFEKQLGQVMLLVLCAGQLLLMRTTWAFCEVLTLATGPILTLWEGNPGRFWNTTIAVSTANIFRGSYLAGAGLAFSLIKNAQTPRRGTGTTGETLGEKWKRQLNSLDRKEFEEYKRSGILEVDRTEAKSALKDGSKIKHAVSRGSSKIRWIVERGMVKPKGKVVDLGCGRGGWSYYMATLKNVTEVKGYTKGGPGHEEPIPMATYGWNLVKLHSGVDVFYKPTEQVDTLLCDIGESSSNPTIEEGRTLRVLKMVEPWLSSKPEFCIKVLNPYMPTVIEELEKLQRKHGGNLVRCPLSRNSTHEMYWVSGASGNIVSSVNTTSKMLLNRFTTRHRKPTYEKDVDLGAGTRSVSTETEKPDMTIIGRRLQRLQEEHKETWHYDQENPYRTWAYHGSYEAPSTGSASSMVNGVVKLLTKPWDVIPMVTQLAMTDTTPFGQQRVFKEKVDTRTPQPKPGTRMVMTTTANWLWALLGKKKNPRLCTREEFISKVRSNAAIGAVFQEEQGWTSASEAVNDSRFWELVDKERALHQEGKCESCVYNMMGKREKKLGEFGRAKGSRAIWYMWLGARFLEFEALGFLNEDHWFGRENSWSGVEGEGLHRLGYILEEIDKKDGDLMYADDTAGWDTRITEDDLQNEELITEQMAPHHKILAKAIFKLTYQNKVVKVLRPTPRGAVMDIISRKDQRGSGQVGTYGLNTFTNMEVQLIRQMEAEGVITQDDMQNPKGLKERVEKWLKECGVDRLKRMAISGDDCVVKPLDERFGTSLLFLNDMGKVRKDIPQWEPSKGWKNWQEVPFCSHHFHKIFMKDGRSLVVPCRNQDELIGRARISQGAGWSLRETACLGKAYAQMWSLMYFHRRDLRLASMAICSAVPTEWFPTSRTTWSIHAHHQWMTTEDMLKVWNRVWIEDNPNMTDKTPVHSWEDIPYLGKREDLWCGSLIGLSSRATWAKNIHTAITQVRNLIGKEEYVDYMPVMKRYSAPSESEGVL